SEQUENCE LISTING (428 sequences)

SEQ ID NO: 1 - DNA, 3510 bp, Acinetobacter baumannii
  rRNA (1)..(3510): 23S rRNA and Internal Transcribed Spacer Region
ccatgggagtttgttgcaccagaagtagctgcctaactgcaaagagggcggttaccacggtgtggccgatgactggggtgaagtcgtaacaaggtagccgtaggggaacctgcggctggatcacctccttaacgaaagattgacgattggtaagaatccacaacaagttgttcttcatagatgtatctgagggtctgtagctcagttggttagagcacacgcttgataagcgtggggtcacaagttcaagtcttgtcagacccaccatgactttgactggttgaagttatagataaaagatacatgactgatgatgtaagctggggacttagcttagttggtagagcgcctgctttgcacgcaggaggtcaggagttcgactctcctagtctccaccagaacttaagagaagttcggattacagaaattagtaaatagagatttagatcttggtttattaacttctgtgatttaagtatcacggtattaagcatgacctgacgaaggcatgtttattcattaacagattggcaaaattgagtctgaaataaattgttcactcaagagtttagattaagcaattaatctagatgaattgagaactagcaaattaactgaatcaagcgttttggtatatgaatttagattgaagctgtacagtgtttaaatacacagtacttcgaactgtatggatgaatgagagatcatttgttctgttgctacaccaacttgtaggtgtaacgactgtttggggttgtatagtcaagtaattaagtgcatgtggtggatgccttggcagtcagaggcgatgaaagacgtgatagcctgcgaaaagctccggggaggcggcaaatatcctttgatccggagatttctgaatgggggaacccacctactttaaggtaggtattgcaacatgaatacatagtgttgcaaggcgaacgaggggaagtgaaacatctcagtacccttaggaaaagaaatcaattgagattccctcagtagcggcgagcgaacggggatcagcccattaagttatgtgtgttttagtggaacgctctgggaagtgcgaacgtagagggtgatattcccgtacacgaaagggcacacataatgatgacgagtagggcgaggcacgtgaaaccttgtctgaatatggggggaccatcctccaaggctaaatactcctgactgaccgatagtgaaccagtaccgtgagggaaaggcgaaaagaacccctgtgaggggagtgaaatagatcctgaaaccgcatgcatacaagcagtgggagcaccttcgtggtgtgactgcgtaccttttgtataatgggtcagcgacttatattcagtagcaaggttaaccgtataggggagccgtagggaaaccgagtcttaatagggcgtttagttgctgggtatagacccgaaaccaggcgatctatccatgagcaggttgaaggttgggtaacactaactggaggaccgaacccactgtcgttgaaaagccaggggatgacttgtggatagggggtgaaaggctaatcaagcctggtgatagctggttctccccgaaagctatttaggtagcgcctcggacgaataccatagggggtagagcactgtttcggctagggggtcatcccgacttaccaaaccgatgcaaactccgaatacctatgagtactatccgggagacagactgcgggtgctaacgtccgtagtcaagaggaaaacaatccagaccgccagctaaggccccaaaatcatagttaagtgggaaacgatgtgggaaggcatagacagctaggaggttggcttagaagcagccaccctttaaagaaagcgtaatagctcactagtcgagtcggcctgcgcggaagatgtaacggggctaaaactatgtgccgaagctgcggatttgacattagtcaagtggtaggggagcgttctgtaagccgatgaaggtgtattgagaagtatgctggaggtatcagaagtgcgaatgctgacgtgagtaacgacaaaacgggtgaaaaacccgttcgccgaaagaccaagggttccagtccaacgttaatcggggctgggtgagtcgacccctaaggcgaggccgaaaggcgtagtcgatgggaaattggttaatattccaatacttctgtgtaatgcgatgagaggacggagaaggttaagtcagcctggcgttggttgtccaggtggaaggatgtaggtatgtatcttaggcaaatccggggtactctatactgagatccgatagcaagctgtacttgtacagtgaagtggctgataccatgcttccaggaaaagtctctaagcttcagttacacaggaatcgtacccgaaaccgacacaggtggtcaggtcgagtagaccaaggcgcttgagagaactctgctgaaggaactaggcaaaatggtaccgtaacttcgggagaaggtacgctgttgttggtgatggaacttgcttcctgagctgacgacagccgcagaaaccaggccgctgcaactgtttattaaaaacatagcactctgcaaacacgaaagtggacgtatagggtgtgatgcctgcccggtgctggaaggttaattgatggggttagcgtaagcgaagctcttgatcgaagccccagtaaacggcggccgtaactataacggtcctaaggtagcgaaattccttgtcgggtaagttccgacctgcacgaatggcataatgatggcggcgctgtctccagcagaggctcagtgaaatcgaaatcgctgtgaagatgcagtgtacccgcggctagacggaaagaccccgtgaacctttactgcagcttgacactgaactttgaccttacttgtgtaggataggtgggaggctttgaagttggaacgctagttccaatggagccgtccttgaaataccaccctggtaatgttgaggttctaactctgtcccgttatccgggacgaggaccgtgtctggtgggtagtttgactggggcggtctcctcctaaagagtaacggaggagtacgaaggtgcgctcagcgtggtcggaaatcacgcgtagagtataaaggcaaaagcgcgcttaactgcgagacccacaagtcgagcaggtacgaaagtaggtcttagtgatccggtggttctgtatggaagggccatcgctcaacggataaaaggtactctggggataacaggctgataccgcccaagagttcatatcgacggcggtgtttggcacctcgatgtcggctcatctcatcctggggctgaagcaggtcccaagggtatggctgttcgccatttaaagaggtacgcgagctgggtttagaacgtcgtgagacagttcggtccctatctaccgtgggcgctggaaatttgagaggatctgctcctagtacgagaggaccagagtggacgaacctctggtgtaccggttgtgacgccagtcgcatcgccgggtagctatgttcggaagggataaccgctgaaagtttttaagca

SEQ ID NO: 2 - DNA, 2940 bp, Anaerobiospirillum succiniciproducens
  rRNA (1)..(2940): 23S rRNA and Internal Transcribed Spacer Region
cttccctcagtgattcaagacagcgcagcatgtgttttgttcttgattcattgcagttaaaaagttctttaaaaatttatcagacaagcttatttaaagtaaaaactcaattagcaactatttaatgggtaaaaagaccaaagtcttgtaaacctttcggcagttgttaaaagttacttgatccaatcatgatcaaggccttacgacctttcggggttgtatggtcaagcatgaaagcgcacatggtggatgccatggcgtcaggaggcgatgaaggacgtgccaatctgcgataagcctaggtaagccgataaggggcgcttgaacctaggatttccgaatggggaaacccgacacttagtgtcatctatgctgaagttatagaagcgaaccaggggaagtgaaacatctcagtaccctgaggaaaagaaatcaaatcagagattccctcagtagcggcgagcgaacggggaacagcccaaaagttgatatgttctagtagaagcagttgggaaactgcagggcacagggtgatactcccgtatacgaaggagcatatttgatgagcgtgagtagggcgggacacgtgatatcctgtctgaatatggggggaccatcctccaaggctaaatactacctgacgaccgatagtgaaccagtaccgtgagggaaaggcgaaaagaaccccgggaggggagtgaaaaagaacctgaaaccgtgtgcgtacaagcagtgggagcctacttgttaggtgactgcgtaccttttgtataatgggtcagcgagttatctgtattagcaggttaacctatatgggggagccgtagagaaatcgagtcctaaccggcgaatagttgatacaggtagacccgaaactgggtgagctagtcctggccaggttgaaagcagggtaacacctgccggaggaccgaacccactaacgttgcaaagttaggggatgagctgggactaggggtgaaaggccaatcaaactcagtgatagcttggttctcctcgaaagctatttaggtagcgttctgcgaggacgtatgggggtagagcactgttatggggaaggggccccatccggggttgccaatccattgcaaactttcgaataccatacagttggaagcagaggacagacggtgggtgctaacgttcatcgtcaagagggaaacaacccggaccgccagctaaggtccccaagttctagttaagtgggaaacgatgtgggaaggcatagacagccaggatgttggcttagaagcagccatcatttaaagaaagcgtaatagctcactggtcgagtcggcctgcgcggaagatgtaacggggctaaactagacaccgaagctgcggattcacacgcaagtgtgagtggtagaggagcgttctgtaaactgatgaaggtgaggcgggagccgagctggaggtaacagaagtgcgaatgctgacgtaagtaacgataaaacacgtgaaaagcgtgttcgccgaaagaccaagggttcctgtccaacgttaatcggggcagggtgagtcggtgcctaaggcgaggctgaaaagcgtagtcgaagggaaacaggtcaatattcctgtactttacttaagtgcgatgagatgacggagaagggaaagtcagccacttattggattgtggtgaaaggctgtaggtagataccggggttaaaagactggtatcagtgatctgagagctgagacgaagcaccattgtgtgaagtgactcgtgcccatgcttcctggaaaagtctctaagcttcagcttaagtagagccgtaccccaaaccgacacaggtggtcgggtagaggataccaaggcgcttgggagaactcaggtgaaggaactaggcaaaattgtaccgtaacttcgggataaggtacgctgttaccggtgagaggacttgctccttaagctgggaacagccgcagtgaaatggtggctgggactgtttaacaaaaacacagcactctgcaaacctgaaaggggaagtatagggtgtgacacctgcccggtgctggaagattaattgatggggtgcaagctcctgatcgaagtcccagtaaacggcggccgtaactataacggtcctaaggtagcgaaattccttgtcgggcaagttccgacctgcacgaatggcataatgatggccacactgtctcctcctgagactcagtgaagttgaaatgtttgtgatgatgcaatctccccgcggctagacggaaagaccccatgaacctttactgcagctttgcattggactttgaaccgatctgtgtaggataggtgggaggcagtgaagctaggacgccagtcttagcggagccaaccttgaaataccaccctgatttgtttgaggttctaacctaggagcgtaatccgcttcggggaccgtgcatggtaggcagtttgactggggcggtctcctcctaaagggtaacggaggagtgcgaaggtacgctaggtacggtcggaaatcgtgctgatagtgcaatggcaaaagcgtgcttgactgcgagaccgacaagtcgagcagatacgaaagtaggtcatagtgatccggtggctctgcatggaagggccatcgctcaacggataaaaggtactctggggataacaggctgataccgcccaagagttcatatcgacggcggtgtttggcacctcgatgtcggctcatctcatcctggggctgtagccggtcccaagggtatggctgttcgccatttaaagaggtacgtgagctgggtttaaaacgtcgtgagacagttttgtccctatctgccgtgggcgttggaagattgacgggggctgctcctagtacgagaggaccggagtggactcacctctggtgtaccggttgtcacgccagtggcatcgccgggtagctatgtggggaaaggataaccgctgaaattttt

SEQ ID NO: 3 - DNA, 3379 bp, Bacteroides fragilis
  rRNA (1)..(3379): 23S rRNA and Internal Transcribed Spacer Region
  MISC_FEATURE (1)..(3379): n can be a, t, c, or g.
gctctgggagccgggggtacctgaagtacgtaaccgcaaggatcgtcctagggtaaaactggtgactggggctaagtcgtaacaaggtagccgtaccggaaggtgcggctggaacacctcctttctggagcgatgttgaaaacgacgttcattggttctaaattgtactactggtacttgtttatttatagaatatagatcaaccatctatagcaatatagatagagataaacaagagaaaaacagaagccgagtctaaaacaggacacggttgaactagtcctatagctcagttggttagagcgctacactgataatgtagaggtcggcagttcaactctgcctgggactaccaacagatagatattttatcttgtatgattgggggattagctcagctggctagagcatctgccttgcacgcagagggtcaacggttcgaaatccgttattctccactccgataccgcgacctaacaggcttgcgtgtaatcaaacgatctttgacatgatgtacagaaaaaagtaaatttagtaagagctaaaagtatatatcgaaccatttggttcagatcctttttaagaaaaggagatgaatctggcgataaaagaaagtaaataagggcgcatggcggatgccttggctctcggaggcgatgaangacgtgataagctgcgatnagcttcgggtaggtgcaaatgacctttgattccgaagatttccgaatgggacaaccccggcattctgaaggaatgtcatccaacttggttggaggctaacgcagggaactgaaacatcttagtacctgcaggaaaagaaaataaataatgattcccctagtagtggcgagcgaacggggattagcccaaaccacccatgttacggcatgtgtggggttgtaggaccacgctctcgcaagacagttattgagaagaaacgactggaaagtcgtgccatagacggtgatagcccggtattctaaggtaaccgaagcgtagtggtatcctgagtagcgcggagcacgaggaattctgcgtgaaccagccgggaccatccggtaaggctaaatactcccgagagaccgatagcgaaccagtactgtgaaggaaaggtgaaaagcacttcgaatagaagagtgaaatagtccctgaaaccatgcgcctacaagcggtcggagcagcttaagctgtgacggcgtgccttttgcataatgaacctacgagttactttttccggcaaggttaagcatcttgagatgtggatccgaagcgaaagcgagtctgaacagggcggatagtcggaaggagtagacgcgaaaccaagtgatctacccttgggcaggttgaaggttaggtaacactaactggaggaccgaaccgataagcgttgaaaagcttccggatgacctgagggtggggggtgaaaggctaatcaaacttggagatagctcgtactccccgaaatgcatttaggtgcagccttgagagttactaatgtgaggtagagcgactgataagatgcgagggcttcaccgcctatcaagtcttgataaactccgaatgcgcattagttctatctcaggagtgagggcatgggtgtaaggtccatgtctaaagagaagaatccagaccatcagtaaggtccccaaataaacattaagttgacctaacgaagtcagattgctaagacagctaggatgttggcttggaagcagccattcatttaaagagtgcgtaacagctcactagtcgaggagtttggcgtggataataatcgggcataagtgttttaccgaagctatgggatccgtatggatcggtaggggagcattccactcagcgttgaaggcgaagcgtgagctttactggagcgtgtggaaaagcaaatgtaggtataagtaacgataaagggggtgagaaaccccctcgccgcaagactaaggtttcctgatcaacgctaatcggatcagggttagtcgggtcctaaggctcagccgaacggtgaggccgatggcagaacaggttaatattcctgtactacctataagagtgatgtggagacggaggagtgacaacgccgcggactgacggaatagtccgttaaagggtgtagatgttgattatcccaggcaaatccgggataagagtcgaacctgacagtataccaatttcctcggaaaacggtaatagtgcgtgtaaacatactcccaagaaaatccgctaaacttaatcttataggtacccgtaccgtaaacggacacacgtagtcgggttgaatatactcaggcgcttgagtgaatcacggttaaggaactaggcaaattgaccctgtaacttcgggataaagggtccctacccggtgacggggagggcgcagaaaaataggtccaggccaacctgtttaacaaaaacacagggctgtgcaaaattgaaagatcacgtttacagccctgacacctgcccggtgctggaaggttaagaggagatgtcatcgcaagagaagcattgaattgaagccccagtaaacggcggccgtaactataacggtcctaaggtagcgaaattccttgtcgggtaagttccgacctgcacgaatggcgtaacgatggccacactgtctccaccagagactcagcgaagttgaaatgtttgtaatgatgcatctccccgcggaaagacggaaagaccccatgaacctttactgtagctttgtattggattttgaacggatctgtgtaggataggtgggaggctttgaagtgaggacgctagttctcatggagccgacgttgaaataccaccctggtgcgttgaggttctaacccaggtcccttatcgggatcggggacagtgcatggtaggcagtttgactggggcggtctcctcccaaagcgtaacggaggagttcgaaggtacgctagttacggtgcggacatcgtgacgatagtgcaatggcataagcgtgcttaactgcgagactgacaagtcgagcagatgcgaaagcaggacatagtgatccggtggttctgtatggaagggccatcgctcaacggataaaaggtactctggggataacaggctgataccgcccaagagttcatatcgacggcggtgtttggcacctcgatgtcggctcatctcatcctggggctgtagtcggtcccaagggtatggctgttcgccatttaaagaggtacgtgagctgggtttaaaacgtcgtgagacagtttggtccctatcttccgtgggcgctgcagatttgaggaagcctgctcctagtacgagaggaccggagtggacacacctctggtgtatcggttgtcacgccagtggcattgccgagtagctaagtgtggaagagataaccgctgaacttttt

SEQ ID NO: 4 - DNA, 3138 bp, Cardiobacterium hominis
  rRNA (1)..(3138): 23S rRNA and Internal Transcribed Spacer Region
cccctgataagggtgaggtcaaagttcaatttttcagaccccccaaaaagggccatagctcagctggagagcgcctgcttttgcacgcagagtcagagttcgatcctcctgggctccccatttgaaacgtagtttgctaaagagagcagaaaattgaacatgaagctaaagctgatgtaagaacacagatggagcacatggtgttcaattttttgttgtcttaggacaatgatctataacaaccggaaacattcaatttaaacaactaaattgagtagctgagaaaaacaagacgttgttacttatgttttcctctgcgtagccaacagatgatgagagagcaaggcgaaagcgaagcatacttcagaatctgaaaacgtagagtagtaaagagagaacagcacaaccgaaagataacttgaggttgtatgatcaagtgaataagtgcacacggtggatgccttggcgacaacaggcgaagaaggacgtggaaatctgcgaaaagccccgggaagctgataacaagcaatgatccggggatctccgaatggggcaacccgaccttagggtcatccgtagttgaatacatagactgcggaagcgaaccgggagaactgaaacatctaagtaccccgaggaaaagaaatcaaccgagattccgcaagtagcggcgagcgaacgcggagaagcccaattaagttgcgtatgagctagtagaacatcctgggaagggtggccatagagggtgatagccccgtatacgaaagccatacacaatgaatcgagtagggcgggacacgtgaaatcctgtctgaagatggggggaccatcctccaaggctaaatactcgttgtcgaccgatagtgaaccagtaccgtgagggaaaggcgaaaagaaccctggtgaagggagtgaaatagaacctgaaaccgtgtgcatacaagcagtcagagcatatgaagatatgtgatggcgtaccttttgtataatgggtcagcgagttacattcagtggcgaggttaaccgaataggggagccgtagcgaaagcgagtcttaatagggcgataagtctctgggtgtagacccgaaaccgggcgatctatccatgggcaggttgaaggttgagtaacatcaactggaggaccgaacccacgcatgttgaaaaatgcggggatgacctgtggataggagtgaaaggctaaacaagctcggagatagctggttctccccgaaaactattgaggtagtgccttgtagattgacttacgggggtagagcactgtttcggctagggggtcataccgacttaccaacccgatgcaaactcgaataccgtaaagttttactacaggagacacacggcgggtgctaacgtccgtcgtgaagagggaaacaacccagaccgccagctaaggtcccgaaatgacagttcagtgggaaacgaagtgggaaggcttagacagctaggaggttggcttagaagcagccatcctttaaagaaagcgtaatagctcactagtcgagtcgtcctgcgcggaagatttaccggggctaaactgtctaccgaagctgcggatttgcatgaagatgcaagtggtaggggagcgttgtgtaagccgaagaaggtgtgttgagaagcatgctggaggtatcacaagtgcgaatgctgacatgagtaacgataatgcgggtgaaaaacccgcacaccgaaaacccaaggtttcctgcgcaacgctaatcggcgcagggtgagtcggcccctaaggcgaggcggaaacgcgtagtcgatgggaaacaggttaatattcctgtactgatcataattgcgatggggggacggagaaagctaggccagcacactgttggatgtgtgtttaagcgcgtaggttggggacttaggcaaatccgggttcctaagactgaaacgtgatgacgagtttctacggaaacgaagtggttgatgctctgcttccaggaaaagcctctaagcttcagattatgacaaccgtaccccaaaccgacacaggtgggtaggaagagaattccaaggtgcttgagagaactcgggtaaaggaactcggcaaaatgataccgtaacttcgggataaggtatgcctctgatggtgaatagctgttggaggccgcagagaataggccgctgcgactgtttatcaaaaacacagcactctgcaaacacgcaagtggacgtatagggtgtgacgcctgcccggtgccggaaggttaattgatggggtgcaagctcttgatcgaagccccggtaaacggcggccgtaactataacggtcctaaggtagcgaaattccttgtcgggtaagttccgacctgcacgaatggcgtaacgatggcggcgctgtctctacccgagactcagtgaagttgaaatcgctgtgaagatgcagtgtacccgcggcaagacggaaagaccccgtgaaccttcactacagcttgacactgaacattgaaataagttgcataggataggtgggaggctttgaagcagagactctggtttctgtggagccgaccttgaaataccaccctgcttattttgatgttctaacgtagacccgtaatccgggttgcagacagtgtctggtgggtagtttgactggggcggtctcctcccaaagagtaacggaggagtgcgaaggtgggctcggtgcggtcggaaatcgcaccaagagtgcaaaggcagaagcccgcttaactgcgagacagacaagtcgagcagatacgaaagtaggtcttagtgatccggtggttctgaatggaagggccatcgctcaacggataaaaggtactccggggataacaggctgattcctcccaagagtccatatcgacggaggagtttggcacctcgatgtcggctcatcacatcctggggctgtagcaggtcccaagggtatggctgttcgccatttaaagtggtacgcgagctgggttcagaacgtcgtgagacagttcggtccctatctgccgtgggcgttggagatttgagggaagctgttcttagtacgagaggaccggaatggacgaacctctggtgtaccggttgtcacgccagtggcacagccgggtagctatgttcggaagggataaccgctgaacttttt

SEQ ID NO: 5 - DNA, 3332 bp, Chryseobacterium meningosepticum
  rRNA (1)..(3332): 23S rRNA and Internal Transcribed Spacer Region
ctctggagctgggggtacctgagtcggtgaccgtaaaggagctgcctagggtaaaactagtaactagggctaagtcgtaacaaggtagccgtaccggaaggtgcggctggaacatctcattttagagcgtcctttggacgataaacaaaacaaagatcacttaggtgatcacttacttaaacaaagcacagctttggttttactttggttgctattacaaaagataaccccttagattagtaacagggaaagagatagtttataattatgaattttaatgaataattataaattaatgaagtctcgtagctcagctggttagagcgctacactgataatgtagaggtcggcagttcgagcctgcccgagactactaatttaaaagatttgaagttagagattggaagtttgaagagataatttcaaacctcaaactaagaatttcaaatttaagagggggaattagctcagctggctagagcgcctgccttgcacgcaggaggtcaagggttcgactcccttattctccacagttttagaagagagacacaagtatacgaatagagccaataacaatattcgtttgttgatcagaagaaaagaacaaaagatcattgacattaacggtaaagacatcacaaagagataaccgagcactccttgagtgcagagtaaaaaataaattaattattaggaaagaaatcgttaagggcgtatggcggatgcctaggctttcagaggcgacgaaggacgtggtaagctgcgaaaagctacggggattggcacacacgaattgatccgtagatatccgaatggggcaacccggcatattgaagatatgtcacccagcaatgggagcaaacccggagaactgaaacatctaagtacccggaggaaaagaaatcgaagagattccgtaagtagtggcgagcgaaagcggattagcccaaaagtctttatatgtttagaagaacacactggaaagcgtggccatagagggtgatagccccgtatttgaaaggcatatttagatgataaatgagtagggcgggacacgtgaaatcctgtctgaatatggggggaccatcctccaaggctaaatactcctgaaagaccgatagtgaacaagtactgtgaaggaaaggtgaaaagcacttcgaatagaagggtgaaatagaacctgaaaccgtacgcctacaagcggtcggagcagctataagctgtgacggcgtgccttttgcataatgagcctacgagttaatcttactagcgaggttaaggacttaaggtccggagccgtagcgaaagcgagtctgaatagggcggatagttagtgggattagacgcgaaaccttgtgatctacccatgggcaggttgaagctttggtaacacaaagtggaggaccgaaccggttgacgttgaaaagtcttcggatgacctgtgggtaggggtgaaaggccaatcaaactgggagatagctcgtactccccgaaatgcatttaggtgcagcgtcgatttagtttattagaggtagagctactgattggatgcgggggtttcatcgcctaccaattcctgacaaactccgaatgctaataaatgtaacttgggagtcagaacatgggtgataaggtccgtgttcgaaagggaaacagcccagaccaccagctaaggtcccaaaatatatgttaagtggaaaaggatgtggcgttgcccagacaactaggatgttggcttagaagcagccatcatttaaagagtgcgtaatagctcactagtcgagtgatccggcatggataataatcgggcataagcatattaccgaagctatggcagtatttatactgggtaggggagcattctatttgcgccgaagcagtactgtgaggtattgtggagcggatagaaaagaaaatgtaggcataagtaacgataaagggggcgagaaaccccctcaccgaaagactaaggtttcctcagccatgctaatcagctgagggttagtcggggcctaacgcgaagccgaatggcgtagtggatggacaacgggttaatattcccgtaccagtatattaataaaagtgacggagttggaaactaggtgcgtactgacggaatagtacgttgaagtaatcgtgaggttacgatagtacagcaaatcttcggatgcgctgataatcctggggacccgcttccaagaaaagcgaaatatactgcccgtaccaaaaccgacacaggtagtcgaggagagaatcctaaggtgctcgagtgagtcgtggctaaggaactaggcaaaatagtctcgtaacttcggaagaagagacgcctccctccggggaggccgcagtgaagaggcccaggcgactgtttatcaaaaacacaggactctgctaaatcgaaagatgctgtatagggtctgacacctgcccggtgctggaaggttaaggaaggtgcttagagttaaatcgaaggcattaactgaagccccagtaaacggcggccgtaactataacggtcctaaggtagcgaaattccttgtcgggtaagttccgacctgcacgaatggtgtaacgatgctgggcactgtctcagccacgagctcggtgaaattgtagtatcggtaaagatgccgattacccgcaatgggacgaaaagaccctgtgaccctttactataacttcgtattgacttcgagtaaacaatgtgtaggataggtggaggctatgaagcaggcacgctagtgtttgtgagccaacgttgaaataccaccctttgtttacttggagcctaacttctacggaaggacattgcgtggtgggtagtttgactggggtggtcgcctccaaaagagtaacggaggctttcaaaggtaccctcagcacgcttggtaaccgtgcgtagagtgtaatggcataagggtgcttgactgtgagacctacaagtcgatcaggtgcgaaagcaggacatagtgatccggtggttccgtatggaagggccatcgctcataggataaaaggtactccggggataacaggctagtctcccccaagagctcacatcgacggggaggttcggcacctcgatgtcggctcgtcacatcctggggctggagaaggtcccaagggttgggctgttcgcccattaaagtggcacgcgagctgggttcagaacgtcgtgagacagttcggtctctatctattgcgggcgttagatgtttgagagggcttgattctagtacgagaggaccgaattgaacaaacctctggtgtatcagttgtaccgccaggtgcaccgctaaatagctatgtttggaagagataagcgctgaaag

SEQ ID NO: 6 - DNA, 3252 bp, Clostridium ramosum
  rRNA (1)..(3252): 23S rRNA and Internal Transcribed Spacer Region
  MISC_FEATURE (1)..(3252): n can be a, t, c, or g.
gcaccgctatagtttgtaacacccgaagtcggtgaggtaaccttttggagccagccgccgaaggtgggaaagaactgattggggatgatattgtctttgtaacaagggtagccgtatccggaaggtgcggctggatcacctcctttctaaggaaattatacggaatgatcgaagacctgatgcggtcttatagacaggtgcgtttngtagatacttgtttagttttgaagtgatcattccttcaaagcgtatgactcagagatcattgattactggataagtagacaatcacattgcaatagaaatagcaaaatnagatcgatcatagtaattgtctatattgacgggttacgttactagaaaacgcactgaaactaagtattttctcctaaaacataaatcgaacggatggatggattttagtaaatagttgatgatagtaatcaagcaacaccatataggtaagtaagaaaaggtatggcggatgcctggcacagagagcgaagaaggacgcagcaacagcaaatgtgacggttagcagtgagcatggcttatctgtggatgtctgaataggggaacccccctggagtagtagtccagcgtatcctcaagtgaatatatagcttcagagaggcgagactcaggggaacctgaaacaatctaaggtcctcaggaaggagaggagaaaataacacgaatgattccctaagtatcggcgagcgaaaggggaagagcccaaaccgatcttaggatggggttgtaggactgtcggcaaagagcaagaaatcattataggcgaacggaatgggaagtccggcgaaacagggtgacagccccgtagccgaaatagtgaagaagcacgagacagcacctgagtacggcgggacacgaggaatccctccttgtcgtgaatccaccaggaccttctggtaaggctaaatactaccttctgtgaccgtatagtgaaccaataccgtgagggaaaggtgaaatagacccccgggaaggggagtgaaatagatcctgaaaccgtatgcttacaagaagttagagcccgttaaagggtgatagcgtgcccttttgtagaatgaaccggcgagttacgatatggagcgaggttaagcaggaatatgccggagccgaagcgaaagcgagttttaacaggggcgaaagttgcatgtcgtagacccgaaaccgagtgatctagccatgaccaggttgaagttggggtaaaacccgatggaggaccgaaccgaccccccgttgaaacgttgtgcggatgatgttggtggctagtgggtgaaattccaatcgtaactcggagatagcttggttctccccgaaatagctttagggctagcagtcgtagtgtaaagtcagtgtgaaggtagagcactgtaatatgtgatggccccatctcggggtactgaacataatcaaactctcgaatgtgcacaaagatatactcggcagtcagacagtgggtgataaggtccattgtcaaaaagggaaacagcccagaccatcagctaaggtcccaaaatatatactaagtggaaaaggatgtggagatgtccagacaactaggcaggttggctcagaatgcagccattcaacctttaatagagtgcgtaacagctcactagtcgaagtgactctgcgccgataatttatccggggctaagtatatttaccgaagctatggatttacgcgttaaagcgtgagtggttagggggagcgttctatgtgcggagaagcggtaccgtaaggagccgtggagcgcatagaagagagaatgccggtgtgagtagcgaaacgtgggtgagaatcccacgccaccgaaaacccaaggtttccagaggaaggttcgtccgcctcagggtaagtcgggacctaaggcgaggccgagaggcgtagtcgatggacaacaggtagagattcctgtacttacggtatgaatgatggagtgacggagaaggctagcggatcctgctgatggaaatgcaggtgcaagcgagggagccgccagccaggcaaatccggctgtcgaaaggccatggcgctgaggcgtatggaaagctgcggcaagtacagaagtccgtgaagccagcttccaagaaaagcttctagtgataatcatacagtaacccgtaccgaaaatggacacacatgggtgaggagagaatacctaaggtgagcgagagaactatagctaaggaactctgcaaaatgactccgtaacttagggataaggagtgctcatagagatatgagccgcagtgaaacggcccaagcgactgtttaccaaaaacacagctctatgctaagtcgaaagacgacgtatatggggtgacgcctgcccggtgctggaaggttaagaggatgtgtcagcgcaagcgaagcattgaattgaagccccagtaaacggcggccgtaactataacggtctaagagggaattcctcaggtaagttaccgccccaaagagagcgatttgtggtgttcatctgtagatggtgaagtattagtacctgtcaagagcaggttaccccccctagacggaaagaccccatggagctttactgtagcttgatattggattctttgatgcaaagatgtacaggcataggtaggagtatgagagacatgcacgccagtgtgtgaggagtcaatgttgggatactactcttccttgtattggagttgctaaccggatgccatggaactggcaacgggcacagtagtcaggtcgggcagtttgactggggcggtcgcctcccaaagagtaacggaggcgcccaaagataccctcagcttggatggaaatcaagcgcagagtgcaaaggcataagggtgtttgactgcgagacctacaagtcgagcagggacgaaagtcgggcttagtgatccggcggtgctgaatggaaaggccgtcgctcaacggataaaagctaccctggggataacaggctgatctcccccaagagttcacatcgacggggaggtttggcacctcgatgtcggctcatcgcatcctggagctgaagtcggttccaagggttgggctgttcgcccattaaagcggtacgcgagctgggttcagaacgtcgtgagacagttcggtccctatctgtcgtgggcgtaggaagtttgagaagatctgtcctcagtacgagaggaccgggaggacatatcaatggtgcaccagttgtcacgccagggcacagcggtagctaaataggaagggaaaaa

SEQ ID NO: 7 - DNA, 3531 bp, Comamonas acidovorans
  rRNA (1)..(3531): 23S rRNA and Internal Transcribed Spacer Region
ctcatgggagcgggtctcgccgagtaggtagcctaaccgcaaggagggcgcttaccacggcggggttcgtgactggggtgaagtcgtaacaaggtagccgtatcggaaggtgcggctggatcacctcctttctggaaaactgctgttcaagttgaacgcccacacttatcggttgttggaacaagccatgtgccctggttgcagggtgggtggactgggtctgtagctcagctggttagagcaccgtcttgataaggcgggggtcgttggttcgagcccaactagacccaccaagattccaatatctggttgtcgaggatcccgggggattagctcagctgggagagcacctgctttgcaagcagggggtcgtcggttcgatcccgtcatcctccaccaagatcgcgctggtggcagcagcttgaaaaagcagctggaggaagcgaaaagagcacgaaaaaagcgtgctataatatttgactcaacactaaagcagtctcgtgtgagactgctttagtgttgatagttatctaactatcaatcggctgttctttaaaaattcatagagtcgaaatcagcgttgctgacggaaagagatttcaatctcaccgtgccgtcagcaacattttgattgcgtcaaaacgaatgaaactttgttttattcaagtaatgacgaattgttctcttgacagaaatgtcaaagaattcattcacattacggcataacgcgcgaggtgagagacctcgcaagtccttgaaagaaaacggcgaaatctcgcaagagagatcaaagttatagggtcaagtgactaagagcatgtggtggatgccttggcgatgataggcgacgaaagacgtgatagcctgcgataagcttcggggagctggcaaattagctttgatccggagatttctgaatggggaaacccacccgcaagggtatcgcatactgaatacataggtatgcgaggcgaacctggagaactgaaacatctaagtacccagaggaaaagacatcaaccgagattccgaaagtagtggcgagcgaaatcggaagagccttctagtgatagtcagacggttaacaaaacggaatggaaagtccggccatagtaggtgatagccctgtatgtgaaaaccgactggtggtactgagctagagaaaagtagggcggggcacgagaaaccctgtctgaatatggggggaccatcctccaaggctaaatactcatcatcgaccgatagtgaaccagtaccgtgagggaaaggcgaaaagaaccccgggaggggagtgaaatagatcctgaaaccgcatgcttacaaaaagtaggagcccgcaagggtgactgcgtaccttttgtataatgggtcagcgacttacattcagtggcaaggttaaccgaataggggagccgtagagaaatcgagtccgaatagggcgtccagtcgctgggtgtagacccgaaaccaagtgatctatccatggccaggatgaaggtgccgtaacaggtactggaggtccgaaccgactagtgttgcaaaactagcggatgagctgtggataggggtgaaaggctaaacaaacttggaaatagctggttctctccgaaaactatttaggtagtgcctcaagtattacctgcgggggtagagcactgtttaggctagggggtcatggcgacttaccaaacctatgcaaactccgaataccgcagagtacagcttgggagacagagcaccgggtgctaacgtccggactcaagagggaaacaacccagaccgccagctaaggtccctaaaattggctaagtgggaaacgaagtgggaaggctaaaacagtcaggatgttggcttagaagcagccatcatttaaagaaagcgtaatagctcactgatcgagtcgtcctgcgcggaagatgtaacggggctaagccagttaccgaagctgcggatttgcaatttattgcaagtggtaggagagcgttctgtaagcctgtgaaggtgcctggtaacgggtgctggaggtatcagaagtgcgaatgctgacatgagtagcgttaaagggggtgaaaagccccctcgccgtaagcgcaaggtttcctacgcaacgttcatcggcgtagggtgagtcggcccctaaggcgaggcagagatgcgtagctgatgggaaacaggtcaatattcctgtaccgatgtgtagtgcgatgtggggacggagaaggttagctcagccaactgttggatatgttggttcaagcctgtagtcgtgcctggtaggcaaatccgccgggcttagatgaggggtgataacgagtctgcttgcagacgaagtgagtgataccctgcttccaggaaaagccactaagcttcagctacacacgaccgtaccgcaaaccgacactggtgcgcgagatgagtattctaaggcgcttgagagaactctggagaaggaactcggcaaattgataccgtaacttcgggagaaggtatgccgcaagtaggtgaacttgaacaaagggagcccaaagcggttgcaaaaaatcggtggctgcgactgtttaataaaaacacagcactctgcaaacacgaaagtggacgtatagggtgtgacgcctgcccggtgctggaagattaaatgatggggtgcaagctcttgattgaagtcccagtaaacggcggccgtaactataacggtcctaaggtagcgaaattccttgtcgggtaagttccgacctgcacgaatggcgtaacgatggccacactgtctcctccagagactcagcgaagttgaaatgtttgtgatgatgcaatctccccgcggaaagacggaaagaccccatgaacctttactgtagctttgtattggattttgaacggatctgtgtaggataggtgggaggctttgaagtgaggacgctagttctcatggagccgacgttgaaataccaccctggtgcgtttgaggttctaacccaggtcccttatcgggatcggggacagtgcatggtaggcagtttgactggggcggtctcctcccaaagcgtaacggaggagttcgaaggtacgctagttacggtcggacatcgtgacgatagtgcaatggcataagcgtgcttaactgcgagactgacaagtcgagcagatgcgaaagcaggacatagtgatccggtggttctgtatggaagggccatcgctcaacggataaaaggtactctggggataacaggctgataccgcccaagagttcatatcgacggcggtgtttggcacctcgatgtcggctcatctcatcctggggctgtagtcggtcccaagggtatggctgttcgccatttaaagaggtacgtgagctgggtttaaaacgtcgtgagacagtttggtccctatcttccgtgggcgctgcagatttgaggaagcctgctcctagtacgagaggaccggagtggacacacctctggtgtatcggttgtcacgccagtggcattgccgagtagctaagtgtggaagagataaccgctgaaattttaacca

SEQ ID NO: 8 - DNA, 3200 bp, Corynebacterium diphtheriae
  rRNA (1)..(3200): 23S rRNA and Internal Transcribed Spacer Region
acaccgcgccgtcacaccacgcagcagtttgtaacaccccgagtcggtagggtaaccctttatggagccagccgccgaaggtgggacagataattggggtgaagtcgtaacaaggtagctgtatcggaaggtgcggctggatcacctcctttctaaggaaaaggaaacctgtgagttttcgttcttctctatttgttcagttttgagaggttagtacttctcagtatgtttgttctttgataactagataagaaagttagtaaagttagcatagataatttattatttatgacacaagtaaccgagaatcatctgaaagtgaatctttcatctgtattggaagtatcatcgctgatacggaaaatcagaaaaacaacctttacttcgtagaagtaaattggttaagttagaaagggcgcacggtggatgccttggcactaggagccgaagaaggacgggactaacaccgatatgctttggggagctgtacgtaagcgttgatccagagatttccgaatgggggaacccactatctttagtcggatagtatccttacgtgaatacatagcgtgaggaaggcagacccagggaactgaaacatctaagtacctggagcaagagaaagaaaaatcgatttcctgagtagcggcgagcgaaacggaaagagcccaaaccaagaagcttgcttcttggggttgtaggacacttctatacggagttacaaaagaaagttataaatgaagcggtctggaaaggcccgccaaaagacgggtaacagcccggtagttgaaatggctttccctccccagagtggatcctgagtacggcggaacacgtgaaattccgtcggaatccgggaggaccatcttcccaaggctaaatactcccctagtgaccgtatagtgaacccagtaccgtgagggaaaggtgaaaagcacccccgcaaggggcagtgaaacagttcctgaaaccgtgtgcctacaagtagttagagcccgttaatgggtgatagcgtgccttttgtagaaatgaaccggcgagttacgatttgttgcaaggttaagcggaaaaagcggagccgtagcgaaagcgagtctgaatagggctgcataagtaacaggtcgtagacccgaaaccaggtgatctacccatgtccaggatgaaggtaaggtaatacttactggaggtccgaacccacgcacgttgaaaagtgcggggatgaggtgtgggtagcggagaaattccaatcgaacttggagatagctggttctctccgaaatagctttagggctagcctcgaggtaaagagtcatggaggtagagcactgtttggactaggggcccttctcgggttaccgaattcagataaactccgaatgccatgtacttatactcgggagtcagactgcgagtgataagatccgtagtcgaaagggaaacagcccagaccaccagttaaggtccccaaatatatgttaagtggaaaaggatgtggggttgcttagacaaccaggatgttggcttagaagcagccaccattgaaaattgaagtgcgtaatagctcactggtccgagtgaccccgcgccgaaaatgtaccggggctaaacatattaccgaaactgtggatgaacctctttagaggttcgtggtaggagagcgttctaagggcggtgaagtcagaccggaaggactggtggagcgcttagaagtgagaatgccggtatgagtagcgaaagaagggtgagaatcccttccaccgaatatctaaggtttcctgaggaaggctcgtccgctcagggttagtcgggacctaagccgaggccgataggcgtaggcgatggacaacaggtagagattcctgtaccagtgctaattgtttaaccgatggggtgacacagaaggatagggaatcgcacgaatggaaatgtgcgtccaagcagtgagtgtgagaagtaggcaaatccgcttctcgcgaagcatgagctgtgatggggaaggaaattaagtacggaagttcctgatttcacgctgtcaagaaaagcctctaggaagagtagtactgcccgtaccgcaaaccgacacaggtagatgaggagagaatacctaaggtgagcgagagaacaatctcgttaaggaactcggcaaaatgaccccgtaacttcgggagaaggggtgctctattagggtgcaagcccgagagagccgcagtgaataggcccaggcgactgtttagcaaaaacacaggtctctgcaaaaccgtaaggtgacgtataggggctgacgcctgcccggtgctggaaggttaagaggagtgcttagcttcggcgaaggtacgaattgaagccccagtaaacggcgagaccgtaactataacgggtcctaaaaggtagcgaaattccttgtcgggtaagttccgacccgcacgaaaggcgcaacgatctgggcactgtctcaacgagagactcggtgaaattatagtacctgtgaagatgcaggttacccgcgacaggacggaaagaccccgtggagctttactgcaacctgatatggaatgtttgtaccgcttgtacaggataggtaggagccgaagagacgtgtgcgctagcatacgaggaggcaatggtgggatactaccctggctgtatgaccattctaacccgccacgcttaggcgcgtggggtagacaagtgtcaggtgggcagtttgactggggcggtcgcctcctaaagagtaacggaggcgcccaaaggttccctcagaatggatggaaatcattcgcagagtgtaaaggcacaagggagcttgactgcgagactgacaagtcgagcagggacgaaagtcgggcttagtgatccggtggttccgcatggaagggccatcgctcaacggataaaagctaccccggggataacaggcttatctcccccaagagtccacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgcgggcgcaggaaatttgagaggagctgtccttagtacgagaggaccgggatggacacaccgctggtgtaccagttgttccgccaggagcatcgctggtagctatgtgggcagggaaaa

SEQ ID NO: 9 - DNA, 1830 bp, Klebsiella oxytoca
  rRNA (1)..(1830): 23S rRNA and Internal Transcribed Spacer Region
gaatgttatcacggagacacacggcgggtgctaacgtccgtcgtgaagagggaaacaacccagaccgccaagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacactatgtgttgttgggtaggggagcgttctgtaagccgttgaaggtggcctgtgagggttgctggaggtatcagaagtgcgaatgctgacataagtaacgataatgcgggtgaaaaacccgcacgccggaagaccaagggttcctgtccaacgttaatcggggcagggtgagtcgacccctaaggcgaggccgaaaggcgtagtcgatgggaaacaggttaatattcctgtactcggtgttactgcgaaggggggacggagaaggctatgttggccgggcgacggttgtcccggtttaagcatgtaggcggatgttccaggtaaatccggaacgttactaacgctgaggtgtgatgacgaggcactacggtgctgaagtgacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatggtaagtgaagtgacttgctctggagctgaaatcagtcgaagataccagctggctgcaactgtttattaaaaacacagcactgtgcaaacacgaaagtggacgtatacggtgtgacgcctgcccggtgccggaaggttaattgatggggttatccgtaaggagaagctcttgagtgaagccccggtaaacggcggccgtaactataacggtcctaaggtagcgaaattccttgtcgggcaagttccgacctgcacgaatggtgtaaccatggccacgctgtctccacctgagacccagtgaaatcgaaatcgctgtgaagatgcagtgtacccgcggctagacggaaagaccccgtgaacctttactgcagcttgacactgaactttgaacctgtttgtgcaggataggcgggagacatagaagcaagagcgccagctcatgtggagtcaaccttgaaataccgccctgacatgtttgaagttctaactcgatgaagacctcaaagaggacagtgtctggtgggaagtttgactggggcggtctcctcctaaagggtaacggaggagcacgaaggtctgctgattacggtcggacatcgtaaggtcagtgcaatggcataagcaggcttgactgcgagagcaacaactcgagcaggtgcgaaagcaggtcatagtgatccggtggttctgaatggaaaggccatcgctcaacggataaaaggtactctggggataacaggctgataccgcccaagagttcatatcgacggcggtgtttggcacctcgatgtcggctcatcacatcctggggctgaagttggtcccaagggtatggctgttcgccatttaaagtggtacgcgagctgggttcaaaacgtcgtgagacagtttggtccctatctaccgcgggcgtaggataattgagagggattgctcctagtacgagaggaccggagtgaacgaaccgctggtttacgggttgtcatgccaatggcacggcccggcagccaagttcggaactggataaccgctgaatttttt

SEQ ID NO: 10 - DNA, 3618 bp, Ochrobactrum anthropi
  rRNA (1)..(3618): 23S rRNA and Internal Transcribed Spacer Region
catcatgggagttggttttacccgaagcgctgtgctaaccgcaaggaggcaggcgaccacggtagggtcagcgactggggtgaagtcgtaacaaggtagccgtaggggaacctgcggctggatcacctcctttctaaggaagatcgagaataggaaagacgcagtcttcgggctgatgatccttctccatcttattagaacatagatcgcaggccagtcagcctgacgatcgctttgcaggcgtgccgccttcgtttctctttcttcattgttgattgacacttgtaccgctcacgagccgtattgcagctgcgctgcttggctctgcgcgagcgcgccgcatgagcggcgacggactagcgtcctgtatttggttctaacggtttgtttgttggttctgatacaagggcttgtagctcagttggttagagcacacgcttgataagcgtggggtccggaggttcaagtcctcccaggcccaccaaattgtgataaggggccatagctcagctgggagagcacctgctttgcaagcagggggtcgtcggttcgatcccgtctggctccaccatcacttttttggtgtcgagtaggacggatagacagtcagtcaacaagagaaagaaccaagtttgcggactttacgaagtctgcgtgtttctgtatgaaatcgtgaaagaagatgtaatcggatcaactatccagttgatgtcgcaatggtttgctcaaaccttgcattatgattggacgctaaccgcgccaccgattgtatctcgagaagctggtctttctgctgatatgatcagagcttaatgctttgatggatattggcaatgagagtgatcaagtgtcttaagggcatttggtggatgccttggcatgcacaggcgatgaaggacgtgatacgctgcgataagcgtcggggaggtgcgaataccctttgatccgacgatttccgaatggggcaacccaccttagatagctagaaaatcaatttagttggagcaacgctgttgggtttaggcccatacagaccgctaggtcgtcggcccatgtgggccgcccccgcggagcgccagcatcgtaagatgcgtacggcgcgtgagcgagaactaaattaatttctagttatcgtaataaggtatctacacctgaatacatagggtgttagaagcgaacctggggaactgaaacatctaagtacccagaggaaaggacatcaaacgagactccgctagtagtggcgagcgaacgcggaccaggccagtggcaatagggaataaagtggaagaacctggaaaggtttgccgaagtgggtgatagccccgtacacgtagaacacctgttgtccttgagtagggcgggacacgtgaaatcctgtctgaacatgggtcgaccacgatccaagcctaagtactcgtgcatgaccgatagcgaaccagtaccgtgagggaaaggtgaaaagcaccccgacgaggggagtgaaacagtacctgaaaccggatgcctacaaacagttggagcccaaggttcgtcctgggtgacagcgtaccttttgtataatgggtcagcgacttagtgtatcgagcaagcttaagccggtaggtgtaggcgcagcgaaagcgagtctgaacagggcgttcagttcgatgcattagacccgaaaccaagtgatctagccatgagcaggttgaaggtacggtaacacgtactggaggaccgaacccatatctgttgcaatagatcgggatgacttgtggctaggggtgaaaggccaatcaaacttggagatagctggttctccgcgaaatctatttaggtagagcgtccagcgaatacccccgggggtagagcactgaatgggctatggggactcaccgtcttactgatcctaatcaaacttcgaataccatacagttggaagcagaggacagacggtgggtgctaacgttcatcgtcaagagggaaacaacccggaccgccagctaaggtccccaagttctagttaagtgggaaacgatgtgggaaggcatagacagccaggatgttggcttagaagcagccatcatttaaagaaagcgtaacagctcactggtctaaataagggtctttgcgccgaaaatgtaccggggctaaagccatacaccgaagctgtggatgcacatttgtgcgtggtagcggagcgttccgtaagcctgtgaagggacagtcgtgagacatcctggaggtatcggaagtgagaatgctgacatgagtaacgataaagggagtgagagactccctcgccgaaagtccaagggttcctgcttaaagttaatctgagcagggttagccggcccctaaggcgaggccgaaaggcgtagtcgatgggaaccacgttaatattcgtgggcctgcaggtagtgacggatcgcgtgtgttgtaaggtcttattggattgatcttgcagcgaagcggttccaggaaatagctcctgcatatagaccgtaccctaaaccgacactggtggactggtagagaataccaaggcgcttgagagaactgcgttgaaggaactcggcaaaatgcacgcgtaacttcggaagaagcgtgacctccatttaggcaactaggtgggggtggcacagaccagggggtagcgactgtttaccaaaaacacagggctctgcgaagtcgcaagacgacgtatagggtctgacgcctgcccggtgctggaaggttaagaggagatgtgcaagcattgaattgaagccccagtaaacggcggccgtaactataacggtcctaaggtagcgaaattccttgtcgggtaagttccgacctgcacgaatggcgtaacgacttcccccgctgtctccaacgcagactcagtgaaattgaattcccccgtgaagatgcggggttcctgcggttagacggaaagaccccgtgcacctttactatagctttacactggcattcgtgtcggcatgtgtaggataggtggtagactttgaagcagtggcgccagccattgtggagtcatccttgaaataccacccttgcctatatggatgtctaactgcggcccgttatccgggtccaggaccgtgtatggtgggtagtttgactggggcggtcgcctcctaaagagtaacggaggcgcgcgatggtaggctcagaacggtcggaaatcgttcgtcgagtgcaatggcataagcctgcctgactgcaagactgacaagtcgagcagagacgaaagtcggtcatagtgatccggtggtcccgcgtggaagggccatcgctcaacggataaaaggtacgccggggataacaggctgatgacccccaagagtccatatcgacggggttgtttggcacctcgatgtcgactcatcgcatcctggggctggagcaggtcccaagggtatggctgttcgccatttaaagcggtacgtgagttgggttcagaacgtcgtgagacagttcggtccctatctgccgtgggtgtaggaatattgataggatctgatccctagtacgagaggaccgggttggacagtctcttctggtggacctgtggcctgccacgcagctgggtagcttatacggacgggataaccgctgaaagcatcttaa

SEQ ID NO: 11 - DNA, 2996 bp, Peptostreptococcus prevotii
  rRNA (1)..(2996): 23S rRNA and Internal Transcribed Spacer Region
ctctcatggagttggcaatacccgagcctgtgagcgaaccttttagggcgcagcagtcgaaggtagggtcagtaactggggtgaagtcgtaacaaggtagccgtatcggaaggtgcggctggatcacctcctttctaaggatgagaagctgatacgtcagcttctcacgtgagatttttatctcttagaaataaaatctcaaagtaactttggattctgtataatagttcaaaatagaaccaacaaaactaaatagcaaaaaatatttccagtcaagaaagaaagggcgcaaggtggatgccttggcacatgaaggcgatgaaggacgtaagtgaacgaaaactagggagagctcacaaaaagcactgacccctaggtctccgaatggggaaacccggctgtggaagacacagtcattactaagtgaatacatagcttagtaaagcaagaccctgtgaactgaaacatctaagtaacaggaggaaaagaaagaaaactcgattttccaagtagcggcgagcgaaaagaaaagagcctaatccattgaggaatatctagttagtcgaatcatctgggaagatgaaccaaagaaagtgaaagtcttgtagacgaaaactaaataaaccagggaggaaagtagcaccggacacgaggaatccgttgtgaagatagggggaccatcccctaaggctaaatactaacatgtgaccgatagcgaacaagtaccgtgagggaaaggtgaaaagaacccccgaaagggggagtgaaacagaacctgaaacctagtgcctacaagcagagagagctctaaagagtgatctcgtaccttttgtagaatgggccagcgagttatcgtatatagcaaggttaaatcttttaagagatgaagccaaagcgaaagcgagtcttaataggggcgaatagttagatgcgatagacccgaaaccgggtgatctatccatggtcagagtgaaggtgaagtaaaattcactggaggctcgaaccgggtcggtttaaaacgtatcggatgaactgtggataggggcaaaaaccaaacgaactcggatatagctggttctcctcgaaatagctttagggctagcctttgattaagatttaaggaggtagagcactgaatggtctagggcggcttaccgtaccaaaacctatcaaactccgaatgccaaaaaatcaatcaaggagtcagacttagggggataagctttaagtcgaaagggaaacagcccaaccgacagcttaaggtcccaaaattggattaagtggaaaacgatgtgggaaggcatagacagcttaggaggttggcttagaagcagccaccctttaaagaaagcgtaatagctcactagtcgagtcggcctgcgcggaagatgtaacggggctaaaactatgtgccgaagctgcggatttgacattagtcaagtggtaggggagcgttctgtaagccgatgaaggtgtattgagaagtatgctggaggtatcagaagtgcgaatgctgacgtgtaagtaacgataaagggggtgagaaaccccctcgccgcaagactaaggtttcctgatcaacgctaatcggatcagggttagtcgggtcctaaggctcagccgaacggtgaggccgatggcagaacaggttaatattcctgtactacctataagagtgatgtggagacggaggagtgacaacgccgcggactgacggaatagtccgttaaagggtgtagatgttgattatcccaggcaaatccgggataagagtcgaacctgaagtataccaatttcctcggaaaacggtaatagtgcgtgtaaacatactcccaagaaaatccgctaaacttaatcttataggtacccgtaccgtaaacggacacacgtagtcgggttgaatatactcaggcgcttgagtgaatcactttgttaaggaactcggcaaaatgtccccgtaacttcgggagaaggggagccagagcgatctggccacagaaaccaggcccaagcgactgtttaccaaaaacacaagtttctgcaaaatcgaaagatgaagtataggagctgacacctgcccggtgctggaaggttaaggggaaggcttagcataagcgaaggctagaacttaagccccagtaaacggcggccgtaactataacggtcctaaggtagcgaaattccttgtcgggtaagttccgacccgcacgaaaggtgtaacgatttgggcactgtctcaacaaaggatcccggtgaaattgtagtagtcgtaaagatgcgaacttaccccacgctaggacggaaagaccccgtggagctttactgtaggctgatattggactttgagattagacgtacaggatagttgggagactttgaaacacgcacgccagtgtatgtggagtcacccttgggataccaaccctctaatattaaagttctaacgatgacccttgaatcagggcatcggacattgtcagttgggcagtttgactggggcggtcgcctcccaaaaagtaacggaggcgttcaaaggttcgctcagaatggacggaaaccattcgtagagtacaaaggcagaagcgagcttaactgcaaaacctacaagttgcgcagagtagaaatacggacttagtgatccggtggcaccgcatggaagggccatcgctcaacggataaaagctaccccggggataacaggcttatctcccccaagagtccacatcgacggggaggtttggcacctcgatgtcggctcgtctcatcctggggctgaagtaggtcccaagggttgggctgttcgcccattaaagaggcacgcgagctgggttcagaacgtcgtgagacagttcggtccctatccagcgtgggcgtaagaaatttgagaggatctgtccctagtacgagaggaccgggatggacacacctctggtgtaccagttgttccgccaggagcatagctgggtagctacgtgtggaattgataagcgctgaac

SEQ ID NO: 12 - DNA, 2047 bp, Porphyromonas gingivalis
  rRNA (1)..(2047): 23S rRNA and Internal Transcribed Spacer Region
atttatgaaagttggtaacacccgaaccggtggcctaaccgttgtgggggagccgtcgaaggtgggactggtgattaggactaagtcgtaacaaggtagccgtaccggaaggtgcggctggatcacctcctttctaaggagtttttgtgagtggaatgttggcgtcctgcctgtgatgggtggggttgagggcatgctgttgggttgtggggtatcacatgtgtggtggcctgtgcggtgctgtgttgcgtgcgtgtgtgcctggtgtggtgttcgtgtggtggttgagaactgtatagtggatgcgagtatctttattgttgtatcgtttgtgcgacgtggtgttgtgcaaagttgtgtagtgcgatcggtggatgccttggcaccaagagccgatgaaggacgttgtgacctgcgataagccctggggagttggtgagcgagctgtgatccgggggtgtccgaatggggaaacctggaatgtccggagtagtgtccggtggccctgccctgaatgtataggggtgtgggtggtaacgcggggaagtgaaacatcttagtacccgtaggaagagaaaacaagtgtgattccgtgagtagtggcgagcgaaagcggaggaggctaaaccgtgtgtgtgttcaagccggcaggtgttgcatgtgcggggttgtgggggccttgtggttctgctgccgcaggattggccagtgagaaatgttgcgtgaaggtgaagcgtctgggaaggcgtaccggagtgggtgagagtcctgtaactgtaagcgtggcactggtgtggggttgccccgagtagcgtgggactcgtggaatttcgtgtgaattggcggggcccatcccgtaaggctaaatactcccgagagaccgatagtgaaccagtaccgtgagggaaggtgaaaaaacctcgaacagaggactgcaatgaccctgaacccgtctgcctacaagcggtaggagcgccattaaggtgtgacttgcgtgccttttgcataatgaacctacgagttactgtttgtggcaaggttaattgttataatcaagacaaggagccgaagcgaaagcgagtcttaaaagggcgcccatttagtcacgagcagtagacgcgaaaccaagtgatctacccttggtcaggttgaaggttagtaacactaactggaggaccgaatcggtagcgttgaaaagctttcgaatgaactgagggtaggggtgaaaaggctaatcaaacttgggagatagctcgtactccccgaaatgcatttaggtagcgcctcggacgaataccatagggggtagagcactgtttcggctagggggtcatcccgacttaccaaaccgatgcaaactccgaatacctatgagtactatccgggagacagactgcgggtgctaacgtccgtagtcaagaggaaaacaatccagaccgccagctaaggccccaaaatcatagttaagtgggaaacgatgtgggaaggcatagacagcttaggaggttggcttagaagcagccaccctttaaagaaagcgtaatagctcactagtcgagtcggcctgcgcggaagatgtaacggggctaaaactatgtgccgaagctgcggatttgacattagtcaagtggtaggggagcgttctgtaagccgatgaaggtgtattgagaagtatgctggaggtatcagaagtgcgaatgctgacgtgagtaacgacaaaacgggtgaaaaacccgttcgccgaaagaccaagggttccagggtcaagttaatctgccctgggttagtcggggcctaaggcgaggccgacaggcgtagtcgatggagaacgggttgatattcccgtaccagtgaaggaccgtccatactgatattgtgatgctaaccatgccgatcacggtgtcatgaagtttttctttgtggtgttgtggggtgtgtgggacccaagctttggaggtaagcgtgttaacaggtgtgacgcagaaggtagccaagccacgcggtggttgtcgtggtctaagcgtgtaggatgactggttgttaaatg

SEQ ID NO: 13 - DNA, 3567 bp, Peptostreptococcus anaerobius
  MISC_FEATURE (1)..(3567): n can be a, t, c, or g.
agcttgacatccctcggaccggtgtttaatcacaccttcccttcggggctgaggtgacaggtggtgcatggttgtcgtcagctcgtgtcgtgagatgttgggttaagtcccgcaacgagcgcaacccttgtctttagttgccagcattcagttgggcactctagagagactgccagggataacctggaggaaggtggggatgacgtcaaatcatcatgccccttatgcttagggctacacacgtgctacaatgggtggtacagagggttgccaaaccgtgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagtcggaaacacccgaagccgattatccaaccgcaaggaggaagtcgtcgaaggtggcgtcgataactggggtgaagtcgtaacaaggtagccgtatcggaaggtgcggctggatcacctcctttctaaggagaattacctgctgttcgattttgaaagttcatactttcaaaatttgtaccttgaaaactgaataatttagtgattacaaaagacatctatcaagttaaacttgataaaatatatgagagaaaactcattaaaaaacaccaattattcttttaacactggtcgaaagaccaaaattagaaaaatctcattaataactggtcaagttattaagggtgcagggcggatgccttggcactaggagccgatgaaagacgtgataagctgcgataagcttgacatccctcggaccggtgtttaatcacaccttcccttcggggctgaggtgacaggtggtgcatggttgtcgtcagctcgtgtcgtgagatgttgggttaagtcccgcaacgagcgcaacccttgtctttagttgccagcattcagttgggcactctagagagactgccagggataacctggaggaaggtggggatgacgtcaaatcatcatgccccttatgcttagggctacacacgtgctacaatgggtggtacagagggttgccaaaccgtgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagtcggaaacacccgaagcctgattatccaacctkysaggaggmagtcagtcgaaggtaggcgtcagataactggggtgaagtcgtaacaaggtagccggyatcggaaggtgcggctggatcacctcctttctaaggagtcaaaktacgwtrctgttcgatttttgccaaaagssmattacttasmaaaactttgtaccttgaraactraagtaaactttagtagaytacaaargttacatggwggtgtagctcaagttgggagagcacytgmcttgmamgcagggggtcatatgagstcgaaasctcattmasasmcaccaagttattcttttamcannctggttnttcgatsagaaccasaattaraattaaatagctcatsaatattscyrgtcaagssaksaagggyrcarggtggatgccttggcactakgaagwcgatgaaaggacgtgataagctgaacgataaactagggtgagctcacaaaaagcactgacccctaggtctccgaatggggcaacccggttgtggaagacacaatcattactaagtgaatacatagtcttagtaaggcgataccctgcgaactgaaacatctaagtagcaggaggaagagaaagaaacatcgattttctaagtagcggcgagcgaaaggaaaagagcctaactcattagcgatatcttaaagttagtcgaattatttgagaagataaaccaaagaaagtgacagtcttgtagacgaaagctttaagatacgagagggaaagtatcaccgggcacgagtaacccggtgtgaagatagggggcccatcccctaaggctaaatactaacatggaccgnatagnnaacaagccatntgatggatgatggtctaaagccgtaggaagaaaggtaggcaaatccgcctttcacaaattctgaaagctgataggtatcgaaaacataagtagagaagttgatgactccatgttgccaagaaaagtcactatccagaccaaagcgcccgtaccaaaaccgacacaggtagggaggtagagaataccaaggacgcgcggnaagaaatcnggctttgttaaggaactcggcaaaatgtccccgtaacttcgggagaaggggagcctgagagatcaggccacagaaaccaggcccaagcgactgtttaccaaaaacacaagtttctgcaaaatcgcaagatgaagtataggagctgacacctgcccggtgctggaaggttaaggggaaggcttagagcaatcgaaggctagaacttaagccccagtaaacggcggccgtaactataacggtcctaagatatatatntatatanatatntntntatanatatatatatatatatctatatagatatatatctatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatatgatagtagtagtagtngtagtaatagtagtagtagtagtagtagtagtagtagtagtagtatgantagtagtagtgattactagtagtagtgagtagtgatcagtagtgatcagtagtactattagttagtnagtgatgagtagtgatgagtaattatgagtagtagtnatgatgagtgagtagtcagtgatnagtnatcagtaatcatgagtgatnagtnagtagtagtgagtagtgagtagtgagtaatnagtagtgagtagtgatnagtagtgagtgagtgagtgatnagtnatnagtgagtnatgagtgagtnatgagtagtnagtgagtgagtnagtnatggatnagtnagtnagtgagtagtgaatnagttaatnagtngtcagtgagtgagtnantcagtcatnantnagtcatgagtgagtgagtgatgagtgactgactcatcagtgatgatcgtgangatgatcatcatannatggtgagtatgatatatgagtactactatagtatatgatatatataccgtatatatatngtatatcgtatnggntggtatagtatngacgctatatcgtgtacggaccnataccgtgnntggtgcggtagtgatatatntntgnanactgtaccntagatngatagntccgggtagatcgctccataggtgtacgtgtagatggcctctagtctggntcggtagncggggttcgggccccanaanngtgncn

SEQ ID NO: 14 - DNA, 3104 bp, Peptostreptococcus magnus
  MISC_FEATURE (1)..(3104): n can be a, t, c, or g.
cccccgccnccncnagagttgataacncccnaagccggtggcntgnccgcaagnaaggaagctgtntaaggtgggatnnatgantngggnngangtggtncnaaggattcccnnccccggnaanggggganggatcacctcctttctangtaagaaacgcatgatcgaagatgatgcacaagagaagtgatgtttcgaagatactgtttagttttgagtgatactcaggtatgactcagagatcattganaactggataatagaaaataaattgcgatagaaatagaaaatgagatcgaaatagtaattgttattattgaggtcnttctagaaaagtcgaaaactaagaattttcatcactaaaaaacataaatcgaaacggatggatggattttagtaaatagttgaaaatagtaatcaagcaaacaccaaaataggtaaagtaagaaagagcgtatggcggatgcctaggcacagagaggcgaagaaggacgcagcaaacagcgaaatgcgacggttagcagtaagcatgcaacgatccgtcgatgtccgaatgggggaacccacctggagtagagaccaggtatcctgaagtgaataaatagcttcagagaggcgagactcagggaactgaaacatctaagtacctgaaggcaagaggaaaataaacgaatgattccctaagtagcggcgagcgaaaggggaagagcccaaaccgatcttaggatcggggttgtaggactgtcggcaaagagcaagaaatcattataggcgaacggaatgggaagtccggcgaaacagggtgacagccccgtagccgaaatagtgaagaagcacgagacagcacctgagtacggcgggacacgagngaatcnngctgtacgraaatmcanccaggnaccatactggtaaggactaaatactacctactgtgnaccgtataggtgaacacagtaccgtgagggaaaggtgaaaagaaccccgngagnggagtganatagaacctganaccatatgcttacaagaagttacgagcccgttaaagggtgatagcgtgcnttttgtataatgaaccggcgagttacnatatggagcgaggttaagcaggatntgcggagccgaancganagcgantcttaacagggcgaaagttgcatgtnntataccnnaaaccnagtgatctatccatgaccaggttgaanttggggtaaaacccgatggaggaccnaaccgncccccgttgaaacgttggcggatgagttgtggctaggggtgaaattncaatcgaactcggagatagctggttctcnccnaaatagcttnagggctagcgtcgaggtaaagtcgtgtgaaggtacagcacgtgaatatgtgatggccccatctcggggtacttgaacataatcaaactccgaatgtcacaaanatatncttancantcngacagcgngtgatnaagttcattgtcnaanngtaaacaccnccataccatcnactnncngtccccaannntatatactaagtnnaagnagatgtgggagantttcnaacnaacntncgnnngtttcccttcccantccntcccattttttnnancagtgcnntanccnccctcatttnggtcgnnntggactctngctccnantattnncccgngncctnagnnnacttacccggaantngntggaanttnccccttaannggagnngannanttnnngggngntnngtnntatnnnnaaanttatgnattttangngtnagcgtgagtggtaggggagcgttntatgtgcggagaaggnggtacngtaaggagcngtggagcgcatagaagagagaatgccggtgtgagtagcgaaacgtgggtgagaatcccacgcaccgaaaacccaaggtttccagaggaaggttcgtccgctctgggtaagtcgggacctaagscgaggccgagaggcgtagtcgatggacaacaggtagagattcctgtacttacggtatgaatgatggagtgacggagaaggctagcggatcctgctgatggaaatgcaggtgcaagcgaggtagccgacagccaggcaaatccggctgtcgaaaggcaaaggcgtgaggcgtatggaaagctgcggcaagtacagaagtccgtgaagccagcttccaagaaaagcttctagtgataatcatacagtaacccgtaccgaaaatggacacacatgggtgaggagagaatcmtaaggtgagcgtagagcaactatagctaaggaactctgcaaaatgactccgtaacttagggataaggagtgctcatagagatatgagccgcagtgaaacggcccaagcgactgtttaccaaaaacacagctctatgctaagtcgaaagacgacgtatatggggtgacgcctgcccggtgctggaaggttaagaggatgtgtcagcgtaagcgaagcattaattaagccccagtaaacggcggccgtaactataacggtctaagtnananacatgcacnccagtgtgtnaggagtcaatgttggnanannactnttctngtattgnagttntanccgnatgccatgnaantggcaacgggacagtgtcaggtgggcagtttgantggggcggtcgcctcccaaagagtaacggaggcgcccaaagataccctcagcttggatggaaatcaagcgcagagtgcaaaggcataagggtgtttgactgcgagacctacaagtcgagcagggacgaaagtcgggcttagtgatccggcggtgctgaatggaaaggccgtcgctcaacggataaaagctaccctggggataacaggctgatctcccccaagagttcacatcgacggggaggtttggcacctcgatgtcggctcatcgcatcctggagctgaagtcggttccaagggttgggctgttcgcccattaaagcggtacgcgagctgggttcagaacgtcgtgagacagttcggtccctatctgtcgtgggcgtaggaagtttgagaagatctgtcctcagtacgagaggaccgggatggacatatcaatggtgcaccagttgtcacgccagggcacagctggtagnctaaatangaagggaaaan

SEQ ID NO: 15 - DNA, 4243 bp, Fusobacterium necrophorum
  MISC_FEATURE (1)..(4243): n can be a, t, c, or g.
cccnccgtcaacaccgacgcanagttggttgcaccctgnaagkagcaggcctaaccttagggaaggatgctccgagggtgtggttagcgattggggtgaagtcgtaacaaggtatccgntacgggaacgtgcggatggatcacctcctttctaaggagtattcttgttgttctttcttctttggaagggttcgcgcatggaccttggaaactgtatagtagatcaaaaaacaaacaagaataaagaacaaagaactctagtttctagagttagctgscaaagaaaamaacmsgdttncaaagtamcaaagggcacacaagggatgcctaggtagaaagagccgaawaaggacgtggtaagctgcgataagcttggcgaagttgcaaacgaacgtggatgccaagatytccgaatggagcaatctgtaaagagtcatgtctttacacgaaagagggaaccgggtgaactgaaacatnctaagtaatnccgnaggaaaagtaaagtaacaacgataccctaagtagcggcgagcgaacggggtagagcctaaaccgtattcatgtcaaggatgcagccgttgtggatatggggtagcngggnaanagagararanaagaactgcaagnctatttcgcagaacgtaagcaaaggaacaagaaagatctgggaaaggtctaccgtagaaggtgaaagtcctgtattggtagnttttnnnncttnnnnnaaanngcgctgntatmtcttctnnnnnncccnnaagggnngntaatgnnnnnnnttggnaacacnngnargaannnnnnnnnnccnnnnttctnnnngcasraatnccccntnggwgnantnnnnnnggacnncaaatnctcgtaaggctanaatactctctgcgaggtacccaamtagctacgtamggtctmratagwyggnncstnasckaacncgatagtcgcaymgtrccgtgtagggaaaggsgaaaagaaccccggwaggggagtgaaagagaacctgaaattgtgtgcttacaagcggtcagagccacttyggtggtgatggcgtgccttttggagaatgatcctgcgagttacgtttcatggcgaggttaagaagaacggagccgaagggaaaccgagtctgaatagggcgcaagagtcgtggagcgtagacgcgaaacccggtgatctaagcctgtccaggatgaagctgtggtaagacacagtgaagtagcaggcctaaccttagggaaggatgctccgagggtgtggttagcgattggggtgaagtcgtaacaaggtatccgtacgggaacgtgcggatggatcacctcctttctaaggagtattcttgttgttctttcttctttggaagggttcgcgcatggaccttggaaactgtatagtagatcaaaaaacaaacaagaataaagaacaaagaactctagtttctagagttagctgacaaagaaacancaaggttaaagtaccaaagggcacacaagggatgcctaggtagaaagagccaacaaggacgtggtagctgcgataagcttgcgaagttgcaaacgaacgtggatgccaagatctccgaatggagcaatctgtaaagagtcatgtctttacacgaaagagggaaccgggtgaactgaaacatctaagtaatccgaggaaaagaaagtaacaacgataccctaagtagcggcgagcgaacggggtagagcctaaaccgtattcatgtcaaggatgcagccgttgtggatatggggtagcgggaaagagaaagaaagaactgcaagctatttcgcagacgtaagcaaaggaacaagaaagatctggaaaggtctaccgtagaaggtgaaagtcctgtattggtagttttcttgcgctgtatctcttctcccaagtaatgtggaacacgaggaattctgcatgaatctgcgaggaccaaatctcgtaaggctaaatactctggaggtncctaacccaccgccgtatgaaaagttgggggatgaggtaggtttankgggtgaaaagccastckaaccngggagatagcctcgttctctccgaaatgcatytacwgtgcmgtccttgcgttgtnttaatgatgggggtagmawcactgactgatactakwggggcstatatgcttactgaanttcaatgcaaactccngaataccstttatntcaagakcgcagtgagtngagacccatggtgnagttmacttnccatcgtnnccgasatggtcgaaaccamtttttcccagaccaccagmnnnttnaagggatcccssattcsgtatcttamgtggtgcaaaagggaggttgtgcakattnctntaaacaantnnaggcagnaaaanngtgttngnnngcntgnattntntantgntntnanttngtttnnnnacntnnnngtttntttttnntntttntccnntantcntnntctccctcnncntnntnnntnagttnntttattatttgatgncnngattctnntttgtntntttctnttngnnnttnnttccnnccnnccccttcgttnnatcgncgtaaggtcccnnatcatatntaagntnnnttgnggaaagnaggtggagmttcttaanacaacntaggaggttggcttagaagcagccattccttgaaagagtgcgtaaatagctcactagtcgagagtctctgcgccgacaatgtaacgggngctaagatatgaaccgaagctgtggatgtcgtaagacatggtaggagagcgttctgtaggccgtcgaaggaggactgaaaggaactctggaggtatcagaagtgagaatgcaggaataagtagcgasaaggggagtgagaatctcccccgctggaagaccaaggttttcagggtaaagcttgtcttccctgagtaagccgggacctaagcccaggctagaatgcgtaggcgtaatggaaaacagnatsaatatttactgtgccagttcctagctttgtgaaggtagggaacgccagaagggtatgcgcgcagacgaacggaagagtctgtagaagcatgtagagtgacttggtaggcaaatccgccaggttagacttgaggtgtgacatatactcgcaagaggaatgcgcaaatcccacgctgccgagaaaagcttctagcggtaaagtagagactgcccgtacttggataaccgacccacaggtggtcaggatgagaaatayttaaagcggtmcmgggctracwywcgttaaaggaactctgccaaaatggccccgtaacttccgggaagaaggggtgcctcttggtgtgagtatacaagcnatacaaagcgcanagaggtcgcagtgaagaggctcaagcaactgtttawcaaaaacacaggtctatgcgaagctgtaaggcgaagtatatgggctgacacctgcccagtgccggaaggttaankaggaggtagtgagartctaccgcaattggaaggccccggtgaacggcggcacgtaactataacggtcstaaggtagcgaaattccttgtcgggtaagttccgacctgcacgaatggtgtaatgatttgagcgctgtcttgacgggaggcctggtgmmattgtattaccggtgaagataccggttacctacagtaggacggaaagaccccatgsagctttactgtagcttggtattgggttttggcatggcatgtataggatagttgggagactgggaaggtatggcgctagctgtaccggagtcatcggtggaataccaaccattccctgctgaaattctaatctgtactttggaggtatggagacagtgctaggtgggcagtttgactggggcggtcgcctmacraaagagtaacggaggcrtwcaaaggttctactcaggttggatggaaatcaaccgcagagtgcaagggcaaaagagagtctkgactgcaagaactgacgggtcgagcmgatgcsaaagcatgacatngngatccggccatttcgnatggaagggtcntnnctcaaaaanaaaaannnctnccctggagatnacaggctgatcctacccgaaaattcatatggacgggnnggtttcncancttgatgtcngctcatccnatgntggggnaagaaaaaannncccaagggntgggctnnnccnnnnaanaaannngnancntnanctnggaannaaaacnnnaaaaaaaannnnccgntccnatnnagngggnngnnnnannaatnnnnnnnannnnctntncnnaaanan

SEQ ID NO: 16 - DNA, 2625 bp, Proteus vulgaris
  MISC_FEATURE (1)..(2625): n can be a, t, c, or g.
nncatgggtagtgggttgcaaaataagtaggtagcttaaccttcgggagggcgcttaccactttgtgattcatgactggggtgaagtcgtaacaaggkaacccgtanggggaacctgcnggttggatcmccycccttmcctaaragnatacgtgttatgtgcmgkgnnnctcacacagacttgtytkatkaagaacgagcaaaangsgsgtytgcgaaagctgacntgaagtcccccttcgtytagaggcctaggmcmccsccctttcacggcggtaacaaggggktcgaatccccntaggggacgccaattgcgcggtatgagtgaaaggsgtcccmcmctatagtytgatgcmaatcaaanaawagttaagataattttagcaagttattttaactattatgctyttttaacmatctggaaccagctgnaaaattgaaaaccaatccatntatccccnaggnataatnatgagtntntccaaaatntcaaactttgnatngttttttgccntcgaagtgggangancgagccatttacngtttgaggcggccngngnncngtnagngccacnnacnttannangntagnnnggcganccctgccccacnacnnaangnnatttgnncnnccntccccncccnnacnngtcattaaaaagaaacatcttcgggttgtgaggttaagcgaataagcgtacacggtggatgcctaggcaatcagaggcgatgaaggacgtgctaatctgcgataagcgtcggtaaggtgatatgaaccgttatacccgacgatttccgaatggggaaacccaatatccaatggatattatcattaactgaatacataggttaatgaagcgaaccgggagaactgaaacatctcagtaccccgaggaaaagaaatcaaccgagattcccctagtagcggcgagcgaacggggaacagcccagagtcttaatcaacagcagcatcaggagaacggtctggaaagtccggcagtaaagggtgatagccccgtatccgaagatgctgttattgtgaactcgacgagtagggcgggacacgtgttccmtccttgwctagaatatggggggaccatcctgccaaggctaaatactncctgawtgaccgnatagtgaacccaangtaccgtgagggaaaggcgaaaagaaccccggcgaggggagtgaaaaagaacctgaaaccgtgtacgtacaagcagtaggagccccancactaagctnntggtgaactcacannnattcttttgcatgataaanagcggnagacgacgcnaagtgacgtccaaccaatccaatcaggcagaggaggcttagtggtggggtgactgcgtaccttttgtataatgggtcancgacttatattctgtagcaaggttaaccgaataggggagccgtagggaaaccgagtcttaactgggcgaatgagttgcanggtatacacccgaaacccggtgatctatccatgggcaggttgaaggttgggtaacactaactggaggaccgaaccgactaatgttgaaaaattaccggatgacttgtggatgggggtgaaaggccaatcaanccgggagatagctggttctncccgaaagctatttaggtacngcctcgtgaactcatcttcgggggtagagcactgtttcgactaggggggtcatcccgacttaccaactcgatgcaaactgctaataccgaagaatgttatcacngnagacacacggcgggtgctaacgttcgtcgtgaaganggaaacaacccanaccgccagcttaggtnccaaagtcatggttaagtgggaaacnaagtgggaaggctcagacagccaggatgttggcttacaaccacccttatttnaaggaaagtcnctgngaagatgnagtgnagccncngcnngacggagagaccccgcgaacctnccntatagcttgacactgaacatngagcctccactgtgtaggnataggtgggagactatgaagtgtggacgccagtctgcatggagtcanccttgccaatannccaccctttaacgtttggatgttactaacctaggcccngtaatcncgggtncgggtwamcgtgtntcgcccgtgggtagtttgactggggcggtctcctcctaaagagtaacggaggagcacgaaggttggctaagcatggtcggacatcatgcggttagtgcaaaggcataagccagcttgactgtgagagtgacggctcgagcaggtacgaaagtaggtcttagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgttggaagattgagaggggttgctcctagtacgagaggaccggagtgaacgcaccactggtgttcggggttgtcatgccaatggcattgcccgagtagctaagtgcggnaagcagataac

SEQ ID NO: 17 - DNA, 4372 bp, Enterobacter aerogenes
  MISC_FEATURE (1)..(4372): n can be a, t, c, or g.
cccggnaaggggccccaannncnngggtnaantngnnannnnantnnggaagggnnnacnnnanccnaggggggnnggncntnntnnnccncccnccnttttaanaaaaggnngaaacnccnttnncnnnannggngcctgcggnannatnncancgggncnttanannaanccgccnntcacgccatcnaccatgwgtagtgggttgctnangaaanaangtaggtaagcttaacmttctwwrcagwgcngctgtaccactttcwygattcatgacttgwwwywanangtcgtracaaggtamccgtaggrgaacctgcggkyggnatcacctcctymccnttaanagarccsgccktygcagygctcacancagattgtctgatgraagtaanagaagcaaggcgtcttgcgattgagmcttcagtgtccccttcgtctawaggcccargacaccagnccnstntcacwwcggtaaccaggggttcnagrtcccctakkgganncgcgcrcatbgctcwtcgttmgtgawtgamagacgcttgaccngaacatattctcaagaaytcmtcttcgggtgnrcgttggnnagakatttgctctttaaaaatctggatcaagctgamaattgaaacgacacacagtctsatgtgtgttacgagtcttctcaaatttttcgcgtacacggatgaatgttttsynaygaaacatcttncggggttgtdgaggttaagcgnactamgacgkacmcggtgngatgccctggcawtcagakgcratgaakgacgtgctaanatactgcgaaaagcgtcggtaaggtgatstgaaccagnttacaaccggcgatgtccccggnaaggggccccaannncnngggtnaantngnnannnnantnnggaagggnnnacnnnanccnaggggggnnggncntnntnnnccncccnccnttttaanaaaaggnngaaacnccnttnncnnnannggngcctgcggnannatnncancgggncnttanannaanccgccnntcacgccatcnaccatgwgtagtgggttgctnangaaanaangtaggtaagcttaacmttctwwrcagwgcngctgtaccactttcwygattcatgacttgwwwywanangtcgtracaaggtamccgtaggrgaacctgcggkyggnatcacctcctymccnttaanagarccsgccktygcagygctcacancagattgtctgatgraagtaanagaagcaaggcgtcttgcgattgagmcttcagtgtccccttcgtctawaggcccargacaccagnccnstntcacwwcggtaaccaggggttcnagrtcccctakkgganncgcgcrcatbgctcwtcgttmgtgawtgamagacgcttgaccngaacatattctcaagaaytcmtcttcgggtgnrcgttggnnagakatttgctctttaaaaatctggatcaagctgamaattgaaacgacacacagtctsatgtgtgttacgagtcttctcaaatttttcgcgtacacggatgaatgttttsynaygaaacatcttncggggttgtdgaggttaagcgnactamgacgkacmcggtgngatgccctggcawtcagakgcratgaakgacgtgctaanatactgcgaaaagcgtcggtaaggtgatstgaaccagnttacaaccggcgatgtccgaatwgggaaacccagtgcaaattcgtygcactatygttaactgaatacatargttaacwaggcgaacccgggggaactgaaacattctaaktaccccgaggaaaaraaakcaaatcaaccwmwattncccccagtagcgarmrarcgaacggggagcagcnnccnannnnnntgagtnnnnactgagatcannnnnnagccaanagtttaaataagtttngtgtttagtggaacggtctggaaarttccgacggtacagggtratagtccsgtacmcccaaaatscmcaggttgtgtaactygaaraagtagggcgggaacacgtggtantcctgtytraatatggggggaccatcctccaaggsttaaataytcctgactgaccgtatagtgamccagtaccngtgaggraaaggcgaaaagaaccccgggcgaggggagtgaaaaagaaccttgaaaccngtgtacgtacaagcagtgggagcacctttcggggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacsaataccgaakaatgttatcacgggagacacacggsgggtgctaacgtccgtckttgaararggaaacaacccaraccgccagctaaggtcccaaagtcatggttaantgnggaaacgatgtggaaggcacangcancnnncngcccngatgttngcttanaanccnccntcnttttaanaaagggtnatnnctcnctggtnnantncncctnnncagaanattnangggnctaaaccntncnccnnanntgnnnnnnnnannctttttctttntngaaagggnncccggannntngtanntagttnnttgatnngtgntngtgnttnaganggtngnggntgccgnaaggatnngaganngtannnacnatnncnatngtgtggngttanntntgaaanagaagagttagcgtacngngttngcggagcanagnngantncttagtanntnnnananagcgnngnaggnagatgngatggncgnntagtnnanatttgnnggngcngtgnntagagnnnngnntnttnggctntnantnaaanaatnnaggnatgttagtgtgtgaannantgnnanangtantnataannnnnnaaaganagagtngnnnntncnnctgnggntttnnttagnggnctngcannnnaaganttctttgttaggtngnagnncnnnatnntnncaggtnnaggggaancntannnnntgtttnccncncnnnnanccanaanantccnnnntaagntttttntcggngggannannncncngggtgggggannacgncncntcnggaggnnggnnaancccnccccnnccacccaaggnncccccnataatcnttattttngnnggaaaatgtgnnanggganngnncacccnagcnannaggttntggtntatnaannanccaccnttnntttnaaaaaaagggtantanggttccttgntgrantnngncctcwcnggaaaaantgnancggggttaaccnanccaccaaagttwcgccaccgancactatgtgttgttgggtaggggagcgttctgtaagcctgcgaaggnnttnngnanncctgtwagnngcgtnntangnctnnggaggtatcagtaagtgcgaatgctgacataagtaacgtataaagcgggtgaaaagcccgctcgccggaaggaccaagggttacctgttccaacgttaakwgggngncagggtgagtcgaccmmsaaggccngrggcacgaaaggcgtagtcgatgggaaacaggttaatattcctgtacttggtgttactgcgaaggggggacggagaaggctatgttagccgggcgacggttgtcccggtttaagcatgtaggctggttatccaggcaaatccggataatcaaggctgaggtgtgatgacgaggcactacggtgctgaagtaacaaatgccctgcttccaggaaaagcctctaagcatcaggtaacatcaaatcgtaccccaaaccgacacaggtggtcaggtagagaataccaaggcgwttkagagaactacggagtgaaggaactaggcaaaatggtgccgtaacttcgggagaaggcacgctggtgtgtaggtgaagtccctgcggatggagctgagaccagtcgaagataccagctggctgcaactgtttattaaaaacacagcactgtgcaaacacgaaagtggacgtatacggtgtgacgcctgcccggtgccggaaggttaattgatggggttatccgtaaggagaagctcttgatcgaagccccggtaaacggcggccgtaactataacggtncctaaag

SEQ ID NO: 18 - DNA, 5502 bp, Streptococcus mutans
  MISC_FEATURE (1)..(5502): n can be a, t, c, or g.
ntntncttancncccggtnnnnnnntatnatttntntatataaaattgtgnncgaccacggttttttgtacngtagnncaanantctantantacantcntatngaanacccgtgttntnannanncgngaattnncttnntagaaangngttntntaaaanaanccnnnggggtannttnttntattagnngncccgtggtaactncaatnnntncggtaggcngcttttgtcnnnccaatantgntgngantnggaatantnntntnttntnnnnngngngggannttttatggggnncaatncnggtggggtgtttnntanngggngnnnnagccanncntttcttaancnnttcccancntttttngnggcccnccccnaacncaagttggaagagngggggagttttttttgggtgagnntngcctncanaaaaaaantnnnnccggtcaagcgaataagcgcacacggtggatgccttggcggtcagaggcgatgaaggacgtggcagcctgcgaaaagtatcggggagctggcaacaagctttgatccggtaatgtccgaatggggaaacccacccgcttgcgggtatcctgcagtgaatacatagctgctggaagcgaacctggtgaactgaaatatctaagtaaccagaggaaaagaaatcaaccgagattccgtaagtagcgacgagcgaacgcggactagcccttaagctgatttggttctaggaaaacactctggaaagagtggccatagaaggtgatagccctgtatctgaaagggccatttcagtgaagacgagtagggcgggcacgtgaaaccctgtctgaacatggggggaccatccttcaaggctaaatactactgccgaccgatagtgaaccagtaccgtgagggaaaggcgaaaagaaccccggagaggggagtgaaatagaacctgaaccgtgtgcgtacaagcagtaggagctccgcaaggagtgactgcgtaccttttgtataatgggtcagcgacttactgttcgtggcaagcttaaccgtataggggaggcgaaggggaaaccgagtctgataagggcgcatagtcgcgggcagtagacccgaaaccgggtgatctagtcatgcccagggtgaaggtgcggtaacacgcactggaggcccgaacccactcccgttgcaaaggtaggggatgaggtgtgattaggagtgaaaagctaatcgaacccggagatagctggttctcctcgaaagctatttaggtagcgcctcatatgtatcctctcgggggtagagcactgttatggctagggggtcatcgcgacttaccaaaccattgcaaactccgaataccgagacggactgtatgggagacacacggcgggtgctaacgtccgtcgtgaaaagggaaacaacccagacccacagctaaggtcccaaattttgtgctaagtggaaaaccatgtggaaaggcacagacagccaggaggttggcttagaagcagccaccctttaaagaaagcgtaatagctcactggtcgagtcggtctgcggggaagatttaacgggctaagcacagaaccgaagcttggggtgcatactttgtatgcgcggtagaggagcgttccgtaagccgttgaaggtggattgagaagtctgctggaggtatcggaagtgcgaatgctgacatgagtaacgataatgcgggtgaagaacccgcacgccgaaagcccaaggtttccttgcgcaacgttaatcggcgcagggtgagtcggcccctaaggcgaggacgaaagtcgtagtcgatgggaagcaggtcantntncttancncccggtnnnnnnntatnatttntntatataaaattgtgnncgaccacggttttttgtacngtagnncaanantctantantacantcntatngaanacccgtgttntnannanncgngaattnncttnntagaaangngttntntaaaanaanccnnnggggtannttnttntattagnngncccgtggtaactncaatnnntncggtaggcngcttttgtcnnnccaatantgntgngantnggaatantnntntnttntnnnnngngngggannttttatggggnncaatncnggtggggtgtttnntanngggngnnnnagccanncntttcttaancnnttcccancntttttngnggcccnccccnaacncaagttggaagagngggggagttttttttgggtgagnntngcctncanaaaaaaantnnnnccagcccccccannaggnntgccaagcagnaanggatgnntnttttcattnggnaannagatnaaagnggnaacaagggcncnntttnancnnnaannantngacnacnnnnaancangttctcangagtcggggctttacgtcatngcgggtgtntcnagaatganaggcancatngtacaattgtcataanagtntnctcgntgngacaacaggntatantcnnccgccactttttcgtacngnngggnggagntnngncannganatatcgnnnncctttaggaccgttatagtgtacggcncgccgytsactggttggcttcaatgtancnngcayctataagccgncttacgcgtagagcatgctcctcttaaccktcwagacamcrgktcaggtcgtctagcccctnatacgttcngccttacrkcttytgcagagacnctcgtgtattttgyataaacawtcgcmtgggcctattcaytgcggmtctmgtcgggcygtgcaccctagnayagagcaymcctgtctcccgnancaaaaatgncnnttnacggggtcattttgccgagttccttaacgagtatgttctctncgatncaccttaggtattctctcctncgcctacctgtgtcggtttgcsgtacggggcacctctcacctcgctagaggcttttcttggsagtgtggaatcaggaacttcgctactatatttcgctcgccatsacagctcacngccttacgggaaacggrtttgcctatttmccagcctaactgcttggacgcggatatccaataccgcgcttgccctatcctcctgcgtccccccattgctcaaatggtganggaggtggtacaggaatatyracctgytgtcccatcgmctacgmctttcgksctcgscttaggksccgactmaccctgmgcsgaykaacstttsckcaaggaaaaccttrggcwttcngngcgwgcgggwtyytcanncccstcattatcgytactcatrycrgcattckcacttcygatagcgctccagcgagwccttctcrrtctcrccttcaacrgcccttacggaacgctcctcctaccactggttcgcataasaacagttatgcacsccaagccttctgtgwtacygtkcttaggcccccggttamatyttycsgcgcasaccgtnacctcgrcccagtgagctattacgctyctcntttaaanggngtggctgcttctaagaccaacmtccctggytgtctgtaaagcaactttccacaatrstttttccactttagcacaggaatactttngggaccttwgccttgctgggtctggggcttktttcccttttmackamgggatckttwgcacctcggccgtgctgwcnntcccaaggggtacagatccktctcggyanttyggggagtttgcaactggwattcggkaagyccggcgrkgrgccccctagycatnnanacagtgctctacccccgagaggatacatatgaggcgctacctaaatagctttcgaggagaaccagctatctccgggttcgattagcttttcactcctaatcacacctcatcccctacctttgcaacgggagtgggttcgggcctccagtgcgtgttaccgcaccttcaccctgggcatgactagatcacccggtttcgggtctactgcccgcgactatgcgcccttatmagactsggtttccccttcgcctcccctatacggttaagcttgccacgaacagtaagtcgytgacccattatacaaaaggtacgcagtcactccttgcggagctcctactgcttgtacgcacacggtttcaggttctatttcactcccctctccggggttcttttcgcctttccctcacggtactggttcactatcggtcggtcagtagtatttagccttgraggatggtccccccatgttcagacagggtttcacgtgccccgccctactcgtcttcactgraatggccctttcagatacagggctatcaccttctatggccrctctttccagagygttttcctagaaccaaatccagcttaagggctagtccgcgttcgctcgtcgctacttacggaatctcggtatcgcatttgctttctccttgctccggktacttagatrtttcagttcaccmggktctgccttctcakcakcctatgwattcactgmtatggatactgctcgcattagcgagcagtgggtttccccattcggamatctaccggatcaaagcttgcttrccagctccccgatagcwtwtcgcagtgytcgtccacgtccttcatcgsctcctgasygccaaggcatccaccgtggyscstcttatstmrncttraccgtcwaaaaggaatcactacgtggatatccttgcattacaattgaatgngnatgtctactntcgtnntanctagttaacaaasancncgctwtgnaggaatgmtccttmanaactmaacaagatnccraccgctcctgntntatacgaccgsgaggtctantattccgtataancntcckwagagaggaggtgatncmgccscnccttccgatgcggcttcsttgctacnacttcacccaannatcatctgtnccacnnctkcggcggstggctcnanaanaggctacmtcaccgactgcnggtngcnacnanantnntggnggnncgacnggngngtncgaacaanatncttannaaagangnnatggcgccgnncttccgntacagctaccttannncaanntcacccgnaannnctgnnccntncntcnggnnacnnggcttccaaaaangggtacnncaacngannnnggnnagcgncnnanntncnacgggggggnnnnnatccngngacggngcnnatacncatncnnaaanannnaannncaccgntangnnntnanngnagngnnnnnnnngatagtncncacnncgananannannncnanacccccnnnncaantatannntnnacncannnaaaccnaannaantnaagntttnacncgnnnnnaannnnaccnccnnnnnncgcngcaannacnannannncnnaagnnnannnnngaacnnnttngangtannangtntnccnnnnccangngnntgngncatnnngcnncnnnggcnngagnnggngnnnnnncactnncnactnttanngnngnngtaanttnnntcannnnnntannngggncttnannnnnnacgcn

SEQ ID NO: 19 - DNA, 4488 bp, Kingella kingae
  MISC_FEATURE (1)..(4488): n can be a, t, c, or g.
gggcngganccccccccttnttannnaaaagaagagatngttagggtncncccccntatcggnnanttagatgaagatgcgaagagcnaanaagnacncaanangggttttgtagctcaggtggttagagcacacgcttgataagcgtggggtngtaggttcaagtcctaccagacccnccaggattagagaagcgaattcaagttgtaaagcttacgagaagtaagaaatactgggggcatagctcagttggtagagcacctgctttgcaagcagggggtncatcggttcgatcccgtttgcctccaccaagattaaataaaatgaaaaattagattgcaaattaaagcaagtttagataaactggcgagcttactttaatttgcgatatattttttataagcgaaagctgagaagaagtataattaaacgcatcgatctttaacaaattggaaagccgaaatcaacaaacaaagacaatgttgtcgatttggtttgggattgctnngnatnnnnangcaacagtatttcaggcagcctgcaaaggcaacaaatcgaataacaaaatttgggtgatgattgtatcaactaatcttgaattcaaaaggcaaggttagtacacaacaagcagtaagctttatcaaaagtagagaatctaagttattttagtagtcaacgctaaaatagcaaagtcagaaggttcttcaaatwataggagtncaagtwaataagtgcaycatgtactttgattcaatgcgatgtggggacggagaaggttaggttagcaaactgttggaatagtttgtttaagccagtaggtggaaagagtaggcaaatccgctctttcttaacaccgagacgtgatgacgagtgtctacggacatgaagtgaccaataccacgcttccaggaaaagccactaagcttcagttgaatcagaaccgggtggatgccttggcgatgataggcgaagaaggacgtgtaagcctgcgaaaagcacggagaagctggcaaaaaagcaatgataccgtgatgtccgaatggggaaacccactgcactcgtgcagtatcctagtctgaatacatagggctagagaagcgaacctggagaactgaaccatctaagtacccagaggaaaagaaatcaaccgagattccgcaagtagtggcgagcgaacgcggaaaagcctgtatatgataatggttaaagatagaaagaagggattggaaancttccgccatagtgggtgatagccccgtatttgaaattyttaaccatggtactaagtatacgagaagtagggcgggacacgtgaaatcctgtctgaatatggggggaccatcctnccaaggctaaatactcatcatcgnaccgnataggtgaacccagtaccgtgagggaaaggcgaaaagatnacccccggaaggggagtgaaatagaacctgaaacctgatgcatacatacagtgggagcatctttgtggtgtgactgcgtaccttttgtataatgggtcaacgacttacattcagtagcgagcttaaccgagtaggggaggcgtagggaaaccgagtcttaataggngcgactagttgctgggtgtagacccgaaaccgagtgatctatccatggccaggatgaaggtgcggtaaaacgcattggaggtccgaacccacgcatgttgcaaaatgcggggatgagctgtggataggggtgaaaggctaaacaaactcggagatnagctggttctccccgaaaactatttaggtagtgcctcgagtatgagactgatgggggtaaagcactgttatggctagggggttattgcaacttaccaacccatggcaaactaagaataccatcaagttgctcctcgggagacagacatcgggtgctaacgtccggtgtcaagagggaaacaacccagaccgccagctaaggtcccaaatgatagattaagtggtaaacgaagtgggaaggcacagacagccaggatgttggcttagaagcagccatcatttaaagaaagcgtaatagctcactggtcgagtcttcctgcgcggaagatgtaacggggctcaaatctataaccgaagctgcggatatacttttgtatatggtaggggagcgttctgtaagcctgtgaaggtgacttgtaaaggttgctggaggtatcagaagtgcgaatgttgacatgagtagcgataaagtgggtgaaaagcccgctcgccgaaagcccaaggtttcctacgcaacgttcatcggcgtagggtgagtcggcccctaaggcgaggcagaaatgcgtagtcgatggaaacaggttaatattccntactttattcaatgcgatgggtgtnanttnttannggnnnntgtgttnntanannnnatnnnttngggtnnnannnnnagttaatgntngattantgggagtgntatntangnaaattannntttgnnnagatgnannnnatnnncgtgnnntannaacnggtcaanttnnngangcangtnanantaatgagnnaggnaanngnnatagngcnngnagaagcgggnggtnnnnnnntnnannggaanancnngggnttancgtngtgaagtggtggnngttgntnntnttnnantattnngtaatgngnnannnggannaggngngtngangngnntannatntnanaaaaagaanananangnnggtantagnnatnggntgnaattantnnnagggnggatngtnngtnnattgntggttngtanatntntnnagnnnttatggnaggangntganaagattnnaaananntnaaagtnttntgatntgnatnggngtnngtnnnnntganntttanngtnaggtagnnantntngganggnnngnngggtnnaaggttangnangnnngttgttnnnannnnggggggnggntggnagnnangtttaatttttantngnntnatgttgggggnggtgngngngnggnnnnanntaggggnaagggcccaagtanagtcantatgngtnaaggagcagngttaatattcctgtacttntgattcaatgcsatgtggggacggagaaggttaggttagcnaaactgttggaatagtttgtttaagccagtaggtggaaagagtaggcaaatccgctctttcttaacaccgagacgtgatgacgagtgtctacggacatgaagtgaccaataccacgcttccaggaaaagccactaagcttcagttgaatcagaaccgtaccgcaaaccgacacaggtgggcaggatgagaattactaaggsgsttgagagaaytmgggagaaggaactcggcaaattgataccgtaacttcgggagaaggtatgccctctaatgttaaggacttgctccgtaagcatctgagggtcgcagagaatcgnagtgngctgcgactgtttattrwaaacacarcactctgctaacacgaaagntggacgtatagggtgtgacgcctgcccggtgctggaaggttrattgaagatrtgcncaagcatcgratmgaagcctccagtaaacggcggccgtanastataacggtnctnaaggganggnnatncctnnttnggtaanttcggaccccgcangaanggcgtaacatatggccacncatgtntcctcccgagactcagcgaagntgaaatnnttgtgaanatgcaatactcnccantgnnggttagaaagacgccgtnancctttactgtagcnnngcantggacntngnagtcacntgngtngtatagnngggaggntttnaagcagacgacgtccagtcnctgtggagaccgtccttnaacanaccatcccnggcgaagttncaggtngctaacccagacccnttacccggntccggggncccnagcaaccagtntnnnngnancngtccttnaannccncccnngnnnantttaggttnnaacccagacccgttatccngggtcggggaccgtgcatggtaggcagtttgactgggncggtctcctcccaaagagtaacggaggagttcgaaggttacntaggtccggtcggaaatcggactgatagtgcaatggcaaaaggtagcttaactgcgagaccgacaagtcgagcaggtgcgaaagcaggacatagtgatccggtggttctgaatggaagggccatcgctcaacggataaaaggtactccggggataacaggctgattccgcccaagagtccatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgaagggggctgctcctagtacgagaggaccggagtggacgaacctctggtgtaccggttgtttcgccagaagcatagccgggtagctaagttcggaagagaaaa

SEQ ID NO: 20 - DNA, 3177 bp, Bacteroides ovatus
  MISC_FEATURE (1)..(3177): n can be a, t, c, or g.
atnnngnggnnnntnnnnnnnnnnnangcccccnnncnaancncnnnaaaaaacncngggyccctraaggtacgttaccscarraaagcktcctangggtaaaactggtaattggggstaaagtsgtaaccaaggwagccggtmccggaaaggtgcggctkgtaamaggnccyccntttctggaagcgnaatgccggtaactgtcmcaaaaacgtggncangccacctaaaaagmctttaaanggtgtcntgttttttntgtactanctggtacttgtttrtttatnanacatatagatcamccatctattagaagtatagatagagataamcaagagaaaaagaagccgagtctagacgaaaggtagacaaggttgaactagtcctatagctcagttggttagagcgctacmctgataatgtagaggtcggcagttcaactctgcctgggactaccaagctcaagaatgaggaacagcgaaagctaaaagctaaaaaacaaaattcttkattaacctcyctggggngattagctcakctggctaragcatctgccttcgcanncgcagaggngkcaacggttssaatcmgtnntrttctccacgatctctknaaaanagagaaacsactcttctkwcatgatgtacraaaagtagaaattttagtaagagctaraagtatatatcaamccgymcgtgttcgagtatgcaatattgatagaaggaagcattccwctaccaayaccagtasgcagaaataaaacgtttgwaagtaaagtaagcaagggcgcatggcggatgccttggctstcgkaggcgatgaaggacgtgataagctgcgataagcttcgggcaggtgcaaataacctttgatccgaagatttcygaatgggacaacccggcattctgaaggaatgtcatccatcctagatggaagctaacgcagggaactgaaacatcttagtacctgtaggaaaagtaaaataataanntgattccctngcnngtagtggcgagcgaacggggwttagcccaanccaatgttgttacggcaatattggggttgtaggaccacgatgtcgcaagaaatttagtgagaagaaccctttggaaaatgggaccatagacggtgatagtccggtattcgaagctaaatgaagcgtagtggtatcctgagtagcgcgggacacgaggaatcttgcgtgaatctgccgggaccatccggtaaggctaaatactcccgakasaccgatagcgaaccantactgtgaaggaaaggtgaaaagtcacttnccgnaatagcaagcagtgaaatagtccctgaaaccgtgcgcctacaagncggtcggagctgtgtaaacagtgacggcgtgccttttgcataatgaacctacgagttactttttccggcaaggttaaggatcttgagatccgcagccgaagcgaaagcgagtcttaacagggcgattagtcggaaggagtagacgcgaaaccaagtgatctacccttggtcaggttgaaggttaggtaacactaactggaggaccgaaccgataagcgttgcaaaagcttccggatgamctgagggtgggggtgaaaggctaatcamactttgkagaanagnytggtactncnccgaaatgcatttagagtgcntgncnttgganaanttnctanctgtgangnnnnncnctgananntncggggcntnccnnnggnngggttatatnnctntnnngcgagggncntntanncccnggcnnnngnnnnggnnnnaacncccnnntnngggnaanaaannntnntnggggggtccccnntwtwttnntanntncagaanaangnatnnattncntaatancannnnngnngngnttttggcannnctanncgtcnantnttncttaaaatnnnaannnantnccatancannantnntnntnnttccnnnanaantattatnntanttactttnnantatttnnncttnangaatatnattattcttnannanttannttttantcnntttttcnnnnnnctanatngnnnnannanattaactcnaatcnnnnananntaactttaaanatctantacatncnannnctanganntattnatanttntntntgtttanttgnnntnntnncntcttgntntncnacttttttantanntttcntancctncanntnctanngtnnttantncnntntccnttattannnttanaangccaanacatnatcannntctattnctnaccatcatntcttnatcnnactnactnctgnnttnatctaccatncccgaattaggtacccaggcaatgcacctggcggcacaactggtaaaccagcggtcagtccaacacggtcctctcgtactagtgtcagagccacgcaaatttcatacgcccacgatagatagagaccgaactgtctcacgacgttctgaacccagctcgcgtgccactttaatgggcgaacagcccaacccttgggaccttctccagccccaggatgtgacgagccgacatcgaggtgccaaacccctccgtcgatatgagctcttgggagggatcagcctgtntatccccggagtaccttttatcctttgagcgatgtcccttccatacggaaacaccggatcanntatgctctagtttcctacctgatnnanttgtctgtctcccantcaagcgcccttatgccattncacntntacggacggatacccaatcngttctgnagggnncnctttagaantcctncgtctanacttttggnggncnttcntcccatntaanancnttccaccaaaaagntgtttctatcntnttacnagtttggaacttttaantatttanaagggntngnnagtttantctatcnnnantcnnnnnaattctttggnancctantttttttnnaaanantttnnggnntantnntgtantnttnagtctncccnaattntttnctntttntaacgttatantgttnatagggtnntcatnnngntnnttntgcgngntntnttntntnnnnngnattttnntnnactntancnntnncntnctaccatctnntcttntttctntcnagnntnntttttattctnncacnaaccttcntctccgntttntttctntnttttncnnctttcctccact

SEQ ID NO: 21 - DNA, 2770 bp, Bacteroides thetaiotaomicron
  MISC_FEATURE (1)..(2770): n can be a, t, c, or g.
ggggggccnnccnatnnggtncctttttaanntccnnaaggccncnantgttttantnccnngaaanaannngcattngggnnaangnnccnntnccnaaaanaaaagnntttggnnncnccngcccgtcgagccctnaaagccgcggggtactgaaggtaggtaaccgcaaggagngtcctagggtaaattntggnaattggggcttaagtygtnntcananggtagccgtaccsgaanggtgsggctggaacmccctcctttctggagcgnatgtsgttacaacgatttcatrggtttctattattgtactactggtacttgtttatttataatatatagatcamccatctatagaagtatagatagagataamcaagagaaataaagaagccgagtctaacatcaggtagacaaggttgaactagtcytatagctcagttggttagagcgctacactgataatgtagaggtcggcagttcaactctgcctgggantactccgaacaaaaattgtcgttgccaatacagtgttggaatgttcaattactcttgggggattagctcagctggctagagcatctgccttgcacgcagagggtcaacggttcgaatccgttattctccaccatctctgaaaagagaaacgatctttgacatgatgtacaaaaagtaaaatttagtaaagagctaaaagtatatatcgaaccgtacgttttaagtacaacactattaagtactttgggttaatacctgatacttgatacctaatacttaaaagtaagtttgaaagaaagtaagcaagggcgcatggcggatgccttggctctcggaggcgatgaaggacgtgataagctgcgataagctctgggtaggtgcaaataacctttgatccagagatttccgaatgggacaacccggcattctgaaggaatgtcatccatctttgatggaagctaacgcagggaactgaaacatcttagtacctgtaggaaaagaaaataataatgattcccctagtagtggcgagcgaacggggaatagcccaaaccacccatgttacggcatgtgtggggttgtaggaccacgatgtcgcaagacatttgatgagtagaatcctctggaaagttgaaccatagacggtgatagtccggtatacgaagtcaaattaagcgtagtggtatcctgagtagcgcgggacacgagaaatcttgcgtgaatnctgccgggaccatnccggtaaggctaaatactncccgagaagnaccgnatagcgcacacccaagtactgtgaaggaaaggnntkwaaascmctattcgaatagcaagnagtgataatagtccctgaaaccgtgccgcctacaagtcggtcggagctgcttaagcagtgacggcgtgccttttgcataatgaacctacgagttactttttccggcaaggttaagcatcttgagatgtgcagccgaagcgaaagcgagtctgaacagggcgtcgagtcggaaggagtagacgcgaaaccaagtgatctacccttggtcaggttgaaggttatrgtaacactaactggaggaccgaaccgatawgcgtatgaaaagcttaccggatgtaacatgakggtgggggtgtaaaggmtaatctawactatrkagtatagtmtmgtactccccgatatatrcatatatakrtryagccmtatrtgagtatactatatrtgakrtakagckactgatamkatgckagggctatcaccgymtatcatagtactntgatatatactccgatatgcgcatntanttmtatcacatakagtgatggcatggrtgmtatatagtccatrtactatatagkakatakatatncataccntcanctataggtcccyagwatawayastnagtatgnactatactatagwcanntatatgmtatatacngctnngatrtatagctatggatagyancctatatatayatatatakatrcrtayanctcactatrtcnnggagtatatggagtggatactatatatcnggcatatatantrtatatatacckatatctatgggatnnnnnatgatnggnanggtacntatmccatatatatatatatatatatatatatatatatatatatatatatatatatatatntncatatatatctatatatatatatnatatatanctatatatatatatatatatatatatatatatatagtatatatatatagtanatatattatatatanagtgtatagnctaccatatatatatangatagactatatatatntncntacanatatatanntatntntctanananatgatatnncnatanngcnntanananntanatatanangtnnatcaggnagtatannagagcatatntgatccgntgattccnatananangtacatngctcnatagnatatatagnatactccgggnatatcngtctntatccctcccatagagntnnatatatcgaangggttnggcacntngatgtcggcncgtcncancctngggctggagaaggtcccaagggtnggnctgttcgcccattaaagtggcacgcgagntgggttcagaacgtcgtgagacagttcggtctctatctatcgtgggcgtatgaaattngcgtggctctgacactagtacgagaggtaccgtgttggactgaccgctggtttaccagttgtgccgccaggtgcattgcctggagtatnctggagttcgggtangcgaaaaaagagnnc

SEQ ID NO: 22 - DNA, 3455 bp, Clostridium difficile
  MISC_FEATURE (1)..(3455): n can be a, t, c, or g.
gngnnccnncnnncnngccnggggggggnnnccccccnccnncgagncnntcnnccnncnnncngnggccnttnttannngggggggncccccnngnnggggnggnnngnannccttcnncgggggggggggncagcccccncntnnnnccggcagnnnggncccccaaaaantngtnantgnggngnaanaagggggggggggccncaccnaccccccnggngtttgttggtttaaccncnccccccncttccntcaanaggggggcgaaagnanccccngngcnanggggtcccccccnccgcagaatttncgngggggggntaatttcttaccccnnggagcngnnggcccccagtananggngggggggggcggctccccacggggtgnngnngcanccccngnnngcctcangtgcacagtttgggngngagaaatggggcncncgagnagcnnatgntggggggngaggncncccncngccnggacncncngggttnccnggnnangntgtcccccccccntggggttagncggccccnnaagctgngcccnaanggcggtaggtgatggccancnggtnaatantccggnacncccgntanctgtntgcnagrrgnngsagtanntcncnnaaggnnnagccntatnagnancngnanananatcncnnccngngttnnnagkrgrayyccncsctgcgnncaatagcangtgnngatnccncgcaattnatttcngttggcgcntngcgttagcactttgaaaactgcatatatatttagtgatatgacatctaatttgtaatatataaagctgataacttttttaaaattawcgaangttgatrgcttctnaatctatcaacnaccntttttaaytggntcaagttattaagggbgcagggcggatgccttggcactanggarccratgaaggamgtkantaacgytgcganntaagctttcggggagttgcamgtwawctttgatcncgaaagatttccgannatgaggaaactcacttnagangtaatgtstaagntatcattaagtraatacatwgcttaatgagngggaastcagggaactgawacatnctaagtacccgangcaagnanaaagaantcgnannaccganttcnttcngtaagnangngngaangangangcggcttagccccaaaccnaakaaagttttgctttattnnggggykgcnggatcatatcataacgaagnanggtwtcgtaattgaagaggtttggwwagacccaccmcascaaggtaakagtcctgtatgttaaacgagaagactttagatatgatccagagtaccacgggacacgtgaaanccctgtssgaagcaggagggaccaccctccawggctaaatactacctagtsaccgaatagccgtatagkaccgtgagggaaaggtgaaaagaaccccgggaggggagtgaaatagaacctgaaaccctgcacttacaagctgtggaagcacatttcttgtgtgaccgcgtnctttttgtanaacgggccaacnanttacnttaagtagcaaggntaatcncttaaggtgcggancctnagcgaatncnattttttnactgaccgttcacttactngnccnngactcnnnaccggnccatctnnncatgnncatagatcnnaattanagnnnntccccncnctggtcctanctnttatttnaagagtgcgtaatagctnactggtyggagtgatcctgcsccgaagatttccggggstwtaaactttactaccgaaagctacgggcatcagtaatgatgggtaggggagcttcccnatacgggttgaagcatgaccgtaaggacatgtggacagtatgggagtgagaatgttggcatgagtagcgagatgtgggtgagaatcccacaggcccgtaaacccaaggtttccaggggaaggttcgtccgcccctgggttagtcgggacctaagctgaggccgaaaggcgtaggtgatggacaacaggttgatattcctgtactaccgataaccgtttgaragaagggatgacacagtaggataagctaagcacactgttggttatgtgtgcccaagcattgaggcagtcaaagtaggcaaatccgctttgataatgctgggatgtgatggggagcgaaatttagtagcgaagtagctgatttcacactgtcaagaaaagtctctatcgaggttaaaggtmcccgtacccgcaaaccgacacaggtgggtgaggagaktwtcctaaggccagcgagagaactgttgttaaggaactcggcaaaatgaccccgtaanngnnncttagggataaggggtgccaccatgcaggtggcccgnccagakaantwggcccwasccgactgtttacccnaaaaacnttnngnttnatnntnnnnnnnnnnnansgnnntnttncanntnantgcctwagtncgcaagacgatgtataggagctgacgcctgcccggtgctggaaggttaaggggatctgttakagcaatcgaagcagtgaacttaagccccagtaaacggcggccgnnactataacggtcctaaggtagcgaaattncttggcgggtaagttccgacccngccacgaaaggcgtaacgatttggggcacttgtntcaacaaacattactcggkgaaaattgtaattcccggtgaagatgccggnataccttgcgacaggacggaaaagacccccatggagctttactgwakgcttgacattgggtcttggtactacatgtacakgataggtgggaggctttgaaacccasgacgccagttttggcggagccatccttgggataccacccttgtagtrctgggactctaactcatasgccatgaatctggtcttgggacactgtcaggtgggcagtntatgactggkgcggtcrcctcccaaaakgtaacggaggcrctcaaaggttstctcagtacrrtackgaaatcgtacgtaragtgtmaaggcccaaatagrgagctatgrtgtgcmagacatcnancatgtgtgcgagcganargatgaaaatcggacctccctrgkgrtcctggycggtctcttgcgtggaagggnnngccrkccgcttcmmcsgataaaagacwatccytggcggataayrggctttttayacyccccccnnaagagtccgaacatncttgrgcnnggggrggcnanaaatttggncaccatcgatgtcggctcatcacatcctggggctgtagtaggtcccaagggttgggctgttcgcccattaaagtggtacgcgagctgggttcagaacgtcgtgagacagttcggtccctatccgtcgcaggcgtaggaaatttgagaagacctgtccttagtacgagaggaccgggatggacgtacctctgtgtaccagtgtcctccaagggcaggcnggagctatncn

SEQ ID NO: 23 - DNA, 2651 bp, Haemophilus aphrophilus
  MISC_FEATURE (1)..(2651): n can be a, t, c, or g.
ccngncncnccannnctgggagtgggttgtcmccgmagyagatagcttaaccgcaawgggggcgtttaccmcggtatgattcatgactggggtwmagtcgtaacaaggkraccgtaggggaacctgcggttggrtcmcctccktaccganaagacgaacytaagtgtccmcacagtttgwctgatgattgtagacaagawagaacaaakcsaangngtannawncnaacnaagntntnnagagcatctttatatgttgtccccatcgtctaraggcctawgnacatcgccctttcmcggcggtarccggggttcgaatccccgtggggacrccatataaagatgattaytcatcttctttataaaacctgttctttaacaaaccggaaacavgctgaaaacgagactttcaagaaagtctgagtgakagtgattgataaaaggtgattactcttaatamattaactgtyactgcttagcrdcdttvvgtgtttayycrattcaatgttgtgattttaaktggattytcvatttaatgttaagytcatkatgttgagttancnatcataagaatacttgaggttgtnatggttaagtgacaanagactgtvcaattttkgtgraatgncnntcnttkkcaatcagaggcgtcatagttaaggacgtgcnccnnttaatnctgccaagnnannaannnatnnnnnagnctnnnnntnnnnggannnnntgagtncncnnnnncgncatanagannntnnncggccccnctgtnnnnttaatccaagatgtccgaatggggaaacccagtgggtgaagaacccactatcattatctgaatccataggataatgaggcgaaccgggagaactgaaacatctaagtaccccgnagcaaaagaaatcaaccgtaganatcttttttaaannnncttttttttnnnnnnccngnnannnnnnnnnnannnnnncttnnaanaanntttnnnnngaatgnatnnanaaggtnantnntcttnaatnaattnnngntncnnannancggngattantttttttnnannnangtngtgatntttagaggntnttncaantnannngtngntnatgatgagtanntnatnanaanaanncntntggtttnnttgnttaangangcagnnnacnaggnngcntcccntgncatangagnagaagaangnngtgatnntntnananaangtttgantagatnataagaggngttttnnncaagangncnnganggggcncaccnggtggngaacccnccnncatnattttnntcnancntatnatnatgngnnccnccaggantaattcatcnnantannccccngannanaaatcanccnncnntttnttantagnngcgagcnnacganaagnccgtaggcatcctgttagtgataatgacagsagnacagaaggcaacaasctgggtragcttgnntgcgacacagggcatgakagcacccgymctcgaagtccaggcttakggtastaagctaacgacaagtaaggcrggacacgtratatcctgtttgaakwtggggggaccatccyccmaggctaaatactacctgnattsaccgatantagnaaccrgwacctgtgaaggaaaggcgaataagtaaccmacggtgaggggagtgaaatasaacctgawaccttgtacgtacaagacagtgggagcctgaaagggtgactgcgtaccttttgtataatgggtcascgacttatattttgtagcgaggttaaccgaataggggagccgaagggaaaccgagtcttaactgggckagtakttgcwaggtatagacccgaaacccgktgatctagccatgggcaggttgaaggttgggtatacactaactggasgaccgaaccgactaatgttgaaannaattagcggatgactcntgtggctgggggtkaataggccaaycwaaccgggattkatagcctgnttcntccyygaaattctatktakgtaaanccctttgtataramamctktcggggggtnaaacaccctgntntcyggyttaggggntncntcatcmccktctnttmcncnataaaannncgttntncnatattttgntcnngaacnancnntnnttnntntcntntgntacanattnnnnntaacncncnnttatttttnttnttnnctctnntttnttnnnttnacntggganacacnccngangggtncncnacanttnnttngtgtgnnnaaggggaaaccnnccccactaccngntaaanttaangtnccnaaaannctttatatntnnantnttcaanancaaaannattnatnctnnnnactntgtttttnntngttnnacntnaanannancaaananacactntctnnagantanntnnnttnntnnnacttnantcnttncncccccnnananacnnnntananantnanactnattnttccnncanatatnnngntaccnnnaaccncctnnncnnattngtanncnntnnnntnanatngntnncnatcacntnnnctnannttnanagagtancgaccaanactncntnctctacatctnanctctactcnntcccncccaatacnnncctacnnnttcttatnaacntacncttcn

SEQ ID NO: 24 - DNA, 3077 bp, Neisseria gonorrhoeae
  MISC_FEATURE (1)..(3077): n can be a, t, c, or g.
ggntntgngtantgncaaanacnnncctcnagngtgnnnngnattattatatgtccngnnagaantgaaacagngnggggnnnngngatnangtanttaatngntgctnnnggcgngntngtnnatgnaaagngnttncngngacancataactgtnactattnctatnttgcgcnctantggtgnagtttatnngngnagttannnttngngaagaanntccntnttaaggannggnaattatnncctatnantngnntacagtngtttntnttttccnannttnncggtnnnnncangnanaaatnntntgannncttattnaaattggngccnttnngnnaaannantncnncngngttttataanagnnncgttngttagnagnggntantctnncctannagtnngcgggntnnaggttccncagacntatnnaatatcccnctanaaccnagnggnnnttgccnnntcgttngntcannnggntanannnanncgcttnatnannnnngggntngnaggttnnantccntcccnnnccccnccaanannnggggcatagntcagtntggtnngnagcaccngctttgcwagcagggggtcmtcgagtwcgatnccgtttgcctccncccnaaactttacaaatgaannagcaagtttgctgtttttagcagcttattttngatttntgcnngatagctagawtanacgacgcgatmkatctttgancaaattkgacaagcgcgmwatcarcamcacamagacaatgagtttgttttgmttttttattctttgcaaaggataawaacctctcgcaagagaaaagaammgcaamcatagtatttgggtgatsattgtatcagacttaatcctgartacacaaaaggcaggattaagacacaacaaagcagtaagctttatcaaagtagggatttcaagtttgcttacttagtcaacgggtaggtaaacgcaagtcaaagtaagttcttsaaatsataggagttcaagtsaataagtgcatcaggcggatgccttggcgatgataggcgacgaaggacgtgtaagcctgcgaaaagcgcgggggagctggcaataaagctatgattccgcgatgtccgaatggggaaacccactgcattctgtgcagtatcctaagttgaatacataggcttagagaagcgaacccggagaactgaaccatnctaagtacccggaggcaaaaggaaatcaaccgcagntaccgcaannggggntnnaatnnanancnnaanntncgnannccgntttnccggcatggtagggnagcgttntgtagnntnannaaggtgcattgtaaagtgtgntgnaggnatcanaagtncgaatgttgacatnagtanngataaagcgggtnaaaagcccgctcncngaaagcccaaggtttcntangcaacgttnatcggngtagggtaagtcggcccctaaggcgaggcagaaatgcgtagtcgatgggaaacaggttaatattcctgtacttgattcaaatgcgatgtggggacggagaaggttaggttggcaagctgttggaatagcttgtttaagccgggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttgcgggagaaggtatgccctctaaggttaaggacttgctccgtaagaccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggtccgtaactataacggtacctaangtaaccaaattccttgtctnntaanttccgancctcnctaatggcgtaacnatgnccacactgtgncctaccgatactcaannatagttgaagtggttgttatagatgcaaaactatcttgcttntgnacctgnnagacaaacntnnaccgnnnactgaagcctttgcattggacttggcnttangagtagccnngaaanttcctttttcctggtaaaaggnnanttcctgaaccccgccacgnaattgggcgtaaccgattngcccacccctgtcctycctccctgagactcagcgaaagttraagtnggnttgtgaagatgcaattctaccccgctgcctagacgggaaaagaccccgtgaaccttttackgsagctttgcattgggactttgaagtcacttgtgtaggataggtgggaggcttggaagcagagacgccagtctnctgtggagtcgttccttkaaataccacncctggtgtcntttgaagggaatttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaana

SEQ ID NO: 25 - DNA, 3540 bp, Eikenella corrodens
  MISC_FEATURE (1)..(3540): n can be a, t, c, or g.
cgccgtcacaccatgggagtgggggataccagaagcaggtagggtamccgcaaggagcccgcttgccncggtatgcttcatgactggggtgaagtcgtaacaaggtagccgtaaggggaacctgcggctggatcacctcctttytagagaaaaagtaggggtagagcatccacacctatcggtaatcagatacagatgcgaagagaatgttgggtttgtagctcagctggttagagcacacgcttgataagcgtggggtcggaggttcaagtcctcccagacccaccagtagtcgtagagcggagcagatccgcccaggtactgggggcatagctcagttggtagagcacctgctttgcaagcagggggtcatcggttcgatcccgtttgcctccaccacacaatactttccaaataaaagtatgctaaatatacgagtaagttgctgcatcttgcagtttgcgcggtagtttatttttatttgcgaagtcaaaacgcatcgatctttaacaaattggcaagccgaaatcaacaaacaaacgaagtagggcttatctttgagataagtcatacagaaatttgggtgatgattgtatcgactgaatcctgaaacacaaaaggcaggattcggacacaacaagcagtaagctttatcaaagtagaggccttaagccccagctgtagtcaacgcagcgcaggcgaagtcaaaaaggcacttcaaatgatagagtcaagtgaataagtgcctcaggnggatgccttggcnatgatnggcnacnaangacntgtancccngcnaangctgnntnttttnngntngggggggntattttttttttttttnggnaangacgaagcgntntttttttnnnnnntnggcggngctgatnccccaancnaagnagggggggtntttttttttaagtaatcaacanaaaatttggggattgttngganngattcatcccncanaanagaaaggannggttggnaccaancagnanggttttttttnagtagaggcgnncnnnnnccccccgtagtnanctcagcncagccgnagtnannaannnagntcttttnatanantnnagtnaataantgcancanctggangcntnccnnatgatagnagacgaagaangtgtnngcatncnnnnagcatcgtggaggtggcaanaaaannttgntttgntnatgncggaatgngggggnccacccngcaatgtgtatcntnnnctgaatacatnagnttanntaagngaacccgcngnantgatccatcnaantanccgcnggaaaagaaatcanccganattcnncnantagtggcgagcgaacgcggaggagcctgtatgtkatagttgtcgggataggagaaggaattggaaacttccgccatacgtgggtgagtagccccgtatccgaaattccggcagtggtactaagcatacgacaagtagggcgggacacgagaaatcctgtctsaagatggggggaccatcctccaaggctaaatactcatcatcgatccncnnncgatagtgaaccagtaccgtgagggaaaggcgaaaagaaccccgggaggggagtgaaatagaacctgaaacctgatgcatacaaacagtgggagcactttatggtgtgactgcgtaccttttgtataatgggtcaacgacttacattcagtagcgagcttaaccggataggggaggcgtagggaaaccgagtcttaatagggcgtcatagttgctgggtgtanacccgaaaccgagtgatctatccatggccangttgaaggtgccgtaacaggtactggaggaccgaacccacgcatgttgcaaaatgcggggatgagctgtggatangggtgaaaggctaaacaaactcggagatagcttggttctccccgaaaactatttaggtngtgcctnatgtntcacttncgggggtaaaacactgtnatggctanggggtcattgnanttaccnacctatggnaaactnnnaatacccnaaagtgcnatcatgggaaacnnaccnnnngntgcaaacgtncnttngncngnanggnaacnncccnanacnccctctnnggnccnaatgnnnnannaaggggnaaaanaaanngnagnatgcncntagngcatgnnanggnagngttnngnaggcntnanaaggtgtttngtannnaatgnngnaggnatcanaagtncnaatgttacatnagtancnataaancgggtnaaaagcccgctcncngaaagcccaaggtttcntgngcaacgttcatcggcgcagggtgagtcggcccctaaggcgaggcagagatgcgtagtcgatgggaaacgggttaatattcccgtacttgatttaagtgcgatgtggggacggagaaggttaggtcagcaaactgttggaatagtttgttcaagccggtaggtggaaggattaggcaaatccggttcttcataacaccgagaagtgataacgagtatctacggatacgaagtgactgataccacgcttccaggaaaagccactaagctctagcttaaatcgaaccgtaccgcaaaccgacacaggtgggcaggatgagaattctaaggcgcttgagagaactcgggagaaggaactcggcaaattgatactcgtaacttncgggnagaaggtatgccctctgaggtgaaggatttactccgtaagctttggagggtcgcagcagaatcggtggctgcgactgtttattaaaaacacagcactctgcaaacacgaaagtggacgtatagggtgtgacgcctgcccggtgctggaaggttaattgaagtatgtgagagcatcggatcgaagccccagtaaacggcggccgtaactataacggtncctaangtagcgaanttccttgtcngntaagttccganccgcacnaatggcgtaacgatggccacnctgtnnnctaccgngactcngcgaagttnaagtggttgtgattcatgcaatctactnntgntgatntacctgaaagacccacctgcacctgntaatgnanctttngcnngcgnaacttggctatttcgcctatntgnactgatcacgtgnggaangntcaaaaagcaccagacgnttcctttnttgngnancncgtnccctggtttggccacgntacctgtcncnagaggnaataaccccccgtaccgcnnnnccnnngnntcccggnanattgnganctgggnnngnaaaccccaaagggcnnncnngncttgacatntccanggncnngngnnntnaaaaancnttnnnttttttttaaannnaaagacctncccccctnntttcttccncaangnntnnctttgcaaaganncaaangncnaaggctcncananaagggtntnnccgcggggncttttctnntccnnacnntgngggntggngnngggnnaactnnnaccannctncncnaaaaagggnntntttcttgggggaaacccgnggggtncnnnnaatnccccnattnttcccngnggntccanannaacnc

SEQ ID NO: 26 - DNA, 2958 bp, Bacteroides vulgatus
  MISC_FEATURE (1)..(2958): n can be a, t, c, or g.
nccnnccnnnnnnaanntnntnnnnnnnannttcnnnnnnggggnnnncnagtttnnnnccnnnnnnnannnnnngggcntnnnnnncccntttttttnnnnaccagccncgtcaaagccnatgggagccngggggtmcctgaagntgcgtaaccscgaaggagcgnccctasscgtaaaactggtsmccctggggctaagtcgtnnaamaaggtagccgtnacnnnnnncggaaaggtgcggctggaacacctccctttcctggtangrggacgaaagatcgacaaggttttttgttcctttgtactgctggtgttgtttattcaaagataatgattccgtaatgatacggaagagagaaagtaagaagccgggtctaatcaaacagactaggttgaacttagtcctatagctcagttggttagagcgctacactgataatgtagaggtcggcagttcaactctgcctgggactacgaacggaatcaagttcgggggattagctcagctggctagagcatctgccttgcacgcagagggtcaacggttcgaatccgttattctccactccatcggaaacgtaagaaacgaagatgaacgatctatgacatattgtacaagcaaacagtaattttagtagtaaagagctgaaagtatatatcatncccgctggcacgcaagtgtgagcgnaatggnataaggaaagtaaaraagggcgcatggcggatgccttggctctcggaggcgatgaaggacgtgataagctgcgataagctttgggnaggtgcaaatgacctttgatccagagatttccgaatgggacaacccaatatcttgaagaggtattatccaacgttgttggaggctaacgcagggaactgaaacatcttagtacctgcagcaaaagnaaataaacaananaccccgtnttnnnnnnnannnangngnancanttntncnncnncantaanngggcatgggkgntaagggncngtggccnaanagnagaagaatcnggngcacncntngntaaggtcctgnaatgacagntaagttnanantaanaaagtatgantgctaakacagctaggatgttggnttgtagrakcmsccmttcnatctctnagaagagntgsgtaacagtctacactagtmcgaggaagtcgggcgtggtataataatmgggtattaagttgtctacygaagcagtgggatcattaatatkatcggtaggggagcattccagtcagcgtcgaaggmgtaccgtsaggtattmtnggagcgtctggwaaagcaaatgtwggtataagtaacgataaagggggcgggnnaaaccccctcgccgaaagamtaaggtttcctkatcaacngctaatcggatcagggtcagtcggstcstaaggctcagccgaacggcgatgccgatggcaraaacggttaatattccgttactaccttcaggagtgacgtggmgacgcagtgangntgacagctgccgccatctracggaagntatgatggttgaagggtgtaggagtyratcatggcmggcawatccaccatgagcatnccgwacctgacangtatgccgtcstccttcgggaacaaggctaatagctscgcrataagcmtgctgccgagaaaanctccgmtmaacttmmtcctgcaggymcccstacmgcaaancggacacacgtagtyggngngatkawtmttctaaggcgcctntngagtgattncackgttaarngaactnagggcaaactngcaccctnktdmcttnctgggnnrwaaarrgtcnnccymcycnttttwtnkkkttaggscscagtakaatttcggtccmgscaaccttktwwtaamnnaaaaamamaagnggctgntgccannnaatmagaaaraatccrktwtmcakcctdacmccnttgcccgggtgctnaganngatanggttaagagragatgtcatcgantaararaagcgttgaattkaagccccagtaamcggcggcsgtaactataacggtcctaaggwagcgnaaattncnttgtcgggtaagttccgmcctgcmcsaatggtgtaatgatctgracgctgtctcaaccgtgagctcagtgaaattgtagtatcggtgaagatgccgattacccscratgggacgaaaagaccccgtgamcctttactatatcgttaatcattgaatctgggcacgtgatgtgtaggatakgtcgaagngcntttgaascaggtacrccagtatttgtggagccgctrttgaaatacgaccctttatkktktgctggattytaacgccgtngnaacggrgacattgnattggtgccnggtastnttgrgctggggatggycngcctmacaaaagcgtaacggrgggcytcntaaaggtgtcgccncntccggcsgattggctwrccggccattccttwgaktktaatgngcatwagcggtcgccttgtactsgktakwctragcaagtcnngmtywggwaaaggaaanckrgagncantattttgtgcwtnccnacgccggntgggganacgtttnttttccrtatggttanaggangmcmtttcsccntccnggtannnaanncggatccccanaaagrttawcbccgtttcggtcgggawaanncaaaggncnntgataaannnccnctcccaararcycatatcgacggagtggtttggcacctcgatgtcggctcgtcacatcctggggctggagaaggtcccaagggttgggctgttcgcccattaaagtggcacgcgagctgggttcagaacgtcgtgagacagttcggtctctatctatcgtgggcgcatgaaatttgcgtggctctgacactagtacgagaggaccgtgttggacagacctncggtttgccggttgtgccgccaggtgcattgccgggtatnctaagtncngtatcggataaaa

SEQ ID NO: 27 - DNA, 3908 bp, Branhamella catarrhalis
  MISC_FEATURE (1)..(3908): n can be a, t, c, or g.
ccccgcccgtcncaccatntgggnagttgatctcaccagaaagtggttagcctaacgcaagagggcgatcaccacggtggggtcgatgactggggtgaagtcgtaacaaggtagccgtnaggggaacctgcggctggatcacctccttaacgaagttatctgattggcaagaatccacaacaagttgttctttggtaagatgtttaaaaacggggtctatagctcagttggttagagcaccgtgttgataacgcgggggtcataagttcaagtcttattagacccaccattttggggccatagctcagttggtagagcgcckgccttgcacgcaggaggtcaggagttcgtactctccttggwtccaccaagcaagtttaaacatwraagcatacakaagcaatttaaataagatttcttatttatgcttttattttataaactgacgaagtttataacattatttaacaacatagtatgagtctgggttaattatttaattccaacaaataattaacctggtgtttgtacccaatacaaacaccaaaaaagtaaagagaactgaatcaagcgtaaacataggtgaatcgttacacattacccatacacaccaaagacttcctagaagtcagactacttggggttgtatggtcaagtaatgaagtgcacatggtggatgccttgncactcagaggcgatgaaanacgtcataacctgcnataagcntnngngaggtggcaatntccccgtgacccnncgatttcntgaatggggaaacccaaccnacatnantttggtttattacccaanntncttgngtaangcatnncccnnnnttttnggngctaaanccntngggnaaangccntncnttncccnnaaaaggnnnnngnttnntttncntggncccccccgncnnnttttcnnngcagncnaaaganncccnnnaanannnnnttttnannccnngttttatttnaaaangnngnngattttatcncttttttncannccangagtngggntnggntattttnttnntttcccataannaanttcnngtnggnttnnnccccaacccccncccnannaaagaaaagtgnttnaagcgtnggnanannngngncgttntttntcacntacccanaccacncaantcttttnagnaagcnnacttntnntggnatngtcgngtaatgaagtgnanatcnnggnngcctncccngncanannngatgaaanaagnnatngcatncnatnagaancggtgnggtggcnnnancatntnnnnngccnanntntttttgngggggnccanccancatnantnggttnttanncagcncantgtgttnggnannccnncnggagtaanncatcncantacccccccnannagacatcanatnatattcngtcagtagcngcgagcgaacacggaggagccgatyaattttwywgtagcaanatggcgtgggargagccaaccatastaggtgagtagtcctgtatgcgaaactgtttaagcgacatattaagtagggcggracacgagaaattactgttctgaagtatggggggaccatcctcccaaggctaaatactacctgactsaccgnatagtgaaccagwaccgtgagggaaaggcgaaaagatatcttcgcgctgttaggsgcagtgaaatagaacctgaaaccgtgtgcatacaagacagtcggagcccgaccacttaatcgttttgaggataacaatgaatttttactacgtctatgtcatacaaaacgtggatagtcatgatgagttttatgttggttttaccaccaatttaaaacaaagaattgatgaccataataatggcacaacttactcaaccagagcaagaacatggcgtattgtttattgtgaagcctacattaatgaacaagtggcaagaaaacgagagcgaaccataaaaaataatggtcgtatgagaactttcttgatgagatcgtgtcaaatcacaatttgacaatcagaacgattaagtggtcgggtswcggcgtaccttttgtataatggggtcagcgancttatattctgtmncaanggttaacncggaatwggggwgccgtanggataaccgagntttctntaantaggkcgaatgagtttgtcagggtatttacccnaaaccgantgatctatccatgancnggttngananagtgccatnagancaggcncctkgaggaccnacccctctgtaccctnttannantataagtaanccgtaatagctacactagtncgagtcggcctgcgcggaaaaatgtaacggggctcaaaaytaggagccgaagctgcggatttaattgttttcaattaaagtggtaggggagcgttgtgtaagcctgtgaaggtgcactgtaaggtgtgctggaggtatcacaagagcgaatgctgacgtgagtaacgacaaaacgggtgaaaagcccgttcgccggaagaccaagggttccagtccaacgttaatcggggctgggtgagtcgacccctaaggcgaggccgaaaggcgtastcgatgggaaatcggttaatattccgatacttgtttatgatgcgatggagggacggagaaggttatgccagcctggckatggttgtccaggtggaaggatgtagtnctatsactgaktakgcataatccgctcggwtattaatgakatctgwtascamgccagtttcacctggcaaagtggyaaatacsctgcttycasgaaaagcttctamkcsatagtnctataacacgaatcgtacccgaatatccgacmcaggtggycaggtmgagamtacgnctncnaaggckctnnntgagmgtaactctngcntagrasgnaactcrsgcwwaatggtacccgctnacnacyycgkgagaaasktacgctgaccgatggtkataagacnttgctsttntngagcnknangtnnatggcagtncnnttgctttagataccaggcttgcnnnntgcaactgtttattaaawacacagcmcttcttntngctaaacacgaaangctgsmcstwtmgggtgtgattnggncctgcccggtgctggaaggttaattgatggggttagcgtaagcgaagtttytgatcgaagccccagtaaacggcggccgtaactataacggtnctaaggtaannaanttncttgtngggtaatcttccgtcctncncgantgncatattgatggcagcgctnnctccagnagactactcggnganaatcaaacccncnnntnataaatgttnngnnccctgcntnatnnnangaaacnnnccccngngnannctttnacnacnanntntttnnnaaannannctncncaccctnacacttctnatacnnnnntcgngnntcggaanncttttcnnaatnnnanttacttcccactcgtacggntntntnatncccncacnctaanncntngcccacgcnnttgtgnngntnntnntggtactttctnacncantnagacnanccannccaangtccnnnanggancccctcntnnnngnncnaanttnacnntttnngatnnccnacannnaancnccccngtnnnaaaanttttngacnnnaatgagctactcgtaacncgctanctacngncctcancnntnantcnnnatnaagtgtggtnncacnnnnctnnaaatnntaattncncntnntntncnnntntttctctctnntaccttcgnnncnnnatananntttnngctcntctcnaattacngtgcntnttacatacnnnatntacaatntcnngcncgctnctngtnttactnngnnttnccnctnnncnactnancattcntatacnngttttgnnccttntccntnactntacctacgnnnttcnnt

SEQ ID NO: 28 - DNA, 3166 bp, Sutterella wadsworthia
  rRNA (1)..(3166): 23S rRNA and Internal Transcribed Spacer Region
ggcgcccaccggggttcaggctgggggtcaactttaacaaggtagccctccggaaggtccggctggatcacctcctttaaacagagtcggccttgaagccgccgcacctacggagtgggcttcaccaggaatcgttggccctcccgaacaaggaggggggttctaggtgggcttcttaaaggatggtaagtagaagcaagggggccagtacttcaagtcccgccagaatcacctcttttcaagaaagtgggccttatgcagaatcgacatcaagaactgttgggggcgttcacacttatcgagttctgttgaattcaattcgcccgattctttaaaaatttggaaaagtaaagcaacagaagctgctgctttcgaagggttgagatgctctttgaaggcgcaaaaaggcagcttcaggggttgtgattgcattcacgaacccgattcattgaaagttcttcaaagaaccgacatgattcaagagaaatacagcgttataggtcgtcttgtgctttgaaggcaaaaggcttcagagttataggatcaagcgactaagtgtatgtggtggatgccttggcaaaatctacatggtcgaagaaggtacgtcgtcatcctgtcgaaaagccgtggggagcctggcaagcgagccactgatgccacggatgtccgaatgagggaactcacccgcaagggtatcttgaagtgaatacatagcttcaagaggcgaacgcagcgaactgaaacatctaagtagctgcaggaaaagaaatcaaccgagattctgaaagtagcggcgagcgaaattggaagagcctgcaagtgatattgaattcgatactggaacggtctggaaagtccggctacagagggtgacagccccgtacgggaaattgggttcaaggtactgagcttgcgagaagtagagcgggacacgtgaatcatccttgctcgaagatggggggaccatcctccaaggctaaatactcgtaatcgaccgatagcgaacccagtactgtgaaggaaaggcgaaaagaaccccgggaggggagtgaaatagatcctgaaaccgcatacatacaaacagtaggagcccgcaagggtgactgcgtaccttttgcataatgggtcagcgacttacgttcaatggcaagcttaaccgagtaggggaggcgcagcgaaagcgagtccgaacagggcgatatgagtcgttgggcgtagacccgaaaccagatgatctatccatggccaggctgaaggtgtggtaacacacactggagggccgaaccgactagtgttgcaaaattagcggatgagctgtggataggggtgaaaggctaaacaaatctggagatagctggttctccccgaaaactattgaggtagtgcctcgtgtatggctccagggggtagagcactgttatggctagggggacatggcgtcttaccaaaccatggcaaactccgaatacttggaagtctgagcacgggagacagagcaccgggtgctaacgtccgggactcaaaagggaaacaacccagaccgccggctaaggtccctaatattgactaagtggaaaaacgaagtgggaaggctaaaacagtcaggaagttggcttaaaccaccccaattcactttcaaagaaaccgtaatagctaactgatcgagtcttcctgcgccggaaaaatgtaacggggctaagtcaataaccgaagccgcggattcgcagcaatgcagtggtaggggaagcgttctgtaagcctgcaaggcggatccgcgaggaccgctggaggtatcagaagtgcgaatgctgacatgagtaacgttaaagcgggtgaaaagcccctcgccagcccaaggggggacggagtcctgaaggtcatccgggtgttggacatcccggtttacgagcgtaggaggttgacaggcaaatccgtcaacggatctccaaggcgaaggatcgaggttgagagatcaactgaagtgactgaatgggcttccaggaaaagcctctaagcatcagcttggaattgaccgtaccgcaaaccgacactggtgggcgagctgaatatgatcaggcgcttgagagaacacaggagaaggaactcggcaaattgacaccgtaacttcgggataaggtgtgcctttgtagtgtgaagagagaaacactccgagcatgaagaggcctcagagaatcggtggctgcaactgtttactaaaaacacagcactcctgcaaagatgaaagtacgacgtatagggtgtgatgcctgcccggtgctggaagattaattgatggggtgcaagctcctgatcgaagtcccagtaaacggcggccgtaactataacggtccctaaaggagcgaaattccttgtcgggcaagttccgacctgcagaatggcaaatgatggccacactgtctcctcctagactcagtaagttgaaatgtttgtgatgatgcaatctcccccggctagacggaaagaccccataaccttactgcagctttgcattggactttgataccgatctgtgtaggataggtgggaggcagtgaagctaggacgccagtcttagcggagccaaccttcaaataccaccctgatttgtttgaggttactaacctaggcagacgtaagccgattgcggggcaccgtgcatcctaggcagtttgatgggcggtctcctcctaaagggtaacggaggagtgcgaaggtacgctaggtacggtcggaaatcgtgctgatagtgcaatggcaaaagcgtgcttgactgcgagaccgacaagtcgagcagatacgaaagtaggtcatagtgatccggtggctctgcatggaagggccatcgctcaacggataaaaggtactctggggataacaggctgataccgcccaagagttcatatcgacggcggtgtttggcacctcgatgtcggctcatctcatcctggggctgtagccggtcccaagggtatggctgttcgccatttaaagaggtacgtgagctgggtttaaaacgtcgtgagacagttttgtccctatctgccgtgggcgttggaagattgacgggggctgctcctagtacgagaggaccggagtggactcacctctggtgtaccggttgtcacgccagggcatcgccgggtaggctatgtgggaaaggaaaaca

SEQ ID NO: 29 - DNA, 15 bp, Artificial Sequence
  Probe Acti004
tgatggaacttgctt

SEQ ID NO: 30 - DNA, 15 bp, Artificial Sequence
  Probe Acti23S01
agggcacacataatg

SEQ ID NO: 31 - DNA, 15 bp, Artificial Sequence
  Probe Acti23S02
acgctgttgttggtg

SEQ ID NO: 32 - DNA, 15 bp, Artificial Sequence
  Probe Acti3(Acti I)
atacacagtacttcg

SEQ ID NO: 33 - DNA, 15 bp, Artificial Sequence
  Probe Acti002
atagtgttgcaaggc

SEQ ID NO: 34 - DNA, 15 bp, Artificial Sequence
  Probe Acti003
tgaaaagccagggga

SEQ ID NO: 35 - DNA, 15 bp, Artificial Sequence
  Probe Acti1(Acti 23)
gggcacacataatga

SEQ ID NO: 36 - DNA, 15 bp, Artificial Sequence
  Probe Acti2(Acti M)
cggggtactctatac

SEQ ID NO: 37 - DNA, 15 bp, Artificial Sequence
  Probe Acti23S03
gtaggtatgtatctt

SEQ ID NO: 38 - DNA, 15 bp, Artificial Sequence
  Probe Acti23S04
tactgagatccgata

SEQ ID NO: 39 - DNA, 15 bp, Artificial Sequence
  Probe Acti001
aggtattgcaacatg

SEQ ID NO: 40 - DNA, 15 bp, Artificial Sequence
  Probe ActiIT01
cagaagtagctgcct

SEQ ID NO: 41 - DNA, 15 bp, Artificial Sequence
  Probe ActiIT02
agaagtagctgccta

SEQ ID NO: 42 - DNA, 15 bp, Artificial Sequence
  Probe ActiIT03
gaagtagctgcctaa

SEQ ID NO: 43 - DNA, 15 bp, Artificial Sequence
  Probe ActiIT04
aagtagctgcctaac

SEQ ID NO: 44 - DNA, 15 bp, Artificial Sequence
  Probe ActiIT05
agtagctgcctaact

SEQ ID NO: 45 - DNA, 15 bp, Artificial Sequence
  Probe Anas001
tgactcgtgcccatg

SEQ ID NO: 46 - DNA, 15 bp, Artificial Sequence
  Probe Anas002
taccggggttaaaag

SEQ ID NO: 47 - DNA, 15 bp, Artificial Sequence
  Probe Anas003
atcagtgatctgaga

SEQ ID NO: 48 - DNA, 15 bp, Artificial Sequence
  Probe Anas004
gagacgaagcaccat

SEQ ID NO: 49 - DNA, 15 bp, Artificial Sequence
  Probe Anas011
agttgatacaggtag

SEQ ID NO: 50 - DNA, 15 bp, Artificial Sequence
  Probe Anas013
ggccccatccggggt

SEQ ID NO: 51 - DNA, 15 bp, Artificial Sequence
  Probe Anas23S03
cagttggaagcagag

SEQ ID NO: 52 - DNA, 15 bp, Artificial Sequence
  Probe Anas005
gttcttgattcattg

SEQ ID NO: 53 - DNA, 15 bp, Artificial Sequence
  Probe Anas008
cagcccaaaagttga

SEQ ID NO: 54 - DNA, 15 bp, Artificial Sequence
  Probe Anas009
aaactgcagggcaca

SEQ ID NO: 55 - DNA, 15 bp, Artificial Sequence
  Probe Anas010
atactacctgacgac

SEQ ID NO: 56 - DNA, 15 bp, Artificial Sequence
  Probe Anas1(Anas7)
aaagtgcagggcaca

SEQ ID NO: 57 - DNA, 15 bp, Artificial Sequence
  Probe Anas2(AnasM)
tggattgtggtgaaa

SEQ ID NO: 58 - DNA, 15 bp, Artificial Sequence
  Probe Anas3(Anas7)
tagcgttctgcgagg

SEQ ID NO: 59 - DNA, 15 bp, Artificial Sequence
  Probe Anas4(AnasM)
ttaaaagactggtat

SEQ ID NO: 60 - DNA, 15 bp, Artificial Sequence
  Probe Anas006
atccaatcatgatca

SEQ ID NO: 61 - DNA, 15 bp, Artificial Sequence
  Probe Anas007
aagcatgaaagcgca

SEQ ID NO: 62 - DNA, 15 bp, Artificial Sequence
  Probe Anas014
gaggcgggagccgag

SEQ ID NO: 63 - DNA, 15 bp, Artificial Sequence
  Probe Anas23001
ccccatccggggttg

SEQ ID NO: 64 - DNA, 15 bp, Artificial Sequence
  Probe Anas23S01
tggcgtcaggaggcg

SEQ ID NO: 65 - DNA, 15 bp, Artificial Sequence
  Probe Anas23S02
ataaggggcgcttga

SEQ ID NO: 66 - DNA, 15 bp, Artificial Sequence
  Probe Anas23S04
tcacacgcaagtgtg

SEQ ID NO: 67 - DNA, 15 bp, Artificial Sequence
  Probe Anas23S05
gctgagacgaagcac

SEQ ID NO: 68 - DNA, 15 bp, Artificial Sequence
  Probe Anas23S06
ataccggggttaaaa

SEQ ID NO: 69 - DNA, 15 bp, Artificial Sequence
  Probe Anas23S03
cagttggaagcagag

SEQ ID NO: 70 - DNA, 15 bp, Artificial Sequence
  Probe Anas012
tagcgttctgcgagg

SEQ ID NO: 71 - DNA, 15 bp, Artificial Sequence
  Probe AnasIT001
acagcgcagcatgtg

SEQ ID NO: 72 - DNA, 15 bp, Artificial Sequence
  Probe AnasIT002
aattagcaactattt

SEQ ID NO: 73 - DNA, 15 bp, Artificial Sequence
  Probe AnasIT01
cttccctcagtgatt

SEQ ID NO: 74 - DNA, 15 bp, Artificial Sequence
  Probe AnasIT02
ttccctcagtgattc

SEQ ID NO: 75 - DNA, 15 bp, Artificial Sequence
  Probe AnasIT03
tccctcagtgattca

SEQ ID NO: 76 - DNA, 15 bp, Artificial Sequence
  Probe AnasIT04
ccctcagtgattcaa

SEQ ID NO: 77 - DNA, 15 bp, Artificial Sequence
  Probe AnasIT05
cctcagtgattcaag

SEQ ID NO: 78 - DNA, 15 bp, Artificial Sequence
  Probe Bf 011
gtcgaacctgacagt

SEQ ID NO: 79 - DNA, 15 bp, Artificial Sequence
  Probe Bacf1(Bf23)
ggtaaccgaagcgta

SEQ ID NO: 80 - DNA, 15 bp, Artificial Sequence
  Probe Bacf2(Bf)
ctcggaaaacggtaa

SEQ ID NO: 81 - DNA, 15 bp, Artificial Sequence
  Probe Bf 001
agcgatgttgaaaac

SEQ ID NO: 82 - DNA, 15 bp, Artificial Sequence
  Probe Bf 002
tcaaccatctatagc

SEQ ID NO: 83 - DNA, 15 bp, Artificial Sequence
  Probe Bf 003
aacaagagaaaaaca

SEQ ID NO: 84 - DNA, 15 bp, Artificial Sequence
  Probe Bf 004
cgataccgcgaccta

SEQ ID NO: 85 - DNA, 15 bp, Artificial Sequence
  Probe Bf 005
tatatcgaaccattt

SEQ ID NO: 86 - DNA, 15 bp, Artificial Sequence
  Probe Bf 006
gaatctggcgataaa

SEQ ID NO: 87 - DNA, 15 bp, Artificial Sequence
  Probe Bf 007
tgcaaatgacctttg

SEQ ID NO: 88 - DNA, 15 bp, Artificial Sequence
  Probe Bf 008
caacttggttggagg

SEQ ID NO: 89 - DNA, 15 bp, Artificial Sequence
  Probe Bf 009
acccatgttacggca

SEQ ID NO: 90 - DNA, 15 bp, Artificial Sequence
  Probe Bf 010
agttgacctaacgaa

SEQ ID NO: 91 - DNA, 15 bp, Artificial Sequence
  Probe Bf 012
tgaacggatctgtgt

SEQ ID NO: 92 - DNA, 15 bp, Artificial Sequence
  Probe Bacf3(Bf I)
ggttcagatcctttt

SEQ ID NO: 93 - DNA, 15 bp, Artificial Sequence
  Probe Car 006
aaccctggtgaaggg

SEQ ID NO: 94 - DNA, 15 bp, Artificial Sequence
  Probe Car 007
atatgaagatatgtg

SEQ ID NO: 95 - DNA, 15 bp, Artificial Sequence
  Probe Car 008
tagattgacttacgg

SEQ ID NO: 96 - DNA, 15 bp, Artificial Sequence
  Probe Car 009
gtaaagttttactac

SEQ ID NO: 97 - DNA, 15 bp, Artificial Sequence
  Probe Car2(CarM)
ccagcacactgttgg

SEQ ID NO: 98 - DNA, 15 bp, Artificial Sequence
  Probe Car3(CarI)
aaagagagaacagca

SEQ ID NO: 99 - DNA, 15 bp, Artificial Sequence
  Probe Car 001
ttggcgacaacaggc

SEQ ID NO: 100 - DNA, 15 bp, Artificial Sequence
  Probe Car 002
gccccgggaagctga

SEQ ID NO: 101 - DNA, 15 bp, Artificial Sequence
  Probe Car 003
tagactgcggaagcg

SEQ ID NO: 102 - DNA, 15 bp, Artificial Sequence
  Probe Car 004
aattaagttgcgtat

SEQ ID NO: 103 - DNA, 15 bp, Artificial Sequence
  Probe Car1(Car23)
ccatacacaatgaat

SEQ ID NO: 104 - DNA, 15 bp, Artificial Sequence
  Probe Car 005
tactcgttgtcgacc

SEQ ID NO: 105 - DNA, 15 bp, Artificial Sequence
  Probe Chr23S04
ggcatatttagatga

SEQ ID NO: 106 - DNA, 15 bp, Artificial Sequence
  Probe Chr001
cttaggtgatcactt

SEQ ID NO: 107 - DNA, 15 bp, Artificial Sequence
  Probe Chr003
taaccccttagatta

SEQ ID NO: 108 - DNA, 15 bp, Artificial Sequence
  Probe Chr004
tcaaacctcaaacta

SEQ ID NO: 109 - DNA, 15 bp, Artificial Sequence
  Probe Chr005
aagaaatcgaagaga

SEQ ID NO: 110 - DNA, 15 bp, Artificial Sequence
  Probe Chry1(Chry23)
atttagatgataaat

SEQ ID NO: 111 - DNA, 15 bp, Artificial Sequence
  Probe Chry2(Chry7)
taatcttactagcga

SEQ ID NO: 112 - DNA, 15 bp, Artificial Sequence
  Probe Chr23S05
atcgtgaggttacga

SEQ ID NO: 113 - DNA, 15 bp, Artificial Sequence
  Probe Chry3(ChryI)
tccttgagtgcagag

SEQ ID NO: 114 - DNA, 15 bp, Artificial Sequence
  Probe Chr002
agcacagctttggtt

SEQ ID NO: 115 - DNA, 15 bp, Artificial Sequence
  Probe C.ramosa04
ccagtgtgtgaggag

SEQ ID NO: 116 - DNA, 15 bp, Artificial Sequence
  Probe C.ramo 004
cccgggaaggggagt

SEQ ID NO: 117 - DNA, 15 bp, Artificial Sequence
  Probe C.ramosa01
ggtgaagtattagta

SEQ ID NO: 118 - DNA, 15 bp, Artificial Sequence
  Probe C.ramosa02
atgtacaggcatagg

SEQ ID NO: 119 - DNA, 15 bp, Artificial Sequence
  Probe C.ramosa03
tgagagacatgcacg

SEQ ID NO: 120 - DNA, 15 bp, Artificial Sequence
  Probe C.ramosa05
gtattggagttgcta

SEQ ID NO: 121 - DNA, 15 bp, Artificial Sequence
  Probe C.ramo 001
tagttgatgatagta

SEQ ID NO: 122 - DNA, 15 bp, Artificial Sequence
  Probe C.ramo 002
gcttatctgtggatg

SEQ ID NO: 123 - DNA, 15 bp, Artificial Sequence
  Probe C.ramo 003
ggaatccctccttgt

SEQ ID NO: 124 - DNA, 15 bp, Artificial Sequence
  Probe Com 004
tagggcgtccagtcg

SEQ ID NO: 125 - DNA, 15 bp, Artificial Sequence
  Probe Com 005
cgcagagtacagctt

SEQ ID NO: 126 - DNA, 15 bp, Artificial Sequence
  Probe Com 006
gtaccgatgtgtagt

SEQ ID NO: 127 - DNA, 15 bp, Artificial Sequence
  Probe Com 007
gaacttgaacaaagg

SEQ ID NO: 128 - DNA, 15 bp, Artificial Sequence
  Probe Coma2(Com 7)
tgagctagagaaaag

SEQ ID NO: 129 - DNA, 15 bp, Artificial Sequence
  Probe Coma3(Com M)
atccgccgggcttag

SEQ ID NO: 130 - DNA, 15 bp, Artificial Sequence
  Probe Coma1(Com23)
aaaaccgactggtgg

SEQ ID NO: 131 - DNA, 15 bp, Artificial Sequence
  Probe Com 001
gctgacggaaagaga

SEQ ID NO: 132 - DNA, 15 bp, Artificial Sequence
  Probe Com 002
ctcttgacagaaatg

SEQ ID NO: 133 - DNA, 15 bp, Artificial Sequence
  Probe Com 003
aagaattcattcaca

SEQ ID NO: 134 - DNA, 15 bp, Artificial Sequence
  Probe Coma4(Com I)
acgcgcgaggtgaga

SEQ ID NO: 135 - DNA, 15 bp, Artificial Sequence
  Probe C.diph003
accatcttcccaagg

SEQ ID NO: 136 - DNA, 15 bp, Artificial Sequence
  Probe C.dipht 01
taactagataagaaa

SEQ ID NO: 137 - DNA, 15 bp, Artificial Sequence
  Probe C.diph001
accacgcagcagttt

SEQ ID NO: 138 - DNA, 15 bp, Artificial Sequence
  Probe C.diph002
cgagtcggtagggta

SEQ ID NO: 139 - DNA, 15 bp, Artificial Sequence
  Probe C.diph004
tgtttgttctttgat

SEQ ID NO: 140 - DNA, 15 bp, Artificial Sequence
  Probe C.diph005
aaaatcagaaaaaca

SEQ ID NO: 141 - DNA, 15 bp, Artificial Sequence
  Probe C.diph006
ggaaaatcagaaaaa

SEQ ID NO: 142 - DNA, 15 bp, Artificial Sequence
  Probe Ko001
gaacgttactaacgc

SEQ ID NO: 143 - DNA, 15 bp, Artificial Sequence
  Probe Koxy1(Ko M)
ccggaacgttactaa

SEQ ID NO: 144 - DNA, 15 bp, Artificial Sequence
  Probe Koxy3(Ko M)
aagagcgccagctca

SEQ ID NO: 145 - DNA, 15 bp, Artificial Sequence
  Probe Ko001
tttgaagttctaact

SEQ ID NO: 146 - DNA, 15 bp, Artificial Sequence
  Probe Ko002
aagagcgccagctac

SEQ ID NO: 147 - DNA, 15 bp, Artificial Sequence
  Probe Ko002
tatctaccgcgggcg

SEQ ID NO: 148 - DNA, 15 bp, Artificial Sequence
  Probe Ko003
gatgaagacctcaaa

SEQ ID NO: 149 - DNA, 15 bp, Artificial Sequence
  Probe Ko003
ttacgggttgtcatg

SEQ ID NO: 150 - DNA, 15 bp, Artificial Sequence
  Probe Koxy2(Ko I)
cgcgacacgacgatg

SEQ ID NO: 151 - DNA, 15 bp, Artificial Sequence
  Probe Ochr04
ggaccaggccagtgg

SEQ ID NO: 152 - DNA, 15 bp, Artificial Sequence
  Probe Ochr05
gaccaggccagtggc

SEQ ID NO: 153 - DNA, 15 bp, Artificial Sequence
  Probe Ochr004
gttgattgacacttg

SEQ ID NO: 154 - DNA, 15 bp, Artificial Sequence
  Probe Ochr005
taccgctcacgagcc

SEQ ID NO: 155 - DNA, 15 bp, Artificial Sequence
  Probe Ochr007
gggtccggaggttca

SEQ ID NO: 156 - DNA, 15 bp, Artificial Sequence
  Probe Ochr1(Ochr 23-1)
cggcgcgtgagcgag

SEQ ID NO: 157 - DNA, 15 bp, Artificial Sequence
  Probe Ochr2(Ochr 7-1)
gaacacctgttgtcc

SEQ ID NO: 158 - DNA, 15 bp, Artificial Sequence
  Probe Ochr4(Ochr 23-2)
tcgtcggcccatgtg

SEQ ID NO: 159 - DNA, 15 bp, Artificial Sequence
  Probe Ochr5(Ochr 7-2)
ttagtgtatcgagca

SEQ ID NO: 160 - DNA, 15 bp, Artificial Sequence
  Probe Ochr002
cttcgggctgatgat

SEQ ID NO: 161 - DNA, 15 bp, Artificial Sequence
  Probe Ochr003
aggccagtcagcctg

SEQ ID NO: 162 - DNA, 15 bp, Artificial Sequence
  Probe Ochr006
gttggttctgataca

SEQ ID NO: 163 - DNA, 15 bp, Artificial Sequence
  Probe Ochr008
cagttggaagcagag

SEQ ID NO: 164 - DNA, 15 bp, Artificial Sequence
  Probe Ochr3(Ochr I)
gatccgacgatttcc

SEQ ID NO: 165 - DNA, 15 bp, Artificial Sequence
  Probe Ochr001
taggaaagacgcagt

SEQ ID NO: 166 - DNA, 15 bp, Artificial Sequence
  Probe Pep002
actagggagagctca

SEQ ID NO: 167 - DNA, 15 bp, Artificial Sequence
  Probe Pep003
gcttagtaaagcaag

SEQ ID NO: 168 - DNA, 15 bp, Artificial Sequence
  Probe Pep004
tactaacatgtgacc

SEQ ID NO: 169 - DNA, 15 bp, Artificial Sequence
  Probe Pep005
aagcagagagagctc

SEQ ID NO: 170 - DNA, 15 bp, Artificial Sequence
  Probe Pep006
cgaacggtgaggccg

SEQ ID NO: 171 - DNA, 15 bp, Artificial Sequence
  Probe Pep007
gtagatgttgattat

SEQ ID NO: 172 - DNA, 15 bp, Artificial Sequence
  Probe Pep23S02
gtcgaatcatctggg

SEQ ID NO: 173 - DNA, 15 bp, Artificial Sequence
  Probe Pep23S03
taaaacgtatcggat

SEQ ID NO: 174 - DNA, 15 bp, Artificial Sequence
  Probe Pep1(Pep23)
actaaataaaccagg

SEQ ID NO: 175 - DNA, 15 bp, Artificial Sequence
  Probe Pep2(PepM)
ataatcaacatctac

SEQ ID NO: 176 - DNA, 15 bp, Artificial Sequence
  Probe Pep3(PepI)
tctgtataatagttc

SEQ ID NO: 177 - DNA, 15 bp, Artificial Sequence
  Probe Pep001
agaagctgatacgtc

SEQ ID NO: 178 - DNA, 15 bp, Artificial Sequence
  Probe Por 003
agttggtgagcgagc

SEQ ID NO: 179 - DNA, 15 bp, Artificial Sequence
  Probe Por23S08
ctgagctgtcgtgca

SEQ ID NO: 180 - DNA, 15 bp, Artificial Sequence
  Probe Por 001
gtttttgtgagtgga

SEQ ID NO: 181 - DNA, 15 bp, Artificial Sequence
  Probe Por 002
tgatgggtggggttg

SEQ ID NO: 182 - DNA, 15 bp, Artificial Sequence
  Probe Por1(Por7)
gttggatgttatcat

SEQ ID NO: 183 - DNA, 15 bp, Artificial Sequence
  Probe Por2(PorM)
cgggcagctaaaacc

SEQ ID NO: 184 - DNA, 15 bp, Artificial Sequence
  Probe Por 004
acctatgagtactat

SEQ ID NO: 185 - DNA, 15 bp, Artificial Sequence
  Probe Por3(PorI)
tgtttgtgcgacgtg

SEQ ID NO: 186 - DNA, 15 bp, Artificial Sequence
  Probe P.anae003
aggaggaagagaaag

SEQ ID NO: 187 - DNA, 15 bp, Artificial Sequence
  Probe P.anae004
gcgaaaggaaaagag

SEQ ID NO: 188 - DNA, 15 bp, Artificial Sequence
  Probe P.anae001
tgcattactaagtga

SEQ ID NO: 189 - DNA, 15 bp, Artificial Sequence
  Probe P.anae002
gtaaggtcgataccc

SEQ ID NO: 190 - DNA, 15 bp, Artificial Sequence
  Probe P.magn002
catgcaacgatccgt

SEQ ID NO: 191 - DNA, 15 bp, Artificial Sequence
  Probe P.magn001
tagttgaaaatagta

SEQ ID NO: 192 - DNA, 15 bp, Artificial Sequence
  Probe P.magn003
cagcacgtgaatatg

SEQ ID NO: 193 - DNA, 15 bp, Artificial Sequence
  Probe f.necro01
tttcgcagacgtaag

SEQ ID NO: 194 - DNA, 15 bp, Artificial Sequence
  Probe f.necro02
gttttcttgcgctgt

SEQ ID NO: 195 - DNA, 15 bp, Artificial Sequence
  Probe f.necro03
ccgtattcatgtcaa

SEQ ID NO: 196 - DNA, 15 bp, Artificial Sequence
  Probe f.necro05
ctgcaagctatttcg

SEQ ID NO: 197 - DNA, 15 bp, Artificial Sequence
  Probe f.necro06
cagacgtaagcaaag

SEQ ID NO: 198 - DNA, 15 bp, Artificial Sequence
  Probe f.necro07
cctgtattggtagtt

SEQ ID NO: 199 - DNA, 15 bp, Artificial Sequence
  Probe P.vulga004
agaggaggcttagtg

SEQ ID NO: 200 - DNA, 15 bp, Artificial Sequence
  Probe P.vulga01
atacgtgttatgtgc

SEQ ID NO: 201 - DNA, 15 bp, Artificial Sequence
  Probe P.vulga03
atatccaatggatat

SEQ ID NO: 202 - DNA, 15 bp, Artificial Sequence
  Probe P.vulga006
ggaaacccaatatcc

SEQ ID NO: 203 - DNA, 15 bp, Artificial Sequence
  Probe P.vulga007
gggaaacccaatatc

SEQ ID NO: 204 - DNA, 15 bp, Artificial Sequence
  Probe P.vulga008
cactgtttcgactag

SEQ ID NO: 205 - DNA, 15 bp, Artificial Sequence
  Probe P.vulga02
ctcacacagacttgt

SEQ ID NO: 206 - DNA, 15 bp, Artificial Sequence
  Probe P.vulga005
gtgggttgcaaaata

SEQ ID NO: 207 - DNA, 15 bp, Artificial Sequence
  Probe E.aero01
ttccgacggtacagg

SEQ ID NO: 208 - DNA, 15 bp, Artificial Sequence
  Probe E.aero03
gtatcagtaagtgcg

SEQ ID NO: 209 - DNA, 15 bp, Artificial Sequence
  Probe E.aero04
ttatccaggcaaatc

SEQ ID NO: 210 - DNA, 15 bp, Artificial Sequence
  Probe E.aero02
gagcggggtagttga

SEQ ID NO: 211 - DNA, 15 bp, Artificial Sequence
  Probe E.aero005
aatcaaggctgaggt

SEQ ID NO: 212 - DNA, 15 bp, Artificial Sequence
  Probe S.mutan001
taggtattctctcct

SEQ ID NO: 213 - DNA, 15 bp, Artificial Sequence
  Probe S.mutans01
gaaaaacgaagggta

SEQ ID NO: 214 - DNA, 15 bp, Artificial Sequence
  Probe S.mutans02
atgactacgtggtcg

SEQ ID NO: 215 - DNA, 15 bp, Artificial Sequence
  Probe S.mutans03
gtaatgcaagatatc

SEQ ID NO: 216 - DNA, 15 bp, Artificial Sequence
  Probe S.mutans004
ttgtatgcgcggtag

SEQ ID NO: 217 - DNA, 15 bp, Artificial Sequence
  Probe S.mutans005
cgaaaagtatcgggg

SEQ ID NO: 218 - DNA, 15 bp, Artificial Sequence
  Probe K.king02
ggttagcaaactgtt

SEQ ID NO: 219 - DNA, 15 bp, Artificial Sequence
  Probe K.king03
ccagtaggtggaaag

SEQ ID NO: 220 - DNA, 15 bp, Artificial Sequence
  Probe K.king04
aacaccgagacgtga

SEQ ID NO: 221 - DNA, 15 bp, Artificial Sequence
  Probe K.king09
tattcaatgcgatgg

SEQ ID NO: 222 - DNA, 15 bp, Artificial Sequence
  Probe K.king01
tgattcaatgcgatg

SEQ ID NO: 223 - DNA, 15 bp, Artificial Sequence
  Probe K.king05
tataattaaacgcat

SEQ ID NO: 224 - DNA, 15 bp, Artificial Sequence
  Probe K.king06
aatgttgtcgatttg

SEQ ID NO: 225 - DNA, 15 bp, Artificial Sequence
  Probe K.king07
aggcaacaaatcgaa

SEQ ID NO: 226 - DNA, 15 bp, Artificial Sequence
  Probe K.king08
tatcaactaatcttg

SEQ ID NO: 227 - DNA, 15 bp, Artificial Sequence
  Probe B.ovatus01
tagaaggaagcattc

SEQ ID NO: 228 - DNA, 15 bp, Artificial Sequence
  Probe B.ovatus02
ccaatgttgttacgg

SEQ ID NO: 229 - DNA, 15 bp, Artificial Sequence
  Probe B.ovatus005
tgtaggaccacgatg

SEQ ID NO: 230 - DNA, 15 bp, Artificial Sequence
  Probe B.ovatus003
ggaccgaaccgataa

SEQ ID NO: 231 - DNA, 15 bp, Artificial Sequence
  Probe B.ovatus004
ggacacgaggaatct

SEQ ID NO: 232 - DNA, 15 bp, Artificial Sequence
  Probe B.ovatus006
tgaaggaatgtcatc

SEQ ID NO: 233 - DNA, 15 bp, Artificial Sequence
  Probe B.ovatus007
cccacgatagataga

SEQ ID NO: 234 - DNA, 15 bp, Artificial Sequence
  Probe B.thetaio006
gctaacgcagggaac

SEQ ID NO: 235 - DNA, 15 bp, Artificial Sequence
  Probe B.thetaio01
ttattgtactactgg

SEQ ID NO: 236 - DNA, 15 bp, Artificial Sequence
  Probe B.thetaio02
atcaggtagacaagg

SEQ ID NO: 237 - DNA, 15 bp, Artificial Sequence
  Probe B.thetaio03
ttgtcgttgccaata

SEQ ID NO: 238 - DNA, 15 bp, Artificial Sequence
  Probe B.thetaio04
cagtgttggaatgtt

SEQ ID NO: 239 - DNA, 15 bp, Artificial Sequence
  Probe B.thetaio05
actatactatagtca

SEQ ID NO: 240 - DNA, 15 bp, Artificial Sequence
  Probe C.diffc005
gttcgtccgcccctg

SEQ ID NO: 241 - DNA, 15 bp, Artificial Sequence
  Probe C.diffc001
gcatatatatttagt

SEQ ID NO: 242 - DNA, 15 bp, Artificial Sequence
  Probe C.diffc002
gatatgacatctaat

SEQ ID NO: 243 - DNA, 15 bp, Artificial Sequence
  Probe C.diffc003
tttcggggagttgca

SEQ ID NO: 244 - DNA, 15 bp, Artificial Sequence
  Probe C.diffc004
catgtggacagtatg

SEQ ID NO: 245 - DNA, 15 bp, Artificial Sequence
  Probe H.aphro003
ggtgaagaacccact

SEQ ID NO: 246 - DNA, 15 bp, Artificial Sequence
  Probe H.aphro001
tgggagtgggttgtc

SEQ ID NO: 247 - DNA, 15 bp, Artificial Sequence
  Probe H.aphro002
taacaaaccggaaac

SEQ ID NO: 248 - DNA, 15 bp, Artificial Sequence
  Probe H.aphro004
atcattatctgaatc

SEQ ID NO: 249 - DNA, 15 bp, Artificial Sequence
  Probe H.aphro005
agaaatcaaccgtag

SEQ ID NO: 250 - DNA, 15 bp, Artificial Sequence
  Probe H.aphro006
attagcggatgactc

SEQ ID NO: 251 - DNA, 15 bp, Artificial Sequence
  Probe H.aphro007
aacccagtgggtgaa

SEQ ID NO: 252 - DNA, 15 bp, Artificial Sequence
  Probe H.aphro008
aaacccagtgggtga

SEQ ID NO: 253 - DNA, 15 bp, Artificial Sequence
  Probe H.aphro009
gaaacccagtgggtg

SEQ ID NO: 254 - DNA, 15 bp, Artificial Sequence
  Probe N.gono005
tatcaaagtagggat

SEQ ID NO: 255 - DNA, 15 bp, Artificial Sequence
  Probe N.gono006
agtcaacgggtaggt

SEQ ID NO: 256 - DNA, 15 bp, Artificial Sequence
  Probe N.gono002
aacctctcgcaagag

SEQ ID NO: 257 - DNA, 15 bp, Artificial Sequence
  Probe N.gono003
catagtatttgggtg

SEQ ID NO: 258 - DNA, 15 bp, Artificial Sequence
  Probe N.gono004
ttgtatcagacttaa

SEQ ID NO: 259 - DNA, 15 bp, Artificial Sequence
  Probe N.gono001
aatgagtttgttttg

SEQ ID NO: 260 - DNA, 15 bp, Artificial Sequence
  Probe N.gono007
caatgagtttgtttt

SEQ ID NO: 261 - DNA, 15 bp, Artificial Sequence
  Probe N.gono008
cgtaactataacggt

SEQ ID NO: 262 - DNA, 15 bp, Artificial Sequence
  Probe E.corro005
ggataggagaaggaa

SEQ ID NO: 263 - DNA, 15 bp, Artificial Sequence
  Probe E.corro006
actcatcatcgatcc

SEQ ID NO: 264 - DNA, 15 bp, Artificial Sequence
  Probe E.corro001
agtcgtagagcggag

SEQ ID NO: 265 - DNA, 15 bp, Artificial Sequence
  Probe E.corro002
agatccgcccaggta

SEQ ID NO: 266 - DNA, 15 bp, Artificial Sequence
  Probe E.corro003
gttgctgcatcttgc

SEQ ID NO: 267 - DNA, 15 bp, Artificial Sequence
  Probe E.corro004
gcaggattcggacac

SEQ ID NO: 268 - DNA, 15 bp, Artificial Sequence
  Probe B.vulga03
agtcagcgtcgaagg

SEQ ID NO: 269 - DNA, 15 bp, Artificial Sequence
  Probe B.vulga07
cgaatgcgcatcagt

SEQ ID NO: 270 - DNA, 15 bp, Artificial Sequence
  Probe B.vulga01
catcttgagatgtgc

SEQ ID NO: 271 - DNA, 15 bp, Artificial Sequence
  Probe B.vulga02
agtcgggcgtggata

SEQ ID NO: 272 - DNA, 15 bp, Artificial Sequence
  Probe B.vulga04
acgctaatcggatca

SEQ ID NO: 273 - DNA, 15 bp, Artificial Sequence
  Probe B.vulga05
gaccgatagagcatg

SEQ ID NO: 274 - DNA, 15 bp, Artificial Sequence
  Probe B.vulga06
tgacacactgtaact

SEQ ID NO: 275 - DNA, 15 bp, Artificial Sequence
  Probe B.vulga08
attgtcatgagccac

SEQ ID NO: 276 - DNA, 15 bp, Artificial Sequence
  Probe B.vulga09
aatttgcgtggctct

SEQ ID NO: 277 - DNA, 15 bp, Artificial Sequence
  Probe B.vulga10
ctccatcggaaacgt

SEQ ID NO: 278 - DNA, 15 bp, Artificial Sequence
  Probe B.vulga11
actccatcggaaacg

SEQ ID NO: 279 - DNA, 15 bp, Artificial Sequence
  Probe B.vulga12
gggactacgaacgga

SEQ ID NO: 280 - DNA, 15 bp, Artificial Sequence
  Probe B.catar005
atatcttcgcgctgt

SEQ ID NO: 281 - DNA, 15 bp, Artificial Sequence
  Probe B.catar001
agtctgggttaatta

SEQ ID NO: 282 - DNA, 15 bp, Artificial Sequence
  Probe B.catar002
acaagttgttctttg

SEQ ID NO: 283 - DNA, 15 bp, Artificial Sequence
  Probe B.catar003
aacataggtgaatcg

SEQ ID NO: 284 - DNA, 15 bp, Artificial Sequence
  Probe B.catar004
aagtaatgaagtgca

SEQ ID NO: 285 - DNA, 15 bp, Artificial Sequence
  Probe B.catar006
gaggataacaatgaa

SEQ ID NO: 286 - DNA, 15 bp, Artificial Sequence
  Probe B.catar007
cgaatgagtttgtca

SEQ ID NO: 287 - DNA, 15 bp, Artificial Sequence
  Probe B.catar008
acccgaatatccgac

SEQ ID NO: 288 - DNA, 15 bp, Artificial Sequence
  Probe B.catar009
gacccaccattttgg

SEQ ID NO: 289 - DNA, 15 bp, Artificial Sequence
  Probe B.catar010
ataatggggtcagcg

SEQ ID NO: 290 - DNA, 15 bp, Artificial Sequence
  Probe B.catar011
agcctgtgaaggtgc

SEQ ID NO: 291 - DNA, 15 bp, Artificial Sequence
  Probe B.catar012
aagaattgatgacca

SEQ ID NO: 292 - DNA, 15 bp, Artificial Sequence
  Probe S.wad 02
gctccgacaagaact

SEQ ID NO: 293 - DNA, 15 bp, Artificial Sequence
  Probe S.wad03
cgagttgttgaattc

SEQ ID NO: 294 - DNA, 15 bp, Artificial Sequence
  Probe S.wad04
gtcgtcttgtgcttt

SEQ ID NO: 295 - DNA, 15 bp, Artificial Sequence
  Probe S.wad 01
ctcagtaagacgttt

SEQ ID NO: 296 - DNA, 15 bp, Artificial Sequence
  Probe Acii1(Acii23)
aacctggctggtggc

SEQ ID NO: 297 - DNA, 15 bp, Artificial Sequence
  Probe Acii2(AciiM)
gacacttttgtgtca

SEQ ID NO: 298 - DNA, 15 bp, Artificial Sequence
  Probe Acii3(Aci I)
gttgggtggttgcct

SEQ ID NO: 299 - DNA, 15 bp, Artificial Sequence
  Probe SeM01
gatagataacaggtg

SEQ ID NO: 300 - DNA, 15 bp, Artificial Sequence
  Probe SeM02
agggttcacgcccag

SEQ ID NO: 301 - DNA, 15 bp, Artificial Sequence
  Probe Se1(Se 23)
ttctctcttgagtgg

SEQ ID NO: 302 - DNA, 15 bp, Artificial Sequence
  Probe Se2(Se M)
cgtgctgttggagtg

SEQ ID NO: 303 - DNA, 15 bp, Artificial Sequence
  Probe Se3(Se I)
gctatttattttgaa

SEQ ID NO: 304 - DNA, 15 bp, Artificial Sequence
  Probe Bur(Bur23)
ttgttagccgaacgc

SEQ ID NO: 305 - DNA, 15 bp, Artificial Sequence
  Probe Bur01
gggtgtggcgcgagc

SEQ ID NO: 306 - DNA, 15 bp, Artificial Sequence
  Probe Bur001
gccaggagggtgaag

SEQ ID NO: 307 - DNA, 15 bp, Artificial Sequence
  Probe Styp(Styp23)
gcctgaatcagcatg

SEQ ID NO: 308 - DNA, 15 bp, Artificial Sequence
  Probe Styp01
gctgaggatacggtt

SEQ ID NO: 309 - DNA, 15 bp, Artificial Sequence
  Probe Styp02
ccgcaaaacaagcag

SEQ ID NO: 310 - DNA, 15 bp, Artificial Sequence
  Probe Styp03
acgattgacggagcg

SEQ ID NO: 311 - DNA, 15 bp, Artificial Sequence
  Probe Sal.typ001
tcgcgccgtcacagt

SEQ ID NO: 312 - DNA, 15 bp, Artificial Sequence
  Probe E coli 003
ctgaagcgacaaatg

SEQ ID NO: 313 - DNA, 15 bp, Artificial Sequence
  Probe Eco1(Eco23)
gagcctgaatcagtg

SEQ ID NO: 314 - DNA, 15 bp, Artificial Sequence
  Probe Eco2(Ecoli)
gttagcggtaacgcg

SEQ ID NO: 315 - DNA, 15 bp, Artificial Sequence
  Probe E coli 001
gttagcggtaacgcg

SEQ ID NO: 316 - DNA, 15 bp, Artificial Sequence
  Probe E coli 002
atgcacatattgtga

SEQ ID NO: 317 - DNA, 15 bp, Artificial Sequence
  Probe Kpneu(Kpneu 23)
gtacaccaaaatgca

SEQ ID NO: 318 - DNA, 15 bp, Artificial Sequence
  Probe K.pneu002
gctgagaccagtcga

SEQ ID NO: 319 - DNA, 15 bp, Artificial Sequence
  Probe K.pneu001
acgctggtgtgtagg

SEQ ID NO: 320 - DNA, 15 bp, Artificial Sequence
  Probe Kpneu01
accttcgggtgtgac

SEQ ID NO: 321 - DNA, 15 bp, Artificial Sequence
  Probe Pm
gttaccaacaatcgt

SEQ ID NO: 322 - DNA, 15 bp, Artificial Sequence
  Probe Pm002
ggcgacggtcgtccc

SEQ ID NO: 323 - DNA, 15 bp, Artificial Sequence
  Probe Pm003
gatgacgaaccacca

SEQ ID NO: 324 - DNA, 15 bp, Artificial Sequence
  Probe Pm004
tgaagcaattgatgc

SEQ ID NO: 325 - DNA, 15 bp, Artificial Sequence
  Probe Pm001
aaggctaggttgtcc

SEQ ID NO: 326 - DNA, 15 bp, Artificial Sequence
  Probe Pm005
taaagtccctcgcgg

SEQ ID NO: 327 - DNA, 15 bp, Artificial Sequence
  Probe Pm01
aggcagagtgattag

SEQ ID NO: 328 - DNA, 15 bp, Artificial Sequence
  Probe Strepp(StreppM)
taggactgcaatgtg

SEQ ID NO: 329 - DNA, 15 bp, Artificial Sequence
  Probe Strepp01
atgtggtacagacac

SEQ ID NO: 330 - DNA, 15 bp, Artificial Sequence
  Probe Strepp02
ggttaaacgctagaa

SEQ ID NO: 331 - DNA, 15 bp, Artificial Sequence
  Probe Strepp03
caggatactgctaag

SEQ ID NO: 332 - DNA, 15 bp, Artificial Sequence
  Probe Strepp04
gagtaaactcttcgg

SEQ ID NO: 333 - DNA, 15 bp, Artificial Sequence
  Probe Vvul02
gttgacgatgcatgt

SEQ ID NO: 334 - DNA, 15 bp, Artificial Sequence
  Probe VvulM
agtaacagccacttg

SEQ ID NO: 335 - DNA, 15 bp, Artificial Sequence
  Probe V.vul001
atagctcaatgaagc

SEQ ID NO: 336 - DNA, 15 bp, Artificial Sequence
  Probe V.vul002
ggcgccatagtctct

SEQ ID NO: 337 - DNA, 15 bp, Artificial Sequence
  Probe Vvul 01
tttacatgtgttaga

SEQ ID NO: 338 - DNA, 15 bp, Artificial Sequence
  Probe Vvul 03
gttctatgaacattg

SEQ ID NO: 339 - DNA, 15 bp, Artificial Sequence
  Probe P.aeru001
gaagtgccgagcatg

SEQ ID NO: 340 - DNA, 15 bp, Artificial Sequence
  Probe Pa03
ggatctttgaagtga

SEQ ID NO: 341 - DNA, 15 bp, Artificial Sequence
  Probe Pa
gtttcccgtgaaggc

SEQ ID NO: 342 - DNA, 15 bp, Artificial Sequence
  Probe P.aeru002
gtgtcacgtaagtga

SEQ ID NO: 343 - DNA, 15 bp, Artificial Sequence
  Probe P.aeru003
agtcgtcttttagat

SEQ ID NO: 344 - DNA, 15 bp, Artificial Sequence
  Probe P.aeru004
actccgtaagctctg

SEQ ID NO: 345 - DNA, 15 bp, Artificial Sequence
  Probe Pa01
taggataacctaggt

SEQ ID NO: 346 - DNA, 15 bp, Artificial Sequence
  Probe Pa02
taagcttcattgatt

SEQ ID NO: 347 - DNA, 15 bp, Artificial Sequence
  Probe Ah
ggcgcctcggtaggg

SEQ ID NO: 348 - DNA, 15 bp, Artificial Sequence
  Probe Ae.hy 001
taagccgtgagcagt

SEQ ID NO: 349 - DNA, 15 bp, Artificial Sequence
  Probe Ae.hy 002
catcttggaagttag

SEQ ID NO: 350 - DNA, 15 bp, Artificial Sequence
  Probe Ae.hy 003
tcaaaccaggcaccg

SEQ ID NO: 351 - DNA, 15 bp, Artificial Sequence
  Probe Ae.hy 004
gattcacgctaagcg

SEQ ID NO: 352 - DNA, 15 bp, Artificial Sequence
  Probe Ae.hy 005
acggtgcggaagcca

SEQ ID NO: 353 - DNA, 15 bp, Artificial Sequence
  Probe Ah01
cacgaaaacaacctt

SEQ ID NO: 354 - DNA, 15 bp, Artificial Sequence
  Probe LM
gggtgcaagcccgag

SEQ ID NO: 355 - DNA, 15 bp, Artificial Sequence
  Probe Lm01
agtatccttacgtga

SEQ ID NO: 356 - DNA, 15 bp, Artificial Sequence
  Probe Lm02
gtgaggaaggcagac

SEQ ID NO: 357 - DNA, 15 bp, Artificial Sequence
  Probe Lm03
ggctttccctccaga

SEQ ID NO: 358 - DNA, 15 bp, Artificial Sequence
  Probe Lm04
ccgcttctcacgaag

SEQ ID NO: 359 - DNA, 15 bp, Artificial Sequence
  Probe E.faecium002
ttacgattgtgtgaa

SEQ ID NO: 360 - DNA, 15 bp, Artificial Sequence
  Probe E.faecium003
atagcacattcgagg

SEQ ID NO: 361 - DNA, 15 bp, Artificial Sequence
  Probe Enfcium1(Enfaeci23)
gttctttcagatagt

SEQ ID NO: 362 - DNA, 15 bp, Artificial Sequence
  Probe Enfcium2(Enfaeci M)
ctgaagaggagtcaa

SEQ ID NO: 363 - DNA, 15 bp, Artificial Sequence
  Probe E.faecium001
gctgatcatacgatc

SEQ ID NO: 364 - DNA, 15 bp, Artificial Sequence
  Probe E.faecium004
cttcttttcttaagg

SEQ ID NO: 365 - DNA, 15 bp, Artificial Sequence
  Probe S.aureus004
gattgcacgtctaag

SEQ ID NO: 366 - DNA, 15 bp, Artificial Sequence
  Probe S.aureus005
aatccggtactcgtt

SEQ ID NO: 367 - DNA, 15 bp, Artificial Sequence
  Probe Saure03
tcttcgagtcgttga

SEQ ID NO: 368 - DNA, 15 bp, Artificial Sequence
  Probe Saur
gttaacgcccagaag

SEQ ID NO: 369 - DNA, 15 bp, Artificial Sequence
  Probe S.aureus001
aggacgacattagac

SEQ ID NO: 370 - DNA, 15 bp, Artificial Sequence
  Probe S.aureus002
aaaatgttgtctctc

SEQ ID NO: 371 - DNA, 15 bp, Artificial Sequence
  Probe S.aureus003
cgaagcgtgcgattg

SEQ ID NO: 372 - DNA, 15 bp, Artificial Sequence
  Probe Saure01
aagcagtaaatgtgg

SEQ ID NO: 373 - DNA, 15 bp, Artificial Sequence
  Probe Saure02
gagaagacattgtgt

SEQ ID NO: 374 - DNA, 15 bp, Artificial Sequence
  Probe Saureus01
atatcagaaggcaca

SEQ ID NO: 375 - DNA, 15 bp, Artificial Sequence
  Probe Saureus02
acaaaggacgacatt

SEQ ID NO: 376 - DNA, 15 bp, Artificial Sequence
  Probe Saureus03
tcttcgagtcgttga

SEQ ID NO: 377 - DNA, 15 bp, Artificial Sequence
  Probe Nm002
agatgtgagagcatc

SEQ ID NO: 378 - DNA, 15 bp, Artificial Sequence
  Probe Nm1(Nm)
ccctggagggtcgca

SEQ ID NO: 379 - DNA, 15 bp, Artificial Sequence
  Probe Nm2(Nm-1)
tttgaattgaaccgt

SEQ ID NO: 380 - DNA, 15 bp, Artificial Sequence
  Probe Nm001
gtttactggcatggt

SEQ ID NO: 381 - DNA, 15 bp, Artificial Sequence
  Probe Nm01
taaagcaatgatccc

SEQ ID NO: 382 - DNA, 15 bp, Artificial Sequence
  Probe Nm02
ccgggtcttcttaac

SEQ ID NO: 383 - DNA, 15 bp, Artificial Sequence
  Probe L.pneu011
tggagagcattttat

SEQ ID NO: 384 - DNA, 15 bp, Artificial Sequence
  Probe L.pneu012
gtgattttgaggtga

SEQ ID NO: 385 - DNA, 15 bp, Artificial Sequence
  Probe L.pneu013
agatggtaaagaaga

SEQ ID NO: 386 - DNA, 15 bp, Artificial Sequence
  Probe L.pneu001
cctcaagatgagttt

SEQ ID NO: 387 - DNA, 15 bp, Artificial Sequence
  Probe L.pneu002
gaagcccgttgaaga

SEQ ID NO: 388 - DNA, 15 bp, Artificial Sequence
  Probe L.pneu003
gcagtaatgcgtgaa

SEQ ID NO: 389 - DNA, 15 bp, Artificial Sequence
  Probe L.pneu004
ttgtcttgaccatat

SEQ ID NO: 390 - DNA, 15 bp, Artificial Sequence
  Probe L.pneu005
accatataatctgag

SEQ ID NO: 391 - DNA, 15 bp, Artificial Sequence
  Probe L.pneu006
tgcccacacagtttg

SEQ ID NO: 392 - DNA, 15 bp, Artificial Sequence
  Probe L.pneu007
caaagtgcccacaca

SEQ ID NO: 393 - DNA, 15 bp, Artificial Sequence
  Probe L.pneu008
tgattttgaggtgat

SEQ ID NO: 394 - DNA, 15 bp, Artificial Sequence
  Probe L.pneu009
ccaccatttaatgat

SEQ ID NO: 395 - DNA, 15 bp, Artificial Sequence
  Probe L.pneu010
agcattttattctgg

SEQ ID NO: 396 - DNA, 15 bp, Artificial Sequence
  Probe C.alic001
tggtagccatttatg

SEQ ID NO: 397 - DNA, 15 bp, Artificial Sequence
  Probe C.alic003
ctggaccagccgagc

SEQ ID NO: 398 - DNA, 15 bp, Artificial Sequence
  Probe C.alic006
tcaagaacgaaagtt

SEQ ID NO: 399 - DNA, 15 bp, Artificial Sequence
  Probe C.alic007
aaggattgacagatt

SEQ ID NO: 400 - DNA, 15 bp, Artificial Sequence
  Probe C.alic008
cattaatcaagaacg

SEQ ID NO: 401 - DNA, 15 bp, Artificial Sequence
  Probe C.alic002
ttttcgatgcgtact

SEQ ID NO: 402 - DNA, 15 bp, Artificial Sequence
  Probe C.alic004
cagatgtcgaaaggt

SEQ ID NO: 403 - DNA, 15 bp, Artificial Sequence
  Probe C.alic005
taggacgttatggtt

SEQ ID NO: 404 - DNA, 15 bp, Artificial Sequence
  Probe C.glab001
ctggaatgcacccgg

SEQ ID NO: 405 - DNA, 15 bp, Artificial Sequence
  Probe C.glab003
tggcttggcggcgaa

SEQ ID NO: 406 - DNA, 18 bp, Artificial Sequence
  Primer 1585Fw
ttgtacacaccgcccgtc

SEQ ID NO: 407 - DNA, 22 bp, Artificial Sequence
  Primer 23BR
ttcgcctttccctcacggtact

SEQ ID NO: 408 - DNA, 22 bp, Artificial Sequence
  Primer 23BFw
agtaccgtgagggaaaggggaa

SEQ ID NO: 409 - DNA, 20 bp, Artificial Sequence
  Primer 37R
tgcttctaagccaacatcct

SEQ ID NO: 410 - DNA, 20 bp, Artificial Sequence
  Primer MS37F
aggatgttggcttagaagca

SEQ ID NO: 411 - DNA, 24 bp, Artificial Sequence
  Primer MS38R
cccgacaaggaatttcgctacctt

SEQ ID NO: 412 - DNA, 20 bp, Artificial Sequence
  Primer fun463F
gtaattggaatgagtacaat

SEQ ID NO: 413 - DNA, 20 bp, Artificial Sequence
  Primer fun986R
ctacgacggtatctgatcat

SEQ ID NO: 414 - DNA, 15 bp, Artificial Sequence
  Primer 520R
gccaaggcatccacc

SEQ ID NO: 415 - DNA, 16 bp, Artificial Sequence
  Primer 23S 750F
agtagcggcgagcgaa

SEQ ID NO: 416 - DNA, 16 bp, Artificial Sequence
  Primer 23S 750F(T)
agtagtggcgagcgaa

SEQ ID NO: 417 - DNA, 17 bp, Artificial Sequence
  Primer 970F
aactggaggaccgaacc

SEQ ID NO: 418 - DNA, 17 bp, Artificial Sequence
  Primer  930R
awtttgcygagttcctt

SEQ ID NO: 419 - DNA, 18 bp, Artificial Sequence
  Primer  2960R(T)
gcttagatgctttcagca

SEQ ID NO: 420 - DNA, 18 bp, Artificial Sequence
  Primer  2960RC
gcttagatgctttcagcg

SEQ ID NO: 421 - DNA, 15 bp, Artificial Sequence
  Control Probe BaP1-01
cacggtggatgccct

SEQ ID NO: 422 - DNA, 15 bp, Artificial Sequence
  Control Probe Bap1-03
agtagcggcgagcga

SEQ ID NO: 423 - DNA, 15 bp, Artificial Sequence
  Control Probe BaP1-06
gaccgatagtgaacc

SEQ ID NO: 424 - DNA, 15 bp, Artificial Sequence
  Control Probe BaP2-01
agaacctgaaaccgt

SEQ ID NO: 425 - DNA, 15 bp, Artificial Sequence
  Control Probe BaP2-03
actggaggaccgaac

SEQ ID NO: 426 - DNA, 15 bp, Artificial Sequence
  Control Probe BaP2-04
agggaaacaacccag

SEQ ID NO: 427 - DNA, 15 bp, Artificial Sequence
  Control Probe BaP3
gtaaacggcggccgt

SEQ ID NO: 428 - DNA, 15 bp, Artificial Sequence
  Position marker
aaaaaaaaaaaaaaa

(19) United States
(12) Patent Application Publication
(10) Pub. No.: US 2007/0065817 A1
(43) Pub. Date: Mar. 22, 2007

(54) Nucleic acid probes for detection of non-viral organisms

(76) Inventors: Sang-Yup Lee, Daejeon (KR); Seung Min Yoo, Seoul (JP); Kyung Hee Chang, Seoul (KR); So Young Yoo, Daejeon (KR); Nae Choon Yo, Seoul (KR); Won Min Yoo, Seoul (KR); Ki Chang Keum, Seoul (KR); June Myung Kim, Seoul (KR); Gene Lee, Gyeonggi-dong (KR)

Correspondence Address: Joseph Hyosuk Kim;JHK Law, PO Box 1078, La Canada, CA, 91012-1078, US

(21) Appl. No.: 10/514,072

(22) PCT Filed: May 9, 2003
(86) PCT No.: PCT/KR03/00923

§ 371 Date: Jun. 30, 2005

(30) Foreign Application Priority Data
May 9, 2002 (KR) 10-2002-0025634
May 9, 2002 (KR) 10-2002-0025569
May 9, 2002 (KR) 10-2002-0025583
May 9, 2002 (KR) 10-2002-0025580
May 9, 2002 (KR) 10-2002-0025579
May 9, 2002 (KR) 10-2002-0025566
May 9, 2002 (KR) 10-2002-0025687
May 9, 2002 (KR) 10-2002-0025562
May 9, 2002 (KR) 10-2002-0025567
May 9, 2002 (KR) 10-2002-0025561
May 9, 2002 (KR) 10-2002-0025582
Aug. 28, 2002 (KR) 10-2002-0051054
Jan. 25, 2003 (KR) 10-2003-0005082
Jan. 27, 2003 (KR) 10-2003-0005344
Jan. 27, 2003 (KR) 10-2003-0005341
Jan. 27, 2003 (KR) 10-2003-0005342

(51) Int. Cl. C12Q 1/68; C12M 3/00; C12M 1/34; C07H 21/04

(52) U.S. Cl. 435/6; 435/287.2; 536/23.7; 257/E51.02; 977/924

(57) ABSTRACT

The present invention relates to nucleic acid probes which are derived from rRNA genes of non-virus organisms and are useful for the detection of said non-virus infectious organisms in a biological sample. In addition, the present invention relates to compositions and chips useful for the diagnosis of one or more types of infectious diseases comprising said nucleic acid probes.

### TECHNICAL FIELD

The present invention relates to nucleic acid probes useful for the detection and identification of non-viral infectious organisms in a biological sample and for the diagnosis of non-viral infectious disease caused by such organisms. More particularly, the present invention relates to nucleic acid probes which are derived from rRNA genes of non-viral infectious organisms and are useful for the detection and identification of non-viral infectious organisms for which they were designed. It also relates to compositions including said nucleic acid probes and to kit in which said probes were immobilized on a solid support.

### BACKGROUND ART

Infectious disease results from the presence and activity of pathogenic organisms in human blood, fluid, and tissue. It may be developed into a fatal disease, if causal organisms fail to be identified and controlled properly. Recently, there has been abuse of antibiotic substances, overuse of immunosuppressants by transplantation and overdose of drugs by anticancer therapy. As results, pathogenic organisms are undergoing successive or alternate changes in genes and culture rate of such organisms is dwindling. The adaptation of pathogenic organisms makes it difficult to diagnose infectious disease using traditional diagnostic methods.

Since some anaerobic organisms exhibit enough pathogenicity to cause severe disease to humans, they must be rapidly detected in a biological sample and accurately identified to diagnose infectious disease. As the rapid detection and accurate identification of pathogenic microbes in a biological sample are considerably of the importance in the treatment of infectious disease, a variety of methods for the detection and identification of pathogenic microbes has been researched and developed over a long time. Although the technology for the detection of microbes including infectious disease has been advanced gradually, it is still laborious and offers low sensitivity and specificity.

With the exception of viruses, all prokaryotic organisms contain rRNA genes encoding homologs of the prokaryotic 5S, 16S and 23S rRNA molecules. In eukaryotic organisms, these rRNA molecules are the 5S rRNA, 5.8S rRNA, 18S rRNA and 28S rRNA which are substantially similar to the prokaryotic molecules. Nucleic acid probes for detecting specifically targeted rRNA subsequences in particular non-viral organisms or groups of non-viral organisms in a biological sample have been described previously. Many of the problems to be confronted with the detection of microbes in a biological sample could be solved by using such nucleic acid probes in combination with well-known polymerase chain reaction (PCR) techniques.

The choice of target genes to be amplified is very important in a diagnostic nucleic acid probe technology. rRNA genes, especially 23S rRNA genes and internal transcribed spacer region (ITS), are usually used as targeted sequences. It has been reported that certain nucleic acid sequences derived from rRNA genes of selected bacterial or fungal species advantageously allow low probability of cross-reacting with nucleic acids originating from microbes other than the targeted species under appropriate stringency conditions (P. Wattiau et. al., Appl. Microbiol. Biotechnol., 56, 816-819, 2001; D, A. Stahlm et. al., J. Bacteriol., 172, 116-124, 1990; Boddinghaus. et. al., J. Clin., Microbiol., 28, 1751-1759, 1990; T. Rogall et al., J. Gen. Microbiol., 136, 1915-1920, 1990; T. Rogall, et. al., Int. J. System. Bacteriol., 40, 323-330, 1990; K. Rantakokko-Jalava et. al., J. Clin., Mirobiol., 38(1), 32-39, 2000 ; Park et. al., J. Clin., Mirobiol., 38(11), 4080-4085, 2000; A. Schmalenberger et. al, Appl. Microbiol. Biotechnol., 67(8), 3557-3563, 2001; International Publication No. W098/55646; U.S. Pat. No. 6,025,132 to Jannes, et al.; and U.S. Pat. No. 6,277,577 to Rossau, et al.).

However, the nucleotide sequences of rRNA genes originating from many pathogenic microbes have not yet been identified. There are still needs to identify the nucleotide sequences of rRNA genes originating from such pathogenic microbes and to develop nucleic acid probes derived from them highly specific to infectious microbes for which they were designed. For some pathogenic microbes, although their rRNA genes have been sequenced fully or partially, there remains a need for a nucleic acid probe to detect them with higher specificity and sensitivity.

It is thus the object of the present invention is to develop nucleic acid probes useful for the detection and identification of the following infectious microbial species:
(1) Acinetobacter baumanii;
(2) Anaerobiospirillum succiniciproducens;
(3) Bacteroides fragilis;
(4) Cardiobacterium hominis;
(5) Chryseobacterium meningosepticum;
(6) Clostridium ramosum;
(7) Comamonas acidovorans;
(8) Corynebacterium diphtheriae;
(9) Klebsiella oxytoca;
(10) Ochrobactrum anthropi;
(11) Peptostreptococcus prevotii;
(12) Porphyromonas gingivalis;
(13) Peptostreptococcus anaerobius;
(14) Peptostreptococcus magnus;
(15) Fusobacterium necrophorum;
(16) Proteus vulgaris;
(17) Enterobacter aerogenes;
(18) Streptococcus mutans;
(19) Kingella kingap;
(20) Bacteroides ovatus;
(21) Bacteroides thetaiotaomicron;
(22) Clostridium diffcile;
(23) Haemohilus aphrophilas;
(24) Neisseria gonorrhea;
(25) Eikenella corrodens;
(26) Bacteroides vulgatus;
(27) Branhamella catarrhalis;
(28) Sutterella wadsworthensis;
(29) Actinomyces israelii;
(30) Staphylococcus epidermidis;
(31) Burkholderia cepacia;
(32) Salmonella enteritidis;
(33) Escherichia coli;
(34) Klebsiella pneumoniae;
(35) Proteus mirabilis;
(36) Streptococcus pneumoniae;
(37) Vibrio vulnificus;
(38) Pseudomonas aeruginosa;
(39) Aeromonas hydrohila;
(40) Listeria monocytogenes;
(41) Enterococcus faecium;
(42) Staphylococcus aureus;
(43) Neisseria meningitidis;
(44) Legionella pneumophila;
(45) Candida albicans; and
(46) Candida glabrata.

### SUMMARY OF INVENTION

We developed nucleic acid probes that hybridize specifically to rRNA genes originating from the aforementioned microbial species (1)-(46) and do not cross-react with nuclic acids originating from those other than the aforementioned microbial species (1)-(46) and achieved the purpose of the present invention by constructing DNA chips in which said probes are spotted on a solid support and confirming the specificity and sensitivity of each probe through clinical trials using said DNA chips. For the above microbial species (1) to (28), full sequences of 23S rRNA genes and internal transcribed spacer regions (ITSS) were first identified by us and are shown as SEQ ID NO: 1 to SEQ ID NO: 28, respectively. Nucleic acid probes for the detection of microbial species (1) to (28) comprise nucleotide sequences which are derived from sequences depicted in SEQ ID NO: 1 to SEQ ID NO: 28 and only hybridize to the target 23S rRNA or ITS genes of interest originating from the microbes for which they were designed and do not cross-react with nucleic acids originating from organisms other than the microbial species of interest. For the detection of microbial species (29) to (44), nucleic acid probes comprise nucleotide sequences which are derived from known 23S rRNA gene and only hybridize to the target 23S rRNA genes of interest originating from the microbial species for which they were designed and do not cross-react with nucleic acids originating from organisms other than the microbial species of interest. For the detection of fungi (45) and (46), nucleic acid probes comprise nucleotide sequences which are derived from known 18S rRNA gene and only hybridize to the target 18S rRNA genes of interest originating from the fungal species for which they were designed and do not cross-react with nucleic acids originating from organisms other than the fungal species of interest.

In one aspect, the present invention provides isolated nucleic acid molecules having nucleotide sequences shown in SEQ ID NO: 1 to SEQ ID NO: 28 which correspond to nucleotide sequences of 23S rRNA genes and ITSs from the aforementioned 28 bacteria species, respectively.

In another aspect (1-i-a), the present invention provides isolated nucleic acid molecules derived from 23S rRNA gene of Acinetobacter baumanii which comprise a nucleotide sequence selected from the group consisting of the following:

TGATGGAACTTGCTT; (Acti004, SEQ ID NO: 29)
AGGGCACACATAATG; (Acti23S01, SEQ ID NO: 30)
and
ACGCTGTTGTTGGTG. (Acti23S02, SEQ ID NO: 31)

In another aspect (1-i-b), the present invention provides isolated nucleic acid molecules derived from ITS gene of Acinetobacter baumanii which comprise a nucleotide sequence selected from the group consisting of the following:

ATACACAGTACTTCG; (Acti3, SEQ ID NO: 32)
ATAGTGTTGCAAGGC; (Acti002, SEQ ID NO: 33)
and
TGAAAAGCCAGGGGA. (Acti003, SEQ ID NO: 34)

In another aspect (1-ii), the present invention provides nucleic acid probes for detecting Acinetobacter baumanii which comprise any one of nucleotide sequences shown in SEQ ID NO: 29 to SEQ ID NO: 34.

In another aspect (1-iii), the present invention provides a composition comprising at least one of nucleic acid probes for detecting Acinetobacter baumanii which comprises any one of nucleotide sequences shown in SEQ ID NO: 29 to SEQ ID NO: 34.

In another aspect (1-iv), the present invention provides a kit for detecting and identifying Acinetobacter baumanii in a biological sample which comprises (a) a composition comprising at least one of nucleic acid probes for detecting said bacteria which comprises any one of nucleotide sequences shown in SEQ ID NO: 29 to SEQ ID NO: 34, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (1-v), the present invention provides a DNA chip in which at least one of nucleic acid molecules comprising any one of nucleotide sequences shown in SEQ ID NO: 29 to SEQ ID NO: 34 is immobilized on a solid support.

In another aspect (1-vi), the present invention provides a method for detection and identification of Acinetobacter baumanii in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 29 to SEQ ID NO: 34 under the appropriate hybridization and wash conditions, (d) detecting the hybrids formed in step (c), and (e) identifying the bacteria present in the sample from the differential hybridization signals obtained in step (d).

In another aspect (2-i-a), the present invention provides isolated nucleic acid molecules derived from 23S rRNA gene of Anaerobiospirillum succiniciproducens which comprise a nucleotide sequence selected from the group consisting of the following:

TGACTCGTGCCCATG; (Anas001, SEQ ID NO: 45)
TACCGGGGTTAAAAG; (Anas002, SEQ ID NO: 46)
ATCAGTGATCTGAGA; (Anas003, SEQ ID NO: 47)
GAGACGAAGCACCAT; (Anas004, SEQ ID NO: 48)
AGTTGATACAGGTAG; (Anas011, SEQ ID NO: 49)
GGCCCCATCCGGGGT; (Anas013, SEQ ID NO: 50)
and
CAGTTGGAAGCAGAG. (Anas23S03, SEQ ID NO: 51)

In another aspect (2-i-b), the present invention provides isolated nucleic acid molecules derived from 23S rRNA gene of Anaerobiospirillum succiniciproducens which comprise a nucleotide sequence selected from the group consisting of the following:

GTTCTTGATTCATTG; (Anas005, SEQ ID NO: 52)
CAGCCCAAAAGTTGA; (Anas008, SEQ ID NO: 53)
AAACTGCAGGGCACA; (Anas009, SEQ ID NO: 54)
and
ATACTACCTGACGAC. (Anas010, SEQ ID NO: 55)

In another aspect (2-ii), the present invention provides nucleic acid probes for detecting Anaerobiospirillum succiniciproducens which comprise any one of nucleotide sequences shown in SEQ ID NO: 45 to SEQ ID NO: 55.

In another aspect (2-iii), the present invention provides a composition comprising at least one of nucleic acid probes for detecting Anaerobiospirillum succiniciproducens which comprises any one of nucleotide sequences shown in SEQ ID NO: 45 to SEQ ID NO: 55.

In another aspect (2-iv), the present invention provides a kit for detecting and identifying Anaerobiospirillum succiniciproducens in a biological sample which comprises (a) a composition comprising at least one of nucleic acid probes for detecting said bacteria which comprises any one of nucleotide sequences shown in SEQ ID NO: 45 to SEQ ID NO: 55, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (2-v), the present invention provides a DNA chip in which at least one of nucleic acid molecules comprising any one of nucleotide sequences shown in SEQ ID NO: 45 to SEQ ID NO: 55 is immobilized on a solid support.

In another aspect (2-vi), the present invention provides a method for detection and identification of Anaerobiospirillum succiniciproducens in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 45 to SEQ ID NO: 55 under the appropriate hybridization and wash conditions, (d) detecting the hybrids formed in step (c), and (e) identifying the bacteria present in the sample from the differential hybridization signals obtained in step (d).

In another aspect (3-i), the present invention provides isolated nucleic acid molecules derived from 23S rRNA gene of Bacteroides fragilis which comprise the following nucleotide sequence:

GTCGAACCTGACAGT. (Bf011, SEQ ID NO: 78)

In another aspect (3-ii), the present invention provides nucleic acid probes for detecting Bacteroides fragilis which comprise the nucleotide sequence shown in SEQ ID NO: 78.

In another aspect (3-iii), the present invention provides a composition comprising at least one of nucleic acid probes for detecting Bacteroides fragilis which comprises the nucleotide sequence shown in SEQ ID NO: 78.

In another aspect (3-iv), the present invention provides a kit for detecting and identifying Bacteroides fragilis in a biological sample which comprises (a) a composition comprising at least one of nucleic acid probes for detecting said bacteria which comprises the nucleotide sequence shown in SEQ ID NO: 78, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (3-v), the present invention provides a DNA chip in which at least one of nucleic acid molecules comprising the nucleotide sequence shown in SEQ ID NO: 78 is immobilized on a solid support.

In another aspect (3-vi), the present invention provides a method for detection and identification of Bacteroides fragilis in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 78 under the appropriate hybridization and wash conditions, (d) detecting the hybrids formed in step (c), and (e) identifying the bacteria present in the sample from the differential hybridization signals obtained in step (d).

In another aspect (4-i-a), the present invention provides isolated nucleic acid molecules derived from 23S rRNA gene of Cardiobacterium hominis which comprise a nucleotide sequence selected from the group consisting of the following:

AACCCTGGTGAAGGG; (Car006, SEQ ID NO: 93)
ATATGAAGATATGTG; (Car007, SEQ ID NO: 94)
TAGATTGACTTACGG; (Car008, SEQ ID NO: 95)
GTAAAGTTTTACTAC; (Car009, SEQ ID NO: 96)
and
CCAGCACACTGTTGG. (Car2, SEQ ID NO: 97)

In another aspect (4-i-b), the present invention provides isolated nucleic acid molecules derived from ITS gene of Cardiobacterium hominis which comprise a nucleotide sequence selected from the group consisting of the following:

AAAGAGAGAACAGCA; (Car3 (CarI), SEQ ID NO: 98)
TTGGCGACAACAGGC; (Car001, SEQ ID NO: 99)
GCCCCGGGAAGCTGA; (Car002, SEQ ID NO: 100)
TAGACTGCGGAAGCG; (Car003, SEQ ID NO: 101)
and
AATTAAGTTGCGTAT. (Car004, SEQ ID NO: 102)

In another aspect (4-ii), the present invention provides nucleic acid probes for detecting Cardiobacterium hominis which comprise any one of nucleotide sequences shown in SEQ ID NO: 93 to SEQ ID NO: 102.

In another aspect (4-iii), the present invention provides a composition comprising at least one of nucleic acid probes for detecting Cardiobacterium hominis which comprises any one of nucleotide sequences shown in SEQ ID NO: 93 to SEQ ID NO: 102.

In another aspect (4-iv), the present invention provides a kit for detecting and identifying Cardiobacterium hominis in a biological sample which comprises (a) a composition comprising at least one of nucleic acid probes for detecting said bacteria which comprises any one of nucleotide sequences shown in SEQ ID NO: 93 to SEQ ID NO: 102, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer or components necessary for producing the solution, (d) a solution for washing hybrids formed under the appropriate wash conditions, and (e) optionally a means for detection of said hybrids.

In another aspect (4-v), the present invention provides a DNA chip in which at least one of nucleic acid molecules comprising any one of nucleotide sequences shown in SEQ ID NO: 93 to SEQ ID NO: 102 is immobilized on a solid support.

In another aspect (4-vi), the present invention provides a method for detection and identification of Cardiobacterium hominis in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 93 to SEQ ID NO: 102 under the appropriate hybridization and wash conditions, (d) detecting the hybrids formed in step (c), and (e) identifying the bacteria present in the sample from the differential hybridization signals obtained in step (d).

In another aspect (5-i-a), the present invention provides isolated nucleic acid molecules derived from 23S rRNA gene of Chryseobacterium meningosepticum which comprise the following nucleotide sequence:
GGCATATTTAGATGA (Chr23S04, SEQ ID NO: 105).

In another aspect (5-i-b), the present invention provides isolated nucleic acid molecules derived from ITS gene of Chryseobacterium meningosepticum which comprise a nucleotide sequence selected from the group consisting of the following:

CTTAGGTGATCACTT; (Chr001, SEQ ID NO: 106)
TAACCCCTTAGATTA; (Chr003, SEQ ID NO: 107)
TCAAACCTCAAACTA; (Chr004, SEQ ID NO: 108)
and
AAGAAATCGAAGAGA. (Chr005, SEQ ID NO: 109)

In another aspect (5-ii), the present invention provides nucleic acid probes for detecting Chryseobacterium meningosepticum which comprise any one of nucleotide sequences shown in SEQ ID NO: 105 to SEQ ID NO: 109.

In another aspect (5-iii), the present invention provides a composition comprising at least one of nucleic acid probes for detecting Chryseobacterium meningosepticum which comprises any one of nucleotide sequences shown in SEQ ID NO: 105 to SEQ ID NO: 109.

In another aspect (5-iv), the present invention provides a kit for detecting and identifying Chryseobacterium meningosepticum in a biological sample which comprises (a) a composition comprising at least one of nucleic acid probes for detecting said bacteria which comprises any one of nucleotide sequences shown in SEQ ID NO: 105 to SEQ ID NO: 109, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (5-v), the present invention provides a DNA chip in which at least one of nucleic acid molecules comprising any one of nucleotide sequences shown in SEQ ID NO: 105 to SEQ ID NO: 109 is immobilized on a solid support.

In another aspect (5-vi), the present invention provides a method for detection and identification of Chryseobacterium meningosepticum in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 105 to SEQ ID NO: 109 under the appropriate hybridization and wash conditions, (d) detecting the hybrids formed in step (c), and (e) identifying the bacteria present in the sample from the differential hybridization signals obtained in step (d).

In another aspect (6-i), the present invention provides isolated nucleic acid molecules derived from 23S rRNA gene of Clostridium ramosum which comprise a nucleotide sequence of the following:

CCAGTGTGTGAGGAG; (C. ramosa04, SEQ ID NO: 115)
or
CCCGGGAAGGGGAGT. (C. ramo004, SEQ ID NO: 116)

In another aspect (6-ii), the present invention provides nucleic acid probes for detecting Clostridium ramosum which comprise the nucleotide sequence shown in SEQ ID NO: 115 or the nucleotide sequence shown in SEQ ID NO: 116.

In another aspect (6-iii), the present invention provides a composition comprising at least one of nucleic acid probes for detecting Clostridium ramosum which comprises the nucleotide sequence shown in SEQ ID NO: 115 or the nucleotide sequence shown in SEQ ID NO: 116.

In another aspect (6-iv), the present invention provides a kit for detecting and identifying Clostridium ramosum in a biological sample which comprises (a) a composition comprising at least one of nucleic acid probes for detecting said bacteria which comprises the nucleotide sequence shown in SEQ ID NO: 115 or the nucleotide sequence shown in SEQ ID NO: 116, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (6-v), the present invention provides a DNA chip in which at least one of nucleic acid molecules comprising the nucleotide sequence shown in SEQ ID NO: 115 or the nucleotide sequence shown in SEQ ID NO: 116 is immobilized on a solid support.

In another aspect (6-vi), the present invention provides a method for detection and identification of Clostridium ramosum in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 115 or the nucleotide sequence shown in SEQ ID NO: 116 under the appropriate hybridization and wash conditions, (d) detecting the hybrids formed in step (c), and (e) identifying the bacteria present in the sample from the differential hybridization signals obtained in step (d).

In another aspect (7-i), the present invention provides isolated nucleic acid molecules derived from 23S rRNA gene of Comamonas acidovorans which comprise a nucleotide sequence selected from the group consisting of the following:

TAGGGCGTCCAGTCG; (Com004, SEQ ID NO: 124)
CGCAGAGTACAGCTT; (Com005, SEQ ID NO: 125)
GTACCGATGTGTAGT; (Com006, SEQ ID NO: 126)
GAACTTGAACAAAGG; (Com007, SEQ ID NO: 127)
TGTGCTAGAGAAAAG; (Coma2, SEQ ID NO: 128)
and
ATCCGCCGGGCTTAG. (Coma3, SEQ ID NO: 129)

In another aspect (7-ii), the present invention provides nucleic acid probes for detecting Comamonas acidovorans which comprise any one of nucleotide sequences shown in SEQ ID NO: 124 to SEQ ID NO: 129.

In another aspect (7-iii), the present invention provides a composition comprising at least one of nucleic acid probes for detecting Comamonas acidovorans which comprises any one of nucleotide sequences shown in SEQ ID NO: 124 to SEQ ID NO: 129.

In another aspect (7-iv), the present invention provides a kit for detecting and identifying Comamonas acidovorans in a biological sample which comprises (a) a composition comprising at least one of nucleic acid probes for detecting said bacteria which comprises any one of nucleotide sequences-shown in SEQ ID NO: 124 to SEQ ID NO: 129, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (7-v), the present invention provides a DNA chip in which at least one of nucleic acid molecules comprising any one of nucleotide sequences shown in SEQ ID NO: 124 to SEQ ID NO: 129 is immobilized on a solid support.

In another aspect (7-vi), the present invention provides a method for detection and identification of Comamonas acidovorans in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 124 to SEQ ID NO: 129 under the appropriate hybridization and wash conditions, (d) detecting the hybrids formed in step (c), and (e) identifying the bacteria present in the sample from the differential hybridization signals obtained in step (d).

In another aspect (8-i), the present invention provides isolated nucleic acid molecules derived from 23S rRNA gene of Corynebacterium diphtheriae which comprise the following nucleotide sequence:

ACCATCTTCCCAAGG. (C. diph003, SEQ ID NO: 135)

In another aspect (8-ii), the present invention provides nucleic acid probes for detecting Corynebacterium diphtheriae which comprise the nucleotide sequence shown in SEQ ID NO: 135.

In another aspect (8-iii), the present invention provides a composition comprising at least one of nucleic acid probes for detecting Corynebacterium diphtheriae which comprises the nucleotide sequence shown in SEQ ID NO: 135.

In another aspect (8-iv), the present invention provides a kit for detecting and identifying Corynebacterium diphtheriae in a biological sample which comprises (a) a composition comprising at least one of nucleic acid probes for detecting said bacteria which comprises the nucleotide sequence shown in SEQ ID NO: 135, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (8-v), the present invention provides a DNA chip in which at least one of nucleic acid molecules comprising the nucleotide sequence shown in SEQ ID NO: 135 is immobilized on a solid support.

In another aspect (8-vi), the present invention provides a method for detection and identification of Corynebacterium diphtheriae in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 135 under the appropriate hybridization and wash conditions, (d) detecting the hybrids formed in step (c), and (e) identifying the bacteria present in the sample from the differential hybridization signals obtained in step (d).

In another aspect (9-i), the present invention provides isolated nucleic acid molecules derived from 23S rRNA gene of Klebsiella oxytoca which comprise the following nucleotide sequence:

GAACGTTACTAACGC. (Ko001, SEQ ID NO: 142)

In another aspect (9-ii), the present invention provides nucleic acid probes for detecting Klebsiella oxytoca which comprise the nucleotide sequence shown in SEQ ID NO: 142.

In another aspect (9-iii), the present invention provides a composition comprising at least one of nucleic acid probes for detecting Klebsiella oxytoca which comprises the nucleotide sequence shown in SEQ ID NO: 142.

In another aspect (9-iv), the present invention provides a kit for detecting and identifying Klebsiella oxytoca in a biological sample which comprises (a) a composition comprising at least one of nucleic acid probes for detecting said bacteria which comprises the nucleotide sequence shown in SEQ ID NO: 142, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (9-v), the present invention provides a DNA chip in which at least one of nucleic acid molecules comprising the nucleotide sequence shown in SEQ ID NO: 142 is immobilized on a solid support.

In another aspect (9-vi), the present invention provides a method for detection and identification of Klebsiella oxytoca in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 142 under the appropriate hybridization and wash conditions, (d) detecting the hybrids formed in step (c), and (e) identifying the bacteria present in the sample from the differential hybridization signals obtained in step (d).

In another aspect (10-i-a), the present invention provides isolated nucleic acid molecules derived from 23S rRNA gene of Ochrobactrum anthropi which comprise a nucleotide sequence of the following:

GGACCAGGCCAGTGG; (Ochr04, SEQ ID NO: 151)
or
GACCAGGCCAGTGGC. (Ochr05, SEQ ID NO: 152)

In another aspect (10-i-b), the present invention provides isolated nucleic acid molecules derived from 23S rRNA gene of Ochrobactrum anthropi which comprise a nucleotide sequence selected from the group consisting of the following:

GTTGATTGACACTTG; (Ochr004, SEQ ID NO: 153)
TACCGCTCACGAGCC; (Ochr005, SEQ ID NO: 154)
and
GGGTCCGGAGGTTCA. (Ochr007, SEQ ID NO: 155)

In another aspect (10-ii), the present invention provides nucleic acid probes for detecting Ochrobactrum anthropi which comprise any one of nucleotide sequences shown in SEQ ID NO: 151 to SEQ ID NO: 155.

In another aspect (10-iii), the present invention provides a composition comprising at least one of nucleic acid probes for detecting Ochrobactrum anthropi which comprises any one of nucleotide sequences shown in SEQ ID NO: 151 to SEQ ID NO: 155.

In another aspect (10-iv), the present invention provides a kit for detecting and identifying Ochrobactrum anthropi in a biological sample which comprises (a) a composition comprising at least one of nucleic acid probes for detecting said bacteria which comprises any one of nucleotide sequences shown in SEQ ID NO: 151 to SEQ ID NO: 155, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (10-v), the present invention provides a DNA chip in which at least one of nucleic acid molecules comprising any one of nucleotide sequences shown in SEQ ID NO: 151 to SEQ ID NO: 155 is immobilized on a solid support.

In another aspect (10-vi), the present invention provides a method for detection and identification of Ochrobactrum anthropi in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 151 to SEQ ID NO: 155 under the appropriate hybridization and wash conditions, (d) detecting the hybrids formed in step (c), and (e) identifying the bacteria present in the sample from the differential hybridization signals obtained in step (d).

In another aspect (11-i), the present invention provides isolated nucleic acid molecules derived from 23S rRNA gene of Peptostreptococcus prevotii which comprise a nucleotide sequence selected from the group consisting of the following:

ACTAGGGAGAGCTCA; (Pep002, SEQ ID NO: 166)
GCTTAGTAAAGCAAG; (Pep003, SEQ ID NO: 167)
TACTAACATGTGACC; (Pep004, SEQ ID NO: 168)
AAGCAGAGAGAGCTC; (Pep005, SEQ ID NO: 169)
CGAACGGTGAGGCCG; (Pep006, SEQ ID NO: 170)
GTAGATGTTGATTAT; (Pep007, SEQ ID NO: 171)
GTCGAATCATCTGGG; (Pep23S02, SEQ ID NO: 172)
and
TAAAACGTATCGGAT. (Pep23S03, SEQ ID NO: 173)

In another aspect (11-ii), the present invention provides nucleic acid probes for detecting Peptostreptococcus prevotii which comprise any one of nucleotide sequences shown in SEQ ID NO: 166 to SEQ ID NO: 173.

In another aspect (11-iii), the present invention provides a composition comprising at least one of nucleic acid probes for detecting Peptostreptococcus prevotii which comprises any one of nucleotide sequences shown in SEQ ID NO: 166 to SEQ ID NO: 173.

In another aspect (11-iv), the present invention provides a kit for detecting and identifying Peptostreptococcus prevotii in a biological sample which comprises (a) a composition comprising at least one of nucleic acid probes for detecting said bacteria which comprises any one of nucleotide sequences shown in SEQ ID NO: 166 to SEQ ID NO: 173, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (11-v), the present invention provides a DNA chip in which at least one of nucleic acid molecules comprising any one of nucleotide sequences shown in SEQ ID NO: 166 to SEQ ID NO: 173 is immobilized on a glass slide.

In another aspect (11-vi), the present invention provides a method for detection and identification of Peptostreptococcus prevotii in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 166 to SEQ ID NO: 173 under the appropriate hybridization and wash conditions, (d) detecting the hybrids formed in step (c), and (e) identifying the bacteria present in the sample from the differential hybridization signals obtained in step (d).

In another aspect (12-i-a), the present invention provides isolated nucleic acid molecules derived from 23S rRNA gene of Porphyromonas gingivalis which comprise a nucleotide sequence of the following:

AGTTGGTGAGCGAGC; (Por003, SEQ ID NO: 178)
or
CTGAGCTGTCGTGCA. (Por23S08, SEQ ID NO: 179)

In another aspect (12-i-b), the present invention provides isolated nucleic acid molecules derived from 23S rRNA gene of Porphyromonas gingivalis which comprise a nucleotide sequence of the following:

GTTTTTGTGAGTGGA; (Por001, SEQ ID NO: 180)
or
TGATGGGTGGGGTTG. (Por002, SEQ ID NO: 181)

In another aspect (12-ii), the present invention provides nucleic acid probes for detecting Porphyromonas gingivalis which comprise any one of nucleotide sequences shown in SEQ ID NO: 178 to SEQ ID NO: 181.

In another aspect (12-iii), the present invention provides a composition comprising at least one of nucleic acid probes for detecting Porphyromonas gingivalis which comprises any one of nucleotide sequences shown in SEQ ID NO: 178 to SEQ ID NO: 181.

In another aspect (12-iv), the present invention provides a kit for detecting and identifying Porphyromonas gingivalis in a biological sample which comprises (a) a composition comprising at least one of nucleic acid probes for detecting said bacteria which comprises any one of nucleotide sequences shown in SEQ ID NO: 178 to SEQ ID NO: 181, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (12-v), the present invention provides a DNA chip in which at least one of nucleic acid molecules comprising any one of nucleotide sequences shown in SEQ ID NO: 178 to SEQ ID NO: 181 is immobilized on a solid support.

In another aspect (12-vi), the present invention provides a method for detection and identification of Porphyromonas gingivalis in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 178 to SEQ ID NO: 181 under the appropriate hybridization and wash conditions, (d) detecting the hybrids formed in step (c), and (e) identifying the bacteria present in the sample from the differential hybridization signals obtained in step (d).

In another aspect (13-i), the present invention provides isolated nucleic acid molecules derived from 23S rRNA gene of Peptostreptococcus anaerobius which comprise a nucleotide sequence of the following:

AGGAGGAAGAGAAAG; (P. anae003, SEQ ID NO: 186)
or
GCGAAAGGAAAAGAG. (P. anae004, SEQ ID NO: 187)

In another aspect (13-ii), the present invention provides nucleic acid probes for detecting Peptostreptococcus anaerobius which comprise the nucleotide sequence shown in SEQ ID NO: 186 or the nucleotide sequence shown in SEQ ID NO: 187.

In another aspect (13-iii), the present invention provides a composition comprising at least one of nucleic acid probes for detecting Peptostreptococcus anaerobius which comprises the nucleotide sequence shown in SEQ ID NO: 186 or the nucleotide sequence shown in SEQ ID NO: 187.

In another aspect (13-iv), the present invention provides a kit for detecting and identifying Peptostreptococcus anaerobius in a biological sample which comprises (a) a composition comprising at least one of nucleic acid probes for detecting said bacteria which comprises the nucleotide sequence shown in SEQ ID NO: 186 or the nucleotide sequence shown in SEQ ID NO: 187, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (13-v), the present invention provides a DNA chip in which at least one of nucleic acid molecules comprising the nucleotide sequence shown in SEQ ID NO: 186 or the nucleotide sequence shown in SEQ ID NO: 187 is immobilized on a glass slide.

In another aspect (13-vi), the present invention provides a method for detection and identification of Peptostreptococcus anaerobius in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 186 or the nucleotide sequence shown in SEQ ID NO: 187 under the appropriate hybridization and wash conditions, (d) detecting the hybrids formed in step (c), and (e) identifying the bacteria present in the sample from the differential hybridization signals obtained in step (d).

In another aspect (14-i), the present invention provides isolated nucleic acid molecules derived from 23S rRNA gene of Peptostreptococcus magnus which comprise the following nucleotide sequence:

CATGCAACGATCCGT. (P. magn002, SEQ ID NO: 190)

In another aspect (14-ii), the present invention provides nucleic acid probes for detecting Peptostreptococcus magnus which comprise the nucleotide sequence shown in SEQ ID NO: 190.

In another aspect (14-iii), the present invention provides a composition comprising at least one of nucleic acid probes for detecting Peptostreptococcus maagnus which comprises the nucleotide sequence shown in SEQ ID NO: 190.

In another aspect (14-iv), the present invention provides a kit for detecting and identifying Peptostreptococcus magnus in a biological sample which comprises (a) a composition comprising at least one of nucleic acid probes for detecting said bacteria which comprises the nucleotide sequence shown in SEQ ID NO: 190, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (14-v), the present invention provides a DNA chip in which at least one of nucleic acid molecules comprising the nucleotide sequence shown in SEQ ID NO: 190 is immobilized on a glass slide.

In another aspect (14-vi), the present invention provides a method for detection and identification of Peptostreptococcus magnus in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 190 under the appropriate hybridization and wash conditions, (d) detecting the hybrids formed in step (c), and (e) identifying the bacteria present in the sample from the differential hybridization signals obtained in step (d).

In another aspect (15-i), the present invention provides isolated nucleic acid molecules derived from 23S rRNA gene of Fusobacterium necrophorum which comprise a nucleotide sequence selected from the group consisting of the following:

TTTCGCAGACGTAAG; (fnecro01, SEQ ID NO: 193)
GTTTTCTTGCGCTGT; (fnecro02, SEQ ID NO: 194)
CCGTATTCATGTCAA; (fnecro03, SEQ ID NO: 195)
CTGCAAGCTATTTCG; (fnecro05, SEQ ID NO: 196)
CAGACGTAAGCAAAG; (fnecro06, SEQ ID NO: 197)
and
CCTGTATTGGTAGTT. (fnecro07, SEQ ID NO: 198)

In another aspect (15-ii), the present invention provides nucleic acid probes for detecting Fusobacterium necrophorum which comprise any one of nucleotide sequences shown in SEQ ID NO: 193 to SEQ ID NO: 198.

In another aspect (15-iii), the present invention provides a composition comprising at least one of nucleic acid probes for detecting Fusobacterium necrophorum which comprises any one of nucleotide sequences shown in SEQ ID NO: 193 to SEQ ID NO: 198.

In another aspect (15-iv), the present invention provides a kit for detecting and identifying Fusobacterium necrophorum in a biological sample which comprises (a) a composition comprising at least one of nucleic acid probes for detecting said bacteria which comprises any one of nucleotide sequences shown in SEQ ID NO: 193 to SEQ ID NO: 198, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (15-v), the present invention provides a DNA chip in which at least one of nucleic acid molecules comprising any one of nucleotide sequences shown in SEQ ID NO: 193 to SEQ ID NO: 198 is immobilized on a solid support.

In another aspect (15-vi), the present invention provides a method for detection and identification of Fusobacterium necrophorum in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 193 to SEQ ID NO: 198 under the appropriate hybridization and wash conditions, (d) detecting the hybrids formed in step (c), and (e) identifying the bacteria present in the sample from the differential hybridization signals obtained in step (d).

In another aspect (16-i-a), the present invention provides isolated nucleic acid molecules derived from 23S rRNA gene of Proteus vulgaris which comprise the following nucleotide sequence:

AGAGGAGGCTTAGTG. (P vulga04, SEQ ID NO: 199)

In another aspect (16-i-b), the present invention provides isolated nucleic acid molecules derived from 23S rRNA gene of Proteus vulgaris which comprise the following nucleotide sequence:

ATACGTGTTATGTGC. (P vulga01, SEQ ID NO: 200)

In another aspect (16-ii), the present invention provides nucleic acid probes for detecting Proteus vulgaris which comprise the nucleotide sequence shown in SEQ ID NO: 199 or the nucleotide sequence shown in SEQ ID NO: 200.

In another aspect (16 iii), the present invention provides a composition comprising at least one of nucleic acid probes for detecting Proteus vulgaris which comprises the nucleotide sequence shown in SEQ ID NO: 199 or the nucleotide sequence shown in SEQ ID NO: 200.

In another aspect (16-iv), the present invention provides a kit for detecting and identifying Proteus vulgaris in a biological sample which comprises (a) a composition comprising at least one of nucleic acid probes for detecting said bacteria which comprises the nucleotide sequence shown in SEQ ID NO: 199 or the nucleotide sequence shown in SEQ ID NO: 200, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (16-v), the present invention provides a DNA chip in which at least one of nucleic acid molecules comprising the nucleotide sequence shown in SEQ ID NO: 199 or the nucleotide sequence shown in SEQ ID NO: 200 is immobilized on a solid support.

In another aspect (16-vi), the present invention provides a method for detection and identification of Proteus vulgaris in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 199 or the nucleotide sequence shown in SEQ ID NO: 200 under the appropriate hybridization and wash conditions, (d) detecting the hybrids formed in step (c), and (e) identifying the bacteria present in the sample from the differential hybridization signals obtained in step (d).

In another aspect (17-i), the present invention provides isolated nucleic acid molecules derived from 23S rRNA gene of Enterobacter aerogenes which comprise a nucleotide sequence selected from the group consisting of the following:

TTCCGACGGTACAGG; (e. aero01, SEQ ID NO: 207)
GTATCAGTAAGTGCG; (e. aero03, SEQ ID NO: 208)
and
TTATCCAGGCAAATC. (e. aero04, SEQ ID NO: 209)

In another aspect (17-ii), the present invention provides nucleic acid probes for detecting Enterobacter aerogenes which comprise any one of nucleotide sequences shown in SEQ ID NO: 207 to SEQ ID NO: 209.

In another aspect (17-iii), the present invention provides a composition comprising at least one of nucleic acid probes for detecting Enterobacter aerogenes which comprises any one of nucleotide sequences shown in SEQ ID NO: 207 to SEQ ID NO: 209.

In another aspect (17-iv), the present invention provides a kit for detecting and identifying Enterobacter aerogenes in a biological sample which comprises (a) a composition comprising at least one of nucleic acid probes for detecting said bacteria which comprises any one of nucleotide sequences shown in SEQ ID NO: 207 to SEQ ID NO: 209, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions, and (e) optionally a means for detection of said hybrids.

In another aspect (17-v), the present invention provides a DNA chip in which at least one of nucleic acid molecules comprising any one of nucleotide sequences shown in SEQ ID NO: 207 to SEQ ID NO: 209 is immobilized on a solid support.

In another aspect (17-vi), the present invention provides a method for detection and identification of Enterobacter aerogenes in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 207 to SEQ ID NO: 209 under the appropriate hybridization and wash conditions, (d) detecting the hybrids formed in step (c), and (e) identifying the bacteria present in the sample from the differential hybridization signals obtained in step (d).

In another aspect (18-i), the present invention provides isolated nucleic acid molecules derived from 23S rRNA gene of Streptococcus mutans which comprise the following nucleotide sequence:

TAGGTATTCTCTCCT. (S. mutan001, SEQ ID NO: 212)

In another aspect (18-ii), the present invention provides nucleic acid probes for detecting Streptococcus mutans which comprise the nucleotide sequence shown in SEQ ID NO: 212.

In another aspect (18-iii), the present invention provides a composition comprising at least one of nucleic acid probes for detecting Streptococcus mutans which comprises the nucleotide sequence shown in SEQ ID NO: 212.

In another aspect (18-iv), the present invention provides a kit for detecting and identifying Streptococcus mutans in a biological sample which comprises (a) a composition comprising at least one of nucleic acid probes for detecting said bacteria which comprises the nucleotide sequence shown in SEQ ID NO: 212, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (18-v), the present invention provides a DNA chip in which at least one of nucleic acid molecules comprising the nucleotide sequence shown in SEQ ID NO: 212 is immobilized on a solid support.

In another aspect (18-vi), the present invention provides a method for detection and identification of Streptococcus mutans in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 212 under the appropriate hybridization and wash conditions, (d) detecting the hybrids formed in step (c), and (e) identifying the bacteria present in the sample from the differential hybridization signals obtained in step (d).

In another aspect (19-i), the present invention provides isolated nucleic acid molecules derived from 23S rRNA gene of Kingella kingap which comprise a nucleotide sequence selected from the group consisting of the following:

GGTTAGCAAACTGTT; (k. king02, SEQ ID NO: 218)
CCAGTAGGTGGAAAG; (k. king03, SEQ ID NO: 219)
AACACCGAGACGTGA; (k. king04, SEQ ID NO: 220)
and
TATTCAATGCGATGG. (k. king09, SEQ ID NO: 221)

In another aspect (19-ii), the present invention provides nucleic acid probes for detecting Kingella kingap which comprise any one of nucleotide sequences shown in SEQ ID NO: 218 to SEQ ID NO: 221.

In another aspect (19-iii), the present invention provides a composition comprising at least one of nucleic acid probes for detecting Kingella kingap which comprises any one of nucleotide sequences shown in SEQ ID NO: 218 to SEQ ID NO: 221.

In another aspect (19-iv), the present invention provides a kit for detecting and identifying Kingella kingap in a biological sample which comprises (a) a composition comprising at least one of nucleic acid probes for detecting said bacteria which comprises any one of nucleotide sequences shown in SEQ ID NO: 218 to SEQ ID NO: 221, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (19-v), the present invention provides a DNA chip in which at least one of nucleic acid molecules comprising any one of nucleotide sequences shown in SEQ ID NO: 218 to SEQ ID NO: 221 is immobilized on a solid support.

In another aspect (19-vi), the present invention provides a method for detection and identification of Kingella kingap in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 218 to SEQ ID NO: 221 under the appropriate hybridization and wash conditions, (d) detecting the hybrids formed in step (c), and (e) identifying the bacteria present in the sample from the differential hybridization signals obtained in step (d).

In another aspect (20-i), the present invention provides isolated nucleic acid molecules derived from 23S rRNA gene of Bacteroides ovatus which comprise a nucleotide sequence selected from the group consisting of the following:

TAGAAGGAAGCATTC; (b. ovatus01, SEQ ID NO: 227)
CCAATGTTGTTACGG; (b. ovatus02, SEQ ID NO: 228)
and
TGTAGGACCACGATG. (b. ovatus05, SEQ ID NO: 229)

In another aspect (20-ii), the present invention provides nucleic acid probes for detecting Bacteroides ovatus which comprise any one of nucleotide sequences shown in SEQ ID NO: 227 to SEQ ID NO: 229.

In another aspect (20-iii), the present invention provides a composition comprising at least one of nucleic acid probes for detecting Bacteroides ovatus which comprises any one of nucleotide sequences shown in SEQ ID NO: 227 to SEQ ID NO: 229.

In another aspect (20-iv), the present invention provides a kit for detecting and identifying Bacteroides ovatus in a biological sample which comprises (a) a composition comprising at least one of nucleic acid probes for detecting said bacteria which comprises any one of nucleotide sequences shown in SEQ ID NO: 227 to SEQ ID NO: 229, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e.) optionally a means for detection of said hybrids.

In another aspect (20-v), the present invention provides a DNA chip in which at least one of nucleic acid molecules comprising any one of nucleotide sequences shown in SEQ ID NO: 227 to SEQ ID NO: 229 is immobilized on a solid support.

In another aspect (20-vi), the present invention provides a method for detection and identification of Bacteroides ovatus in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 227 to SEQ ID NO: 229 under the appropriate hybridization and wash conditions, (d) detecting the hybrids formed in step (c), and (e) identifying the bacteria present in the sample from the differential hybridization signals obtained in step (d).

In another aspect (21-i), the present invention provides isolated nucleic acid molecules derived from 23S rRNA gene of Bacteroides thetaiotaomicron which comprise the following nucleotide sequence:

GCTAACGCAGGGAAC. (b. thetaio006, SEQ ID NO: 234)

In another aspect (21-ii), the present invention provides nucleic acid probes for detecting Bacteroides thetaiotaomicron which comprise the nucleotide sequence shown in SEQ ID NO: 234.

In another aspect (21-iii), the present invention provides a composition comprising at least one of nucleic acid probes for detecting Bacteroides thetaiotaomicron which comprises the nucleotide sequence shown in SEQ ID NO: 234.

In another aspect (21-iv), the present invention provides a kit for detecting and identifying Bacteroides thetaiotaomicron in a biological sample which comprises (a) a composition comprising at least one of nucleic acid probes for detecting said bacteria which comprises the nucleotide sequence shown in SEQ ID NO: 234, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (21-v), the present invention provides A DNA chip in which at least one of nucleic acid molecules comprising the nucleotide sequence shown in SEQ ID NO: 234 is immobilized on a glass slide.

In another aspect (21-vi), the present invention provides a method for detection and identification of Bacteroides thetaiotaomicron in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 234 under the appropriate hybridization and wash conditions, (d) detecting the hybrids formed in step (c), and (e) identifying the bacteria present in the sample from the differential hybridization signals obtained in step (d).

In another aspect (22-i), the present invention provides isolated nucleic acid molecules derived from 23S rRNA gene of Clostridium diffcile which comprise the following nucleotide sequence:

GTTCGTCCGCCCCTG. (C. diffc005, SEQ ID NO: 240)

In another aspect (22-ii), the present invention provides nucleic acid probes for detecting Clostridium diffcile which comprise the nucleotide sequence shown in SEQ ID NO: 240.

In another aspect (22-iii), the present invention provides a composition comprising at least one of nucleic acid probes for detecting Clostridium diffcile which comprises the nucleotide sequence shown in SEQ ID NO: 240.

In another aspect (22-iv), the present invention provides a kit for detecting and identifying Clostridium diffcile in a biological sample which comprises (a) a composition comprising at least one of nucleic acid probes for detecting said bacteria which comprises the nucleotide sequence shown in SEQ ID NO: 240, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (22-v), the present invention provides a DNA chip in which at least one of nucleic acid molecules comprising the nucleotide sequence shown in SEQ ID NO: 240 is immobilized on-a glass slide.

In another aspect (22-vi), the present invention provides a method for detection and identification of Clostridium diffcile in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 240 under the appropriate hybridization and wash conditions, (d) detecting the hybrids formed in step (c), and (e) identifying the bacteria present in the sample from the differential hybridization signals obtained in step (d).

In another aspect (23-i-a), the present invention provides isolated nucleic acid molecules derived from 23S rRNA gene of Haemophilus aphrophilas which comprise the following nucleotide sequence:

GGTGAAGAACCCACT. (H. aphro003, SEQ ID NO: 245)

In another aspect (23-i-b), the present invention provides isolated nucleic acid molecules derived from ITS gene of Haemohilus aphrophilas which comprise a nucleotide sequence of the following:

TGGGAGTGGGTTGTC; (H. aphro001, SEQ ID NO: 246)
or
TAACAAACCGGAAAC. (H. aphro002, SEQ ID NO: 247)

In another aspect (23-ii), the present invention provides nucleic acid probes for detecting Haemohilus aphrophilas which comprise any one of nucleotide sequences shown in SEQ ID NO: 245 to SEQ ID NO: 247.

In another aspect (23-iii), the present invention provides a composition comprising at least one of nucleic acid probes for detecting Haemohilus aphrophilas which comprises any one of nucleotide sequences shown in SEQ ID NO: 245 to SEQ ID NO: 247.

In another aspect (23-iv), the present invention provides a kit for detecting and identifying Haemohilus aphrophilas in a biological sample which comprises (a) a composition comprising at least one of nucleic acid probes for detecting said bacteria which comprises any one of nucleotide sequences shown in SEQ ID NO: 245 to SEQ ID NO: 247, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (23-v), the present invention provides a DNA chip in which at least one of nucleic acid molecules comprising any one of nucleotide sequences shown in SEQ ID NO: 245 to SEQ ID NO: 247 is immobilized on a solid support.

In another aspect (23-vi), the present invention provides a method for detection and identification of Haemohilus aphrophilas in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 245 to SEQ ID NO: 247 under the appropriate hybridization and wash conditions, (d) detecting the hybrids formed in step (c), and (e) identifying the bacteria present in the sample from the differential hybridization signals obtained in step (d).

In another aspect (24-i-a), the present invention provides isolated nucleic acid molecules derived from 23S rRNA gene of Neisseria gonorrhea which comprise a nucleotide sequence of the following:

TATCAAAGTAGGGAT; (N.gono005, SEQ ID NO: 254)
or
AGTCAACGGGTAGGT. (N. gono006, SEQ ID NO: 255)

In another aspect (24-i-b), the present invention provides isolated nucleic acid molecules derived from ITS gene of Neisseria gonorrhea which comprise the following nucleotide sequence:

AACCTCTCGCAAGAG. (N. gono002, SEQ ID NO: 256)

In another aspect (24-ii), the present invention provides nucleic acid probes for detecting Neisseria gonorrhea which comprise any one of nucleotide sequences shown in SEQ ID NO: 254 to SEQ ID NO: 256.

In another aspect (24-iii), the present invention provides a composition comprising at least one of nucleic acid probes for detecting Neisseria gonorrhea which comprises any one of nucleotide sequences shown in SEQ ID NO: 254 to SEQ ID NO: 256.

In another aspect (24-iv), the present invention provides a kit for detecting and identifying Neisseria gonorrhea in a biological sample which comprises (a) a composition comprising at least one of nucleic acid probes for detecting said bacteria which comprises any one of nucleotide sequences shown in SEQ ID NO: 254 to SEQ ID NO: 256, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (24-v), the present invention provides a DNA chip in which at least one of nucleic acid molecules comprising any one of nucleotide sequences shown in SEQ ID NO: 254 to SEQ ID NO: 256 is immobilized on a solid support.

In another aspect (24-vi), the present invention provides a method for detection and identification of Neisseria gonorrhea in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 254 to SEQ ID NO: 256 under the appropriate hybridization and wash conditions, (d) detecting the hybrids formed in step (c), and (e) identifying the bacteria present in the sample from the differential hybridization signals obtained in step (d).

In another aspect (25-i-a), the present invention provides isolated nucleic acid molecules derived from 23S rRNA gene of Eikenella corrodens which comprise a nucleotide sequence of the following:

GGATAGGAGAAGGAA; (E. corro005, SEQ ID NO: 262)
or
ACTCATCATCGATCC. (E. corro006, SEQ ID NO: 263)

In another aspect (25-i-b), the present invention provides isolated nucleic acid molecules derived from 23S rRNA gene of Eikenella corrodens which comprise the following nucleotide sequence:

AGTCGTAGAGCGGAG. (E. corro001, SEQ ID NO: 264)

In another aspect (25-ii), the-present invention provides nucleic acid probes for detecting Eikenella corrodens which comprise any one of nucleotide sequences shown in SEQ ID NO: 262 to SEQ ID NO: 264.

In another aspect (25-iii), the present invention provides a composition comprising at least one of nucleic acid probes for detecting Eikenella corrodens which comprises any one of nucleotide sequences shown in SEQ ID NO: 262 to SEQ ID NO: 264.

In another aspect (25-iv), the present invention provides a kit for detecting and identifying Eikenella corrodens in a biological sample which comprises (a) a composition comprising at least one of nucleic acid probes for detecting said bacteria which comprises any one of nucleotide sequences shown in SEQ ID NO: 262 to SEQ ID NO: 264, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (25-v), the present invention provides a DNA chip in which at least one of nucleic acid molecules comprising any one of nucleotide sequences shown in SEQ ID NO: 262 to SEQ ID NO: 264 is immobilized on a solid support.

In another aspect (25-vi), the present invention provides a method for detection and identification of Eikenella corrodens in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 262 to SEQ ID NO 264 under the appropriate hybridization and wash conditions, (d) detecting the hybrids formed in step (c), and (e) identifying the bacteria present in the sample from the differential hybridization signals obtained in step (d).

In another aspect (26-i), the present invention provides isolated nucleic acid molecules derived from 23S rRNA gene of Bacteroides vulgatus which comprise a nucleotide sequence of the following:

AGTCAGCGTCGAAGG; (b. vulga03, SEQ ID NO: 268)
or
CGAATGCGCATCAGT. (b. vulga07, SEQ ID NO: 269)

In another aspect (26-ii), the present invention provides nucleic acid probes for detecting Bacteroides vulgatus which comprise the nucleotide sequence shown in SEQ ID NO: 268 or the nucleotide sequence shown in SEQ ID NO: 269.

In another aspect (26-iii), the present invention provides a composition comprising at least one of nucleic acid probes for detecting Bacteroides vulgatus which comprises the nucleotide sequence shown in SEQ ID NO: 268 or the nucleotide sequence shown in SEQ ID NO: 269.

In another aspect (26-iv), the present invention provides a kit for detecting and identifying Bacteroides vulgatus in a biological sample which comprises (a) a composition comprising at least one of nucleic acid probes for detecting said bacteria which comprises the nucleotide sequence shown in SEQ ID NO: 268 or the nucleotide sequence shown in SEQ ID NO: 269, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (26-v), the present invention provides a DNA chip in which at least one of nucleic acid molecules comprising the nucleotide sequence shown in SEQ ID NO: 268 or the nucleotide sequence shown in SEQ ID NO: 269 is immobilized on a solid support.

In another aspect (26-vi), the present invention provides a method for detection and identification of Bacteroides vulgatus in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 268 or the nucleotide sequence shown in SEQ ID NO: 269 under the appropriate hybridization and wash conditions, (d) detecting the hybrids formed in step (c), and (e) identifying the bacteria present in the sample from the differential hybridization signals obtained in step (d).

In another aspect (27-i), the present invention provides isolated nucleic acid molecules derived from 23S rRNA gene of Branhamella catarrhalis which comprise the following nucleotide sequence:

ATATCTTCGCGCTGT. (B. catar005, SEQ ID NO: 280)

In another aspect (27-ii), the present invention provides nucleic acid probes for detecting Branhamella catarrhalis which comprise the nucleotide sequence shown in SEQ ID NO: 280.

In another aspect (27-iii), the present invention provides a composition comprising at least one of nucleic acid probes for detecting Branhamella catarrhalis which comprises the nucleotide sequence shown in SEQ ID NO: 280.

In another aspect (27-iv), the present invention provides a kit for detecting and identifying Branhamella catarrhalis in a biological sample which comprises (a) a composition comprising at least one of nucleic acid probes for detecting said bacteria which comprises the nucleotide sequence shown in SEQ ID NO: 280, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (27-v), the present invention provides a DNA chip in which at least one of nucleic acid molecules comprising the nucleotide sequence shown in SEQ ID NO: 280 is immobilized on a solid support.

In another aspect (27-vi), the present invention provides a method for detection and identification of Branhamella catarrhalis in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 280 under the appropriate hybridization and wash conditions, (d) detecting the hybrids formed in step (c), and (e) identifying the bacteria present in the sample from the differential hybridization signals obtained in step (d).

In another aspect (28-i), the present invention provides isolated nucleic acid molecules derived from 23S rRNA gene of Sutterella wadsworthensis which comprise a nucleotide sequence selected from the group consisting of the following:

TTCGGGTCCGTAATT; (Swad02, SEQ ID NO: 292)
AATCAAGGCCGAGGC; (Swad03, SEQ ID NO: 293)
and
GCCGAGGCGTGATGA. (Swad04, SEQ ID NO: 294)

In another aspect (28-ii), the present invention provides nucleic acid probes for detecting Sutterella wadsworthensis which comprise any one of nucleotide sequences shown in SEQ ID NO: 292 to SEQ ID NO: 294.

In another aspect (28-iii), the present invention provides a composition comprising at least one of nucleic acid probes for detecting Sutterella wadsworthensis which comprises any one of nucleotide sequences shown in SEQ ID NO: 292 to SEQ ID NO: 294.

In another aspect (28-iv), the present invention provides a kit for detecting and identifying Sutterella wadsworthensis in a biological sample which comprises (a) a composition comprising at least one of nucleic acid probes for detecting said bacteria which comprises any one of nucleotide sequences shown in SEQ ID NO: 292 to SEQ ID NO: 294, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (28-v), the present invention provides a DNA chip in which at least one of nucleic acid molecules comprising any one of nucleotide sequences shown in SEQ ID NO: 292 to SEQ ID NO: 294 is immobilized on a solid support.

In another aspect (28-vi), the present invention provides a method for detection and identification of Bacteroides ovatus in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 292 to SEQ ID NO: 294 under the appropriate hybridization and wash conditions, (d) detecting the hybrids formed in step (c), and (e) identifying the bacteria present in the sample from the differential hybridization signals obtained in step (d).

In another aspect. (29-i), the present invention provides isolated nucleic acid molecules derived from 23S rRNA gene of Actinomyces israelii which comprise the following nucleotide sequence:

AACCTGGCTGGTGGC. (Acii1, SEQ ID NO: 296)

In another aspect (29-ii), the present invention provides nucleic acid probes for detecting Actinomyces israelii which comprise the nucleotide sequence shown in SEQ ID NO: 296.

In another aspect (29-iii), the present invention provides a composition comprising at least one of nucleic acid probes for detecting Actinomyces israelii which comprises the nucleotide sequence shown in SEQ. ID. NO: 296.

In another aspect (29-iv), the present invention provides a kit for detecting and identifying Actinomyces israelii in a biological sample which comprises (a) a composition comprising at least one of nucleic acid probes for detecting said bacteria which comprises the nucleotide sequence shown in SEQ ID NO: 296, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (29-v), the present invention provides a DNA chip in which at least one of nucleic acid molecules comprising the nucleotide sequence shown in SEQ ID NO: 296 is immobilized on a solid support.

In another aspect (29-vi), the present invention provides a method for detection and identification of Actinomyces israelii in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 296 under the appropriate hybridization and wash conditions, (d) detecting the hybrids formed in step (c), and (e) identifying the bacteria present-in the sample from the differential hybridization signals obtained in step (d).

In another aspect (30-i), the present invention provides isolated nucleic acid molecules derived from 23S rRNA gene of Staphylococcus epidermidis which comprise a nucleotide sequence of the following:

GATAGATAACAGGTG; (SeM01, SEQ ID NO: 299)
or
AGGGTTCACGCCCAG. (SeM02, SEQ ID NO: 300)

In another aspect (30-ii), the present invention provides nucleic acid probes for detecting Staphylococcus epidermidis which comprise the nucleotide sequence shown in SEQ ID NO: 299 or the nucleotide sequence shown in SEQ ID NO: 300.

In another aspect (30-iii), the present invention provides a composition comprising at least one of nucleic acid probes for detecting Staphylococcus epidermidis which comprises the nucleotide sequence shown in SEQ ID NO: 299 or the nucleotide sequence shown in SEQ ID NO: 300.

In another aspect (30-iv), the present invention provides a kit for detecting and identifying Staphylococcus epidermidis in a biological sample which comprises (a) a composition comprising at least one of nucleic acid probes for detecting said bacteria which comprises the nucleotide sequence shown in SEQ ID NO: 299 or the nucleotide sequence shown in SEQ ID NO: 300, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (30-v), the present invention provides a DNA chip in which at least one of nucleic acid molecules comprising the nucleotide sequence shown in SEQ ID NO: 299 or the nucleotide sequence shown in SEQ ID NO: 300 is immobilized on a solid support.

In another aspect (30-vi), the present invention provides a method for detection and identification of Staphylococcus epidermidis in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 299 or the nucleotide sequence shown in SEQ ID NO: 300 under the appropriate hybridization and wash conditions, (d) detecting the hybrids formed in step (c), and (e) identifying the bacteria present in the sample from the differential hybridization signals obtained in step (d).

In another aspect (31-i), the present invention provides isolated nucleic acid molecules derived from 23S rRNA gene of Burkholderia cepacia which comprise a nucleotide sequence of the following:

TTGTTAGCCGAACGC; (Bur23, SEQ ID NO: 304)
or
GGGTGTGGCGCGAGC. (Bur01, SEQ ID NO: 305)

In another aspect (31-ii), the present invention provides nucleic acid probes for detecting Burkholderia cepacia which comprise the nucleotide sequence shown in SEQ ID NO: 304 or the nucleotide sequence shown in SEQ ID NO: 305.

In another aspect (31-iii), the present invention provides a composition comprising at least one of nucleic acid probes for detecting Burkholderia cepacia which comprises the nucleotide sequence shown in SEQ ID NO: 304 or the nucleotide sequence shown in SEQ ID NO: 305.

In another aspect (31-iv), the present invention provides a kit for detecting and identifying Burkholderia cepacia in a biological sample which comprises (a) a composition comprising at least one of nucleic acid probes for detecting said bacteria which comprises the nucleotide sequence shown in SEQ ID NO: 304 or the nucleotide sequence shown in SEQ ID NO: 305, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for-detection of said hybrids.

In another aspect (31-v), the present invention provides a DNA chip in which at least one of nucleic acid molecules comprising the nucleotide sequence shown in SEQ ID NO: 304 or the nucleotide sequence shown in SEQ ID NO: 305 is immobilized on a solid support.

In another aspect (31-vi), the present invention provides a method for detection and identification of Burkholderia cepacia in a biological sample which comprises the steps of (a) if appropriate, isolating and/or concentrating the polynucleic acids present in the sample, (b) if appropriate, amplifying the polynucleic acids with-a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 304 or the nucleotide sequence shown in SEQ ID NO: 305 under the appropriate hybridization and wash conditions, (d) detecting the hybrids formed in step (c), and (e) identifying the bacteria present in the sample from the differential hybridization signals obtained in step (d).

In another aspect (32-i), the present invention provides isolated nucleic acid molecules derived from 23S rRNA gene of Salmonella spp. (enteritidis) which comprise the following nucleotide sequence:

GCCTGAATCAGCATG. (Styp23, SEQ ID NO: 307)

In another aspect (32-ii), the present invention provides nucleic acid probes for detecting Salmonella spp. (enteritidis) which comprise the nucleotide sequence shown in SEQ ID NO: 307.

In another aspect (32-iii), the present invention provides a composition comprising at least one of nucleic acid probes for detecting Salmonella spp. (enteritidis) which comprises the nucleotide sequence shown in SEQ ID NO: 307.

In another aspect (32-iv), the present invention provides a kit for detecting and identifying Salmonella spp. (enteritidis) in a biological sample which comprises (a) a composition comprising at least one of nucleic acid probes for detecting said bacteria which comprises the nucleotide sequence shown in SEQ ID NO: 307, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (32-v), the present invention provides a DNA chip in which at least one of nucleic acid molecules comprising the nucleotide sequence shown in SEQ ID NO: 307 is immobilized on a solid support.

In another aspect (32-vi), the present invention provides a method for detection and identification of Salmonella spp. (enteritidis) in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 307 under the appropriate hybridization and wash conditions, (d) detecting the hybrids formed in step (c), and (e) identifying the bacteria present in the sample from the differential hybridization signals obtained in step (d).

In another aspect (33-i), the present invention provides isolated nucleic acid molecules derived from 23S rRNA gene of Escherichia coli which comprise the following nucleotide sequence:

CTGAAGCGACAAATG. (E coli003, SEQ ID NO: 312)

In another aspect (33-ii), the present invention provides nucleic acid probes for detecting Escherichia coli which comprise the nucleotide sequence shown in SEQ ID NO: 312.

In another aspect (33-iii), the present invention provides a composition comprising at least one of nucleic acid probes for detecting Escherichia coli which comprises the nucleotide sequence shown in SEQ ID NO: 312.

In another aspect (33-iv), the present invention provides a kit for detecting and identifying Escherichia coli in a biological sample which comprises (a) a composition comprising at least one of nucleic acid probes for detecting said bacteria which comprises the nucleotide sequence shown in SEQ ID NO: 312, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (33-v), the present invention provides a DNA chip in which at least one of nucleic acid molecules comprising the nucleotide sequence shown in SEQ ID NO: 312 is immobilized on a solid support.

In another aspect (33-vi), the present invention provides a method for detection and identification of Escherichia coli in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 312 under the appropriate hybridization and wash conditions, (d) detecting the hybrids formed in step (c), and (e) identifying the bacteria present in the sample from the differential hybridization signals obtained in step (d).

In another aspect (34-i), the present invention provides isolated nucleic acid molecules derived from 23S rRNA gene of Klebsiella pneumoniae which comprise a nucleotide sequence of the following:

GTACACCAAAATGCA; (K pneu23, SEQ ID NO: 317)
or
GCTGAGACCAGTCGA. (K. pneu002, SEQ ID NO: 318)

In another aspect (34-ii), the present invention provides nucleic acid probes for detecting Klebsiella pneumoniae which comprise the nucleotide sequence shown in SEQ ID NO: 317 or the nucleotide sequence shown in SEQ ID NO: 318.

In another aspect (34-iii), the present invention provides a composition comprising at least one of nucleic acid probes for detecting Klebsiella pneumoniae which comprises the nucleotide sequence shown in SEQ ID NO: 317 or the nucleotide sequence shown in SEQ ID NO: 318.

In another aspect (34-iv), the present invention provides a kit for detecting and identifying Klebsiella pneumoniae in a biological sample which comprises (a) a composition comprising at least one of nucleic acid probes for detecting said bacteria which comprises the nucleotide sequence shown in SEQ ID NO: 317 or the nucleotide sequence shown in SEQ ID NO: 318, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (34-v), the present invention provides a DNA chip in which at least one of nucleic acid molecules comprising the nucleotide sequence shown in SEQ ID NO: 317 or the nucleotide sequence shown in SEQ ID NO: 318 is immobilized on a solid support.

In another aspect (34-vi), the present invention provides a method for detection and identification of Klebsiella pneumoniae in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 317 or the nucleotide sequence shown in SEQ ID NO: 318 under the appropriate hybridization and wash conditions, (d) detecting the hybrids formed in step (c), and (e) identifying the bacteria present in the sample from the differential hybridization signals obtained in step (d).

In another aspect (35-i), the present invention provides isolated nucleic acid molecules derived from 23S rRNA gene of Proteus mirabilis which comprise a nucleotide sequence selected from the group consisting of the following:

GTTACCAACAATCGT; (Pm, SEQ ID NO: 321)
GGCGACGGTCGTCCC; (Pm002, SEQ ID NO: 322)
GATGACGAACCACCA; (Pm003, SEQ ID NO: 323)
and
TGAAGCAATTGATGC. (Pm004, SEQ ID NO: 324)

In another aspect (35-ii), the present invention provides nucleic acid probes for detecting Proteus mirabilis which comprise any one of nucleotide sequences shown in SEQ ID NO: 321 to SEQ ID NO: 324.

In another aspect (35-iii), the present invention provides a composition comprising at least one of nucleic acid probes for detecting Proteus mirabilis which comprises any one of nucleotide sequences shown in SEQ ID NO: 321 to SEQ ID NO: 324.

In another aspect (35-iv), the present invention provides a kit for detecting and identifying Proteus mirabilis in a biological sample which comprises (a) a composition comprising at least one of nucleic acid probes for detecting said bacteria which comprises any one of nucleotide sequences shown in SEQ ID NO: 321 to SEQ ID NO: 324, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (35-v), the present invention provides a DNA chip in which at least one of nucleic acid molecules comprising any one of nucleotide sequences shown in SEQ ID NO: 321 to SEQ ID NO: 324 is immobilized on a solid support.

In another aspect (35-vi), the present invention provides a method for detection and identification of Proteus mirabilis in a biological sample which comprises the steps of (a) if appropriate, isolating and/or concentrating the polynucleic acids present in the sample, (b) if appropriate, amplifying the polynucleic acids with a pair of forward-and reverse primers, (c) contacting the polynucleic acids of step (a) or (b) with a composition comprising at least one of nucleic acid probes for detecting said bacteria which comprises any one of nucleotide sequences shown in SEQ ID NO: 321 to SEQ ID NO: 324 under the appropriate hybridization and wash conditions, (d) detecting the hybrids formed in step (c), and (e) identifying the bacteria present in the sample from the differential hybridization signals obtained in step (d).

In another aspect (36-i), the present invention provides isolated nucleic acid molecules derived from 23S rRNA gene of Streptococcus pneumoniae which comprise the following nucleotide sequence:

TAGGACTGCAATGTG. (StreppM, SEQ ID NO: 328)

In another aspect (36-ii), the present invention provides nucleic acid probes for detecting Streptococcus pneumoniae which comprise the nucleotide sequence shown in SEQ ID NO: 328.

In another aspect (36-iii), the present invention provides a composition comprising at least one of nucleic acid probes for detecting Streptococcus pneumoniae which comprises the nucleotide sequence shown in SEQ ID NO: 328.

In another aspect (36-iv), the present invention provides a kit for detecting and identifying Streptococcus pneumoniae in a biological sample which comprises (a) a composition comprising at least one of nucleic acid probes for detecting said bacteria which comprises the nucleotide sequence shown in SEQ ID NO: 328, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (36-v), the present invention provides a DNA chip in which at least one of nucleic acid molecules comprising the nucleotide sequence shown in SEQ ID NO: 328 is immobilized on a solid support.

In another aspect (36-vi), the present invention provides a method for detection and identification of Streptococcus pneumoniae in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 328 under the appropriate hybridization and wash conditions, (d) detecting the hybrids formed in step (c), and (e) identifying the bacteria present in the sample from the differential hybridization signals obtained in step (d).

In another aspect (37-i), the present invention provides isolated nucleic acid molecules derived from 23S rRNA gene of Vibrio vulnificus which comprise the following nucleotide sequence:

GTTGACGATGCATGT. (Vvu102, SEQ ID NO: 333)

In another aspect (37-ii), the present invention provides nucleic acid probes for detecting Vibrio vulnificus which comprise the nucleotide sequence shown in SEQ ID NO: 333.

In another aspect (37-iii), the present invention provides a composition comprising at least one of nucleic acid probes for detecting Vibrio vulnificus which comprises the nucleotide sequence shown in SEQ ID NO: 333.

In another aspect (37-iv), the present invention provides a kit for detecting and identifying Vibrio vulnificus in a biological sample which comprises (a) a composition comprising at least one of nucleic acid probes for detecting said bacteria which comprises the nucleotide sequence shown in SEQ ID NO: 333, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (37-v), the present invention provides a DNA chip in which at least one of nucleic acid molecules comprising the nucleotide sequence shown in SEQ ID NO: 333 is immobilized on a solid support.

In another aspect (37-vi), the present invention provides a method for detection and identification of Vibrio vulnificus in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 333 under the appropriate hybridization and wash conditions, (d) detecting the hybrids formed in step (c), and (e) identifying the bacteria present in the sample from the differential hybridization signals obtained in step (d).

In another aspect (38-i), the present invention provides isolated nucleic acid molecules derived from 23S rRNA gene of Pseudomonas aeruginosa which comprise a nucleotide sequence of the following:

GAAGTGCCGAGCATG; (P. aeru001, SEQ ID NO: 339)
or
GGATCTTTGAAGTGA. (Pa03, SEQ ID NO: 340)

In another aspect (38-ii), the present invention provides nucleic acid probes for detecting Pseudomonas aeruginosa which comprise the nucleotide sequence shown in SEQ ID NO: 339 or the nucleotide sequence shown in SEQ ID NO: 340.

In another aspect (38-iii), the present invention provides a composition comprising at least one of nucleic acid probes for detecting Pseudomonas aeruginosa which comprises the nucleotide sequence shown in SEQ ID NO: 339 or the nucleotide sequence shown in SEQ ID NO: 340.

In another aspect (38-iv), the present invention provides a kit for detecting and identifying Pseudomonas aeruginosa in a biological sample which comprises (a) a composition comprising at least one of nucleic acid probes for detecting said bacteria which comprises the nucleotide sequence shown in SEQ ID NO: 339 or the nucleotide sequence shown in SEQ ID NO: 340, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (38-v), the present invention provides a DNA chip in which at least one of nucleic acid molecules comprising the nucleotide sequence shown in SEQ ID NO: 339 or the nucleotide sequence shown in SEQ ID NO: 340 is immobilized on a solid support.

In another aspect (38-vi), the present invention provides a method for detection and identification of Pseudomonas aeruginosa in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 339 or the nucleotide sequence shown in SEQ ID NO: 340 under the appropriate hybridization and wash conditions, (d) detecting the hybrids formed in step (c), and (e) identifying the bacteria present in the sample from the differential hybridization signals obtained in step (d).

In another aspect (39-i), the present invention provides isolated nucleic acid molecules derived from 23S rRNA gene of Aeromonas hydrophila which comprise the following nucleotide sequence:

GGCGCCTCGGTAGGG. (Ah, SEQ ID NO: 347)

In another aspect (39-ii), the present invention provides nucleic acid probes for detecting Aeromonas hydrophila which comprise the nucleotide sequence shown in SEQ ID NO: 347.

In another aspect (39-iii), the present invention provides a composition comprising at least one of nucleic acid probes for detecting Aeromonas hydrophila which comprises the nucleotide sequence shown in SEQ ID NO: 347.

In another aspect (39-iv), the present invention provides a kit for detecting and identifying Aeromonas hydrophila in a biological sample which comprises (a) a composition comprising at least one of nucleic acid probes for detecting said bacteria which comprises the nucleotide sequence shown in SEQ ID NO: 347, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (39-v), the present invention provides a DNA chip in which at least one of nucleic acid molecules comprising the nucleotide sequence shown in SEQ ID NO: 347 is immobilized on a solid support.

In another aspect (39-vi), the present invention provides a method for detection and identification of Aeromonas hydrophila in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 347 under the appropriate hybridization and wash conditions, (d) detecting the hybrids formed in step (c), and (e) identifying the bacteria present in the sample from the differential hybridization signals obtained in step (d).

In another aspect (40-i), the present invention provides isolated nucleic acid molecules derived from 23S rRNA gene of Listeria monocytogenes which comprise the following nucleotide sequence:

GGGTGCAAGCCCGAG. (LM, SEQ ID NO: 354)

In another aspect (40-ii), the present invention provides nucleic acid probes for detecting Listeria monocytogenes which comprise the nucleotide sequence shown in SEQ ID NO: 354.

In another aspect (40-iii), the present invention provides a composition comprising at least one of nucleic acid probes for detecting Listeria monocytogenes which comprises the nucleotide sequence shown in SEQ ID NO: 354.

In another aspect (40-iv), the present invention provides a kit for detecting and identifying Listeria monocytogenes in a biological sample which comprises (a) a composition comprising at least one of nucleic acid probes for detecting said bacteria which comprises the nucleotide sequence shown in SEQ ID NO: 354, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (40-v), the present invention provides a DNA chip in which at least one of nucleic acid molecules comprising the nucleotide sequence shown in SEQ ID NO: 354 is immobilized on a solid support.

In another aspect (40-vi), the present invention provides a method for detection and identification of Listeria monocytogenes in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 354 under the appropriate hybridization and wash conditions, (d) detecting the hybrids formed in step (c), and (e) identifying the bacteria present in the sample from the differential hybridization signals obtained in step (d).

In another aspect (41-i), the present invention provides isolated nucleic acid molecules-derived from 23S rRNA gene of Enterococcus faecium which comprise a nucleotide sequence of the following:

TTACGATTGTGTGAA; (E. faecium002, SEQ ID NO: 359)
or
ATAGCACATTCGAGG. (E. faecium003, SEQ ID NO: 360)

In another aspect (41-ii), the present invention provides nucleic acid probes for detecting Enterococcus faecium which comprise the nucleotide sequence shown in SEQ ID NO: 359 or the nucleotide sequence shown in SEQ ID NO: 360.

In another aspect (41-iii), the present invention provides a composition comprising at least one of nucleic acid probes for detecting Enterococcus faecium which comprises the nucleotide sequence shown in SEQ ID NO: 359 or the nucleotide sequence shown in SEQ ID NO: 360.

In another aspect (41-iv), the present invention provides a kit for detecting and identifying Enterococcus faecium in a biological sample which comprises (a) a composition comprising at least one of nucleic acid probes for detecting said bacteria which comprises the nucleotide sequence shown in SEQ ID NO: 359 or the nucleotide sequence shown in SEQ ID NO: 360, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (41-v), the present invention provides a DNA chip in which at least one of nucleic acid molecules comprising the nucleotide sequence shown in SEQ ID NO: 359 or the nucleotide sequence shown in SEQ ID NO: 360 is immobilized on a solid support.

In another aspect (41-vi), the present invention provides a method for detection and identification of Enterococcus faecium in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 359 or the nucleotide sequence shown in SEQ ID NO: 360 under the appropriate hybridization and wash conditions, (d) detecting the hybrids formed in step (c), and (e) identifying the bacteria present in the sample from the differential hybridization signals obtained in step (d).

In another aspect (42-i), the present invention provides isolated nucleic acid molecules derived from 23S rRNA gene of Staphylococcus aureus which comprise a nucleotide sequence selected from the group consisting of the following:

GATTGCACGTCTAAG; (S. aureus004, SEQ ID NO: 365)
AATCCGGTACTCGTT; (S. aureus005, SEQ ID NO: 366)
and
TCTTCGAGTCGTTGA. (S aure03 (S aureus03),
SEQ ID NO: 367)

In another aspect (42-ii), the present invention provides nucleic acid probes for detecting Staphylococcus aureus which comprise any one of nucleotide sequences shown in SEQ ID NO: 365 to SEQ ID NO: 367.

In another aspect (42-iii), the present invention provides a composition comprising at least one of nucleic acid probes for detecting Staphylococcus aureus which comprises any one of nucleotide sequences shown in SEQ ID NO: 365 to SEQ ID NO: 367.

In another aspect (42-iv), the present invention provides a kit for detecting and identifying Staphylococcus aureus in a biological sample which comprises (a) a composition comprising at least one of nucleic acid probes for detecting said bacteria which comprises any one of nucleotide sequences shown in SEQ ID NO: 365 to SEQ ID NO: 367, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (42-v), the present invention provides a DNA chip in which at least one of nucleic acid molecules comprising any one of nucleotide sequences-shown in SEQ ID NO: 365 to SEQ ID NO: 367 is immobilized on a solid support.

In another aspect (42-vi), the present invention provides a method for detection and identification of Staphylococcus aureus in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 365 to SEQ ID NO: 367 under the appropriate hybridization and wash conditions, (d) detecting the hybrids formed in step (c), and (e) identifying the bacteria present in the sample from the differential hybridization signals obtained in step (d).

In another aspect (43-i), the present invention provides isolated nucleic acid molecules derived from 23S rRNA gene of Neisseria meningitidis which comprise the following nucleotide sequence:

AGATGTGAGAGCATC. (Nm002, SEQ ID NO: 377)

In another aspect (43-ii), the present invention provides nucleic acid probes for detecting Neisseria meningitidis which comprise the nucleotide sequence shown in SEQ ID NO: 377.

In another aspect (43-iii), the present invention provides a composition comprising at least one of nucleic acid probes for detecting Neisseria meningitidis which comprises the nucleotide sequence shown in SEQ ID NO: 377.

In another aspect (43-iv), the present invention provides a kit for detecting and identifying Neisseria meningitidis in a biological sample which comprises (a) a composition comprising at least one of nucleic acid probes for detecting said bacteria which comprises the nucleotide sequence shown in SEQ ID NO: 377, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (43-v), the present invention provides a DNA chip in which at least one of nucleic acid molecules comprising the nucleotide sequence shown in SEQ ID NO: 377 is immobilized on a solid support.

In another aspect (43-vi), the present invention provides a method for detection and identification of Neisseria meningitidis in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 377 under the appropriate hybridization and wash conditions, (d) detecting the hybrids formed in step (c), and (e) identifying the bacteria present in the sample from the differential hybridization signals obtained in step (d).

In another aspect (44-i), the present invention provides isolated nucleic acid molecules derived from 23S rRNA gene of Legionella pneumophila which comprise a nucleotide sequence selected from the group consisting of the following:

TGGAGAGCATTTTAT; (L. pneu011, SEQ ID NO: 383)
GTGATTTTGAGGTGA; (L. pneu012, SEQ ID NO: 384)
and
AGATGGTAAAGAAGA. (L.pneu013, SEQ ID NO: 385)

In another aspect (44-ii), the present invention provides nucleic acid probes for detecting Legionella pneumophila which comprise any one of nucleotide sequences shown in SEQ ID NO: 383 to SEQ ID NO: 385.

In another aspect (44-iii), the present invention provides a composition comprising at least one of nucleic acid probes for detecting Legionella pneumophila which comprises any one of nucleotide sequences shown in SEQ ID NO: 383 to SEQ ID NO: 385.

In another aspect (44-iv), the present invention provides a kit for detecting and identifying Legionella pneumophila in a biological sample which comprises (a) a composition comprising at least one of nucleic acid probes for detecting said bacteria which comprises any one of nucleotide sequences shown in SEQ ID NO: 383 to SEQ ID NO: 385, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (44-v), the present invention provides a DNA chip in which at least one of nucleic acid molecules comprising any one of nucleotide sequences shown in SEQ ID NO: 383 to SEQ ID NO: 385 is immobilized on a solid support.

In another aspect (44-vi), the present invention provides a method for detection and identification of Legionella pneumophila in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 383 to SEQ ID NO: 385 under the appropriate hybridization and wash conditions, (d) detecting the hybrids formed in step (c), and (e) identifying the bacteria present in the sample from the differential hybridization signals obtained in step (d).

In another aspect (45-i), the present invention provides isolated nucleic acid molecules derived from 23S rRNA gene of Candida albicans which comprise a nucleotide sequence selected from the group consisting of the following:

TGGTAGCCATTTATG; (C. alic001, SEQ ID NO: 396)
CTGGACCAGCCGAGC; (C. alic003, SEQ ID NO: 397)
TCAAGAACGAAAGTT; (C. alic006, SEQ ID NO: 398)
AAGGATTGACAGATT; (C. alic007, SEQ ID NO: 399)
and
CATTAATCAAGAACG. (C. alic008, SEQ ID NO: 400)

In another aspect (45-ii), the present invention provides nucleic acid probes for detecting Candida albicans which comprise any one of nucleotide sequences shown in SEQ ID NO: 396 to SEQ ID NO: 400.

In another aspect (45-iii), the present invention provides a composition comprising at least one of nucleic acid probes for detecting Candida albicans which comprises any one of nucleotide sequences shown in SEQ ID NO: 396 to SEQ ID NO: 400.

In another aspect (45-iv), the present invention provides a kit for detecting and identifying Candida albicans in a biological sample which comprises (a) a composition comprising at least one of nucleic acid probes for detecting said bacteria which comprises any one of nucleotide sequences shown in SEQ ID NO: 396 to SEQ ID NO: 400, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (45-v), the present invention provides a DNA chip in which at least one of nucleic acid molecules comprising any one of nucleotide sequences shown in SEQ ID NO: 396 to SEQ ID NO: 400 is immobilized on a solid support.

In another aspect (45-vi), the present invention provides a method for detection and identification of Candida albicans in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 396 to SEQ ID NO: 400 under the appropriate hybridization and wash conditions, (d) detecting the hybrids formed in step (c), and (e) identifying the bacteria present in the sample from the differential hybridization signals obtained in step (d).

In another aspect (46-i), the present invention provides isolated nucleic acid molecules derived from 23S rRNA gene of Candida glabrata which comprise a nucleotide sequence of the following:

CTGGAATGCACCCGG; (C. glab001, SEQ ID NO: 404)
or
TGGCTTGGCGGCGAA. (C. glab003, SEQ ID NO: 405)

In another aspect (46-ii), the present invention provides nucleic acid probes for detecting Candida glabrata which comprise the nucleotide sequence shown in SEQ ID NO: 404 or the nucleotide sequence shown in SEQ ID NO: 405.

In another aspect (46-iii), the present invention provides a composition comprising at least one of nucleic acid probes for detecting Candida glabrata which comprises the nucleotide sequence shown in SEQ ID NO: 404 or the nucleotide sequence shown in SEQ ID NO: 405.

In another aspect (46-iv), the present invention provides a kit for detecting and identifying Candida glabrata in a biological sample which comprises (a) a composition comprising at least one of nucleic acid probes for detecting said bacteria which comprises the nucleotide sequence shown in SEQ ID NO: 404 or the nucleotide sequence shown in SEQ ID NO: 405, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

In another aspect (46-v), the present invention provides a DNA chip in which at least one of nucleic acid molecules comprising the nucleotide sequence shown in SEQ ID NO: 404 or the nucleotide sequence shown in SEQ ID NO: 405 is immobilized on a solid support.

In another aspect (46-vi), the present invention provides a method for detection and identification of Candida glabrata in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises the nucleotide sequence shown in SEQ ID NO: 404 or the nucleotide sequence shown in SEQ ID NO: 405 under the appropriate hybridization and wash conditions, (d) detecting the hybrids formed in step (c), and (e) identifying the bacteria present in the sample from the differential hybridization signals obtained in step (d).

In additional aspect, the present invention provides a composition comprising at least two probe types selected from the above-listed nucleic acid probes.

In another additional aspect, the present invention provides a kit for simultaneously detecting and identifying at least two microbial-species selected from the above-mentioned microbes in a biological sample which comprises (a) a composition comprising at least two probe types selected from the above nucleic acid probes, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the buffer, and (e) optionally a means for detection of said hybrids.

In another additional aspect, the present invention provides a DNA chip in which at least two probe types selected from the above-listed probes are immobilized on a solid support.

In another additional aspect, the present invention provides a method for simultaneous detection and identification of at least two microbial species selected from the above-mentioned microbes in a biological sample which comprises the steps of (a) if appropriate, isolating and/or concentrating-the polynucleic acids present in the sample, (b) if appropriate, amplifying the polynucleic acids with a pair of forward and reverse primers, (c) contacting the polynucleic acids of step (a) or (b) with a composition comprising at least two probe types selected from the above probes under the appropriate hybridization and wash conditions, (d) detecting the hybrids formed in step (c), and (e) identifying the bacteria present in the sample from the differential hybridization signals obtained in step (d).

### BRIEF DESCRIPTION OF THE DRAWINGS

FIG. 1 shows a schematic representation of the DNA chip designed for a blind test of a sample including the microbes Staphylococcus aureus, Pseudomonas aeruginosa, Proteus mirabilis, Klebsiella pneumoniae, Acinetobacter baumanii, Escherichia coli or Enterococcus faecium.

FIG. 2 shows the result of hybridization on the DNA chip of FIG. 1 in a blind sample including the microbe Staphylococcus aureus, assayed using Scanarray 5000.

FIG. 3 shows the result of hybridization on the DNA chip of FIG. 1 in a blind sample including the microbe Pseudomonas aeruginosa, assayed using Scanarray 5000.

FIG. 4 shows the result of hybridization on the DNA chip of FIG. 1 in a blind sample including the microbe Proteus mirabilis, assayed using Scanarray 5000.

FIG. 5 shows the result of hybridization on the DNA chip of FIG. 1 in a blind sample including the microbe Klebsiella pneumoniae, assayed using Scanarray 5000.

FIG. 6 shows the result of hybridization on the DNA chip of FIG. 1 in a blind sample including the microbe Acinetobacter baumanii, assayed using Scanarray 5000.

FIG. 7 shows the result of hybridization on the DNA chip of FIG. 1 in a blind sample including the microbe Escherichia coli, assayed using Scanarray 5000.

FIG. 8 shows the result of hybridization on the DNA chip of FIG. 1 in a blind sample including the microbe Enterococcus faecium, assayed using Scanarray 5000.

FIG. 9 shows a schematic representation of the DNA chip designed for a blind sample including the microbe Staphylococcus epidermidis.

FIG. 10 shows the result of hybridization on the DNA chip of FIG. 9 in a blind sample including the microbe Staphylococcus epidermidis, assayed using Scanarray 5000.

FIG. 11 shows a schematic representation of the DNA chip designed for a blind sample including the microbes Salmonella Group E or Salmonella Group B.

FIG. 12 shows the result of hybridization on the DNA chip of FIG. 11 in a blind sample including the microbe Salmonella Group E, assayed using Scanarray 5000.

FIG. 13 shows the result of hybridization on the DNA chip of FIG. 11 in a blind sample including the microbe Salmonella Group B, assayed using Scanarray 5000.

FIG. 14 shows a schematic representation of the DNA chip designed for a blind sample including the microbes Klebsiella oxytoca or Burkholderia cepacia.

FIG. 15 shows the result of hybridization on the DNA chip of FIG. 14 in a blind sample including the microbe Klebsiella oxytoca, assayed using Scanarray 5000.

FIG. 16 shows the result of hybridization on the DNA chip of FIG. 14 in a blind sample including the microbe Burkholderia cepacia, assayed using Scanarray 5000.

FIG. 17 shows the location of primers used for the amplification of polynucleic acid in a biological sample (16S: 16S rRNA; ITS: internal transcribed spacer region; 23S: 23S rRNA).

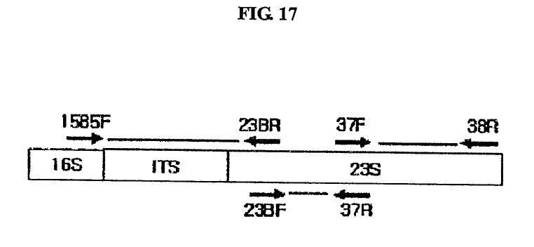

### DETAILED DESCRIPTION OF THE INVENTION

Definitions

The following definitions serve to illustrate the terms and expressions used in the different embodiments of the present invention as set out below.

An “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated.

The term “probe” or “nucleic acid probe” refers to single stranded sequence-specific oligonucleotides which have a base sequence which is sufficiently complementary to hybridize to the target base sequence to be detected.

By “composition”, it is meant that probes complementary to bacterial or fungal rRNA may be in a pure state or in combination with other probes. In addition, the probes may be in combination with salts or buffers, and may be in a dried state, in an alcohol solution as a precipitate, or in an aqueous solution.

The term “target” refers to nucleic acid molecules originating from a biological sample which have a base sequence complementary to the nucleic acid probe of the invention. The target nucleic acid can be single- or double-stranded DNA (if appropriate, obtained following amplification) or RNA and contains a sequence which has at least partial complementarity with at least one probe oligonucleotide.

The phrase “a biological sample” refers to a specimen such as a clinical sample (pus, sputum, blood, urine, etc.), an environmental sample, bacterial colonies, contaminated or pure cultures, purified nucleic acid, etc. in which the target sequence of interest is sought.

The term “polynucleic acid” corresponds to either double-stranded or single-stranded cDNA or genomic DNA or RNA, containing at least 10, 20, 30, 40 or 50 contiguous nucleotides. A polynucleic acid which is smaller than 100 nucleotides in length is often also referred to as an oligonucleotide. Single stranded polynucleic acid sequences are always represented in the present invention from the 5′ end to the 3′ end.

By “oligonucleotide” is meant a nucleotide polymer generally about 10 to about 100 nucleotides in length, but which may be greater than 100 or shorter than 10 nucleotides in length.

By “nucleotide” is meant a subunit of a nucleic acid consisting of a phosphate group, a 5-carbon sugar and a nitrogen containing base. In RNA the 5-carbon sugar is ribose. In DNA, it is a 2-deoxyribose. For a 5-nucleotide, the sugar contains a hydroxyl group (—OH) at the carbon-5. The term also includes analogs of such subunits.

The term “homologous” is synonymous for identical and means that polynucleic acids which are said to be e.g. 90% homologous show 90% identical base pairs in the same position upon alignment of the sequences.

“Hybridization” involves the annealing of a complementary sequence to the target nucleic acid (the sequence to be detected). The ability of two polymers of nucleic acid containing complementary sequences to find each other and anneal through base pairing interaction is a well-recognized phenomenon.

The term “primer” refers to a single stranded DNA oligonucleotide sequence capable of acting as a point of initiation for synthesis of a primer, extension product which is complementary to the nucleic acid strand to be copied. The length and the sequence of the primer must be such that they allow to prime the synthesis of the extension products. Preferably the primer is about 5-50 nucleotides long. Specific length and sequence will depend on the complexity of the required DNA or RNA targets, as well as on the conditions of primer use such as temperature and ionic strength.

The term “stringency” indicates one used to describe the temperature and solvent composition existing during hybridization and the subsequent processing steps. Under high stringency conditions only highly complementary nucleic acid hybrids will form; hybrids without a sufficient degree of complementarity will not form.

Accordingly, the stringency of the assay conditions determines the amount of complementarity needed between two nucleic acid strands forming a hybrid. Stringency is chosen to maximize the difference in stability between the hybrid formed with the target and the nontarget nucleic acid.

By “complementary” is meant a property conferred by the base sequence of a single strand of DNA or RNA which may form a hybrid or double stranded DNA:DNA, RNA:RNA or DNA:RNA through hydrogen bonding between Watson-Crick base pairs on the respective strands. Adenine (A) usually complements thymine (T) or uracil (U), while guanine (G) usually complements cytosine (C).

By “mismatch” is meant any pairing, in a hybrid, of two nucleotides which do not form canonical Watson-Crick hydrogen bonds.

The term “label” as used herein refers to any atom or molecule which can be used to provide a detectable (preferably quantifiable) signal, and which can be attached to a nucleic acid. Labels may provide signals detectable by fluorescence, radioactivity, colorimetry, gravimetry, X-ray diffraction or absorption, magnetism, and the like.

By “hybrid” is meant the complex formed between two single stranded nucleic acid sequences by Watson-Crick base pairings or non-canonical base pairings between the complementary bases.

The phrase “probe specificity” refers to characteristic of a probe which describes its ability to distinguish between target and non-target sequences. In this regard, the term “specific” means that a nucleotide sequence will hybridize to a defined target sequence and will substantially not hybridize to a non-target sequence, or that hybridization to a non-target sequence will be minimal. Probe specificity is dependent on sequence and assay conditions.

The term “Tm” refers to temperature at which 50% of the probe is converted from the hybridized to the unhybridized form. The phrase “standard strain” includes those commercially or readily available in the art.

Identification of Probes

Each probe needs to be specific for the microbe of interest. The specific probes according to the present invention are designed as follows. First, specific nucleotide sequences solely present in the microbe of interest are identified by performing multiple alignment of nucleotide sequences possibly derived from all microorganism species. The multiple alignment is carried out of 23S rRNA gene and/or ITS from bacteria and 18S rRNA gene from fungi. A lot of segments from 23S rRNA gene, ITS and 18S rRNA are selected as candidate probes. Second, the specificity of the candidate probe is confirmed by comparison to public databases containing nucleotide sequences using the BLAST analyses well known to those skilled in the art. Third, the sensitivity of the. candidate probe is assayed by applying it for clinical trials on a variety of biological samples.

The probe of the present invention include at least 15-mer oligonucleotide and are preferably 70%, 80%, 90% or more than 95% homologous to the exact complement of the target sequence to be detected. Those probes are about 15 to 50 nucleotides long. Of course, probes consisting of more than 50 nucleotides can be used. The nucleotides as used in the present invention may be ribonucleotides, deoxyribonucleotides and modified nucleotides such as inosine or nucleotides containing modified groups which do not essentially alter their hybridization characteristics.

Use of Probe

The probes of the invention can be used, for diagnostic purposes, in investigating the presence or the absence of a target nucleic acid in a biological sample, according to all the known hybridization techniques and especially the techniques of point deposition on filter called “DOT-BLOT” (MANIATIS et al., Molecular Cloning, Cold Spring Harbor, 1982), the DNA transfer techniques called “SOUTHERN BLOT” (SOUTHERN, E. M., J. Mol. Biol., 98, 503 (1975)), or the RNA transfer techniques called “NORTHERN BLOT”.

The probes of the invention can also be used in a sandwich hybridization system which enhances the specificity of a nucleic acid probe-based assay. The principle and the use of sandwich hybridizations in a nucleic acid probe-based assay have been already described (e.g.: DUNN and HASSEL, Cell, 12: 23-36; 1977; RANKI et al., Gene, 21: 77-85; 1983). The sandwich hybridization technique uses a capture probe and/or a detection probe, said probes being capable of hybridizing with two different regions of the target nucleic acid, and at least one of said probes (generally the detection probe) being capable of hybridizing with a region of the target which is specific for the species or the group of species investigated. It is understood that the capture probe and the detection probe must have nucleotide sequences which are at least partly different. Although direct hybridization assays have favorable kinetics, sandwich hybridizations are advantageous with respect to a higher signal-to-noise ratio. Moreover, sandwich hybridizations can enhance the specificity of a nucleic acid probe based assay. The incubation and subsequent washing stages which constitute the key stages of the sandwich hybridization process are each carried out at a constant temperature, between about 20° C. and 65° C. It is known that nucleic acid hybrids have a dissociation temperature which depends on the number of hybridized bases (the temperature increasing with the size of the hybrid) and which also depends on the nature of the hybridized bases and, for each hybridized base, on the nature of the adjacent bases. The hybridization temperature used in the sandwich hybridization technique should obviously be chosen below the half-dissociation temperature of the hybrid formed by a given probe with the target of complementary sequence, by simple routine experiment.

The probes of the invention can also be used in a competition hybridization protocol. In a competition hybridization, the target molecule competes with the hybrid formation between a specific probe and its complement. The more target is present, the lower the amount of hybrid formed between the probe and its complement. A positive signal, which indicates that the specific target was present, is seen by a decrease in hybridization reaction as compared with a system to which no target was added. In a particular embodiment, the specific oligonucleotide-probe, conveniently labeled, is hybridized with the target molecule. Next, the mixture is transferred to a recipient (e.g. a microtiter dish well) in which a oligonucleotide complementary to the specific probe is fixed and the hybridization is continued. After washing, the hybrids between the complementary oligonucleotide and the probe are measured, preferably quantitatively, according to the label used.

In addition, the probes of the invention can be used in a reversed hybridization (Proc. Natl. Acad. Sci. USA, 86:6230-6234, 1989). In this case, the target sequences can first be enzymatically amplified using PCR with 5′ biotinylated primers. In a second step, the amplified products are detected upon hybridization with specific oligonucleotides immobilized on a solid support. Reversed hybridization may also be carried out without an amplification step. In that particular case, the nucleic acids present in the sample have to be labeled or modified, specifically or not, for instance, chemically or by addition of specific dyes, prior to hybridization.

The nucleic acid probes of this invention can be included in a kit which can be used to rapidly determine the presence or absence of pathogenic species of interest.

The kit includes all components necessary to assay for the presence of these pathogens. In the universal concept, the kit includes a stable preparation of labeled probes, hybridization solution in either dry or liquid form for the hybridization of target and probe polynucleotides, as well as a solution for washing and removing undesireable and nonduplexed polynucleotides, a substrate for detecting the labeled duplex, and optionally an instrument for the detection of the label.

A more specific embodiment of this invention embraces a kit that utilizes the concept of the sandwich assay. This kit would include a first component for the collection of samples from patients, such as a scraping device or paper points, vials for containment, and buffers for the dispersement and lysis of the sample. A second component would include media in either dry or liquid form for the hybridization of target and probe polynucleotides, as well as for the removal of undesireable and nonduplexed forms by washing. A third component includes a solid support upon which is fixed or to which is conjugated unlabeled nucleic acid probe(s) that is(are) complementary to a part of the target polynucleotide. In the case of multiple target analysis more than one capture probe, each specific for its own ribosomal RNA, will be applied to different discrete regions of the dipstick. A fourth component would contain labeled probe that is complementary to a second and different region of the same rRNA strand to which the immobilized, unlabeled nucleic acid probe of the third component is hybridized. The probe components described herein include combinations of probes in dry form, such as lyophylized nucleic acid or in precipitated form, such as alcohol precipitated nucleic acid or in buffered solutions. The label may be any of the labels described above. For example, the probe can be biotinylated using conventional means and the presence of a biotinylated probe can be detected by adding avidin conjugated to an enzyme, such as horseradish peroxidase, which can then be contacted with a substrate which, when reacted with peroxidase, can be monitored visually or by instrumentation using by a colorimeter or spectrophotometer. This labeling method and other enzyme-type labels have the advantage of being economical, highly sensitive, and relatively safe compared to radioactive labeling methods. The various reagents for the detection of labeled probes and other miscellaneous materials for the kit, such as instructions, positive and negative controls, and containers for conducting, mixing, and reacting the various components, would complete the assay kit.

DNA Chip

The probes of the invention are also used in a DNA chip. In a preferred embodiment, the present invention provides a DNA chip in which nucleic acid probes are immobilized on a solid support. The DNA chip which is formed by arranging DNA fragments of variety of base sequences on the surface of a narrow substrate in high density is used in finding out the information on DNA of an unknown sample by hybridization between an immobilized DNA and unknown DNA sample complementary thereto. Examples of the solid carrier on which the probe oligonucleotides are fixed include inorganic materials such as glass and silicon and polymeric materials such as acryl, polyethylene terephtalate (PET), polystyrene, polycarbonate and polypropylene. The surface of the solid substrate can be flat or have a multiple of hole. The probes are immobilized on the substrate by covalent bond of either 3′ end or 5′ end. The immobilization can be achieved by conventional techniques, for example, using electrostatic force, binding between aldehyde coated slide and amine group attached on synthetic oligomeric phase or sptting on amine coated slide, L-lysine coated slide or nitrocellulose coated slide. One embodiment of the present invention includes incorporating base with amino residue on 3′ position of the probe upon synthesizing it, followed by covalently binding it on aldehyde coated glass slide.

The immobilization and the arrangement of various probes onto the solid substrate are carried out by pin microarray, inkjet, photolithography, electric array, etc. In an embodiment of the invention, probes are separately dissolved in a buffer solution and the resulting solution is spotted onto the substrate by using a microarrayer prepared by a known method (Yoon et al., J. Microbiol. Biotechnol., 10(1), 21-26, 2000). The basis principle of the microarrayer is that minutely constructed pin picks probe DNAs from a plate and transfers it to the site that is appointed by a computer. For the fixing of the probe transferred by a microarrayer, the immobilization reaction is allowed for at least one hour under humidity of from 45% to 65%, preferably, from 50% to 55%, and it stands up for at least 6 hours to facilitate the reaction between the amine group at 3′ position of the probe and the aldehyde group coated onto the glass slide.

For detecting cells derived from or themselves being living organisms, the RNA and/or DNA of these cells, if need be, is made accessible by partial or total lysis of the cells using chemical or physical processes, and contacted with one or several probes of the invention which can be detected. This contact can be carried out on an appropriate support such as a nitrocellulose, cellulose, or nylon filter in a liquid medium or in solution. This contact can take place under suboptimal, optimal conditions, or under restrictive conditions (i.e. conditions enabling hybrid formation only if the sequences are perfectly homologous on a length of molecule). Such conditions include temperature, concentration of reactants, the presence of substances lowering the optimal temperature of pairing of nucleic acids (e.g. formamide, dimethylsulfoxide and urea) and the presence of substances apparently lowering the reaction volume and/or accelerating hybrid formation (e.g. dextran sulfate, polyethyleneglycol or phenol).

Preparation of Probes

To obtain large quantities of nucleic acid probes, one can either clone the desired sequence using traditional cloning methods, such as described in Maniatis, T., et al. Molecular Cloning: A Laboratory Manual, Cold Spring Harbor, New York, 1982, or one can produce the probes by chemical synthesis using commercially available DNA synthesizers.

The probes of the invention can be prepared by conventional methods. Two methods are typically introduced.

A first method is a preparation of a single-stranded probe.

A representive example of preparing a single-stranded probe consisting of the desired number of nucleotides includes a dimethoxytrityl (DMT) off method by an automated DNA synthesizer which comprises removing the DMT group to free the 5′ hybroxyl for the coupling reaction, coupling and capping. The probes obtained thereby is labeled with a fluorescent dye (fluorescein isothiocyanate, FITC) to confirm the presence or the absence of nucleic acids of interest. Alternatively, the DNA probe complementary to single-stranded DNA template is prepared by annealing the primer to the template DNA and performing extension reactions from the primer/template complex using Klenow fragment and dNTP labeled with fluorescent dye. The probe made thus exhibits high sensitivity and specificity owing to its fluorescent dye.

A second method is a preparation of double-stranded probe. It is possible to make a probe having the desired region of a gene or a base segment by digesting genomic DNA or plasmid DNA with specific restriction enzymes. A random priming method is a synthesis of fluorescent-labeled probes with various lengths by hybridizing six random hexamer with template DNA. Alternatively, fluorescent-labeled probes can be synthesized by transferring ³²p to the 5′ end of DNA by T4 polynucleotide kinase. In addition, the probe can be synthesized by breaking down double-stranded DNA molecules with DNase I and performing DNA replication using DNA polymerase I and fluorescent-labeled DNTP. The double-stranded probe obtained thereby is denatured to form single-stranded DNAs which are then used in a hybridization reaction.

The probes of the invention are advantageously labeled. Any conventional label can be used. The probes can be labeled by means of radioactive tracers such as ³²p, ³⁵S, ^{125,} ³H and ¹⁴C. The radioactive labeling can be carried out according to any conventional method such as terminal labeling at the 3′ or 5′ position with the use of a radiolabeled nucleotide, a polynucleotide kinase (with or without dephosphorylation by a phosphatase), a terminal transferase, or a ligase (according to the extremity to be labeled). Another method for radioactive labeling is a chemical iodination of the probes of the invention which leads to the binding of several ¹²⁵I atoms on the probes.

If one of the probes of the invention is made radioactive to be used for hybridization with a nonradioactive RNA or DNA, the method of detecting hybridization will depend on the radioactive tracer used. Generally, autoradiography, liquid scintillation, gamma counting or any other conventional method enabling one to detect an ionizing ray issued by the radioactive tracer can be used. Nonradioactive labeling can also be used by associating the probes of the invention with residues having: immunological properties (e.g. antigen or hapten), a specific affinity for some reagents (e.g. ligand), properties providing a detectable enzymatic reaction (e.g. enzyme, co-enzyme, enzyme substrate or substrate taking part in an enzymatic reaction), or physical properties such as fluorescence, emission or absorption of light at any wavelength. Antibodies which specifically detect the hybrids formed by the probe and the target can also be used. A nonradioactive label can be provided when chemically synthesizing a probe of the invention, the adenosine, guanosine, cytidine, thymidine and uracyl residues thereof being liable to be coupled to other chemical residues enabling the detection of the probe or the hybrids formed between the probe and a complementary DNA or RNA fragment.

Target

To provide nucleic acid substrates for use in the detection and identification of microorganisms in clinical samples using the structure probing assay, nucleic acid is extracted from the sample. The nucleic acid may be extracted from a variety of clinical samples using a variety of standard techniques or commercially available kits. For example, kits which allow the isolation of RNA or DNA from tissue samples are available from Qiagen, Inc. (Chatsworth, Calif.) and Stratagene (La Jolla, Calif.). For example, the QIAamp Blood kits permit the isolation of DNA from blood (fresh, frozen or dried) as well as bone marrow, body fluids or cell suspensions. QIAamp tissue kits permit the isolation of DNA from tissues such as muscles, organs and tumors. In a preferred method of determining whether a biological sample contains rRNA or rDNA that would indicate the presence of the desired pathogens, nucleic acids may be released from cells by sonic disruption, for example according to the method disclosed by Murphy et al., in U.S. Pat. No. 5,374,522. Other known methods for disrupting cells include the use of enzymes, osmotic shock, chemical treatment, and vortexing with glass beads. Other methods suitable for liberating from microorganisms the nucleic acids that can be subjected to the hybridization methods disclosed herein have been described by Clark et al., in U.S. Pat. No. 5,837,452 and by Kacian et al., in U.S. Pat. No. 5,364,763. Following or concurrent with the release of rRNA, labeled probe may be added in the presence of accelerating agents and incubated at the optimal hybridization temperature for a period of time necessary to a achieve significant hybridization reaction. In the case of a double-stranded nucleic target, it is advisable to carry out its denaturation before carrying out the process of detection. The denaturation of a double-stranded nucleic acid may be carried out by known methods of chemical, physical or enzymatic denaturation, and in particular by heating at an appropriate temperature, greater than 80° C.

In addition, target DNA hybridizing to the probe is usually prepared by two methods. A first method is one used in Southern blot or Northern blot. Genomic DNA or plasmid DNA are digested with appropriate restriction enzymes and the resulting DNA fragments are separated by agarose gel electroporesis and used. A second method is an amplification of the desired DNA region by PCR. Examples of the PCR include most typical PCR using the same amounts of forward and reverse primers, asymmetric PCR in which double-stranded and single-stranded bands can be obtained by adding primers asymmetrically, multiplex PCR in which a multiple of target DNAs can be amplified at once by adding various primers simultaneously, ligase chain reaction (LCR) in which target DNA is amplified using specific 4 primers and ligase and the amount of fluorescence is measured by ELIA (Enzyme Linked Immunosorbent Assay), and the other PCR such as Hot Start PCR, Nest-PCR, DOP-PCR (degenerate oligonucleotide primer PCR), RT-PCR (reverse transcription PCR), Semi-quantitative RT-PCR, Real time PCR, RACE (rapid amplification of cDNA ends), Competitive PCR, STR (short tandem repeats), SSCP (single strand conformation polymorphism), DDRT-PCR (differential display reverse transcriptase), etc.

It has been found that crude extracts from relatively homogenous specimens (such as blood, bacterial colonies, viral plaques, or cerebral spinal fluid) are better suited to severing as templates for the amplification of unique PCR products than are more composite specimens (such as urine, sputum or feces) (Shibata in PCR: The Polymerase Chain Reaction, Mullis et al., eds., Birkhauser, Boston [1994], pp. 47-54). Samples which contain relatively few copies of the material to be amplified (i.e., the target nucleic acid), such as cerebral spinal fluid, can be added directly to a PCR. Blood samples have posed a special problem in PCRs due to the inhibitory properties of red blood cells. The red blood cells must be removed prior to the use of blood in a PCR; there are both classical and commercially available methods for this purpose (e.g., QIAamp Blood kits, passage through a Chelex 100 column [BioRad], etc.). Extraction of nucleic acid from sputum, the specimen of choice for the direct detection of M. tuberculosis, requires prior decontamination to kill or inhibit the growth of other bacterial species. This decontamination is typically accomplished by treatment of the sample with N-acetyl L-cysteine and NaOH (Shinnick and Jones, supra). This decontamination process is necessary only when the sputum specimen is to be cultured prior to analysis.

A preferred embodiment of the present invention includes preparing gene fragments by an asymmetric PCR using DNA of isolated sample as a template. The gene fragments are obtained by performing the PCR at once with addition of forward and reverse primers at the ratio of 1:5. The used primers correspond to the regions of the base sequence universally present on bacteria (Pirkko K. et al., Clin. Microbiol., 36(8), 2205-2209, 1999) and are as follows:

Primer 1. (sense):
TTGTACACACCGCCCGTC (SEQ ID NO: 406, 1585Fw)
and
Primer 2 (antisense):
F-TTCGCCTTTCCCTCACGGTACT (SEQ ID NO: 407, 23Br);
Primer 3 (sense):
AGTACCGTGAGGGAAAGGGGAA (SEQ ID NO: 408, 23BFw)
and
Primer 4 (antisense):
F-TGCTTCTAAGCCAACATCCT (SEQ ID NO: 409, 37R);
and
Primer 5 (sense):
AGGATGTTGGCTTAGAAGCA (SEQ ID NO: 410, MS37F)
and
Primer 6 (antisense):
F-CCCGACAAGGAATTTCGCTACCTT (SEQ ID NO: 411,
MS38R).

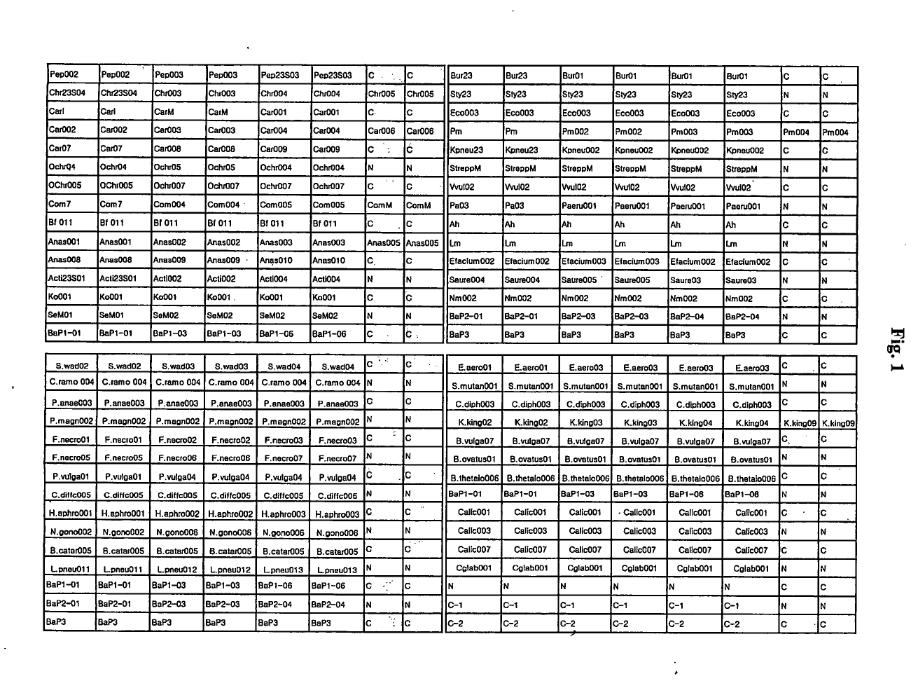

In the above primers, the locations are shown in FIG. 1 and the letter “F” conjugated to 5′ end indicates fluorescein isothicyanate (FITC). The target DNAs are amplified using 5-FITC conjugated primers, and then the hybridization between the amplified target DNAs and the nucleic acid probes is determined by fluorescence to confirm the identity of the infectious agent. In order to obtain the regions which cannot be amplified by the above primers, additional primers are designed through multiple alignment and BLAST.

The primers used for the fungi have been designed directly by the inventors from partial regions of 18S rRNA and have the following base sequences:

Primer 1 (sense):
GTAATTGGAATGAGTACAAT (SEQ ID NO: 412, fun4E3F)
and
Primer 2 (antisense):
F-CTACGACGGTATCTGATCAT (SEQ ID NO: 413, fun986R).

In a preferred embodiment of the PCR, 5 ul of 10X PCR buffer solution (100 mM Tris-HCl, pH 8.3, 500 mM KCl, 15 mM MgCl₂), 4 ul of dNTP mixture (dATP, dGTP, dCTP, dTTP, each 2.5 mM), 0.5 ul of 10 pmole forward primer, 2.5 ul of 10 pmole reverse primer, 1 ul of 1/10 diluted template DNA (100 ng) and 0.5 ul of Taq polymerase (5 unit/ul, Takara Shuzo Co., Shiga, Japan) are mixed and a water is added to the resulting mixture to be a total volume of 50 ul. The asymmetric PCR is conducted by 10 cycles, each consisting of first denaturation at 94° C. for 7 minutes, second denaturation at 94° C. for 1 minute, annealing at 52° C. for 1 minute and extension at 72° C. for 1 minute, and 30 cycles, each consisting of third denaturation at 94° C. for 1 minute, annealing at 52° C. for 1 minute and extension at 72° C. for 1 minute, followed by one final extension at 72° C. for 5 minutes. The PCR products are confirmed by agarose gel electrophoresis.

Hybridization and Wash

The particular hybridization technique is not essential to the invention. Hybridization techniques are generally described in Nucleic Acid Hybridization: A Practical Approach, Ed. Hames, B. D. and Higgins, S. J., IRL Press, 1987; Gall and Pardue (1969), Proc. Natl. Acad. Sci., U.S.A., 63:378-383, and John, Burnsteil and Jones (1969) Nature, 223:582-587.

The hybridization conditions are determined by the “stringency”, that is to say the strictness of the operating conditions. The hybridization is all the more specific when it is carried out with greater stringency.

The stringency is a function especially of the base composition of a probe/target duplex, as well as by the degree of mismatching between two nucleic acids. The stringency can likewise be a function of parameters of the hybridization reaction, such as the concentration and the type of ionic species present in the hybridization solution, the nature and the concentration of denaturing agents and/or the hybridization temperature. The stringency of the conditions under which a hybridization reaction must be carried out depends especially on the probes used. All these data are well known and the appropriate conditions can possibly be determined in each case by routine experiments. In general, depending on the length of the probes used, the temperature for the hybridization reaction is between approximately 20° C. and 65° C. in particular between 35° C. and 65° C. in a saline solution at a concentration of approximately 0.8 to 1M.

Nucleic acid hybridization between labeled oligonucleotide probes and nucleic acid targets can be enhanced by the use of “unlabeled Helper Probes” as disclosed in U.S. Pat. No. 5,030,557 to Hogan et al. Helper probes are oligonucleotides which bind to a portion of the target nucleic acid other than that being targeted by the assay probe, and which imposed new secondary and tertiary structure on the targeted region of the single stranded nucleic acid whereby the rate of binding of the assay probe is accelerated.

It will be appreciated by those skilled in the art that factors which affect the thermal stability can affect probe specificity and therefore, must be controlled. Thus, the melting profile, including the melting temperature (Tm) of the oligonucleotide/target hybrids should be determined. The preferred method is described in U.S. Pat. No. 5,283,174 to Arnold et al. For Tm measurement using a Hybridization Protection Assay the following technique is used. A probe:target hybrid is formed in target excess in a lithium succinate buffered solution containing lithium lauryl sulfate. Aliquots of this “preformed” hybrid are diluted in the hybridization buffer and incubated for five minutes at various temperatures starting below that of the anticipated Tm (typically 55° C.) and increasing in 2-5° C. increments. This solution is then diluted with a mildly alkaline borate buffer and incubated at a lower temperature (for example 50° C.) for ten minutes. Under these conditions the acridinium ester attached to a single stranded probe is hydrolyzed while that attached to hybridized probe is relatively “protected”. This is referred to as the hybridization protection assay (“HPA”). The amount of chemiluminescence remaining is proportional to the amount of hybrid and is measured in a luminometer by addition of hydrogen peroxide followed by alkali. The data is plotted as percent of maximum signal (usually from the lowest temperature) versus temperature. The Tm is defined as the point at which 50% of the maximum signal remains.

In addition to the above method, oligonucleotide/target hybrid melting temperature may also be determined by isotopic methods well known to those skilled in the art. It should be noted that the Tm for a given hybrid will vary depending on the hybridization solution being used because the thermal stability depends upon the concentration of different salts, detergents, and other solutes which affect relative hybrid stability during thermal denaturation. (Molecular Cloning: A Laboratory Manual Sambrook et al., eds. Cold Spring Harbor Lab Publ., 9.51 (2d ed 1989)).

The hybridization conditions can be monitored relying upon several parameters, e.g. hybridization temperature, the nature and concentration of the components of the media, and the temperature under which the hybrids formed are washed. The hybridization and wash temperature is limited in upper value, according to the probe (its nucleic acid composition, kind and length) and the maximum hybridization or wash temperature of the probes described herein is about 30° C. to 60° C. At higher temperatures duplexing competes with the dissociation (or denaturation) of the hybrid formed between the probe and the target. A preferred hybridization medium contains about 3×SSC (1×SSC=0.15 M NaCl, 0.015 M sodium citrate, pH 7.0), about 25 mM of phosphate buffer pH 7.1, and 20% deionized formamide, 0.02% Ficoll, 0.02% bovine serum albumin, 0.02% polyvinylpyrrolidone and about 0.1 mg/ml sheared denatured salmon sperm DNA. A preferred wash medium contains about 3×SSC, 25 mM phosphate buffer pH 7.1 and 20% deionized formamide. However, when modifications are introduced, be it either in the probes or in the media, the temperatures at which the probes can-be used to obtain the required specificity should be changed according to known relationships, such as those described in the following reference: B. D. HAMES and S. J. HIGGINS, (eds.). Nucleic acid hybridization. A practical approach, IRL Press, Oxford, U.K., 1985. In this respect it should also be noted that, in general, DNA:DNA hybrids are less stable than RNA:DNA or RNA:RNA hybrids. Depending on the nature of the hybrid to be detected, the hybridization conditions should be adapted accordingly to achieve specific detection.

In a preferred embodiment of the present invention, a hybridization buffer solution (6×SSPE (0.15M NaCl, 5mM C6HSNa307, pH 7.0), 20% (v/v) formamide) is mixed with PCR amplified target genes, the resulting mixture is applied onto a glass slide to which probes are immobilized, and then the reaction is kept at 30° C. for 6 hours so that the said probes can complementarily hybridize with the said targets. The glass slide is washed sequentially with 3×SSPE, 2×SSPE and 1×SSPE by 2 minutes.

The formed hybrids can be quantified by labelling the target with a fluorescence or radioactive isotope in accordance to conventional methods. The labelling may be carried out by the use of labelled primers or the use of labelled nucleotides incorporated during the polymerase step of the amplification.

Diagnostic Use

The nucleic acid probes of the present invention can be used for accurate diagnosis of one type of infection diseases. As such, probe(s) originating from one particular pathogenic microorganism are applied to a kit, preferably fixed onto DNA chip. Alternatively, the nucleic acids of the present invention can be used in combination of two or more species to a kit, preferably onto DNA chip, for the simultaneous detection of multiple pathogen species possibly present in a particular type of a biological sample, for example a panel of pathogens possibly present in the same type of biological sample or a panel of pathogens possibly causing the same type of disease symptoms. The infection diseases caused by the above-mentioned pathogenic microorganisms of the present invention are well reported by medical literature and examplifed as follows:

Acinetobacter baumanii causes purulent infection in all organs of humans (Glew R H. et al., Medicine (Baltimore). 56, 79-97, (1977)), cystopyelonephritis and cystitis in relation with calculus in urethra (Glew R H et al., Medicine (Baltimore), 56, 79-97, (1977)), meningitis (Berk S L et al., Arch. Neurol. 38, 95-98, (1981)), cellulitis (Gervich D H et al, Am. J. Infect. Control. 13, 210-215, (1985)), wound infection (Tong M J. JAMA. 219, 1044-1047, (1972)), necrotizing fasciitis (Amsel M B et al., Curr. Surg. 42, 370-372, (1985)), endophthalmitis (Peyman G A et al., Am. J. Ophthalmol. 80, 764-765, (1975)), endocarditis (Gradon J D, et al., Clin. Infect. Dis. 14, 1145-1148, (1992)), osteomyelitis, bacterial arthritis, liver abscess, pancreatic abscess (Henricksen S D. Bacteriol. Rev. 37, 522-561, (1973)), etc.;

Anaerobiospirillum succiniciproducens causes clinically significant bacteremia (Tee W et al., J. Clin. Microbiol. 36(5), 1209-1213, (1998)), sepsis (Marcus L et al., Eur. J. Clin. Microbiol. Infect. Dis. 15(9), 741-744, (1996)), diarrhea (Malnick H et al., J. Clin. Pathol., 36(10), 1097, (1983), etc.;

Bacteroides fragilis causes meningitis in central nervous system (Feder H M. Rev. Infect. Dis. 9, 783-786, (1987)), brain tumor, subdural empyema or extradural abscess (Swartz M N. In: Finegold S M, George W L, eds. Anaerobic infections in humans. New York: Academic; 155-212, 1989), chronic sinusitis (Frederick J et al., N. Engl. J. Med. 290, 135-137, (1974)), intraperitoneal abscess (Gorbach S L. Clin. Infect. Dis. 17, 961-967, (1993)), liver abscess (Rubin R H et al., Am. J. Med. 57, 601-610, (1974)), bacteremia (Lombardi D P et al., Am. J. Med. 92, 53-60, (1992); Chow A W. et al., Medicine (Baltimore) 53, 93-123, (1974); and Redondo M C et al., Clin. Infect. Dis., 20, 1492-1496, (1995)), endocarditis (Felner A M et al., N. Engl. J. Med. 282, 1188-1192, (1970) and Nastro L J et al., Am. J. Med., 54, 482-496, (1973)), wound infection, necrotizing fasciitis, diabetic ulcer or cellulites (Gerding D. Clin. Infect. Dis. 20(Suppl 2), S283-S288, (1995)), chronic osteomyelitis or bacterial arthritis (Rosenkranz P et al., Rev. Infect. Dis. 12, 20-30, (1990)), etc.;

Cardiobacterium hominis causes endocarditis (Traveras J. Md. et al., South. Med. J., 86, 1439-1440, (1993)), and it leads to embolism of whole body, cerebral aneurysm, cardiac insufficiency.;

Chryseobacterium meningosepticum causes neonatal meningitis (Plotkin S. A. et al., JAMA, 198, 194-196, (1966); Pokrywka M. et al., Am. J. Infect. Control, 21, 139-145, (1993)), respiratory infection (Brown R. B. et al., Am. J. Infect. Control, 17, 121-125, (1989)), sepsis, endocarditis, celluitis, wound infection, abdominal abscess, peritonitis, endophthoalmitis (Olsen H. et al., Lancet, 1, 1294-1296, (1965); Sheridan R. L. et al., Clin. Infect. Dis., 17, 185-187, (1993)), etc.;

Clostridium ramosum causes inflammatory intestinal deaseses (Senda S, et al., Microbial Immunol 1985; 29(11):1019-28), brain tumor (An Med Interna. 1998 July; 15(7):392-3), arterial sepsis/embolism to renal transplanted patients (Transplant Proc. 1983 June; 15(2):1715-9);

Comamonas acidovorans causes endocarditis to medical addict (Horowitz H. et al., J. Clin. Microbiol., 28, 143-145, (1990));

Corynebacterium diphtheriae causes respiratory diphtheria (Dobie RA, et al., JAMA. 1979; 242:2197-2201), myocarditis (Boyer NH, et al., N Engl J Med. 1948; 239:913.), paralysis of eyeball's movement ciliary (Kallick C A, et al., III Med J. 1970; 137:505-512; and Naiditch M J, et al., Am J Med. 1954; 17:229-245), functional disorder of face, pharynx, larynx, pleurisy peripheral nerve, skin diphtheria (Koopman J S, et al., J Infect Dis. 1975; 131:239-244), endocarditis (Tiley SM, et al., Clin Infect Dis. 1993; 16:271-275), fungous aneurism (Gruner E, et al., Clin Infect Dis. 1994; 18:94-96), Osteomyelitis (Patey O, et al., J Clin Microbiol. 1997; 35:441-445) and arthritis (Patey O, et al., J Clin Microbiol. 1997; 35:441-445.);

Klebsiella oxytoca causes pneumonia (Korvick J A et al., South. Med. J. 84(2), 200-204, (1991); and Al-Moamary M S et al., Clin. Infect. Dis. 26(3), 765-766, (1998)), acute cystopyelonephritis in children (Ghiro L et al., Nephron. 90(1), 8-15, (2002)), sudden neonatal deaths (outbreak) (Jeong S H et al., J. Hosp. Infect. 48(4), 281-288, (2001)), enteritis (Soussi F et al., Gastroenterol. Clin. Biol. 25(8-9), 814-816, (2001);

Ochrobactrum anthropi causes bacteremia that related to vascular tissues (Kern W. V. et al., Infection, 21, 306-310, (1993)), endocarditis (Mahmood M. S. et al., J. Infect., 40, 287-290, (2000)), endophthalmitis (Berman A. J. et al., Am. J. Ophthalmol., 123, 560-562, (1997)), pancreatic abscess, necrotizing fasciitis (Brivet F. et al., Clin. Infect. Dis., 17, 516-518, (1993)), chondirtis (Barson W. J. et al., J. Clin. Microbiol., 25, 2014-2016, (1987)). etc.;

Peptostreptococcus prevotii causes many kinds of abscess (e.g.: brain tumor), chronic otitis media, acute mastoiditis, chronic sinusits, pneumonia, lung abscess, pleunal empyema, female genital infection (Murdoch D A et al., J. Med. Microbiol. 41, 36-44, (1987)), bacteremia (Brook I, J. Infect. Dis. 160, 1071-1075, (1989)), osteomyelitis, spinal osteomyelitis, mastitis, cellulites, necrotizing fasciitis (Murdoch D A, Clin. Microbiol. Rev., 11, 81-120, (1998)), diabetic foot infection (Wren M W D, Br. J. Biomed. Sci., 53, 294-301, (1996)), postpartum sepsis, bacterial arthritis of artificial joints (Brook I et al., Am. J. Med. 94, 21-28, (1993)), endocarditis, abscesses around the valves of the heart, bacterial pericarditis, mediastinitis (Murdoch D A, Clin. Microbiol. Rev., 11, 81-120, (1998), oral infection (Finegold S M, New York: Academic; 1977);

Porphyromonas gingivalis causes oral infection, Periodontal abscess, periodontitis, acute necrotizing ulcerative periodontitis, (Darby I et al., Periodontol. 2000, 26, 33-53, (2001)), breast abscess (Edmiston C E et al., J. Infect. Dis., 162, 695-699, (1990)), chronic osteomyelitis (Brook I. et al., Am. J. Med. 94(1), 21-28, (1993)), sore throat (Brook I., J. Fam. Pract., 38(2), 175-179, (1994)), pneumonia, lung abscess, pleunal empyema, wound infection, otitis media, peritonitis, Paronychia, chronic sinusits (Brook I., J. Med. Microbiol. 42(5), 340-347, (1995)), vaginitis, infections in inner pelvis (Buerden B I, FEMS Immunol. Med. Microbiol. 6(2-3), 223-227, (1993)), bacteremia (Lee S C et al., J. Microbiol. Immunol. Infect. 32(3), 213-216, (1999)), endocarditis (van Winkelhoff A J et al., Periodontol. 2000, 20, 122-135, (1999)), etc.;

Peptostreptococcus anaerobius causes abscess (Murdoch D. A. et al., J Med Microbiol 1994; 41: 36-44; Brook I., J Urol 1989; 141: 889-893; Brook I., Ann Otol Rhin Laryngol 1998; 107: 959-960; and Civen R. et al., J Oral Pathol Med 2000; 29: 507-513), infections in hemorrhoids (Brook I. and Frazier E. H., Am J Gastroenterol 1996; 91: 333-335), infections in soft tissues (Brook I. and Frazier E. H., Arch Surg 1990; 125: 1445-1451), endocarditis (Montejo M. et al., Clin Infect Dis 1995; 20: 1431), gingivitis, paradentitis (Moore L V H, et al., J Dent Res 1987; 66: 989-995; and Wade W G, et al., J Clin Periodontol 1992; 19: 127-134), etc.;

Peptostreptococcus magnus causes festering nasopharyngitis (Brook, I., et al., Arch. Otolaryngol. Head eck Surg. 122:4184, 1996), pleural empyema (Civen, R., H. et al., Clin. Infect. Dis. 20(Suppl. 2):S224S229, 1995; Marina, M., C. et al., Clin. Infect. Dis. 16(Suppl. 4) S256S262, 1993; [symbol] Murdoch, D. A., et al., J. Med. Microbiol. 41:3644, 1987), necrotizing pneumonia, hepatic abscess (Brook, I. and E. H. Frazier. Pediatr. Infect. Dis. J. 12:743747, 1993), infections in surfaces of body (Brook, I. and E. H. Frazier. Arch. Surg. 125:144514, 1990), diabetic foot disorders (Sanderson, P. J., Clin. Pathol. 30:266268, 1977), cute and Chronic types of Non-gestational mammary abscess, cellulites (Brook, I., and E. H. Frazier., Arch. Surg. 130:7B6792, 1995), endocarditis (Cofsky, R. D., and S. J. Seligman. 1985. Peptococcus magnus endocarditis. South. Med. J. 78:361362; Pouedras, P., et al., Clin. Infect. Dis. 15:185), meningitis (Brown, M. A., et al., Am. J. Med. Sci. 308:18418, 1994), osteomyelitis (Brook, I., and E. H. Frazier., Am. J. Med. 94:22128, 1993), septic arthritis (Fitzgerald, R. H., et al., Clin. Orthoped. 164:14114, 1982), festering pericarditis (Phelps, R., et al., JAMA 254:9479, 1985), sinusits, child otitis media (Brook, I. 1994. Peptostreptococcal infection in children. Scand. J. Infect. Dis. 26:503510. and Clin. Microbiol. Rev. 8:4794), etc.;

Fusobacterium necrophorum causes infections in the mouth, intestinal canal, vagina (Mandell: Principles and Practice of Infectious Diseases, 5th ed., Copyright 2000 Churchill Livingstone, Inc p.2564-2566), Lemierre's syndrome (Bilateral Lemierre's syndrome: a case report and literature review. Ear Nose Throat J. 2002 April; 81(4):234-6, 238-40, 242);

Proteus vulgaris causes infections in the urinary track (Silverblatt F J. J Exp Med. 1974; 140:1696; Wray S K, Hull S I, Cook R G, et al. Proteus mirabilis. Infect Immun. 1986;54:43-49; Mobley H L, Chippendale G R. J Infect Dis. 1990; 161:525-530), meningitis, phlebothrombitis in the corpus spongiosum (Bodur H, Colpan A, Gozukuck R et al. Scand J Infect Dis. 2002; 34(9):694-6);

Enterobacter aerogenes causes deep infections (De Gheldre Y, Maes N, Rost F, et al. J Clin Microbiol. 1997; 35:152-160), atypical pneumonia (Holden D A, Stoller J K. Department of Pulmonary Disease, Cleveland Clinic Foundation, Ohio. West J Med 1992 January; 156(1):79-824), etc.;

Streptococcus mutans causes endocarditis (Infective Endocarditis in Adults Eleftherios Mylonakis, M. D., and Stephen B. Calderwood, M. D. In New England Journal of medicine Volume 345:1318-1330 Nov. 1, 2001), bacteremia (Elting L S, Bodey G P, Keefe B H. Clin Infect Dis. 1992; 14:1201-1207), meningitis (Hoyne A L, Herzon H. Ann Intern-Med. 1950; 33:879-902), pneumonia (Lorber B, Swenson R M. Ann Intern Med. 1974; 81:329-331), acute festering mumps (Raad II, Sabbagh M F, Caranasos G J. Clin Infect Dis. 1990; 12:591-601), orofacial odontogenic infections (Gill Y, Scully C. Oral Surg Oral Med Oral Pathol. 1990; 70:155-158), endophthalmitis (Principles and Practice of Infectious Diseases. 5th edition. Mandell, Churchill Livingstone p.217), otitis media, sinusits (Gaudreau C, Delage G. Rousseau D, et al. Can Med Assoc J. 1981; 125:1246-1249), etc.;

Kingella kingap causes infectious arthritis, osteomyelitis (Amir J, Schockelford P G. J Clin Microbiol. 1991; 29:1083-1086; Woolfrey B F, Lally R T, Faville R J. Am J Clin Pathol. 1986;85:745-749), endocarditis (Wolff A H, Ullman R F, Strampfer M J, Cunha B A. Heart Lung. 1987; 16:579-583; Rabin R L, Wong P, Noonan J A, Plumley D D. Am J Dis Child. 1983; 137:403-404; Verbruggen A-M, Hauglustaine D, Schildermans F. et al. J Infect. 1986; 13:133-142), bacteremia (Yagupsky P, Dagan R. Pediatr Infect Dis J. 1994; 13:1148-1149; Birgisson H, Steingrimsson O, Gudnason T. Scand J Infect Dis. 1997; 29:495-498; Roiz M P, Peralta F G, Arjona R. J Clin Microbiol. 1997; 35:1916; Yagupsky P, Dagan R. Clin Infect Dis. 1997; 24:860-866; Redfield D C, Overturf G D, Ewing N, Powars D. Arch Dis Child. 1980; 55:411-414), pneumonia, epiglottitis, meningitis, abscess, infections in eyes (Yagupsky P, Dagan R, Howard C W, et al. J Clin Microbiol. 1992; 30:1278-1281; Kennedy C A, Rosen H. Am J Med. 1988; 85:701-702; Mollee T, Kelly P, Tilse M. J Clin Microbiol. 1992; 30:2516-2517), etc.;

Bacteroides ovatus causes meningitis, brain tumor, pharyngitis, mumps, abdominal infections, diarrhea, female genital infections, osteomyelitis, septic arthritis (Mandell 5th chapter 237 Bacteroides, Prevotella. Porphyromonas, and Fusobacterium Species and Other Medically Important Anaerobic Gram-Negative Bacilli, p. 2561- 2570), etc.;

Bacteroides thetaiotaomicron causes enteritis that related to antibiotics (George W L, Rolfe R D, Finegold S M. J Clin Microbiol. 1982; 15:1049-1053; Smith J A, Cooke D L, Hyde S, et al. J Med Microbiol. 1997; 46:953-958);

Clostridium difficile causes watery and nosocomial diarrhea.

Haemohilus aphrophilas causes localized brain or respiratory infections, sinusitis, otitis media, pneumonia (Kiddy K, Webberley J. J Infect. 1987; 15:161-163), abscess, bacteremia, endocarditis (Geraci J E, Wilkowske C J, Wilson W R, et al. Mayo Clin Proc. 1977; 52:209-215), infectious arthritis, osteomyelitis (Petty B G, Burrow C R, Robinson R A, et al. Am J Med. 1985; 78:159-162), abscesses in the soft tissues, wound infections, necrotizing fascitis, meningitis (Petty B G, Burrow C R, Robinson R A, et al. Am J Med. 1985; 78:159-162), brain tumor (Kilian M., J Gen Microbiol. 1976;93:9-62; Page M I, King E O. Engl J Med. 1966; 275:181-188; Sutter V L, Finegold S M. Ann N Y Acad Sci. 1970; 174:468-487; Kraut M S, Attebery H R, Finegold S M, et al. J Infect Dis. 1972; 126:189-192; Elster S K, Mattes L M, Meyers B R, et al. Am J Cardiol. 1975; 35:72-79; Bieger R C, Brewer N S, Washington JA II. Medicine (Baltimore) . 1978; 57:345-355);

Neisseria gonorrhea causes following disorders in male reproductive system such as acute urethritis, acute epididymitis, lymphadenitis around penis, abscesses abround urethra, acute prostatitis, infections in Tysons's gland and Cowper's gland (Cohen M S, Cannon J G, Jerse A E, et al. J Infect Dis. 1994; 169:532-537), cervicitis, urethritis, Salpingitis in female reproductive system (Platt R, Rice P A, McCormack W M. JAMA. 1983; 250:3205-3209), anorectal gonorrhea, pharyngeal gonorrhea in homosexuals (Handsfield H H, Knapp J S, Diehr P K, et al. Sex Transm Dis. 1980; 7:1-5), ophthalmitis, acute palatitis, oral ulcer, skin infections, oral abscess, pelvic inflammatory disorders (Quinn T C, Stamm W E, Goodell S E, et al. N Engl J Med. 1983; 309:576-582) etc.;

Eikenella corrodens causes bite wounds and infection (Goldstein E J C. Clin Infect Dis. 1992; 14:633-640), odontogenic head and neck infection (Tveteras K, Kristensten S, Bach V, et al. J Laryngol Otol. 1987; 101:592-594), respiratory infection (Suwanagool S, Rothkopf M M, Smith S M, et al. Arch Intern Med. 1983; 143:2265-2268), gynecologic infection (Jeppson K G, Reimer L G. Obstet Gynecol. 1991; 78:503-505; Drouet E, De Montclos H, Boude M, et al. Lancet. 1987; 2:1089), lung infection (Joshi N, O'BryanT, Appelbaum P C. Rev Infect Dis. 1991; 13:1207-1212), endocarditis (Decker M D, Graham B S, Hunter E B, et al. Am J Med Sci. 1986;292:209-212), etc.;

Bacteroides vulgatus causes meningitis, brain tumor, pharyngitis, mumps, abdominal infections, diarrhea, infections in female genital organs, osteomyelitis, septic arthritis (Mandell 5th, chapter 237 Bacteroides, Prevotella. Porphyromonas, and Fusobacterium Species and Other Medically Important Anaerobic Gram-Negative Bacilli, p. 2561-2570); and

Branhamella catarrhalis causes otitis media (Stenfors L-E, Raisanen S. J Laryngol Otol. 1990; 104:749-757), infections in the lower respiratory track, aggravating chronic obstructive respiratory disease to acute (Verghese A, Roberson D, Kalbfleisch JH, Sarubbi F. Antimicrob Agents Chemother. 1990; 34:1041-1044), pneumonia (Collazos J. de Miguel J, Ayarza R. Eur J Clin Microbiol Infect Dis. 1992; 11:237-240), respiratory infection (McKenzie H, Morgan M G, Jordens J Z, et al. J Med Microbiol. 1992; 37:70-76), sinusitis (Pentilla M, Savolainen S, Kuikaanniemi H, et al. Acta Otolaryngol (Stockh). 1997; (Suppl) 529:S165-S168), bacteriemia (Ioannidis J P A, Worthington M, Griffiths J K, Snydman D R. Clin Infect Dis. 1995; 21:390-397);

Sutterella wadsworthensis causes acute appendicitis, peritonitis, abdominal abscesses (Clin Infect Dis. 1997; 25(Suppl 2) :S88-S93)

Candida albicans causes aphtha (Schultz, F. W 1925. Am. J. Dis. Child, 29; 283-285), glossitis (Bassiouny, A et al. 1984. J. Laryngol. Otol.,98; 609-611), stomatitis (Olsen, et al. 1978. Scand. J. Dent. Res, 86; 392-398), vaginitis (Ryley, J. F, J. Med. Vet. Mycol., 24; 5-22, 1986), bronchopneumonia (Plummers,N. S. 1966 Symposium on Candida infection. London, Churchill Livingstone, pp 214-220), esophagitis, gastritis, enteritis (Trier, J. S 1984. Am. J. Med., 77; 39-43), chronic mucocutaneous candidosis (Jorizzo, J. L. 1982. Arch. Dermatol., 118; 963-965), Onychomycosis (Ray, T. L et al.1978. Int. J. Dermatol.,17; 603-690), diaper related diseases (Leyden, J. J 1978. Arch. Dermatol.,114; 56-59), candidal granuloma (Imperator, P. J 1968. Arch. Dermatol., 97; 139-146), endocarditis (Ben Joseph, 1985. Harefuah, 108; 72-73), infections in the urinary organs (Goldberg, P. K 1979. J. Am. Med. Assoc., 241; 582-584), meningitis (Roessman.1967. Arch. Pathol., 84; 495-498), sepsis (Ashcraft, K 1970. J. Am. Med. Assoc., 217; 454-456), eczema (Drouet, M. 1985. Allergie Immunol., 17; 13-18), asthma (Wengrower, D et al. 1985. Respiration, 47; 209-213), etc.;

Candida glabrata causes mycosis (Block, C. S., Young, C. N. and Myers, R. A. M. 1977. S. Afr. Med. J 51, 632-636), necrotizing purulent inflammation, granulating reactions (Francis W. Chandler, Williams Kaplan et al. A Colour Atlas and Textbook of the Histopathology of Mycotic Disease. Wolfe Medical Publications Ltd. pp 45), sepsis (Minkowitz, S., D. Koffler, et al. 1963. Am. J. Med., 34:252-255), cystopyelonephritis (Newman, D. M., and J. M. Hogg, et al. 1969. J. Urol., 102:547-548), respiratory infections (Oldfiekld, F. S. J., L. Kapica, et al. 1968. Can. Med. Assoc. J., 98:165-168), endocarditis (Carmody, T. J., K. K. Kane. 1986. Heart Lung, 15:40-42; Heffner, D. K., and W. A. Franklin. 1978. Am. J. Clin. Pathol., 70:420-423; Lees, A. W., S. S. Rau, et al. 1971. Lancet, 1:943-944), cerebrospinal meningitis (Wickerham, L. J. 1957. J. Am. Med. Assoc., 165:47-48), Endophthalmitis (Larson, P. A., R. L. Lindhstrum, et al. 1978. Arch. Ophthalmol., 96:1019-1022).

The following examples serve to illustrate certain preferred embodiments and aspects of the present invention and are not to be construed as limiting the scope thereof.

### EXAMPLE 1

Nucleotide Sequencing of 23S rDNA and ITS

Full nucleotide sequences of microbes as listed in Table 1 below were determined according to the present invention.

TABLE 1

| | Gram | | |
|---|---|---|---|
| Species | staining | Source | SEQ ID NO |
| Acinetobacter baumanii | + | KCTC 2771 | 1 |
| Anaerobiospirillum | − | ATCC 29305 | 2 |
| Succiniciproducens | | | |
| Bacteroides fragilis | − | ATCC 25282 | 3 |
| Cardiobacterium hominis | − | ATCC14900 | 4 |
| Chryseobacterium | − | ATCC 13253 | 5 |
| Meningosepticum | | | |
| Clostridium ramosum | + | ATCC 25582 | 6 |
| Comamonas acidovorans | − | ATCC 9355 | 7 |
| Corynebacterium diphtheriae | + | ATCC 51696 | 8 |
| Klebsiella oxytoca | − | ATCC 43863 | 9 |
| Ochrobactrum anthropi | − | ATCC 49188 | 10 |
| Peptostreptococcus prevotii | + | KCTC 3319 | 11 |
| Porphyromonas gingivalis | + | ATCC 33277 | 12 |
| Peptostreptococcus anaerobius | + | ATCC 27337 | 13 |
| Peptostreptococcus magnus | + | ATCC 29328 | 14 |
| Fusobacterium necrophorum | − | ATCC 25286 | 15 |
| Proteus vulgaris | − | KCCM 11539 | 16 |
| Enterobacter aerogenes | − | KCCM 11783 | 17 |
| | | (ATCC 29751) | |
| Streptococcus mutans | + | KCCM 11823 | 18 |
| | | (ATCC 25175) | |
| Kingella kingap | − | ATCC 23330 | 19 |
| Bacteroides ovatus | − | ATCC 8483 | 20 |
| Bacteroides thetaiotaomicron | − | KCTC 5015 | 21 |
| | | (ATCC 29741) | |
| Clostridium diffcile | + | ATCC 9689 | 22 |
| Haemohilus aphrophilas | − | ATCC 13252 | 23 |
| Neisseria gonorrhea | − | ATCC 10150 | 24 |
| Eikenella corrodens | − | ATCC 51724 | 25 |
| Bacteroides vulgatus | − | KCCM 11423 | 26 |
| | | (KCCM 8482) | |
| Branhamella catarrhalis | − | KCCM 40056 | 27 |
| | | (ATCC 43617) | |
| Sutterella wadsworthensis | − | ATCC 51579 | 28 |

KCTC: Korean Collection for Type Cultures, Taejon, Korea
KCCM: Korean Culture Center of Microorganisms, Seoul, Korea
ATCC: American Type Culture Collection, Virginia, USA

Each microbial species was cultured in a manner known per se and chromosomal DNA was extracted from the culture using QIAamp DNA mini-kit (QIAGEN, USA). For the determination of nucleotide sequence of ITS-23S rDNA region, universal primers were first prepared by performing multiple alignment and BLAST of 16S rDNA, ITS and 23S rDNA originating from each species of all bacteria using the extracted DNA as a template. Table 30 below summarizes the nucleotide sequences of universal primers constructed and used for the nucleotide sequencing and the locations thereof. Among the constructed primers, universal primer for 16S rDNAs (i.e., 1585Fw) with the ability to amplify ITS region and several universal primers for 23S rRNA (i.e., 520R, 23S 750F(T), 23S 750F, 970F, 930R, 2960R(T) and 2960RC) were constructed directly by the inventors. The other universal primers, i.e., 23BFw, 23BR, MS37F and MS38R, correspond to those for 23S rDNA described by Anthony, R.M., et al. in J. Clin. Microbiol. 38(2), 781-788, 2000.

TABLE 2

| | Direct- | | SEQ ID | |
|---|---|---|---|---|
| Ref. | ion | Sequence (5′→3′) | NO | Location* |
| 1585F | Forward | TTGTACACACCGCCCGTC | 406 | 16S rRNA |
| | | | | (1390-1407) |
| 520R | Reverse | GCCAAGGCATCCACC | 414 | 23S rRNA |
| | | | | (20-34) |
| 23S 750F | Forward | AGTAGCGGCGAGCGAA | 415 | 235 rRNA |
| | | | | (238-253) |
| 23S | Forward | AGTAGTGGCGAGCGAA | 416 | 23S rRNA |
| 750F (T) | | | | (238-253) |
| 23BFw | Reverse | AGTACCGTGAGGGAAAGG | 408 | 23S rRNA |
| | | | | (454-477) |
| 23BR | Reverse | TTTCGCCTTTCCCTCACGGTACT | 407 | 23S rRNA |
| | | | | (454-477) |
| 970F | Forward | AACTGGAGGACCGAACC | 417 | 23S rRNA |
| | | | | (701-734) |
| MS37F | Forward | AGGATGTTGGCTTAGAAGCA | 410 | 23S rRNA |
| | | | | (1050-1073) |
| 930R | Reverse | AWTTTGCYGAGTTCCTT | 418 | 23S rRNA |
| | | | | (1658-1675) |
| MS38R | Reverse | CCCGACAAGGAATTTCGCTACCTTA | 411 | 23S rRNA |
| | | | | (1923-1946) |
| 2690R | Reverse | GCTTAGATGCTTTCAGCA | 419 | 23S rRNA |
| (T) | | | | (2739-2756) |
| 2960RC | Reverse | GCTTAGATGCTTTCAGCG | 420 | 23S rRNA |
| | | | | (2739-2756) |

*The location of nucleotide sequence is based on 16S rRNA-23S rRNA region of Escherichia coli.
W = Adenine (A) or Thymine (T) and Y = Cytosine (C) or Thymine (T)

To determine nucleotide sequence, PCR was performed using the above universal primers with templates of chromosomal DNA from the microbial species and PCR products were purified. In order to determine the nucleotide sequence of the ITS region and 3′ end of the 23S rDNA, multiple alignment and BLAST were performed of 16S rDNAs from all known microbial species to select a universally conserved sequence. This universally conserved sequence is referred to as the primer 1585Fw. The primer 1585Fw was used along with the primer 23BR for the PCR.

The PCR was conducted by repeating 10 cycles each consisting of first -denaturation at 94° C. for 7 minutes and second denaturation at 94° C. for 1 minute, annealing at 52° C. for 1 minute and extension at 72° C. for 1 minute in that order and then 20 cycles each consisting of third denaturation at 94° C. for 1 minute, annealing at 54° C. for 1 minute and extension at 72° C. for 1 minute in that order.

The resulting PCR products were fractionated by agarose gel electrophoresis and purified. The amplified PCR products were sequenced by DNA-Analyzer (ABI Prism 3700, Perkin Elmer) using primers suitable to the region to be sequenced. To determine a partial sequence of the 23S rDNA, the second PCR was performed with primers 520R, 23S 750F and 23S 750F(T). To determine another partial sequence of the 23S rDNA, the third PCR was performed with primers 23BFw and MS38R. The PCR conditions were the same as those described above. Based on the nucleotide sequence determined therefrom, the fourth PCR was performed with primers 970F and 930R. In addition, multiple alignment and BLAST were performed of 23S rDNAs from all known microbial species to select a universally conserved sequence which is referred to as primers 2960R(T) or 29GORC. The fifth PCR was performed with primers 2960R(T) or 2960RC and MS37F under the same conditions as described above. The full nucleotide sequences of ITS-23S rDNA regions from microbial species listed on Table 1 above are shown in SEQ ID NO: 1 through SEQ ID NO: 28, respectively.

### EXAMPLE 2

Screening of Candidate DNA Probe for the Identification of Microbial Species

For the detection and identification of each species, probes specific to it were constructed. The nucleotide sequence of 23S rRNA and ITS of each species first identified in the above Example 1 or recorded in GenBank was compared to those of all other microorganisms using-a multiple alignment to find a group of nucleotide sequences specifically conserved in the species. These specific nucleotide sequences were chosen as candidate probes specific to the species. For bacteria, candidate probes were selected within 23S rRNA gene and/or ITS region. For fungi, candidate probes were selected within 18S rRNA gene.

The specificity of candidate probes was confirmed by the BLAST analyses. The candidate probes screened thereby are shown in Table 3 below.

### EXAMPLE 3

Synthesis of Nucleic Acid Probes

For the construction of DNA chip, candidate probes screened in the above Example 2 were chemically synthesized. Mononucleotides (Proligo Biochemie GmbH Hamburg Co.) were introduced into an Expedite 8900 nucleic acid synthesis system (PE Biosystems Co.) with input of the desired nucleotide sequence and scale to afford 0.05 umole of pure nucleic acid probes. The resulting probes were confirmed by an electrophoresis.

### EXAMPLE 4

Construction of DNA Chip

In order to immobilize DNA probes on a solid support, amine-aldyhyde covalent bonds were used. The 3′ termini of synthetic oligonucleotide probes was modified with amine residues using an amino linker column (Cruachem, Glasgow, Scotland) for the immobilization on the aldehyde-coated glass slide (CEL Associates, Huston, Tex.). The probes were dissolved in 3×SSC (0.45M NaCl, 15mM C₆H₅Na₃O₇, pH 7.0) spotting solution. The resulting solution was spotted on the slide glass surface using KAIST MBEL DNA microarrayer constructed as described in Yoon. S. H., et al., J. Microbiol. Biotechnol. 10(1), 21-26, 2000, the entire content of which is incorporated therein by reference. The slide glass were kept under about 55% humidity for 1 hour and then air-dried for 6 hours so that the DNA probes could be immobilized on the glass slide. All probes were spotted with intervals of 258 μm at the concentration of 100 pmole. To evaluate efficiency of immobilization, the glass slide was dyed with SYBRO green II (Molecular Probe, Inc., Leiden, Netherlands).

### EXAMPLE 5

Isolation and amplification of Target DNA sample

Genomic DNAs were extracted from 28 bacterial species given in the above Example 1 and 31 known species listed in Table 3 below.

TABLE 3

| Species | Gram Staining | Source |
|---|---|---|
| Actinomyces israelii | − | ATCC 12101 |
| Staphylococcus epidermidis | + | KCTC 1917 |
| Burkholderia cepacia | − | ATCC 25416 |
| Salmonella enteritidis | − | KCCM 12021 |
| Escherichia coli | − | ATCC 25922 |
| Klebsiella pneumoniae | − | ATCC 700603 |
| Proteus mirabilis | − | KCCM 11381 |
| Streptococcus pneumoniae | + | KCCM 40410 |
| Vibrio vulnificus | − | KCTC 2962 |
| Pseudomonas aeruginosa | − | KCTC 1636 |
| Aeromonas hydrohila | − | KCCM 32586 |
| Listeria monocytogenes | + | ATCC 700603 |
| Enterococcus faecium | + | ATCC 19434 |
| Staphylococcus aureus | + | KCTC 1621 |
| Neisseria meningitides | − | ATCC 13100 |
| Legionella pneumophila | − | clinically isolated |
| Candida albicans | Fungus | KCCM 11474 |
| Candida glabrata | Fungus | KCCM 50701 |
| Stomatococcus mucilaginosus | + | ATCC 17931 |
| Shigella sonnei | − | KCCM 11903 |
| Morganella morganii | − | ATCC 25830 |
| Streptococcus pyogen | + | KCCM 11817 |
| Vibrio cholerae | − | KCTC 2715 |
| Haemophilus influenzae | − | ATCC 51907 |
| Stenotrophomonas maltophilla | − | ATCC 13637 |
| Shigella flexneri | − | ATCC 11836 |
| Enterococcus faecalis | + | ATCC 19433 |
| Streptococcus viridans | + | ATCC 35037 |
| Serratia marcences | − | KCTC 1299 |
| Citrobacter freundii | − | ATCC 51579 |

The microbial species was grown on a suitable medium and suspended in 200 μl of sterilized distilled water. The suspension was centrifuged at 14,000 rpm for 10 minutes. The supernatant was discarded to obtain a pellet.

For gram-negative species, the pellet was put into 180 μl of ATL solution (Tissue Lysis Solution, DNeasy Tissue Kit, QIAGEN). 20 μl of proteinase K was added to the solution to lyse cells. The resulting lysate was cultured at 55° C. for 1 hour. The culture was vortexed for 15 seconds and mixed with 200 μl of AL solution (Lysis Solution, DNeasy Tissue Kit, QIAGEN). The resulting mixture was cultured at 70° C. for 10 minutes. The culture was mixed with 200 μl of ethanol (100%). The resulting solution was loaded onto the DNeasy mini column sitting in a 2 ml tube and centrifuged at 8,000 rpm or more for 1 minute. The solution collected in the tube was discarded. 500 μl of AW1 solution (Wash Solution 1, DNeasy Tissue Kit, Qiagen) was pipetted into the column which was then centrifuged at 8,000 rpm for 1 minute. The elute was discarded and 500 μl of AW2 solution (Wash Solution 2, DNeasy Tissue Kit, Qiagen) was again pipetted into the column which was then centrifuged at a full speed for 3 minutes. The DNeasy membrane was dried and the elute was discarded. The dry DNeasy mini column was placed in the tube and stood at room temperature for 15 minutes, and then centrifuged at 8,000 rpm for 1 minute to elute genomic DNAs.

For gram-positive species, the pellet was suspended into 180 μl of lysozyme solution (20 mM Tris-Cl, pH 8.0, 2 mM EDTA, 1.2% Triton X-100, 20 mg/ml lysozyme) and cultured at 37° C. for 30 minutes. The culture obtained thereby was mixed with 25 μl of proteinase K and 200 μl of AL solution (Lysis Solution, DNeasy Tissue Kit, QIAGEN).

The resulting mixture was cultured at 70° C. for 30 minutes. The culture was mixed with 200 μl of ethanol (100%). The resulting solution was loaded onto the DNeasy mini column sitting in a 2 ml tube and centrifuged at 8,000 rpm or more for 1 minute. The solution collected in the tube was discarded. 500 μl of AW1 solution (Wash Solution 1, DNeasy Tissue Kit, Qiagen) was pipetted into the column which was then centrifuged at 8,000 rpm for 1 minute. The elute was discarded and 500 μl of AW2 solution (Wash Solution 2, DNeasy Tissue Kit, Qiagen) was again pipetted into the column which was then centrifuged at a full speed for 3 minutes. The DNeasy membrane was dried and the elute was discarded. The dry DNeasy mini column was placed in the tube and stood at room temperature for 15 minutes, and then centrifuged at 8,000 rpm for 1 minute to elute genomic DNAs.

For fungi, the pellet was mixed with 200 μl of SDS TE buffer solution (10% SDS, 100 mM Tris-Cl, 20 mM EDTA, pH 8.0) and 20 μl of proteinase K (contained in DNeasy Tissue Kit). The resulting mixture was cultured at 55° C. for 2 hours and then at 95° C. for 10 minutes. The culture was mixed with 200 μl of ethanol (100%). The resulting solution was loaded onto the DNeasy mini column sitting in a 2 ml tube and centrifuged at 8,000 rpm or more for 1 minute. The solution collected in the tube was discarded. 500 μl of AW1 solution (Wash Solution 1, DNeasy Tissue Kit, Qiagen) was pipetted into the column which was then centrifuged at 8,000 rpm for 1 minute. The elute was discarded and 500 μl of AW2 solution (Wash Solution 2, DNeasy Tissue Kit, Qiagen) was again pipetted into the column which was then centrifuged at a full speed for 3 minutes. The DNeasy membrane was dried and the elute was discarded. The dry DNeasy mini column was transferred to 1.5 ml tube. 100 μl of AE solution (eluent, DNeasy Tissue Kit, QIAGEN) was put into the tube, stood at room temperature for 15 minutes, and then centrifuged at 8,000 rpm for 1 minute to elute genomic DNAs. 100 μl of AE solution (eluent, DNeasy Tissue Kit, QIAGEN) was put into the tube, stood at room temperature for 15 minutes, and then centrifuged at 8,000 rpm for 1 minute to elute genomic DNAs. Again, 100 μl of AE solution (eluent, DNeasy Tissue Kit, QIAGEN) was put into the tube, stood at room temperature for 15 minutes, and then centrifuged at 8,000 rpm for 1 minute to elute genomic DNAs.

To prepare single-stranded DNA, asymmetric PCR was carried out using DNAs isolated from microbial species as described above as a template. The single-stranded DNA was synthesized by one cycle of PCR with addition of forward primer and reverse primer at a ratio of 1:5. The reverse primers which were used to amplify the strand complementary to the probes were labeled with fluorescein isothiocyanate (FITC) for detection.

Where DNAs wer isolated from bacterial species, the following three sets of primers were simultaneously used:

Primer 1 (sense):
TTGTACACACCGCCCGTC (SEQ ID NO: 406, 1585Fw)
and
Primer 2 (antisense):
F-TTTCGCCTTTCCCTCACGGTACT (SEQ ID NO: 407, 23BR)
Primer 3 (sense):
AGTACCGTGAGGGAAAGGCGAA (SEQ ID NO: 408, 23BFw)
and
Primer 4 (antisense):
F-TGCTTCTAAGCCAACATCCT (SEQ ID NO: 409, 37R);
and
Primer 5 (sense):
AGGATGTTGGCTTAGAAGCA (SEQ ID NO: 410, MS37F)
and
Primer 6 (antisense):
F-CCCGACAAGGAATTTCGCTACCTT (SEQ ID NO: 411, MS38R)
(F = FITC labeled at 5′-terminus).

Where DNAs were isolated from fungal species, the following set of primers were used:

Primer 1 (sense):
GTAATTGGAATGAGTACAAT (SEQ ID NO: 412, fun4G3F)
and
Primer 2 (antisense):
F-CTACGACGGTATCTGATCAT (SEQ ID NO: 413, fun986R)

(F=FITC labeled at 5′-terminus).

The asymmetric PCR were performed as follows: PCR mixtures contained 50 ul of 10×PCR buffer (100 mM Tris-HCl, pH 8.3, 500 mM KCl, 15 mM MgCl₂), 4 ul of 0.2 mM dNTP, 0.5 ul of 10 pmol forward primer, 2.5 ul of 10 pmol reverse primer, 1 ul of 1/10 diluted DNA template (100 ng), 0.5 ul of Taq polymerase (5 units/ul, Takara Shuzo Co., Shiga, Japan) and water to final volume of 100 ul. The PCR cycling conditions were: 10 cycles of first denaturation at 94° C. for 7 minutes, second denaturation at 94° C. for 1 minute, annealing at 52° C. for 1 minute and extension at 72° C. for 1 minute, 30 cycles of third denaturation at 94° C. for 1 minute, annealing at 52° C. for 1 minute and extension at 72° C. for 1 minute, followed by one final extension at 72° C. for 5 minutes. The PCR products were analyzed by agarose gel electrophoresis. The analysis showed that double-stranded DNA and single-stranded DNA for each species were synthesized together.

### EXAMPLE 6

Hybridization and Wash

To confirm the specificity and sensitivity of the candidate probes, hybridization was performed by applying the PCR products prepared in the above Example 5 to the DNA chip prepared in the above Example 4 to which the candidate probes were immobilized. If a candidate probe showed positive hybridization signals for the species thereof, then it was additionally tested for cross-reactions (specificity) with genomic DNAs from the other species.

The DNA chip was hydrated with a water vapor and then soaked in 70% ethnol to remove any probes which had not yet been immobilized on a glass slide of the DNA chip. During a hybridization reaction, fluorescence would incur the augmentation of a hybridization signal by attaching to aldehyde groups on the glass slide surface and consequently diminish the hybridization signal with the specific probe immobilized on the chip. To prevent any reduction in a hybridization signal, the DNA chip was transferred to a blocking solution (1.3 g of NaBH₄, 375 ml of PBS, 125 ml of 100% ethnol) and then shaken for 5 minutes. The DNA chip was washed with 0.2% SDS for 5 minutes and then twice or three times with a sterile water for 1 minute each. The DNA chip was centrifuged at 1,000 rpm for 2 minutes to remove water on the glass slide.

30 μl of the asymmetric PCR products was mixed with 170 μl of 6×SSPE hybridization buffer solution (20×SSPE:. 3M NaCl, 0.2M NaH₂PO₄H₂O, 0.02M EDTA, pH7.4, Sigma Co., St. Louis, Mo.). The resulting mixture was applied on a glass slide onto which the probes were immobilized and covered with a probe-clip press-seal incubation chamber (Sigma Co., St. Louis, Mo.).

The hybridization reaction was continued for 6 hours in a shaking incubator at 30° C.. After the completion of hybridization, the slides was washed with 3×SPE (0.45 M NaCl, 15 mM C₆H₅Na₃O₇, pH 7.0), 2×SSPE (0.3 M NaCl, 10 mM C₆H₅Na₃O₇, pH 7.0) and then 1×SSPE (0.15 M NaCl, 10 mM C₆H₅Na₃O₇, pH 7.0) for 5 minutes each.

### EXAMPLE 7

Detection of Hybrids

The hybrids were detected using ScanArray 5000 (GSI Lumonics In., Bedford, Mass.). The hybridization results are given in Tables 4 through 49 below.

TABLE 4

| | Nucleotide | SEQ | Loca- | |
|---|---|---|---|---|
| Ref. | Sequence | ID NO | tion | Specificity |
| Acti1 | GGGCACACATAATGA | 35 | 23S | Upon four applications |
| (Acti23) | | | | of KCTC 2771 genomic |
| Acti2 | | | | genes, no positive |
| (ActiM) | CGGGGTACTCTATAC | 36 | 23S | hybridization signals |
| | | | | were obtained. |
| Acti3 | ATACACAGTACTTCG | 32 | ITS | cross-reacted with |
| (ActiI) | | | | genomic genes of |
| | | | | Cardiobacterium |
| | | | | hominis, |
| | | | | Actinomycesisraelii, |
| | | | | Rothia, and Kiebsiella |
| | | | | oxytoca but not cross- |
| | | | | reacted with genomic |
| | | | | genes of the other 55 |
| | | | | species |
| Acti001 | AGGTATTGCAACATG | 39 | ITS | Upon four applications |
| | | | | of KCTC2771 genomic |
| | | | | genes, no positive |
| | | | | hybridization signals |
| | | | | were obtained |
| Acti002 | ATAGTGTTGCAAGGC | 33 | ITS | not cross-reacted with |
| | | | | genomic genes of all 59 |
| | | | | species |
| Acti003 | TGAAAAGCCAGGGGA | 34 | ITS | cross-reacted with |
| | | | | genomic genes of |
| | | | | Salmonella spp. but not |
| | | | | cross-reacted with |
| | | | | genomic genes of the |
| | | | | other 58 species |
| Acti004 | TGATGGAACTTGCTT | 29 | 23S | not cross-reacted with |
| | | | | genomic genes of all 59 |
| | | | | species |
| ActiIT01 | CAGAAGTAGCTGCCT | 40 | ITS | Upon four applications |
| ActiIT02 | AGAAGTAGCTGCCTA | 41 | ITS | of KCTC2771 genomic |
| ActiIT03 | GAAGTAGCTGCCTAA | 42 | ITS | genes, no positive |
| ActiIT04 | AAGTAGCTGCCTAAC | 43 | ITS | hybridization signals |
| ActiIT05 | AGTAGCTGCCTAACT | 44 | ITS | were obtained. |
| Acti23S01 | AGGGCACACATAATG | 30 | 23S | not cross-reacted with |
| | | | | genomic genes of all 59 |
| | | | | species |
| Acti23S02 | ACGCTGTTGTTGGTG | 31 | 23S | cross-reacted with |
| | | | | genomic genes of |
| | | | | Cardiobacterium hominis, |
| | | | | Actinomyces israelii, |
| | | | | Stomatococcus |
| | | | | mucilaginosa |
| | | | | (hereinafter, Rothia) |
| | | | | and Lebsiella oxytoca |
| | | | | but not cross-reacted |
| | | | | with genomic genes of |
| | | | | the other 55 species |
| Acti23S03 | GTAGGTATGTATCTT | 37 | 23S | Upon four applications |
| Acti23S04 | TACTGAGATCCGATA | 38 | 23S | of KCTC2771 genomic |
| | | | | genes, no positive |
| | | | | hybridization signals |
| | | | | were obtained. |

TABLE 5

| | Nucleotide | SEQ | Loca- | |
|---|---|---|---|---|
| Ref. | Sequence | ID NO | tion | Specificity |
| Anas1 | AAAGTGCAGGGCACA | 56 | 23S | Upon four applications |
| (Anas7) | | | | of ATCC 29305 genomic |
| Anas2 | TGGATTGTGGTGAAA | 57 | 23S | genes, no positive |
| (AnasM) | | | | hybridization signals |
| Anas3 | TAGCGTTCTGCGAGG | 58 | 23S | were obtained. |
| (Anas7) | | | | |
| Anas4 | TTAAAAGACTGGTAT | 59 | 23S | |
| (AnasM) | | | | |
| Anas001 | TGACTCGTGCCCATG | 45 | 23S | not cross-reacted with |
| Anas002 | TACCGGGGTTAAAAG | 46 | 23S | genomic genes of all 59 |
| Anas003 | ATCAGTGATCTGAGA | 47 | 23S | species |
| Anas004 | GAGACGAAGCACCAT | 48 | 23S | cross-reacted with |
| | | | | genomic genes of |
| | | | | Bacteroides fragilis, |
| | | | | and Serratia marcescens |
| | | | | but not cross-reacted |
| | | | | with geriomic genes of |
| | | | | the other 57 species |
| Anas005 | GTTCTTGATTCATTG | 52 | ITS | not cross-reacted with |
| | | | | genomic genes of all 59 |
| | | | | species |
| Anas006 | ATCCAATCATGATCA | 60 | 23S | Upon four applications |
| Anas007 | AAGCATGAAAGCGCA | 61 | 23S | of ATCC 29305 genomic |
| | | | | genes, no positive |
| | | | | hybridization signals |
| | | | | were obtained. |
| nas008 | CAGCCCAAAAGTTGA | 53 | ITS | Not cross-reacted with |
| Anas009 | AAACTGCAGGGCACA | 54 | ITS | genomic genes of all 59 |
| Anas010 | ATACTACCTGACGAC | 55 | ITS | species |
| Anas011 | AGTTGATACAGGTAG | 49 | 23S | cross-reacted with |
| | | | | genomic genes of |
| | | | | Serratia marcescens, and |
| | | | | Salmonella spp. but not |
| | | | | cross-reacted with |
| | | | | genomic genes of the |
| | | | | other 57 species |
| Anas012 | TAGCGTTCTGCGAGG | 70 | ITS | Upon four applications |
| | | | | of ATCC29305 genomic |
| | | | | genes, no positive |
| | | | | hybridization signals |
| | | | | were obtained. |
| Anas013 | GGCCCCATCCGGGGT | 50 | 23S | cross-reacted with |
| | | | | genomic genes of E. coli |
| | | | | but not cross-reacted |
| | | | | with genomic genes of |
| | | | | the other 58 species |
| Anas014 | GAGGCGGGAGCCGAG | 62 | 23S | Upon four applications |
| Anas23001 | CCCCATCCGGGGTTG | 63 | 23S | of ATCC 29305 genomic |
| Anas23S01 | TGGCGTCAGGAGGCG | 64 | 23S | genes, no positive |
| nas23502 | ATAAGGGGCGCTTGA | 65 | 23S | hybridization signals |
| | | | | were obtained. |
| Anas23S03 | CAGTTGGAAGCAGAG | 51 | 239 | cross-reacted with |
| | | | | genomic genes of |
| | | | | Cardiobacterium hominis, |
| | | | | Bacteroides fragilis and |
| | | | | Strentrophomonas |
| | | | | maltophila but not |
| | | | | cross-reacted with |
| | | | | genomic genes of the |
| | | | | other 56 species |
| Anas23S04 | TCACACGCAAGTGTG | 66 | 23S | Upon four applications |
| Anas23S05 | GCTGAGACGAAGCAC | 67 | 23S | of ATCC 29305 genomic |
| Anas23S06 | ATACCGGGGTTAAA | 68 | 23S | genes, no positive |
| AnasIT001 | ACAGCGCAGCATGTG | 71 | ITS | hybridization signals |
| AnasIT002 | AATTAGCAACTATTT | 72 | ITS | were obtained. |
| AnasIT01 | CTTCCCTCAGTGATT | 73 | ITS | |
| AnasIT02 | TTCCCTCAGTGATTC | 74 | ITS | |
| AnasIT03 | TCCCTCAGTGATTCA | 75 | ITS | |
| AnasIT04 | CCCTCAGTGATTCAA | 76 | ITS | |
| AnasIT05 | CCTCAGTGATTCAAG | 77 | ITS | |
| Anas23S03 | CAGTTGGAAGCAGAG | 69 | 23S | |

TABLE 6

| | Nucleotide | SEQ | Loca- | |
|---|---|---|---|---|
| Reference | Sequence | ID No. | tion | Specificity |
| Bacf1 | GGTAACCGAAGCGTA | 79 | 23S | not cross-reacted with |
| (Bf23) | | | | genomic genes of all 59 |
| | | | | species |
| Bacf2 (Bf) | CTCGGAAAACGGTAA | 80 | 23S | Upon four applications |
| Bacf3 | GGTTCAGATCCTTTT | 92 | ITS | of ATCC 25282 genomic genes, |
| (BfI) | | | | two positive hybridization |
| Bf001 | AGCGATGTTGAAAAC | 81 | 23S | signals were obtained. |
| Bf002 | TCAACCATCTATAGC | 82 | 23S | |
| Bf003 | AACAAGAGAAAAACA | 83 | 23S | |
| Bf004 | CGATACCGCGACCTA | 84 | 23S | |
| Bf005 | TATATCGAACCATTT | 85 | 23S | |
| Bf006 | GAATCTGGCGATAAA | 86 | 23S | cross-reacted with genomic |
| | | | | genes of Porphylomonas |
| | | | | gingivalis, Chryseobacterium |
| | | | | meningosepticum, |
| | | | | Ochrobactrum anthropi, |
| | | | | Actinomyces israelii and |
| | | | | Rothia but not cross-reacted |
| | | | | with genomic genes of the |
| | | | | other 54 species |
| Bf007 | TGCAAATGACCTTTG | 87 | 23S | Upon four applications of |
| Bf008 | CAACTTGGTTGGAGG | 88 | 23S | ATCC25282 genornic genes, |
| Bf009 | ACCCATGTTACGGCA | 89 | 23S | no positive hybridization |
| Bf010 | AGTTGACCTAACGAA | 90 | 23S | signals were obtained. |
| Bf012 | GTCGAACCTGACAGT | 78 | 23S | not cross-reacted with |
| | | | | genomic genes of all 59 |
| | | | | species |
| Bf012 | TGAACGGATCTGTGT | 91 | 23S | Upon four applications of |
| | | | | ATCC25282 genomic genes, |
| | | | | no positive hybridization |
| | | | | signals were obtained. |

TABLE 7

| | | | | |
|---|---|---|---|---|
| | Nucleotide | SEQ | Loca- | |
| Reference | Sequence | ID NO | tion | Specifity |

| | | | | |
|---|---|---|---|---|
| Car1 | CCATACACAATGAAT | 103 | 23S | Upon four applications of |
| (Car23) | | | | ATCC 14900 genomic genes, |
| | | | | no positive hybridization |
| | | | | signals were obtained. |
| Car2 | CCAGCACACTGTTGG | 97 | 23S | not cross-reacted with |
| (CarM) | | | | genomic genes of all 59 |
| Car3 (CarI) | AAAGAGAGAACAGCA | 98 | ITS | species |
| Car001 | TTGGCGACAACAGGC | 99 | ITS | |
| Car002 | GCCCCGGGAAGCTGA | 100 | ITS | |
| Car003 | TAGACTGCGGAAGCG | 101 | ITS | |
| Car004 | AATTAAGTTGCGTAT | 102 | ITS | |
| Car005 | TACTCGTTGTCGACC | 104 | 23S | Upon four applications of |
| | | | | ATCC 14900 genomic genes, |
| | | | | no positive hybridization |
| | | | | signals were obtained. |
| Car006 | AACCCTGGTGAAGGG | 93 | 23S | not cross-reacted with |
| Car007 | ATATGAAGATATGTG | 94 | 23S | genomic genes of all 59 |
| Car008 | TAGATTGACTTACGG | 95 | 23S | species |
| Car009 | GTAAAGTTTTACTAC | 96 | 23S | |

TABLE 8

| | Nucleotide | SEQ | Loca- | |
|---|---|---|---|---|
| Reference | Sequence | ID NO | tion | Specificity |
| Chry1 | ATTTAGATGATAAAT | 110 | 23S | Upon four applications of |
| (Chry23) | | | | ATCC13253 genomic genes, |
| Chry2 | TAATCTTACTAGCGA | 111 | 23S | no positive hybridization |
| (Chry7) | | | | signals were obtained. |
| Chry3 | TCCTTGAGTGCAGAG | 113 | ITS | |
| (ChryI) | | | | |
| Chr001 | CTTAGGTGATCACTT | 106 | ITS | cross-reacted with genomic |
| | | | | genes of Actinomyces israeiii |
| | | | | and Porphylomonas gingivalis |
| | | | | but not cross-reacted with |
| | | | | genomic genes of the other 57 |
| | | | | species |
| Chr002 | AGCACAGCTTTGGTT | 114 | ITS | Upon four applications of |
| | | | | ATCC13253 genomic genes, |
| | | | | no positive hybridization |
| | | | | signals were obtained. |
| Chr003 | TAACCCCTTAGATTA | 107 | ITS | not cross-reacted with genomic |
| Chr004 | TCAAACCTCAAACTA | 108 | ITS | genes of all 59 species |
| Chr005 | AAGAAATCGAAGAGA | 109 | ITS | |
| Chr23S04 | GGCATATTTAGATGA | 105 | 23S | |
| Chr23S05 | ATCGTGAGGTTACGA | 112 | 23S | cross-reacted with genomic |
| | | | | genes of Cardiobacterium |
| | | | | hominis, Ochrobacterium, Rothia, |
| | | | | Porphylomonas gingivails, |
| | | | | Peptostreptococcus prevotii, |
| | | | | Actinomyces israelii, |
| | | | | Haemophilus influenza and |
| | | | | Burkholderia cepacia |
| | | | | but not cross-reacted with |
| | | | | genomic genes of the other 51 |
| | | | | species |

TABLE 9

| | Nucleotide | SEQ | Loca- | |
|---|---|---|---|---|
| Reference | Sequence | ID NO | tion | Specificity |
| C. ramosa01 | GGTGAAGTATTAGTA | 117 | 23S | Upon four applications of |
| C. ramosa02 | ATGTACAGGCATAGG | 118 | 23S | ATCC25582 genomic genes, |
| C. ramosa03 | TGAGAGACATGCACG | 119 | 23S | no positive hybridization |
| | | | | signals were obtained. |
| C. ramosa04 | CCAGTGTGTGAGGAG | 115 | 23S | cross-reacted with |
| | | | | genomic genes of F. |
| | | | | necrophorum, E. aerogenes |
| | | | | and C. diphtheria but not |
| | | | | cross-reacted with |
| | | | | genomic genes of the |
| | | | | other 17 species* |
| C. ramosa05 | GTATTGGAGTTGCTA | 120 | 23S | Upon four applications of |
| C. ramo001 | TAGTTGATGATAGTA | 121 | 23S | ATCC25582 genomic genes, |
| C. ramo002 | GCTTATCTGTGGATG | 122 | 23S | no positive hybridization |
| C. ramo003 | GGAATCCCTCCTTGT | 123 | 23S | signals were obtained. |
| C. ramo004 | CCCGGGAAGGGGAGT | 116 | 23S | cross-reacted with |
| | | | | genomic genes of E. coil |
| | | | | but not cross-reacted |
| | | | | with genomic genes of the |
| | | | | other 19 species* |

TABLE 10

| | Nucleotide | SEQ | Loca- | |
|---|---|---|---|---|
| Reference | Sequence | ID NO | tion | Specificity |
| Coma1 | AAAACCGACTGGTGG | 130 | 23S | Upon four applications |
| (Com23) | | | | of ATCC 9355 genornic |
| | | | | genes, no positive |
| | | | | hybridization signals |
| | | | | were obtained. |
| Coma2 (Com7) | TGAGCTAGAGAAAAG | 128 | 23S | not.cross-reacted with |
| Coma3 (ComM) | ATCCGCCGGGCTTAG | 129 | 23S | genomic genes of all 59 |
| | | | | species |
| Coma4 | ACGCGCGAGGTGAGA | 134 | ITS | Upon four applications |
| (ComI) | | | | of ATCC 9355 genornic |
| Com001 | GCTGACGGAAAGAGA | 131 | 23S | genes, no positive |
| Com002 | CTCTTGACAGAAATG | 132 | 23S | hybridization signals |
| Com003 | AAGAATTCATTCACA | 133 | 23S | were obtained. |
| Com004 | TAGGGCGTCCAGTCG | 124 | 23S | not cross-reacted with |
| Com005 | CGCAGAGTACAGCTT | 125 | 23S | genomic genes of all 59 |
| | | | | species |
| Com006 | GTACCGATGTGTAGT | 126 | 23S | cross-reacted with |
| | | | | genomic genes of |
| | | | | Chryseobacterium |
| | | | | meningosepticum |
| | | | | but not cross-reacted |
| | | | | with genomic genes of |
| | | | | the other 58 species |
| Com007 | GAACTTGAACAAAGG | 127 | 23S | cross-reacted with |
| | | | | genomic genes of |
| | | | | Salmonella spp. and |
| | | | | Serratia marcescens but |
| | | | | not cross-reacted with |
| | | | | genomic genes of the |
| | | | | other 57 species |

TABLE 11

| | Nucleotide | SEQ | Loca- | |
|---|---|---|---|---|
| Reference | Sequence | ID NO | tion | Specificity |
| C. dipht01 | TAACTAGATAAGAAA | 136 | 23S | Upon four applications of |
| C. diph001 | ACCACGCAGCAGTTT | 137 | 23S | ATCC 51696 genomic genes, |
| C.diph002 | CGAGTCGGTAGGGTA | 138 | 23S | one positive |
| | | | | hybridization signals |
| | | | | were obtained. |
| C. diph003 | ACCATCTTCCCAAGG | 135 | 23S | not cross-reacted with |
| | | | | genomic genes of 20 |
| | | | | species* |
| C. diph004 | TGTTTGTTCTTTGAT | 139 | 23S | Upon four applications of |
| C. diph005 | AAAATCAGAAAAACA | 140 | 23S | ATCC 51696 genomic genes, |
| C. diph006 | GGAAAATCAGAAAA | 141 | 23S | no positive hybridization |
| | | | | signals were obtained. |

TABLE 12

| | Nucleotide | SEQ | Loca- | |
|---|---|---|---|---|
| Reference | Sequence | ID NO | tion | Specifity |
| Koxy1 | CCGGAACGTTACTAA | 143 | 23S | Upon four applications of |
| (KoM) | | | | ATCC 43863 genomic genes, |
| Koxy2 | CGCGACACGACGATG | 150 | ITS | no positive hybridization |
| (KoI) | | | | signals were obtained. |
| Koxy3 | AAGAGCGCCAGCTCA | 144 | 23S | |
| (KoM) | | | | |
| Ko001 | GAACGTTACTAACGC | 142 | 23S | not cross-reacted with |
| | | | | genomic genes of all 59 |
| | | | | Species |
| Ko001 | TTTGAAGTTCTAACT | 145 | 23S | Upon four applications of |
| Ko002 | AAGAGCGCCAGCTAC | 146 | 23S | ATCC 43863 genomic genes, |
| Ko002 | TATCTACCGCGGGCG | 147 | 23S | no positive hybridization |
| Ko003 | GATGAAGACCTCAAA | 148 | 23S | signals were obtained. |
| Ko003 | TTACGGGTTGTCATG | 149 | 23S | |

TABLE 13

| | Nucleotide | SEQ | Loca- | |
|---|---|---|---|---|
| Reference | Sequence | ID NO | tion | Specificity |
| Ochr1 | CGGCGCGTGAGCGAG | 156 | 23S | Upon four applications |
| (Ochr23-1) | | | | of ATCC 49188 genomic |
| Ochr2 | GAACACCTGTTGTCC | 157 | 23S | genes, no positive |
| (Ochr7-1) | | | | hybridization signals |
| Ochr3 | GATCCGACGATTTCC | 164 | ITS | were obtained. |
| (OchrI) | | | | |
| Ochr4 | TCGTCGGCCCATGTG | 158 | 23S | |
| (Ochr23-2) | | | | |
| Ochr5 | TTAGTGTATCGAGCA | 159 | 23S | |
| (Ochr7-2) | | | | |
| Ochr001 | TAGGAAAGACGCAGT | 165 | ITS | |
| Ochr002 | CTTCGGGCTGATGAT | 160 | 23S | cross-reacted with |
| | | | | genomic genes of |
| | | | | Chryseobacterium |
| | | | | Meningosepticum, but |
| | | | | not cross-reacted with |
| | | | | genomic genes of the |
| | | | | other 58 species |
| Ochr003 | AGGCCAGTCAGCCTG | 161 | 23S | Upon four applications |
| | | | | of ATCC 49188 genomic |
| | | | | genes, no positive |
| | | | | hybridization signals |
| | | | | were obtained. |
| Ochr004 | GTTGATTGACACTTG | 153 | ITS | not cross-reacted with |
| Ochr005 | TACCGCTCACGAGCC | 154 | ITS | genomic genes of all 59 |
| | | | | species |
| Ochr006 | GTTGGTTCTGATACA | 162 | 23S | Upon four applications |
| Ochr008 | CAGTTGGAAGCAGAG | 163 | 23S | of ATCC 49188 genomic |
| | | | | genes, no positive |
| | | | | hybridization signals |
| | | | | were obtained. |
| Ochr007 | GGGTCCGGAGGTTCA | 155 | ITS | not cross-reacted with |
| Ochr04 | GGACCAGGCCAGTGG | 151 | 23S | genomic genes of all 59 |
| Ochr05 | GACCAGGCCAGTGGC | 152 | 23S | species |

TABLE 14

| | Nucleotide | SEQ | Loca- | |
|---|---|---|---|---|
| Reference | Sequence | ID NO | tion | Specificity |
| Pep1 | ACTAAATAAACCAGG | 174 | 23S | Upon four applications |
| (Pep23) | | | | of KCTC 3319 genomic |
| Pep2 | ATAATCAACATCTAC | 175 | 23S | genes, no positive |
| (PepM) | | | | hybridization signals |
| Pep3 | TCTGTATAATAGTTC | 176 | ITS | were obtained. |
| (PepI) | | | | |
| Pep001 | AGAAGCTGATACGTC | 177 | ITS | |
| Pep002 | ACTAGGGAGAGCTCA | 166 | 23S | not cross-reacted with |
| Pep003 | GCTTAGTAAAGCAAG | 167 | 23S | genomic genes of all 59 |
| | | | | species |
| Pep004 | TACTAACATGTGACC | 168 | 23S | cross-reacted with |
| Pep005 | AAGCAGAGAGAGCTC | 169 | 23S | genomic genes of |
| | | | | Chryseobactrium |
| | | | | meningosepticum but not |
| | | | | cross-reacted with |
| | | | | genomic genes of the |
| | | | | other 58 species. |
| Pep006 | CGAACGGTGAGGCCG | 170 | 23S | cross-reacted with |
| | | | | genomic genes of |
| | | | | Morganella morganii and |
| | | | | Bacteroides fragilis but |
| | | | | not cross-reacted with |
| | | | | genomic genes of the |
| | | | | other 57 species |
| Pep007 | GTAGATGTTGATTAT | 171 | 23S | cross-reacted with |
| | | | | genomic genes of |
| | | | | Bacteroides fragilis |
| | | | | but not cross-reacted |
| | | | | with genomic genes of |
| | | | | the other 58 species |
| Pep23S02 | GTCGAATCATCTGGG | 172 | 23S | cross-reacted with |
| | | | | genomic genes of |
| | | | | Morganella morganii |
| | | | | but not cross-reacted |
| | | | | with genomic genes of |
| | | | | the other 58 species |
| Pep23S03 | TAAAACGTATGGAT | 173 | 23S | not cross-reacted with |
| | | | | genomic genes of all 59 |
| | | | | species |

TABLE 15

| | Nucleotide | SEQ | Loca- | |
|---|---|---|---|---|
| Reference | Sequence | ID NO | tion | Specificity |
| Por1 | GTTGGATGTTATCAT | 182 | 23S | Upon four applications |
| (Por7) | | | | of ATCC 33277 genomic |
| Por2 | CGGGCAGCTAAAACC | 183 | 23S | genes, no positive |
| (PorM) | | | | hybridization signals |
| Por3 | TGTTTGTGCGACGTG | 185 | ITS | were obtained. |
| (PorI) | | | | |
| Por001 | GTTTTTGTGAGTGGA | 180 | ITS | cross-reacted with |
| Por002 | TGATGGGTGGGGTTG | 181 | ITS | genomic genes |
| Por003 | AGTTGGTGAGCGAGC | 178 | 23S | Actinomyces israelii and |
| | | | | Rothia but not cross |
| | | | | reacted with genomic |
| | | | | genes of the other |
| | | | | 57 species |
| Por004 | ACCTATGAGTACTAT | 184 | 23S | Upon four applications |
| | | | | of ATCC 49188 genomic |
| | | | | genes, no positive |
| | | | | hybridization signals |
| | | | | were obtained. |
| Por23S08 | CTGAGCTGTCGTGCA | 179 | 23S | cross-reacted with |
| | | | | genomic genes of |
| | | | | Acinomyces israelii and |
| | | | | Rothia but not cross- |
| | | | | reacted with genomic |
| | | | | genes of the other 57 |
| | | | | species |

TABLE 16

| | Nucleotide | SEQ | Loca- | |
|---|---|---|---|---|
| Reference | Sequence | ID NO | tion | Specificity |
| P. anae001 | TGCATTACTAAGTGA | 188 | 23S | Upon four applications |
| P. anae002 | GTAAGGTCGATACCC | 169 | 23S | of ATCC 27337 genomic |
| | | | | genes, no positive |
| | | | | hybridization signals |
| | | | | were obtained. |
| P. anae003 | AGGAGGAAGAGAAAG | 186 | 23S | not cross-reacted with |
| | | | | genomic genes of 20 |
| | | | | species* |
| P. anae004 | GCGAAAGGAAAAGAG | 187 | 23S | cross-reacted with |
| | | | | genomic genes of P. |
| | | | | magnus but not cross |
| | | | | reacted with genomic |
| | | | | genes of the other 19 |
| | | | | species* |

TABLE 17

| | Nucleotide | SEQ | Loca- | |
|---|---|---|---|---|
| Reference | Sequence | ID NO | tion | Specificity |
| P. magn001 | TAGTTGAAAATAGTA | 191 | 23S | Upon four applications |
| | | | | of ATCC 29328 genomic |
| | | | | genes, no positive |
| | | | | hybridization signals |
| | | | | were obtained. |
| P. magn002 | CATGCAACGATCCGT | 190 | 23S | not cross-reacted with |
| | | | | genomic genes of 20 |
| | | | | species* |
| P. magn003 | CAGCACGTGAATATG | 192 | 23S | Upon four applications |
| | | | | of ATCC 29328 genomic |
| | | | | genes, no positive |
| | | | | hybridization signals |
| | | | | were obtained. |

TABLE 18

| | Nucleotide | SEQ | Loca- | |
|---|---|---|---|---|
| Reference | Sequence | ID NO | tion | Specificity |
| f. necro01 | TTTCGCAGACGTAAG | 193 | 23S | not cross-reacted with |
| f. necro02 | GTTTTCTTGCGCTGT | 194 | 23S | genomic genes of 20 |
| f. necro03 | CCGTATTCATGTCAA | 195 | 23S | species* |
| f. necro05 | CTGCAAGCTATTTCG | 196 | 23S | |
| f. necro06 | CAGACGTAAGCAAAG | 197 | 23S | |
| f. necro07 | CCTGTATTGGTAGTT | 198 | 23S | |

TABLE 19

| | Nucleotide | SEQ | Loca- | |
|---|---|---|---|---|
| Reference | Sequence | ID NO | tion | Specificity |
| P. vulga01 | ATACGTGTTATGTGC | 200 | ITS | cross-reacted with |
| | | | | genomic genes of P. |
| | | | | aeruginosa but not |
| | | | | cross-reacted with |
| | | | | genomic genes of the |
| | | | | other 19 Species* |
| P. vulga02 | CTCACACAGACTTGT | 205 | ITS | Upon four applications |
| P. vulga03 | ATATCCAATGGATAT | 201 | 23S | of ATCC 11539 genomic |
| | | | | genes, no positive |
| | | | | hybridization signals |
| | | | | were obtained. |
| P. vulga004 | AGAGGAGGCTTAGTG | 199 | 23S | cross-reacted with |
| | | | | genomic genes of C. |
| | | | | diphtheria and P. |
| | | | | aeroginosa but not |
| | | | | cross-reacted with |
| | | | | genomic genes of the |
| | | | | other 18 species* |
| P. vulga005 | GTGGGTTGCAAAATA | 206 | ITS | Upon four applications |
| P. vulga006 | GGAAACCCAATATCC | 202 | 23S | of ATCC 11539 genomic |
| P. vulga007 | GGGAAACCCAATATC | 203 | 23S | genes, no positive |
| P. vulga008 | CACTGTTTCGACTAG | 204 | 23S | hybridization signals |
| | | | | were obtained. |

TABLE 20

| | Nucleotide | SEQ | Loca- | |
|---|---|---|---|---|
| Reference | Sequence | ID N0. | tion | Specificity |
| E. aero01 | TTCCGACGGTACAGG | 207 | 23S | not cross-reacted with |
| | | | | genomic genes of 20 |
| | | | | species* |
| E. aero02 | GAGCGGGGTAGTTGA | 210 | 23S | Upon four applications |
| | | | | of ATCC 11783 genomic |
| | | | | genes, no positive |
| | | | | hybridization signals |
| | | | | were obtained. |
| E. aero03 | GTATCAGTAAGTGCG | 208 | 23S | not cross-reacted with |
| | | | | genomic genes of 20 |
| | | | | species* |
| E. aero04 | TTATCCAGGCAAATC | 209 | 23S | cross-reacted with |
| | | | | genomic genes of |
| | | | | Bacteroids ovatus |
| | | | | but not cross-reacted |
| | | | | with genomic genes of |
| | | | | the other 19 species* |
| E. aero005 | AATCAAGGCTGAGGT | 211 | 23S | Upon four applications |
| | | | | of ATCC 11783 genomic |
| | | | | genes, no positive |
| | | | | hybridization signals |
| | | | | were obtained. |

TABLE 22

| | Nucleotide | SEQ | Loca- | |
|---|---|---|---|---|
| Reference | Sequence | ID No. | Tion | Specificity |
| S. mutans01 | GAAAAACGAAGGGTA | 213 | 23S | Upon four applications |
| S. mutans02 | ATGACTACGTGGTCG | 214 | 23S | of ATCC 11823 genomic |
| S. mutans03 | GTAATGCAAGATATC | 215 | 23S | genes, no positive |
| S. mutans004 | TTGTATGCGCGGTAG | 216 | 23S | hybridization signals |
| S. mutans005 | CGAAAAGTATCGGGG | 217 | 23S | were obtained. |
| S. mutan001 | TAGGTATTCTCTCC | T 212 | 23S | not cross-reacted with |
| | | | | genomic genes of 20 |
| | | | | species* |

TABLE 22

| | Nucleotide | SEQ | Loca- | |
|---|---|---|---|---|
| Reference | Sequence | ID No. | tion | Specificity |
| K. king01 | TGATTCAATGCGATG | 222 | 23S | Upon four applications of |
| | | | | ATCC 23330 genomic genes, |
| | | | | no positive hybridization |
| | | | | signals were obtained. |
| K. king02 | GGTTAGCAAACTGTT | 218 | 23S | not cross-reacted with |
| K. king03 | CCAGTAGGTGGAAAG | 219 | 23S | genomic genes of 20 |
| K. king04 | AACACCGAGACGTGA | 220 | 23S | species* |
| K. king05 | TATAATTAAACGCAT | 223 | 23S | Upon four applications of |
| K. king06 | AATGTTGTCGATTTG | 224 | 23S | ATCC 23330 genomic genes, |
| K. king07 | AGGCAACAAATCGAA | 225 | 23S | no positive hybridization |
| K. king08 | TATCAACTAATCTTG | 226 | 23S | signals were obtained. |
| K. king09 | TATTCAATGCGATGG | 233 | 23S | No cross reactions with |
| | | | | genomic genes of 20 |
| | | | | species* |

TABLE 23

| | Nucleotide | SEQ | Loca- | |
|---|---|---|---|---|
| Reference | Sequence | ID NO | tion | Specificity |
| B. ovatus01 | TAGAAGGAAGCATTC | 227 | 23S | not cross-reacted with |
| | | | | genomic genes of 20 |
| | | | | species* |
| B. ovatus02 | CCAATGTTGTTACGG | 228 | 23S | cross-reacted with |
| | | | | genomic genes of H. |
| | | | | aphrophilas but not |
| | | | | cross-reacted with |
| | | | | genomic genes of the |
| | | | | other 19 Species* |
| B. ovatus003 | GGACCGAACCGATAA | 230 | 23S | cross-reacted with |
| | | | | genomic genes of B. |
| | | | | catarrhalis but not |
| | | | | cross-reacted with |
| | | | | genomic genes of the |
| | | | | other 19 Species* |
| B. ovatus004 | GGACACGAGGAATCT | 231 | 23S | Upon four applications |
| | | | | of KCTC 8483 genomic |
| | | | | genes, no positive |
| | | | | hybridization signals |
| | | | | were obtained |
| B. ovatus005 | TGTAGGACCACGATG | 229 | 23S | cross-reacted with |
| | | | | genomic genes of |
| | | | | B. thetaiotaomiron |
| | | | | but not cross-reacted |
| | | | | with genomic genes of |
| | | | | the other 19 Species* |
| B. ovatus006 | TGAAGGAATGTCATC | 232 | 23S | Upon four applications |
| B. ovatus007 | CCCACGATAGATAGA | 233 | 23S | of KCTC 8483 genomic |
| | | | | genes, no positive |
| | | | | hybridization signals |
| | | | | were obtained. |

TABLE 24

| | Nucleotide | SEQ | Loca- | |
|---|---|---|---|---|
| Reference | Sequence | ID NO | tion | Specificity |
| B. thetaio01 | TTATTGTACTACTGG | 235 | 23S | Upon four applications |
| B. thetaio02 | ATCAGGTAGACAAGG | 236 | 23S | of KCTC 5015 genomic |
| B. thetaio03 | TTGTCGTTGCCAATA | 237 | 23S | genes, no positive |
| B. thetaio04 | CAGTGTTGGAATGTT | 238 | 23S | hybridization signals |
| B. thetaio05 | ACTATACTATAGTCA | 239 | 23S | were obtained. |
| B. thetaio006 | GCTAACGCAGGGAAC | 234 | 23S | not cross-reacted with |
| | | | | genomic genes of 20 |
| | | | | species* |

TABLE 25

| | Nucleotide | SEQ | Loca- | |
|---|---|---|---|---|
| Reference | Sequence | ID NO | tion | Specificity |
| C. diffc001 | GCATATATATTTAGT | 241 | 23S | cross-reacted with |
| | | | | genomic genes of P. |
| | | | | magnus but not cross |
| | | | | reacted with genomic |
| | | | | genes of the other 19 |
| | | | | species* |
| C. diffc002 | GATATGACATCTAAT | 242 | 23S | Upon four applications |
| C. diffc003 | TTTCGGGGAGTTGCA | 243 | 23S | of ATCC 9689 genomic |
| C. diffc004 | CATGTGGACAGTATG | 244 | 23S | genes, no positive |
| | | | | hybridization signals |
| | | | | were obtained |
| C. diffc005 | GTTCGTCCGCCCCTG | 240 | 23S | not cross-reacted with |
| | | | | genomic genes of 20 |
| | | | | species* |

TABLE 26

| | Nucleotide | SEQ | Loca- | |
|---|---|---|---|---|
| Reference | Sequence | ID NO | tion | Specificity |
| H. aphro001 | TGGGAGTGGGTTGTC | 246 | ITS | not cross-reacted with |
| H. aphro002 | TAACAAACCGGAAAC | 247 | ITS | genomic genes of 20 |
| H. aphro003 | GGTGAAGAACCCACT | 245 | 23S | species* |
| H. aphro004 | ATCATTATCTGAATC | 248 | 23S | Upon four applications |
| H. aphro005 | AGAAATCAACCGTAG | 249 | 23S | of KCTC 13252 genomic |
| H. aphro006 | ATTAGCGGATGACTC | 250 | 23S | genes, no positive |
| H. aphro007 | AACCCAGTGGGTGAA | 251 | 23S | hybridization signals |
| H. aphro008 | AAACCCAGTGGGTGA | 252 | 23S | were obtained. |
| H. aphro009 | GAAACCCAGTGGGTG | 253 | 23S | |

TABLE 27

| | Nucleotide | SEQ | Loca- | |
|---|---|---|---|---|
| Reference | Sequence | ID NO | tion | Specificity |
| N. gono001 | AATGAGTTTGTTTTG | 259 | ITS | Upon four applications |
| | | | | of ATCC 10150 genomic |
| | | | | genes, no positive |
| | | | | hybridization signals |
| | | | | were obtained |
| N. gono002 | AACCTCTCGCAAGAG | 256 | ITS | not cross-reacted with |
| | | | | genomic genes of 20 |
| | | | | species * |
| N. gono003 | CATAGTATTTGGGTG | 257 | 23S | Upon four applications |
| N. gono004 | TTGTATCAGACTTAA | 258 | 23S | of ATCC 10150 genomic |
| | | | | genes, no positive |
| | | | | hybridization signals |
| | | | | were obtained |
| N. gono005 | TATCAAAGTAGGGAT | 254 | 23S | cross-reacted with |
| | | | | genomic genes of H. |
| | | | | aprophilus but not |
| | | | | cross-reacted with |
| | | | | genomic genes of the |
| | | | | other 19 species* |
| N. gono006 | AGTCAACGGGTAGGT | 255 | 23S | not cross-reacted with |
| | | | | genomic genes of 20 |
| | | | | species* |
| N. gono007 | CAATGAGTTTGTTTT | 260 | ITS | Upon four applications |
| N. gono008 | CGTAACTATAACGGT | 261 | ITS | of ATCC 10150 genomic |
| | | | | genes, no positive |
| | | | | hybridization signals |
| | | | | were obtained |

TABLE 28

| | Nucleotide | SEQ | Loca- | |
|---|---|---|---|---|
| Reference | Sequence | ID NO | tion | Specificity |
| E. corro001 | AGTCGTAGAGCGGAG | 264 | ITS | cross-reacted with |
| | | | | genomic genes of N. |
| | | | | gonorrhoeae but not |
| | | | | cross-reacted with |
| | | | | genomic genes of the |
| | | | | other 19 species* |
| E. corro002 | AGATCCGCCCAGGTA | 265 | ITS | Upon four applications |
| E. corro003 | GTTGCTGCATCTTGC | 266 | ITS | of ATCC 51724 genomic |
| E. corro004 | GCAGGATTCGGACAC | 267 | ITS | genes, no positive |
| | | | | hybridization signals |
| | | | | were obtained. |
| E. corro005 | GGATAGGAGAAGGAA | 262 | 23S | cross-reacted with |
| | | | | genomic genes of N. |
| | | | | gonorrhoeae but not |
| | | | | cross-reacted with |
| | | | | genomic genes of the |
| | | | | other 19 species* |
| E. corro006 | ACTCATCATCGATCC | 263 | 23S | not cross-reacted with |
| | | | | genomic genes of 20 |
| | | | | species* |

TABLE 29

| | Nucleotide | SEQ | Loca- | |
|---|---|---|---|---|
| Reference | Sequence | ID NO | tion | Specificity |
| B. vulga01 | CATCTTGAGATGTGC | 270 | 23S | Upon four applications |
| | | | | of KCCM 11423 genomic |
| | | | | genes, no positive |
| | | | | hybridization signals |
| | | | | were obtained. |
| B. vulga02 | AGTCGGGCGTGGATA | 271 | 23S | cross-reacted with |
| | | | | genomic genes of |
| | | | | Bacteroides ovatus and |
| | | | | C. diphtheria but not |
| | | | | cross-reacted with |
| | | | | genomic genes of the |
| | | | | other 18 species* |
| B. vulga03 | AGTCAGCGTCGAAGG | 268 | 23S | cross-reacted with |
| | | | | genomic genes of F. |
| | | | | necrophorum, |
| | | | | S. mutans, B. ovatus, |
| | | | | H. actinomycetmcom and |
| | | | | B. thetaiotaomicron |
| | | | | but not cross-reacted |
| | | | | with genomic genes of |
| | | | | the other 15 species* |
| B. vulga04 | ACGCTAATCGGATCA | 272 | 23S | cross-reacted with |
| | | | | genomic genes of |
| | | | | H. aphrophilas but not |
| | | | | cross-reacted with |
| | | | | genomic genes of the |
| | | | | other 19 species* |
| B. vulga05 | GACCGATAGAGCATG | 273 | 23S | Upon four applications |
| B. vulga06 | TGACACACTGTAACT | 274 | 23S | of KCCM 11423 genomic |
| | | | | genes, no positive |
| | | | | hybridization signals |
| | | | | were obtained |
| B. vulga07 | CGAATGCGCATCAGT | 269 | 23S | not cross-reacted with |
| | | | | genomic genes of 20 |
| | | | | species* |
| B. vulga08 | ATTGTCATGAGCCAC | 275 | 23S | Upon four applications |
| B. vulga09 | AATTTGCGTGGCTCT | 276 | 23S | of KCCM 11423 genomic |
| B. vulga10 | CTCCATCGGAAACGT | 277 | 23S | genes, no positive |
| B. vulga11 | ACTCCATCGGAAACG | 278 | 23S | hybridization signals |
| B. vulga12 | GGGACTACGAACGGA | 279 | 23S | were obtained |

TABLE 30

| | Nucleotide | SEQ | Loca- | |
|---|---|---|---|---|
| Reference | Sequence | ID NO | tion | Specificity |
| B. catar001 | AGTCTGGGTTAATTA | 281 | 23S | Upon four applications |
| B. catar002 | ACAAGTTGTTCTTTG | 282 | 23S | of KCCM 40056 genomic |
| B. catar003 | AACATAGGTGAATCG | 283 | 23S | genes, no positive |
| B. catar004 | AAGTAATGAAGTGCA | 284 | 23S | hybridization signals |
| | | | | were obtained. |
| B. catar005 | ATATCTTCGCGCTGT | 280 | 23S | not cross-reacted with |
| | | | | genomic genes of 20 |
| | | | | species* |
| B. catar006 | GAGGATAACAATGAA | 285 | 23S | Upon four applications |
| B. catar007 | CGAATGAGTTTGTCA | 286 | 23S | of KCCM 40056 genomic |
| B. catar008 | ACCCGAATATCCGAC | 287 | 23S | genes, no positive |
| B. catar009 | GACCCACCATTTTGG | 288 | 23S | hybridization signals |
| B. catar010 | ATAATGGGGTCAGCG | 289 | 23S | were obtained. |
| B. catar011 | AGCCTGTGAAGGTGC | 290 | 23S | |
| B. catar012 | AAGAATTGATGACCA | 291 | 23S | |

TABLE 31

| | Nucleotide | SEQ | Loca- | |
|---|---|---|---|---|
| Reference | Sequence | ID NO | tion | Specificity |
| S. wad01 | CTCAGTAAGACGTTT | 295 | ITS | No signals shown upon |
| | | | | applications of ATCC |
| | | | | 51579 genomic genes |
| S. wad02 | GCTCCGACAAGAACT | 292 | ITS | not cross-reacted |
| S. wad03 | CGAGTTGTTGAATTC | 293 | ITS | with genomic genes of |
| S. wad04 | GTCGTCTTGTGCTTT | 294 | ITS | 20 species* |

TABLE 32

| | Nucleotide | SEQ | Loca- | |
|---|---|---|---|---|
| Reference | Sequence | ID NO | tion | Specificity |
| Acii1 | AACCTGGCTGGTGGC | 296 | 23S | cross-reacted with genomic |
| (Acii23) | | | | genes of Rothia and |
| | | | | porphylomonas gingivalis |
| | | | | but not cross-reacted |
| | | | | with genomic genes of the |
| | | | | other 57 species |
| Acii2 | GACACTTTTGTGTCA | 297 | 23S | Upon four applications of |
| (AciiM) | | | | ATCC 12102 genomic genes, |
| Acii3 | GTTGGGTGGTTGCCT | 298 | ITS | no positive hybridization |
| (AciI) | | | | signals were obtained |

TABLE 33

| | Nucleotide | SEQ | Loca- | |
|---|---|---|---|---|
| Reference | Sequence | ID NO | tion | Specificity |
| Se1 | TTCTCTCTTGAGTGG | 301 | 23S | Upon four applications |
| (Se23) | | | | of KCTC 1917 (ATCC 1228) |
| Se2 | CGTGCTGTTGGAGTG | 302 | 23S | genomic genes, no |
| (SeM) | | | | positive hybridization |
| Se3 | GCTATTTATTTTGAA | 303 | ITS | signals were obtained |
| (SeI) | | | | |
| SeM01 | GATAGATAACAGGTG | 299 | 23S | No cross reactions with |
| SeM02 | AGGGTTCACGCCCAG | 300 | 23S | genomic genes of all 59 |
| | | | | species |

TABLE 34

| | Nucleotide | SEQ | Loca- | |
|---|---|---|---|---|
| Reference | Sequence | ID NO | tion | Specificity |
| Bur | TTGTTAGCCGAACGC | 304 | 23S | not cross-reacted with genomic |
| (Bur23) | | | | genes of all 59 species |
| Bur001 | GCCAGGAGGGTGAAG | 306 | 23S | cross-reacted with genomic |
| | | | | genes of Cardiobacterium, |
| | | | | E. coli, K. pneuznoniae, |
| | | | | oxytoca, Burkholderia, Salmonella |
| | | | | spp., .P. mirabilis, .facium and |
| | | | | S. marcescens but not cross- |
| | | | | reacted with genomic genes of |
| | | | | the other 50 species |
| Bur01 | GGGTGTGGCGCGAGC | 305 | 23S | not cross-reacted with genomic |
| | | | | genes of all 59 species |

TABLE 35

| | Nucleotide | SEQ | Loca- | |
|---|---|---|---|---|
| Reference | Sequence | ID NO | tion | Specificity |
| Styp | GCCTGAATCAGCATG | 307 | 23S | not cross-reacted with |
| (Styp23) | | | | genomic genes of all 59 |
| | | | | species |
| Styp01 | GCTGAGGATACGGTT | 308 | 23S | Upon four applications |
| Styp02 | CCGCAAAACAAGCAG | 309 | 23S | of KCCM 12021 genomic |
| Styp03 | ACGATTGACGGAGCG | 310 | 23S | genes, no positive |
| Sal.typ001 | TCGCGCCGTCACAGT | 311 | 23S | hybridization signals |
| | | | | were obtained. |

TABLE 36

| | Nucleotide | SEQ | Loca- | |
|---|---|---|---|---|
| Reference | Sequence | ID NO | tion | Specificity |
| Eco1 | GAGCCTGAATCAGTG | 313 | 23S | cross-reacted with |
| (Eco23) | | | | genomic genes S. |
| | | | | flexneri but not cross- |
| | | | | reacted with genomic |
| | | | | genes of the other 58 |
| | | | | species |
| Eco2 | GTTAGCGGTAACGCG | 314 | 23S | Upon four applications |
| (E coli) | | | | of ATCC 25922 genomic |
| | | | | genes, no positive |
| | | | | hybridization signals |
| | | | | were obtained. |
| E coli001 | GTTAGCGGTAACGCG | 315 | 23S | cross-reacted with |
| | | | | genomic genes of S. |
| | | | | maltophila but not |
| | | | | cross-reacted with |
| | | | | genomic genes of the |
| | | | | other 58 species |
| E coli002 | ATGCACATATTGTGA | 316 | 23S | Upon four applications |
| | | | | of ATCC 25922 genomic |
| | | | | genes, no positive |
| | | | | hybridization signals |
| | | | | were obtained |
| E coli003 | CTGAAGCGACAAATG | 312 | 23S | not cross-reacted with |
| | | | | genomic genes of all 59 |
| | | | | species |

TABLE 37

| | Nucleotide | SEQ | Loca- | |
|---|---|---|---|---|
| Reference | Sequence | ID NO | tion | Specificity |
| K. pneu | GTACACCAAAATGCA | 317 | 23S | not cross-reacted with |
| (K. pneu23) | | | | genomic genes of all 59 |
| | | | | species |
| K. pneu001 | ACGCTGGTGTGTAGG | 319 | 23S | cross-reacted with |
| | | | | genomic genes of C. |
| | | | | hominis, A. israelii, |
| | | | | Rothia, H. influenza, |
| | | | | E. coil and P. |
| | | | | mirabiiis but not |
| | | | | cross-reacted with |
| | | | | genomic genes of the |
| | | | | other 53 species |
| K. pneu002 | GCTGAGACCAGTCGA | 318 | 23S | not cross-reacted with |
| | | | | genomic genes of all 59 |
| | | | | species |
| K. pneu01 | ACCTTCGGGTGTGAC | 320 | 23S | Upon seven applications |
| | | | | of ATCC 700603 genomic |
| | | | | genes, six positive |
| | | | | hybridization signals |
| | | | | were obtained (14.3%) |

TABLE 38

| | Nucleotide | SEQ | Loca- | |
|---|---|---|---|---|
| Reference | Sequence | ID NO | tion | Specificity |
| Pm | GTTACCAACAATCGT | 321 | 23S | not cross-reacted with |
| | | | | genomic genes of all 59 |
| | | | | species |
| Pm001 | AAGGCTAGGTTGTCC | 325 | 23S | Upon seven applications |
| | | | | of KCCM 11381 genomic |
| | | | | genes, six cross- |
| | | | | reactions occurred |
| | | | | (86%) |
| Pm002 | GGCGACGGTCGTCCC | 322 | 23S | not cross-reacted with |
| Pm003 | GATGACGAACCACCA | 323 | 23S | genomic genes of all 59 |
| Pm004 | TGAAGCAATTGATGC | 324 | 23S | species |
| Pm005 | TAAAGTCCCTCGCGG | 326 | 23S | Upon seven applications |
| Pm01 | AGGCAGAGTGATTAG | 327 | 23S | of KCCM 11381 genomic |
| | | | | genes, three positive |
| | | | | hybridization signals |
| | | | | were obtained. cross- |
| | | | | reacted with E. coli |

TABLE 39

| | Nucleotide | SEQ | Loca- | |
|---|---|---|---|---|
| Reference | Sequence | ID NO | tion | Specificity |
| Streep | TAGGACTGCAATGTG | 328 | 23S | not cross-reacted with |
| (StreppM) | | | | genomic genes of all 59 |
| | | | | species |
| Strepp01 | ATGTGGTACAGACAC | 329 | 23S | Upon four applications |
| Strepp02 | GGTTAAACGCTAGAA | 330 | 23S | of KCTC 40410, 41568, |
| Strepp03 | CAGGATACTGCTAAG | 331 | 23S | 41569 and 41570 genomic |
| Strepp04 | GAGTAAACTCTTCGG | 332 | 23S | genes, no positive |
| | | | | hybridization signals |
| | | | | were obtained |

TABLE 40

| | Nucleotide | SEQ | Loca- | |
|---|---|---|---|---|
| Reference | Sequence | ID NO | tion | Specificity |
| VvulM | AGTAACAGCCACTTG | 334 | 23S | Upon four applications |
| | | | | of KCTC 2962 (ATCC 3385) |
| | | | | genomic genes, three |
| | | | | positive hybridization |
| | | | | signals were obtained |
| V. vul001 | ATAGCTCAATGAAGC | 335 | 23S | not cross-reacted with |
| | | | | genomic genes of all 59 |
| | | | | species |
| V. vul002 | GGCGCCATAGTCTCT | 336 | 23S | Upon four applications |
| V vul01 | TTTACATGTGTTAGA | 337 | 23S | of KCTC 2962 (ATCC 3385) |
| | | | | genomic genes, no |
| | | | | positive hybridization |
| | | | | signals were obtained. |
| V vul02 | GTTGACGATGCATGT | 333 | 23S | not cross-reacted with |
| | | | | genomic genes of all 59 |
| | | | | species |
| V vul03 | GTTCTATGAACATTG | 338 | 23S | Upon four applications |
| | | | | of KCTC 2962 (ATCC 3385) |
| | | | | genomic genes, no |
| | | | | positive hybridization |
| | | | | signals were obtained |

TABLE 41

| | Nucleotide | SEQ | Loca- | |
|---|---|---|---|---|
| Reference | Sequence | ID NO | tion | Specificity |
| Pa | GTTTCCCGTGAAGGC | 341 | 23S | Upon four applications |
| | | | | of KCTC 1636 genomic |
| | | | | genes, no positive |
| | | | | hybridization signals |
| | | | | were obtained. |
| P. aeru001 | GAAGTGCCGAGCATG | 339 | 23S | not cross-reacted with |
| | | | | genomic genes of all 59 |
| | | | | species |
| P. aeru002 | GTGTCACGTAAGTGA | 342 | 23S | cross-reacted with |
| | | | | genomic genes of M. |
| | | | | morganii but not cross- |
| | | | | reacted with genomic |
| | | | | genes of. the other 58 |
| | | | | species |
| P. aeru003 | AGTCGTCTTTTAGAT | 343 | 23S | Upon four applications |
| P. aeru004 | ACTCCGTAAGCTCTG | 344 | 23S | of KCTC 1636 genomic |
| Pa01 | TAGGATAACCTAGGT | 345 | 23S | genes, no positive |
| Pa02 | TAAGCTTCATTGATT | 346 | 23S | hybridization signals |
| | | | | were obtained. |
| Pa03 | GGATCTTTGAAGTGA | 340 | 23S | not cross-reacted with |
| | | | | genomic genes of all 59 |
| | | | | species |

TABLE 42

| | Nucleotide | SEQ | Loca- | |
|---|---|---|---|---|
| Reference | Sequence | ID NO | tion | Specificity |
| Ah | GGCGCCTCGGTAGGG | 347 | 23S | not cross-reacted with |
| | | | | genomic genes of all 59 |
| | | | | species |
| Ae.hy001 | TAAGCCGTGAGCAGT | 348 | 23S | Upon four applications |
| Ae.hy002 | CATCTTGGAAGTTAG | 349 | 23S | of KCCM 32586 (ATCC |
| Ae.hy003 | TCAAACCAGGCACCG | 350 | 23S | 11163) genomic genes, no |
| Ae.hy004 | GATTCACGCTAAGCG | 351 | 23S | positive hybridization |
| Ae.hy005 | ACGGTGCGGAAGCCA | 352 | 23S | signals were obtained |
| Ah01 | CACGAAAACAACCTT | 353 | 23S | |

TABLE 43

| | Nucleotide | SEQ | Loca- | |
|---|---|---|---|---|
| Reference | Sequence | ID NO | tion | Specificity |
| LM | GGGTGCAAGCCCGAG | 354 | 23S | not cross-reacted with |
| | | | | genomic genes of all 59 |
| | | | | species |
| Lm01 | AGTATCCTTCGTGA | 355 | 23S | Upon four applications |
| Lm02 | GTGAGGAAGGCAGAC | 356 | 23S | of ATCC 700603 genomic |
| Lm03 | GGCTTTCCCTCCAGA | 357 | 23S | genes, no positive |
| Lm04 | CCGCTTCTCACGAAG | 358 | 23S | hybridization signals |
| | | | | were obtained. |

TABLE 44

| | Nucleotide | SEQ | Loca- | |
|---|---|---|---|---|
| Reference | Sequence | ID NO | tion | Specificity |
| Enfcium1 | GTTCTTTCAGATAGT | 361 | 23S | Upon four applications |
| (Enfaeci23) | | | | of ATCC 19434 genomic |
| Enfcium2 | CTGAAGAGGAGTCAA | 362 | 23S | genes, no positive |
| (Enfaeci M) | | | | hybridization signals |
| E. faecium001 | GCTGATCATACGATC | 363 | 23S | were obtained. |
| E. faecium002 | TTACGATTGTGTGAA | 359 | 23S | not cross-reacted with |
| E. faecium003 | ATAGCACATTCGAGG | 360 | 23S | genomic genes of all 59 |
| | | | | species |
| E. faecium004 | CTTCTTTTCTTAAGG | 364 | 23S | Upon four applications |
| | | | | of ATCC 19434 genomic |
| | | | | genes, no positive |
| | | | | hybridization signals |
| | | | | were obtained. |

TABLE 45

| | Nucleotide | SEQ | Loca- | |
|---|---|---|---|---|
| Reference | Sequence | ID NO | tion | Specificity |
| Saur | GTTAACGCCCAGAAG | 368 | 23S | Upon four applications |
| S. aureus001 | AGGACGACATTAGAC | 369 | 23S | of KCTC 1621 genomic |
| S. aureus002 | AAAATGTTGTCTCTC | 370 | 22S | genes, no positive |
| S. aureus003 | CGAAGCGTGCGATTG | 371 | 23S | hybridization signals |
| | | | | were obtained. |
| S. aureus004 | GATTGCACGTCTAAG | 365 | 23S | not cross-reacted with |
| | | | | genomic genes of all 59 |
| | | | | species |
| S. aureus005 | AATCCGGTACTCGTT | 366 | 23S | not cross-reacted with |
| | | | | genomic genes of all 59 |
| | | | | species |
| S aure01 | AAGCAGTAAATGTGG | 372 | 23S | Upon four applications |
| S aure02 | GAGAAGACATTGTGT | 373 | 23S | of KCTC 1621 genomic |
| | | | | genes, no positive |
| | | | | hybridization signals |
| | | | | were obtained |
| S aure03 | TCTTCGAGTCGTTGA | 367 | 235 | not cross-reacted with |
| | | | | genomic genes of all 59 |
| | | | | species |
| S aureus01 | ATATCAGAAGGCACA | 374 | 23S | Upon four applications |
| S aureus02 | ACAAAGGACGACATT | 375 | 23S | of KCTC 1621 genomic |
| S aureus03 | TCTTCGAGTCGTTGA | 376 | 23S | genes, no positive |
| | | | | hybridization signals |
| | | | | were obtained |

TABLE 46

| | Nucleotide | SEQ | Loca- | |
|---|---|---|---|---|
| Reference | Sequence | ID NO | tion | Specificity |
| Nm1 | CCCTGGAGGGTCGCA | 378 | 23S | Upon four applications |
| (Nm) | | | | of ATCC 13100 genomic |
| Nm2 | TTTGAATTGAACCGT | 379 | 23S | genes, no positive |
| (Nm-1) | | | | hybridization signals |
| Nm001 | GTTTACTGGCATGGT | 380 | 23S | were obtained |
| | | | | cross-reacted with |
| | | | | genomic genes of S. |
| | | | | sonnei but not cross- |
| | | | | reacted with genomic |
| | | | | genes of the other 58 |
| | | | | species |
| Nm002 | AGATGTGAGAGCATC | 377 | 23S | not cross-reacted with |
| | | | | genomic genes of all 59 |
| | | | | species |
| Nm01 | TAAAGCAATGATCCC | 381 | 23S | Upon four applications |
| | | | | of ATCC 13100 genomic |
| | | | | genes, no positive |
| | | | | hybridization signals |
| | | | | were, obtained |
| Nm02 | CCGGGTCTTCTTAAC | 382 | 23S | cross-reacted with |
| | | | | genomic genes of N. |
| | | | | gonorrhoea but not |
| | | | | cross-reacted with |
| | | | | genomic genes of the |
| | | | | other 58 species |

TABLE 47

| | Nucleotide | SEQ | Loca- | |
|---|---|---|---|---|
| Reference | Sequence | ID NO | tion | Specificity |
| L. pneu001 | CCTCAAGATGAGTTT | 386 | 23S | Upon four applications of |
| L. pneu002 | GAAGCCCGTTGAAGA | 387 | 23S | genomic genes of |
| L. pneu003 | GCAGTAATGCGTGAA | 388 | 23S | clinically isolated |
| L. pneu004 | TTGTCTTGACCATAT | 389 | 23S | Legionella pneumophila, |
| L. pneu005 | ACCATATAATCTGAG | 390 | 23S | no positive hybridization |
| L. pneu006 | TGCCCACACAGTTTG | 391 | 23S | signals were obtained |
| L. pneu007 | CAAAGTGCCCACACA | 392 | 23S | |
| L. pneu008 | TGATTTTGAGGTGAT | 393 | 23S | |
| L. pneu009 | CCACCATTTAATGAT | 394 | 23S | |
| L. pneu010 | AGCATTTTATTCTGG | 395 | 23S | |
| L. pneu011 | TGGAGAGCATTTTAT | 383 | 23S | not cross-reacted with |
| L. pneu012 | GTGATTTTGAGGTGA | 384 | 23S | genomic genes of |
| L. pneu013 | AGATGGTAAAGAAGA | 385 | 23S | 20 species* and |
| | | | | L. sainthelensi |
| | | | | and L. gormanii |

TABLE 48

| | Nucleotide | SEQ | Loca- | |
|---|---|---|---|---|
| Reference | Sequence | ID NO | tion | Specificity |
| C. alic001 | TGGTAGCCATTTATG | 396 | 18S | not cross-reacted with |
| | | | | genomic genes of all 59 |
| | | | | species |
| C. alic002 | TTTTCGATGCGTACT | 401 | 18S | Upon four applications |
| | | | | of KCCM 11474 genomic |
| | | | | genes, no positive |
| | | | | hybridization signals |
| | | | | were obtained |
| C. alic003 | CTGGACCAGCCGAGC | 397 | 188 | not cross-reacted with |
| | | | | genomic genes of all 59 |
| | | | | species |
| C. alic004 | CAGATGTCGAAAGGT | 402 | 18S | Upon four applications |
| C. alic005 | TAGGACGTTATGGTT | 403 | 18S | of KCCM 11474 genomic |
| | | | | genes, no positive |
| | | | | hybridization signals |
| | | | | were obtained |
| C. alic006 | TCAAGAACGAAAGTT | 398 | 185 | not cross-reacted with |
| C. alic007 | AAGGATTGACAGATT | 399 | 18S | genomic genes of all 59 |
| | | | | species |
| C. alic008 | CATTAATCAAGAACG | 400 | 18S | Upon four applications |
| | | | | of KCCM 11474 genomic |
| | | | | genes, no positive |
| | | | | hybridization signals |
| | | | | were obtained |

TABLE 49

| | Nucleotide | SEQ | Loca- | |
|---|---|---|---|---|
| Reference | Sequence | ID NO | tion | Specificity |
| glab001 | CTGGAATGCACCCGG | 404 | 18S | not cross-reacted with |
| | | | | genomic genes of all 59 |
| | | | | species |
| C. glab002 | CTAACCCCAAGTCCT | 406 | 18S | Upon four applications |
| C. glab003 | TGGCTTGGCGGCGAA | 405 | 18S | of KCCM 50701 genomic |
| | | | | genes, no positive |
| | | | | hybridization signals |
| | | | | were obtained. |
| C. glab005 | TCAAGAACGAAAGTT | 407 | 18S | cross-reacted with |
| C. glab006 | CATTAATCAAGAACG | 408 | 18S | genomic genes of |
| | | | | C. kruzei, C. albicans |
| | | | | and C. tropicalis but |
| | | | | not cross-reacted with |
| | | | | genomic genes of the |
| | | | | other 56 species |
| C. glab007 | AAACTTAAAGGAATT | 409 | 18S | Upon four applications |
| | | | | of KCCM 50701 genomic |
| | | | | genes, no positive |
| | | | | hybridization signals |
| | | | | were obtained |
| *20 species: Sutterella wadsworthensis, Clostridium ramosum, Peptostreptococcus anaerobius, Peptostreptococcus magnus, Fusobacterium necrophorum, Proteus vulgaris, Enterobacter aerogenes, Streptococcus mutans, Corynebacterium diphtheriae, Kingella kingap, Bacteroides vulgatus, Bacteroides ovatus, Haemohilus aphrophilas, Neisseria gonorrhea, Branhamella catarrhalis, Eikenella corrodens, Haemophilus actinomycetemcomitans, Bacteroides thetaiotaomicron, Clostridium | | | | |
| # difficile, Legionella pneumoniae | | | | |

### EXAMPLE 8

Blind Test

A blind test was performed with 12 microbial species, i.e., Staphylococcus aureus, Pseudomonas aeruginosa, Proteus mirabilis, Klebsiella pneumoniae, Acinetobacter baumanii, Escherichia coli, Enterococcus faecium, Staphylococcus epidermidis, Salmonella Group E, Salmonella Group B, Klebsiella oxytoca, and Burkholderia cepacia. The DNA chips designed for the blind test are shown in FIGS. 1, 9, 11 and 14 in which marks refer to probes listed in the above Tables 4 through 49. The mark “C” refers to a position marker which corresponds to the following sequence: Amine 3′-AAAAAAAAAAAAAAA-5′-FITC (SEQ ID NO: 428). The mark “N” refers to a negative control which corresponds to a buffer (3×SSC) in which probe is dissolved. The universal bacterial probes listed in Table 50 below were used as a positive control.

TABLE 50

| | | |
|---|---|---|
| Reference | Nucleotide Sequence | SEQ ID NO |

| | | |
|---|---|---|
| BaP1-01 | CAC GGT GGA TGC CCT | 421 |
| BaP1-03 | AGT AGC GGC GAG CGA | 422 |
| BaP1-06 | GAC CGA TAG TGA ACC | 423 |
| BaP2-01 | AGA ACC TGA AAC CGT | 424 |
| BaP2-03 | ACT GGA GGA CCG AAC | 425 |
| BaP2-04 | AGG GAA ACA ACC CAG | 426 |
| BaP3 | GTA AAC GGC GGC CGT | 427 |

Three hundreds patients infected with pathogens were enrolled in the blind test. The infection of samples collected from patients was confirmed by a culture method.

Genomic DNAs were isolated from cultured samples as follows. For body fluid sample, 10 ml of body fluid was collected in EDTA tube or plain tube. When the amount of sample was more than 10 ml, it was centrifuged at 5,000 rpm for 15 minutes. When the amount of sample was less than 10 ml, it was centrifuged at 14,000 rpm for 15 minutes and the precipitates formed thereby were collected in one or two tubes. The body fluid sample was suspended in 180 ul of lysozyme solution (20 mM Tris-Cl, pH 8.0, 2 mM EDTA, 1.2% Triton X-100, 20 mg/ml lysozyme). The resulting suspension was cultured for 37° C. for 30 minutes.

The culture was gently mixed with 20 ul of Proteinase K and 200 ul of AL solution (lysis solution, QIAamp DNA Blood Mini Kit, QIAGEN). The mixture was cultured at 55° C. for 2 hours and then at 95° C. for 10 minutes. The culture was mixed with 200 ul of 100% ethanol.

The resulting solution was loaded onto the QIAamp spin column sitting in a 2 ml tube and centrifuged at 8,000 rpm for 1 minute. The solution collected in the tube was discarded. 500 μl of AW1 solution (Wash Solution 1, QIAamp DNA Blood Mini Kit, Qiagen) was pipetted into the column which was then centrifuged at 8,000 rpm for 1 minute. The elute was discarded and 500 μl of AW2 solution (Wash Solution 2, QIAamp DNA Blood Mini Kit, Qiagen) was again pipetted into the column which was then centrifuged at 14,000 rpm for 1 minute. The elute was discarded and the QIAamp spin column was transferred to a 1.5 ml tube.

300 ul of AE solution (elution solution, DNA Blood Mini Kit, QIAGEN) was placed in the tube and stood at room temperature for 15 minutes, and then centrifuged at 8,000 rpm for 3 minutes. The eluted genomic DNAs were mixed with 750 ul of 100% ethanol and stood at −20° C. for 1 hour. The mixture was centrifuged at 14,000 rpm for 20 minutes. The ethanolic supernatant was discarded and the residue was dried. The pellet obtained thereby was dissolved in 20 ul of steriled distilled water and concentrated.

For blood sample, 10 ml of blood was placed in EDTA tube and centrifuged at 1,800 rpm at 4° C. for 10 minutes.

The plasma layer was transferred to a 1.5 ml tube and centrifuged at 14,000 rpm for 10 minutes. The resulting precipitate was transferred to a 1.5 ml. It was suspended in 180 ul of lysozyme solution (20 mM Tris-Cl, pH 8.0, 2 mM EDTA, 1.2% Triton X-100, 20 mg/ml lysozyme). The resulting suspension was cultured for 37° C. for 30 minutes.

The culture was gently mixed with 20 ul of Proteinase K and 200 ul of AL solution (lysis solution, QIAamp DNA Blood Mini Kit, QIAGEN). The mixture was cultured at 55° C. for 30 minutes and then at 95° C. for 10 minutes. The culture was mixed with 200 ul of 100% ethanol.

The resulting solution was loaded onto the QIAamp spin column sitting in a 2 ml tube and centrifuged at 8,000 rpm for 1 minute. The solution collected in the tube was discarded. 500 μl of AW1 solution (Wash Solution 1, QIAamp DNA Blood Mini Kit, Qiagen) was pipetted into the column which was then centrifuged at 8,000 rpm for 1 minute. The elute was discarded and 500 μl of AW2 solution (Wash Solution 2, QIAamp DNA Blood Mini Kit, Qiagen) was again pipetted into the column which was then centrifuged at 14,000 rpm for 1 minute. The elute was discarded and the QiAamp spin column was transferred to a 1.5 ml tube.

300 ul of AE solution (elution solution, DNA Blood Mini Kit, QIAGEN) was placed in the tube and stood at room temperature for 15 minutes, and then centrifuged at 8,000 rpm for 3 minutes. The eluted genomic DNAs were mixed with 750 ul of 100% ethanol and stood at −20 ° C. for 1 hour. The mixture was centrifuged at 14,000 rpm for 20 minutes. The ethanolic supernatant was discarded and the residue was dried. The pellet obtained thereby was dissolved in 20 ul of steriled distilled water and concentrated.

The procedures for amplification, hybridization, washing, and hybrid detection were performed in accordance with the same manners as described in the above Examples 5 through 7. The results are shown in Table 51 below, in which denominator is the number of sample application and numerator is the number of hybridization signal occurred.

TABLE 51

| | | cerebral | | | | |
|---|---|---|---|---|---|---|
| | | spinal | | | | |
| Probes | blood | fluid | abscess | saliva | feces | urine |
| Acti003 | 3/5 | 0/1 | 2/2 | 1/4 | | 0/1 |
| Acti23S01 | 1/5 | 1/1 | 2/2 | 1/ | | 1/1 |
| Acti23S02 | 1/5 | 0/1 | 1/2 | 0/4 | | 1/1 |
| Bur23 | | | | | | 1/1 |
| Bur01 | | | | | | 1/1 |
| Efacium002 | 2/2 | 0/1 | 0/1 | | 1/1 | 5/12 |
| Efacium003 | 2/2 | 0/1 | 1/1 | | 1/1 | 6/12 |
| Eco001 | 4/8 | 1/2 | 2/2 | 5/10 | 1/1 | 4/13 |
| Eco003 | 3/8 | 1/2 | 1/2 | 6/10 | | 3/13 |
| Kpneu002 | 2/2 | | 2/2 | 10/11 | 2/2 | 8/16 |
| Kpneu23 | 2/2 | | 2/2 | 9/11 | 2/2 | 9/16 |
| Ko001 | 1/4 | | | 1/4 | | 0/1 |
| Pa03 | 4/4 | 0/1 | 1/3 | 12/14 | | 4/4 |
| Paeru001 | 4/4 | 1/1 | 3/3 | 13/14 | | 4/4 |
| Pm | 0/1 | | 2/2 | 1/1 | | 20/20 |
| Pm002 | 0/1 | | 2/2 | 1/1 | | 20/20 |
| Pm003 | 2/2 | | 2/2 | 1/1 | | 20/20 |
| Pm004 | 2/2 | | 2/2 | 1/1 | | 20/20 |
| Saure03 | 10/11 | 0/1 | 4/8 | 3/10 | | 1/1 |
| Saure004 | 10/11 | 0/1 | 3/8 | 3/10 | | 1/1 |
| Saure005 | 10/11 | 1/1 | 4/8 | 3/10 | | 1/1 |
| SeM01 | 10/10 | | 3/4 | 2/2 | | 0/1 |
| SeM02 | 10/10 | | 3/4 | 2/2 | | 1/1 |
| StreppM | | | | 1/1 | | |
| Styp23 | | | | | 1/1 | 1/1 |

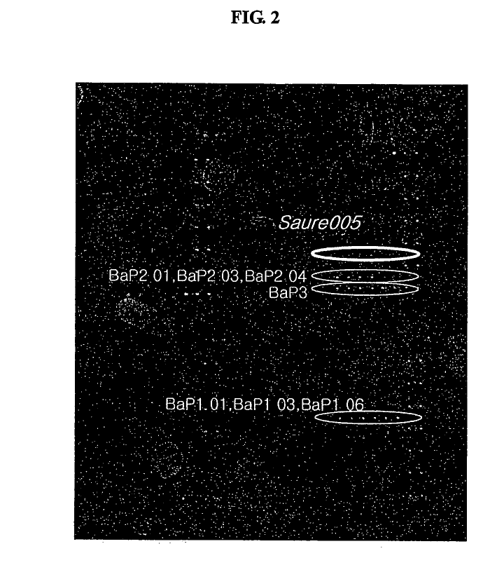

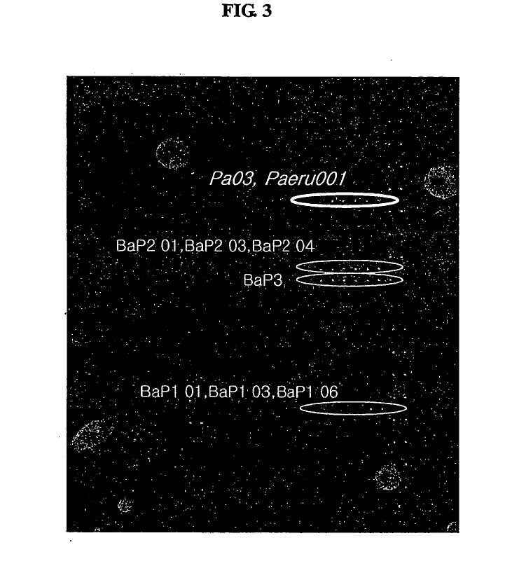

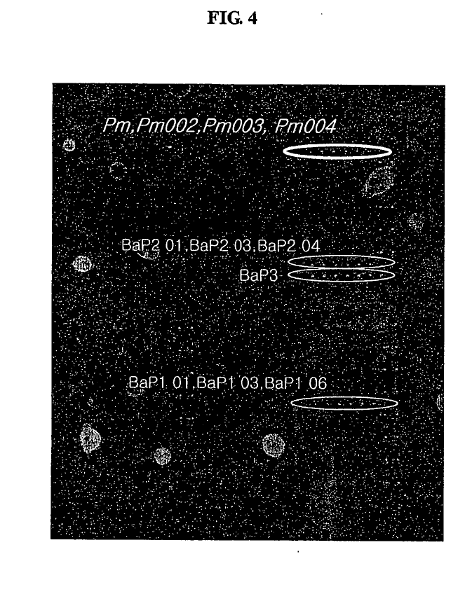

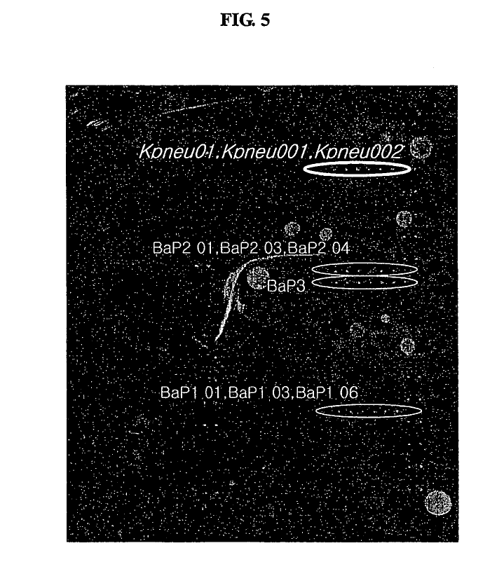

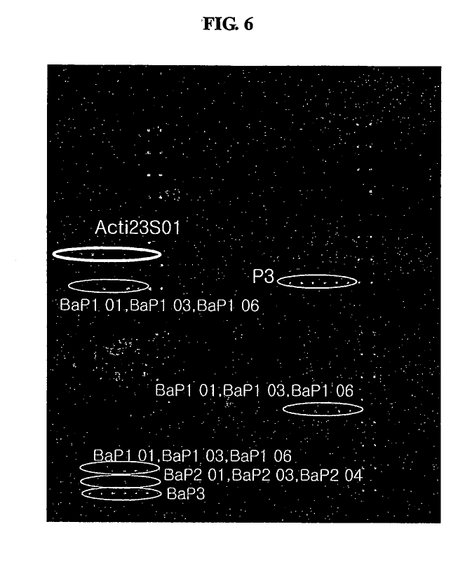

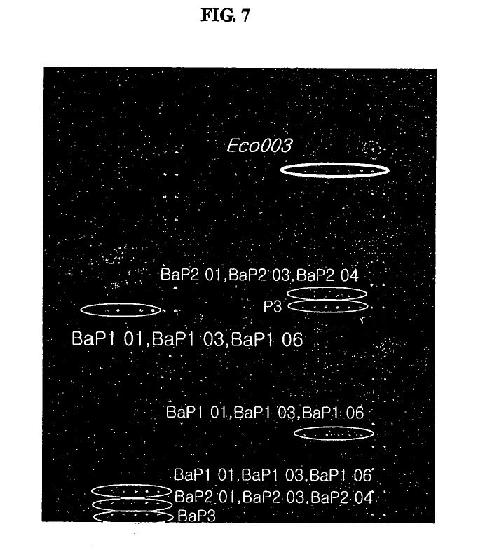

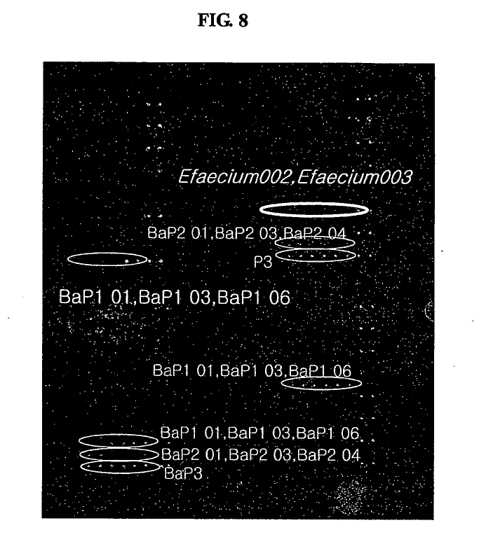

FIGS. 2 through 8 show the results of hybridization on the DNA chip of FIG. 1 in a blind sample including Staphylococcus aureus, Pseudomonas aeruginosa, Proteus mirabilis, Klebsiella pneumoniae, Acinetobacter baumanii, Escherichia coli or Enterococcus faecium, assayed using Scanarray 5000, respectively.

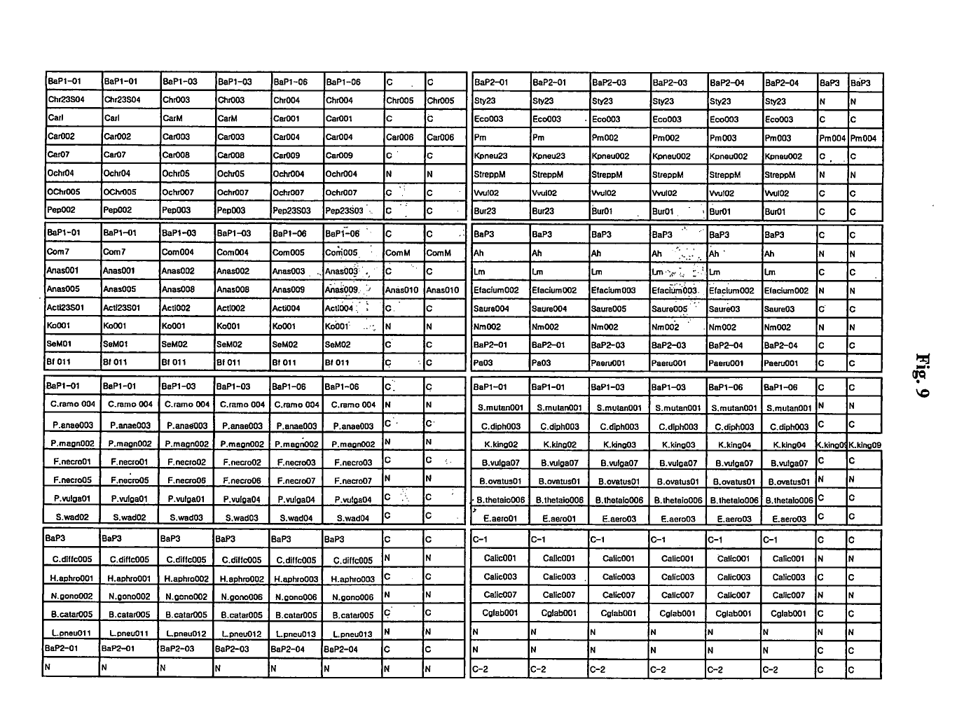

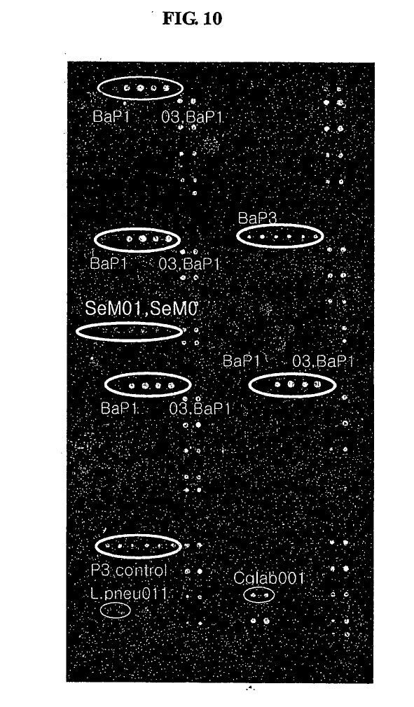

FIG. 10 shows the result of hybridization on the DNA chip of FIG. 9 in a blind sample including Staphylococcus epidermidis, assayed using Scanarray 5000.

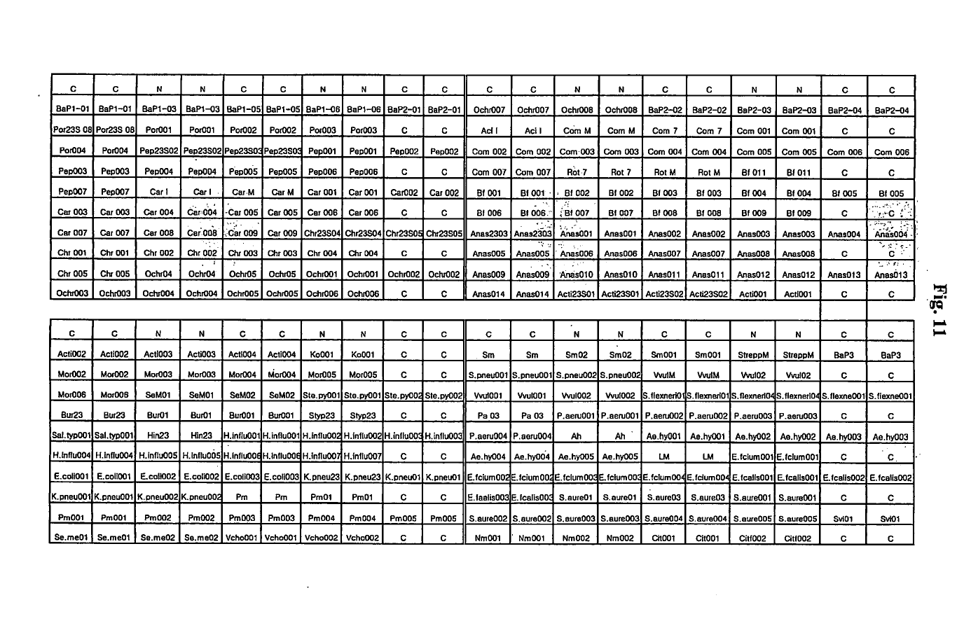

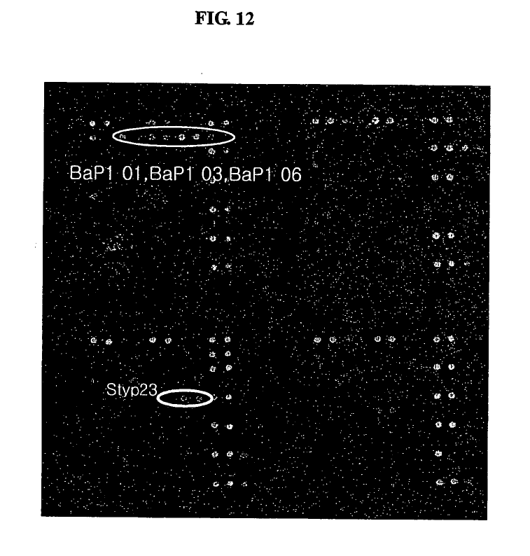

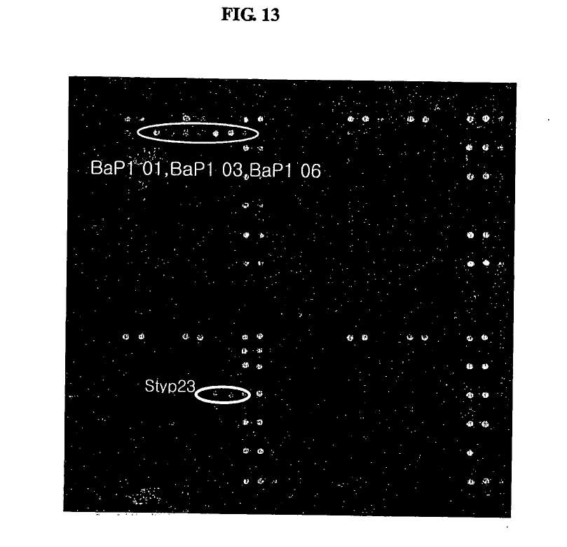

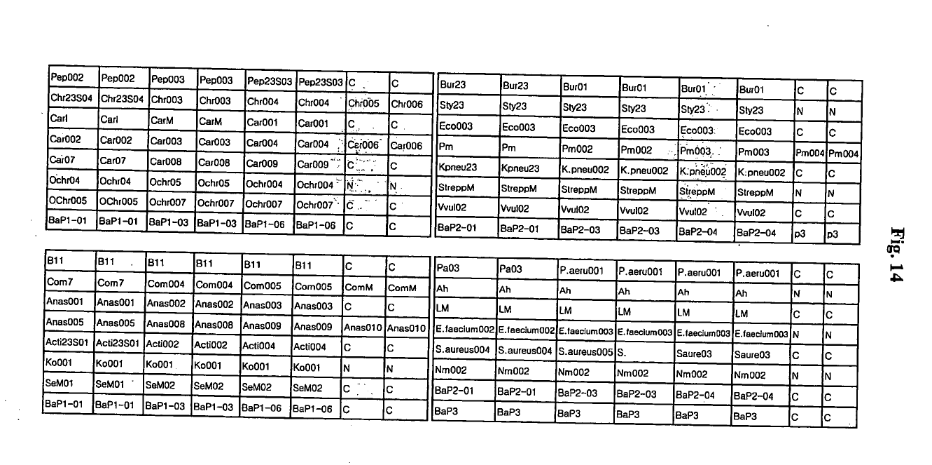

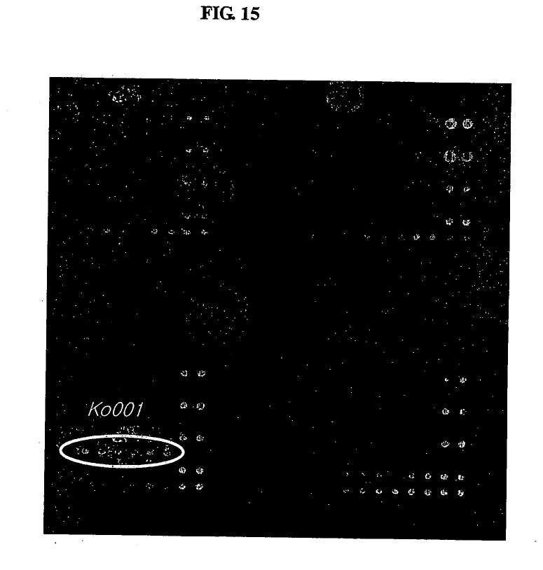

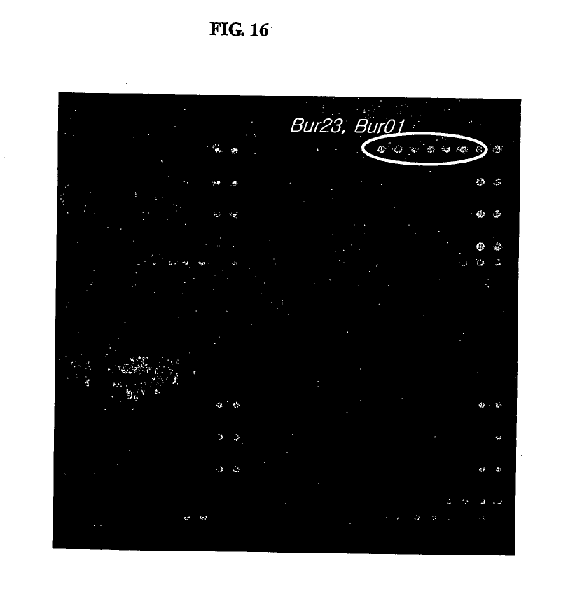

FIGS. 12 and 13 show the results of hybridization on the DNA chip of FIG. 11 in a blind sample including Salmonella Group E or Salmonella Group B, assayed using Scanarray 5000, respectively. FIGS. 15 and 16 show the results of hybridization on the DNA chip of FIG. 14 in a blind sample including Klebsiella oxytoca or Burkholderia cepacia, assayed using Scanarray 5000, respectively.

1. An isolated nucleic acid molecule having any one of nucleotide sequences shown in SEQ ID NO: 1 to SEQ ID NO: 28.

2. An isolated nucleic acid molecule comprising a nucleotide sequence selected from the group consisting of the following:
TGATGGAACTTTGCTT; (Acti004, SEQ ID NO: 29)
AGGGCACACATAATG; (Acti23S01, SEQ ID NO: 30)
ACGCTGTTGTTGGTG; (Acti23S02, SEQ ID NO: 31)
ATACACAGTACTTCG; (Acti3, SEQ ID NO: 32)
ATAGTGTTGCAAGGC; (Acti002, SEQ ID NO: 33)
and
TGAAAAGCCAGGGGA. (Acti003, SEQ ID NO: 34)

3. A nucleic acid probe for detecting Acinetobacter baumanii which comprise any one of nucleotide sequences shown in SEQ ID NO: 29 to SEQ ID NO: 34 according to claim 2.

4. A composition comprising at least one of nucleic acid probes for detecting Acinetobacter baumanii which comprises any one of nucleotide sequences shown in SEQ ID NO: 29 to SEQ ID NO: 34 according to claim 2.

5. A kit for detecting and identifying Acinetobacter baumanii in a biological sample which comprises (a) a composition comprising at least one of nucleic acid probes for detecting said bacteria which comprises any one of nucleotide sequences shown in SEQ ID NO: 29 to SEQ ID NO: 33 according to claim 2, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

6. A DNA chip in which at least one of nucleic acid molecules comprising any one of nucleotide sequences shown in SEQ ID NO: 29 to SEQ ID NO: 34 according to claim 2 is immobilized on a solid support.

7. A method for detection and identification of Acinetobacter baumanii in a biological sample which comprises the steps of (a) if appropriate, isolating and/or concentrating the polynucleic acids present in the sample, (b) if appropriate, amplifying the polynucleic acids with a pair of forward and reverse primers, (c) contacting the polynucleic acids of step (a) or (b) with a composition comprising at least one of nucleic acid probes for detecting said bacteria which comprises any one of nucleotide sequences shown in SEQ ID NO: 29 to SEQ ID NO: 34 according to claim 2 under the appropriate hybridization and wash conditions, (d) detecting the hybrids formed in step (c), and (e) identifying the bacteria present in the sample from the differential hybridization signals obtained in step (d).

8. An isolated nucleic acid molecule comprising a nucleotide sequence selected from the group consisting of the following:
TGACTCGTGCCCATG; (Anas001, SEQ ID NO: 45)
TACCGGGGTTAAAAG; (Anas002, SEQ ID NO: 46)
ATCAGTGATCTGAGA; (Anas003, SEQ ID NO: 47)
GAGACGAAGCACCAT; (Anas004, SEQ ID NO: 48)
AGTTGATACAGGTAG; (Anas011, SEQ ID NO: 49)
GGCCCCATCCGGGGT; (Anas013, SEQ ID NO: 50)
CAGTTGGAAGCAGAG; (Anas23S03, SEQ ID NO: 51)
GTTCTTGATTCATTG; (Anas005, SEQ ID NO: 52)
CAGCCCAAAAGTTGA; (Anas008, SEQ ID NO: 53)
AAACTGCAGGGCACA; (Anas009, SEQ ID NO: 54)
and
ATACTACCTGACGAC. (Anas010, SEQ ID NO: 55)

9. A nucleic acid probe for detecting Anaerobiospirillum succiniciproducens which comprise any one of nucleotide sequences shown in SEQ ID NO: 45 to SEQ ID NO: 55 according to claim 8.

10. A composition comprising at least one of nucleic acid probes for detecting Anaerobiospirillum succiniciproducens which comprises any one of nucleotide sequences shown in SEQ ID NO: 45 to SEQ ID NO: 51 according to claim 8.

11. A kit for detecting and identifying Anaerobiospirillum succiniciproducens in a biological sample which comprises (a) a composition comprising at least one of nucleic acid probes for detecting said bacteria which comprises any one of nucleotide sequences shown in SEQ ID NO: 45 to SEQ ID NO: 51 according to claim 8, (b) optionally a pair of forward and reverse primers used for the amplification of polynucleic acids in said biological sample, (c) a buffer enabling hybridization reaction between the probes contained in said composition and the polynucleic acids present in said biological sample or amplified products therefrom or components necessary for producing the buffer, (d) a solution for washing hybrids formed under the appropriate wash conditions or components necessary for producing the solution, and (e) optionally a means for detection of said hybrids.

12. A DNA chip in which at least one of nucleic acid molecules comprising any one of nucleotide sequences shown in SEQ ID NO: 45 to SEQ ID NO: 51 according to claim 8 is immobilized on a solid support.

13-281. (canceled)
